(19) United States
(12) Patent Application Publication
(10) Pub. No.: US 2026/0297209 A1
(43) Pub. Date: Oct. 1, 2026

(54) CD137 BINDING MOLECULES AND USES THEREOF

(71) Applicant: MACROGENICS, INC., Rockville, MD (US)

(72) Inventors: Alexey Yevgenyevich BEREZHNOY, Rockville, MD (US); Gundo DIEDRICH, Rockville, MD (US); Paul A. MOORE, Rockville, MD (US); Ezio BONVINI, Rockville, MD (US); Kalpana SHAH, Rockville, MD (US)

(21) Appl. No.: 19/320,320

(22) Filed: Sep. 5, 2025

Related U.S. Application Data
(63) Continuation of application No. 17/798,285, filed on Feb. 16, 2021, now Pat. No. 12,435,155.
(60) Provisional application No. 62/980,000, filed on Feb. 21, 2020.
(60) Provisional application No. 63/104,685, filed on Oct. 23, 2020.
(60) Provisional application No. 63/147,565, filed on Feb. 9, 2021.

(51) Int. Cl. C07K 16/28; A61K 39/00; A61P 35/00

(52) U.S. Cl. CPC C07K 16/2896; A61P 35/00; C07K 16/2827; A61K 2039/505; C07K 2317/24; C07K 2317/31; C07K 2317/35; C07K 2317/524; C07K 2317/565; C07K 2317/64

(57) ABSTRACT

The present invention is directed to binding molecules that possess one or more epitope-binding sites specific for an epitope of CD137, including antibodies, and molecules comprising epitope-binding fragments thereof. The invention is further directed to multispecific binding molecules comprising one or more epitope-binding sites specific for an epitope of CD137 and one or more epitope-binding sites specific for an epitope of a tumor antigen (“TA”) (e.g., a “CD137×TA Binding Molecule”). In one embodiment, such CD137×TA Binding Molecules will be bispecific molecules, especially bispecific tetravalent diabodies possessing two epitope-binding sites each specific for an epitope of CD137 and two epitope-binding sites each specific for an epitope of a TA. Alternatively, such CD137×TA Binding Molecules will be bispecific molecules possessing two epitope-binding sites each specific for an epitope of CD137 and one epitope-binding sites each specific for an epitope of a TA. The invention also provides novel Binding Molecules, as well as derivatives thereof and uses thereof. The invention is directed to pharmaceutical compositions that contain such CD137 molecules. The invention is additionally directed to methods for the use of such molecules in the treatment of cancer and other diseases and conditions.

### CROSS-REFERENCE TO RELATED APPLICATIONS

This patent application is a continuation of U.S. patent application Ser. No. 17/798,285, filed Aug. 8, 2022, which is a national stage of International Patent Application No. PCT/US2021/018177, filed Feb. 16, 2021, which claims the benefit of U.S. Provisional Application Nos. 62/980,000, filed Feb. 21, 2020, 63/104,685, filed Oct. 23, 2020, and 63/147,565, filed Feb. 9, 2021, each of which is incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML copy, created on Sep. 3, 2025, is named 0260-0007_SL_ST26 as filed 2025 Sep. 5.xml and is 228,370 bytes in size.

### FIELD

The present technology is directed to CD137 Binding Molecules, such as monospecific antibodies, and molecules comprising epitope-binding fragments thereof, that are capable of binding to an epitope of CD137. The technology is further directed to multispecific CD137 Binding Molecules (e.g., bispecific antibodies, bispecific diabodies, BiTEs, trivalent binding molecules, etc.) that are capable of binding to both an epitope of CD137 and to an epitope of a second antigen, particularly a tumor antigen (“TA”) (e.g., a “CD137×TA Binding Molecule”). The technology also provides novel PD-L1 Binding Molecules, such as monospecific antibodies, and molecules comprising epitope-binding fragments thereof, that are capable of binding to an epitope of PD-L1, as well as derivatives thereof and uses thereof. The present technology is also directed to pharmaceutical compositions that comprise such molecules. The technology also includes the use of such molecules in the treatment of disease, especially cancer or a disease or condition associated with or characterized by the presence of a suppressed immune system.

### BACKGROUND

CD137 (also known as 4-1BB and “TNF receptor superfamily member 9” (“TNFRSF9”)) is a costimulatory receptor member of the tumor necrosis factor receptor superfamily, mediating CD28-dependent and independent T-cell costimulation (Vinay, D. S. and Kwon, B. S. (1998) “Role of 4-IBB in immune responses,” Semin Immunol. 10:481-489; Croft, M. (2009) “The Role Of TNF Superfamily Members In T-Cell Function And Diseases,” Nat. Rev. Immunol. 9:271-285). CD137 is inducibly expressed by T cells, natural killer (NK) cells, dendritic cells (DC), B cells, and other cells of the immune system. Ligation of CD137 by its ligand CD137L (4-1BBL; TNFSF9), or agonist antibodies evokes various T cell responses such as cell expansion, increased cytokine secretion and the prevention of activation-induced cell death. Thus, antibodies stimulating CD137 can induce survival and proliferation of T cells, thereby enhancing the anti-tumor immune response. Such recognitions have led to the proposal that antibodies that are immunospecific for CD137 could be used to activate the immune system and thereby provide a therapy for cancer (Li, S. Y. et al. (2013) “Immunotherapy Of Melanoma With The Immunecostimulatory Monoclonal Antibodies Targeting CD137,” Clin. Pharmacol. 5:47-53; Bartkowiak, T. et al. (2015) “4-IBB Agonists: Multi-Potent Potentiators Of Tumor Immunity,” Frontiers Oncol. 5:117). The anti-CD137 antibodies utomilumab and urelumab have been described but their clinical development was hampered by low efficacy (utomilumab) or severe liver toxicity (urelumab).

### SUMMARY

Provided are improved compositions capable of more vigorously stimulating and directing the body's immune system to attack cancer cells while avoiding toxicities associated with antibodies that exhibit high activity in the absence of cross-linking. For although the adaptive immune system can be a potent defense mechanism against cancer and disease, it is often hampered by immune suppressive/evasion mechanisms in the tumor microenvironment, mediated by the reduced/absent co-stimulatory activity of CD137. Furthermore, co-inhibitory molecules expressed by tumor cells, immune cells, and stromal cells in the tumor milieu can dominantly attenuate T-cell responses against cancer cells.

Provided are CD137 Binding Molecules, particularly CD137×TA Binding Molecules that are capable of binding to both an epitope of CD137 and to an epitope of a tumor antigen. Such bispecific molecules are capable of binding to tumor antigens that are expressed on the surfaces of tumor cells, and of co-localizing CD137-expressing immune cells to such tumor cells. Such co-localization upregulates the immune cells so as to promote the activation or continued activation of the immune system (e.g., stimulating a cytotoxic T cell response, against tumor cells). These attributes permit such bispecific molecules to have utility in stimulating the immune system and particularly in the treatment of cancer. The present technology is directed to these and other goals.

Thus, provided in certain aspects are CD137 Binding Molecules, such as monospecific antibodies, and molecules comprising epitope-binding fragments thereof, that are capable of binding to an epitope of CD137. The invention is further directed to multispecific CD137 Binding Molecules (e.g., bispecific antibodies, bispecific diabodies, BiTEs, trivalent binding molecules, etc.) that are capable of binding to both an epitope of CD137 and to an epitope of a second antigen, particularly a tumor antigen (“TA”) (e.g., a “CD137×TA Binding Molecule”). The invention also provides novel PD-L1 Binding Molecules, such as monospecific antibodies, and molecules comprising epitope-binding fragments thereof, that are capable of binding to an epitope of PD-L1, as well as derivatives thereof and uses thereof. The present invention is also directed to pharmaceutical compositions that comprise such molecules. The invention also includes the use of such molecules in the treatment of disease, especially cancer or a disease or condition associated with or characterized by the presence of a suppressed immune system.

The present invention provides novel CD137 Binding Molecules that exhibit desirable characteristics particularly when incorporated into multispecific molecules. The present invention is also directed to multispecific CD137×TA Binding Molecules that are composed of polypeptide chains that associate with one another to form two binding sites each specific for an epitope of CD137 and two binding sites each specific for an epitope of a TA. Such CD137×TA Binding Molecules of the invention are termed “bispecific tetravalent.” The present invention is also directed to CD137×TA Binding Molecules that are composed of polypeptide chains that associate with one another to form two binding sites each specific for an epitope of CD137 and one binding site specific for an epitope of a TA. Such CD137×TA Binding Molecules of the invention are termed “bispecific trivalent.” The binding molecules of the invention (e.g., CD137 Binding Molecules) sometimes include a first binding site without including a second binding site that immunospecifically binds to an antigen different than the antigen to which the first binding site binds. The binding molecules of the invention therefore sometimes include only a first binding site, and a first Light Chain Variable Domain and a first Heavy Chain Variable Domain, and not a second binding site, second Light Chain Variable Domain or second Heavy Chain Variable Domain that bind to a different antigen than the first binding site, and non-limiting examples of such binding molecules include scFv, antibody and Fab binding molecules.

The present invention provides CD137×TA Binding Molecules that comprise four polypeptide chains (a “first,” “second,” “third,” and “fourth” polypeptide chain), wherein the first and second polypeptide chains are covalently bonded to one another, the third and fourth polypeptide chains are covalent bonded to one another, and the first and third polypeptide chains are covalently bonded to one another. Also provided are CD137×TA Binding Molecules of the invention comprising five polypeptide chains (a “first,” “second,” “third,” “fourth,” and “fifth” polypeptide chain), wherein the first and second polypeptide chains are covalently bonded to one another, the third and fourth polypeptide chain are covalent bonded to one another, the third and fifth polypeptide chains are covalent bonded to one another, and the first and third polypeptide chains are covalently bonded to one another.

In detail, the invention provides a CD137 Binding Molecule comprising a first binding site that immunospecifically binds to an epitope of CD137, wherein the first binding site comprises a first Light Chain Variable Domain that comprises a CDR_{L}1, CDR_{L}2 and CDR_{L}3, and a first Heavy Chain Variable Domain that comprises a CDR_{H}1, CDR_{H}2 and CDR_{H}3; and wherein,
(A) the first Light Chain Variable Domain CDR_{L}1, CDR_{L}2, and CDR_{L}3 are the Light Chain CDRs of CD137 MAB-6 VL1 (SEQ ID NO:50); and
(B) the first Heavy Chain Variable Domain CDR_{H}1, CDR_{H}2, and CDR_{H}3 are the Heavy Chain CDRs of CD137 MAB-6 VH1 (SEQ ID NO:46).

The invention further concerns the embodiment of such a CD137 Binding Molecule, wherein the first Heavy Chain Variable Domain comprises the amino acid sequence of: hCD137 MAB-6 VH1 (SEQ ID NO:46).

The invention further concerns the embodiments of such a CD137 Binding Molecule, wherein the first Light Chain Variable Domain comprises the amino acid sequence of:
(A) hCD137 MAB-6 VLx (SEQ ID NO:54);
(B) hCD137 MAB-6 VL1 (SEQ ID NO:50);
(B) hCD137 MAB-6 VL2 (SEQ ID NO:55); or
(C) hCD137 MAB-6 VL3 (SEQ ID NO:56).

The invention further concerns the embodiment of such a CD137 Binding Molecule, wherein:
(A) the first Heavy Chain Variable Domain comprises the amino acid sequence of: hCD137 MAB-6 VH1 (SEQ ID NO:46); and
(B) the first Light Chain Variable Domain comprises the amino acid sequence of: hCD137 MAB-6 VL3 (SEQ ID NO:56).

The invention further concerns all the above embodiments of such CD137 Binding Molecules, wherein the molecule is a bispecific molecule comprising a second binding site that immunospecifically binds a TA, and wherein the second binding site comprises a second Light Chain Variable Domain that comprises a CDR_{L}1, CDR_{L}2 and CDR_{L}3, and a second Heavy Chain Variable Domain that comprises a CDR_{H}1, CDR_{H}2 and CDR_{H}3.

The invention further concerns the embodiment of such a CD137 Binding Molecule, wherein the TA is selected from the tumor antigens presented in Tables 1-2.

The invention further concerns the embodiment of such a CD137 Binding Molecule, wherein the TA is PD-L1 and wherein:
(A) the second Light Chain Variable Domain CDR_{L}1, CDR_{L}2, and CDR_{L}3 are the Light Chain CDRs of hPD-L1 MAB-2 VLx (SEQ ID NO:63); and
(B) the second Heavy Chain Variable Domain CDR_{H}1, CDR_{H}2, and CDR_{H}3 are the Heavy Chain CDRs of hPD-L1 MAB-2 VHx (SEQ ID NO:59).

The invention further concerns the embodiment of such a CD137 Binding Molecule, wherein:
(A) (1) the second Light Chain Variable Domain CDR_{L}1, CDR_{L}2, and CDR_{L}3 are the Light Chain CDRs of hPD-L1 MAB-2 VL1 (SEQ ID NO:58);
or
(2) the second Light Chain Variable Domain CDR_{L}1, CDR_{L}2, and CDR_{L}3 are the Light Chain CDRs of hPD-L1 MAB-2 VL2 (SEQ ID NO:72);
and
(B) (1) the second Heavy Chain Variable Domain CDR_{H}1, CDR_{H}2, and CDR_{H}3 are the Heavy Chain CDRs of hPD-L1 MAB-2 VH1 (SEQ ID NO:57);
(2) the second Heavy Chain Variable Domain CDR_{H}1, CDR_{H}2, and CDR_{H}3 are the Heavy Chain CDRs of hPD-L1 MAB-2 VH2 (SEQ ID NO:67);
(3) the second Heavy Chain Variable Domain CDR_{H}1, CDR_{H}2, and CDR_{H}3 are the Heavy Chain CDRs of hPD-L1 MAB-2 VH3 (SEQ ID NO:68);
(4) the second Heavy Chain Variable Domain CDR_{H}1, CDR_{H}2, and CDR_{H}3 are the Heavy Chain CDRs of hPD-L1 MAB-2 VH2 (SEQ ID NO:69);
(5) the second Heavy Chain Variable Domain CDR_{H}1, CDR_{H}2, and CDR_{H}3 are the Heavy Chain CDRs of hPD-L1 MAB-2 VH2 (SEQ ID NO:70);
or
(6) the second Heavy Chain Variable Domain CDR_{H}1, CDR_{H}2, and CDR_{H}3 are the Heavy Chain CDRs of hPD-L1 MAB-2 VH2 (SEQ ID NO:71).

The invention further concerns the embodiment of such a CD137 Binding Molecule, wherein the second Heavy Chain Variable Domain comprises the amino acid sequence of:
(A) hPD-L1 MAB-2 VH1 (SEQ ID NO:57);
(B) hPD-L1 MAB-2 VH2 (SEQ ID NO:67);
(C) hPD-L1 MAB-2 VH3 (SEQ ID NO:68);
(D) hPD-L1 MAB-2 VH4 (SEQ ID NO:69);
(E) hPD-L1 MAB-2 VH5 (SEQ ID NO:70); or
(F) hPD-L1 MAB-2 VH6 (SEQ ID NO:71).

The invention further concerns the embodiments of such CD137 Binding Molecules, wherein the second Light Chain Variable Domain comprises the amino acid sequence of:
(A) hPD-L1 MAB-2 VL1 (SEQ ID NO:58); or
(B) hPD-L1 MAB-2 VL2 (SEQ ID NO:72).

The invention further concerns the embodiment of such a CD137 Binding Molecule, wherein the TA is 5T4 and wherein:
(A) (1) the second Light Chain Variable Domain CDR_{L}1, CDR_{L}2, and CDR_{L}3 are the Light Chain CDRs of 5T4 MAB-1 VL (SEQ ID NO:93); and
(2) the second Heavy Chain Variable Domain CDR_{H}1, CDR_{H}2, and CDR_{H}3 are the Heavy Chain CDRs of 5T4 MAB-1 VH (SEQ ID NO:92); or
(B) (1) the second Light Chain Variable Domain CDR_{L}1, CDR_{L}2, and CDR_{L}3 are the Light Chain CDRs of 5T4 MAB-2 VL (SEQ ID NO:95); and
(2) the second Heavy Chain Variable Domain CDR_{H}1, CDR_{H}2, and CDR_{H}3 are the Heavy Chain CDRs of 5T4 MAB-2 VH (SEQ ID NO:96).

The invention further concerns the embodiment of such a CD137 Binding Molecule, wherein the second Heavy Chain Variable Domain comprises the amino acid sequence of: 5T4 MAB-1 VH (SEQ ID NO:92).

The invention further concerns the embodiments of such CD137 Binding Molecules, wherein the second Light Chain Variable Domain comprises the amino acid sequence of: 5T4 MAB-1 VL (SEQ ID NO:93).

The invention further concerns such a CD137 Binding Molecule, wherein the TA is HER2 and wherein:
(A) the second Light Chain Variable Domain CDR_{L}1, CDR_{L}2, and CDR_{L}3 are the Light Chain CDRs of hHER2-MAB-1 VLx (SEQ ID NO:79); and
(B) the second Heavy Chain Variable Domain CDR_{H}1, CDR_{H}2, and CDR_{H}3 are the Heavy Chain CDRs of hHER2-MAB-1 VHx (SEQ ID NO:78);

The invention further concerns the embodiment of such a CD137 Binding Molecule, wherein:
(A) (1) the second Light Chain Variable Domain CDR_{L}1, CDR_{L}2, and CDR_{L}3 are the Light Chain CDRs of hHER2-MAB-1 VL1 (SEQ ID NO:83);
   (2) the second Light Chain Variable Domain CDR_{L}1, CDR_{L}2, and CDR_{L}3 are the Light Chain CDRs of hHER2-MAB-1 VL2 (SEQ ID NO:84); or
   (3) the second Light Chain Variable Domain CDR_{L}1, CDR_{L}2, and CDR_{L}3 are the Light Chain CDRs of hHER2-MAB-1 VL3 (SEQ ID NO:85); and
(B) (1) the second Heavy Chain Variable Domain CDR_{H}1, CDR_{H}2, and CDR_{H}3 are the Heavy Chain CDRs of hHER2-MAB-1 VH1 (SEQ ID NO:80);
   (2) the second Heavy Chain Variable Domain CDR_{H}1, CDR_{H}2, and CDR_{H}3 are the Heavy Chain CDRs of hHER2-MAB-1 VH2 (SEQ ID NO:81); or
   (3) the second Heavy Chain Variable Domain CDR_{H}1, CDR_{H}2, and CDR_{H}3 are the Heavy Chain CDRs of hHER2-MAB-1 VH3 (SEQ ID NO:82).

The invention further concerns the embodiment of such a CD137 Binding Molecule, wherein the second Heavy Chain Variable Domain comprises the amino acid sequence of:
(A) hHER2-MAB-1 VHx (SEQ ID NO:78);
(B) hHER2-MAB-1 VH1 (SEQ ID NO:80);
(C) hHER2-MAB-1 VH2 (SEQ ID NO:81); or
(D) hHER2-MAB-1 VH3 (SEQ ID NO:82).

The invention further concerns the embodiments of such CD137 Binding Molecules, wherein the second Light Chain Variable Domain comprises the amino acid sequence of:
(A) hHER2-MAB-1 VLx (SEQ ID NO:79);
(B) hHER2-MAB-1 VL1 (SEQ ID NO:83);
(C) hHER2-MAB-1 VL2 (SEQ ID NO:84); or
(D) hHER2-MAB-1 VL3 (SEQ ID NO:85).

The invention further concerns all the above embodiments of such CD137 Binding Molecules, wherein the molecule is an antibody, a bispecific tetravalent Fc-bearing diabody, or a bispecific trivalent molecule.

The invention further concerns the embodiment of such a CD137 Binding Molecule, wherein the molecule is bispecific and tetravalent, and comprises a first, a second, a third, a fourth, and optionally a fifth polypeptide chain, wherein the polypeptide chains form a covalently bonded complex.

The invention further concerns the embodiment of such a CD137 Binding Molecule, wherein the molecule is bispecific and trivalent, and comprises a first, a second, a third, and a fourth, polypeptide chain, wherein the polypeptide chains form a covalently bonded complex.

The invention additionally concerns the embodiment of all such CD137 Binding Molecules wherein the molecule comprises an Fc Region of the IgG1, IgG2, IgG3, or IgG4 isotype and optionally wherein the molecule further comprises a Hinge Domain.

The invention additionally concerns the embodiment of all such CD137 Binding Molecules wherein the Fc Region is a variant Fc Region that comprises one or more amino acid modifications that reduces the affinity of the variant Fc Region for an FcTR and/or enhances the serum half-life, and more particularly, wherein the modifications comprise at least one amino acid substitution selected from the group consisting of:
(A) L234A; L235A;
(B) L234A and L235A;
(C) M252Y; M252Y and S254T;
(D) M252Y and T256E;
(E) M252Y, S254T and T256E; or
(F) K288D and H435K;
wherein the numbering is that of the EU index as in Kabat.

The invention further concerns the embodiment of such a CD137 Binding Molecule, wherein the TA is PD-L1 and wherein:
(A) the first and third polypeptide chains comprise the amino acid sequence of SEQ ID NO:116, SEQ ID NO:118, SEQ ID NO:120; and
(B) the second and fourth polypeptide chains comprise the amino acid sequence of SEQ ID NO:117, SEQ ID NO:119, SEQ ID NO:121, SEQ ID NO:122, SEQ ID NO:123, SEQ ID NO:124, SEQ ID NO:125, SEQ ID NO:126, or SEQ ID NO:139.

The invention further concerns the embodiment of such a CD137 Binding Molecule, wherein the molecule comprises:
(A) SEQ ID NO:116 and SEQ ID NO:117;
(B) SEQ ID NO:118 and SEQ ID NO:119;
(C) SEQ ID NO:120 and SEQ ID NO:119;
(D) SEQ ID NO:118 and SEQ ID NO:121;
(E) SEQ ID NO:120 and SEQ ID NO:121;
(F) SEQ ID NO:120 and SEQ ID NO:122;
(G) SEQ ID NO:120 and SEQ ID NO:123;
(H) SEQ ID NO:120 and SEQ ID NO:124;
(I) SEQ ID NO:120 and SEQ ID NO:125;
(J) SEQ ID NO:120 and SEQ ID NO:126; or
(K) SEQ ID NO:120 and SEQ ID NO:139.

The invention further concerns the embodiment of such a CD137 Binding Molecule, wherein the TA is PD-L1 and wherein:
(A) the first polypeptide chain comprises the amino acid sequence of SEQ ID NO:127, SEQ ID NO:133, or SEQ ID NO:135;
(B) the second polypeptide chain comprises the amino acid sequence of SEQ ID NO:128, SEQ ID NO:134, or SEQ ID NO:136;
(C) the third polypeptide chain comprises the amino acid sequence of SEQ ID NO:129, or SEQ ID NO:131; and
(D) the fourth polypeptide chain comprises the amino acid sequence of SEQ ID NO:130, SEQ ID NO:132.

The invention further concerns the embodiment of such a CD137 Binding Molecule, wherein the molecule comprises:
(A) SEQ ID NO:127, SEQ ID NO:128, SEQ ID NO:129, and SEQ ID NO:130;
(B) SEQ ID NO:127, SEQ ID NO:128, SEQ ID NO:131, and SEQ ID NO:132;
(C) SEQ ID NO:133, SEQ ID NO:134, SEQ ID NO:131, and SEQ ID NO:132; or
(D) SEQ ID NO:135, SEQ ID NO:136, SEQ ID NO:131, and SEQ ID NO:132.

The invention additionally concerns a pharmaceutical composition comprising any of the above-described CD137 Binding Molecules, and a physiologically acceptable carrier.

The invention additionally concerns the use of such CD137 Binding Molecules, or such a pharmaceutical composition, in the treatment of cancer characterized by the expression of the TA.

The invention additionally concerns a PD-L1 Binding Molecule that comprises a Light Chain Variable Domain that comprises a CDR_{L}1, CDR_{L}2 and CDR_{L}3, and a Heavy Chain Variable Domain that comprises a CDR_{H}1, CDR_{H}2 and CDR_{H}3; wherein:
(A) the Light Chain Variable Domain CDR_{L}1, CDR_{L}2, and CDR_{L}3 are the Light Chain CDRs of hPD-L1 MAB-2 VL2 (SEQ ID NO:72);
and
(B) (1) the Heavy Chain Variable Domain CDR_{H}1, CDR_{H}2, and CDR_{H}3 are the Heavy Chain CDRs of hPD-L1 MAB-2 VH2 (SEQ ID NO:67);
   (2) the Heavy Chain Variable Domain CDR_{H}1, CDR_{H}2, and CDR_{H}3 are the Heavy Chain CDRs of hPD-L1 MAB-2 VH3 (SEQ ID NO:68)
   (3) the second Heavy Chain Variable Domain CDR_{H}1, CDR_{H}2, and CDR_{H}3 are the Heavy Chain CDRs of hPD-L1 MAB-2 VH4 (SEQ ID NO:69)
(4) the second Heavy Chain Variable Domain CDR_{H}1, CDR_{H}2, and CDR_{H}3 are the Heavy Chain CDRs of hPD-L1 MAB-2 VH5 (SEQ ID NO:70); or
(5) the second Heavy Chain Variable Domain CDR_{H}1, CDR_{H}2, and CDR_{H}3 are the Heavy Chain CDRs of hPD-L1 MAB-2 VH6 (SEQ ID NO:71).

The invention further concerns the embodiment of such a PD-L1 Binding Molecule, wherein the Heavy Chain Variable Domain comprises the amino acid sequence of:
(A) hPD-L1 MAB-2 VH2 (SEQ ID NO:67);
(B) hPD-L1 MAB-2 VH3 (SEQ ID NO:68);
(C) hPD-L1 MAB-2 VH4 (SEQ ID NO:69);
(D) hPD-L1 MAB-2 VH5 (SEQ ID NO:70); or
(E) hPD-L1 MAB-2 VH6 (SEQ ID NO:71).

The invention further concerns the embodiment of such a PD-L1 Binding Molecule, wherein the Light Chain Variable Domain comprises the amino acid sequence of hPD-L1 MAB-2 VL2 (SEQ ID NO:72).

The invention further concerns the embodiment of such PD-L1 Binding Molecules, wherein the molecule is an antibody or an antigen binding fragment thereof.

The invention additionally concerns a pharmaceutical compositions comprising any of the above-described PD-L1 Binding Molecules, and a physiologically acceptable carrier.

The invention additionally concerns the use of such PD-L1 Binding Molecules, or such pharmaceutical compositions, in the treatment of a disease or condition associated with a suppressed immune system or characterized by the expression of PD-L1.

The invention further concerns such a use, wherein the condition associated with a suppressed immune system or characterized by the expression of PD-L1 is cancer.

The invention additionally concerns the embodiment of all such uses, wherein the cancer is selected from the group consisting: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer.

The invention further concerns a method of enhancing the activity of a tumor targeting agent comprising administering the tumor target agent in combination with any of the above-described CD137 Binding Molecules, any of the above-described PD-L1 Binding Molecules, or any of the above-described pharmaceutical compositions.

The invention additionally concerns a method of treating a disease or condition associated with a suppressed immune system or characterized by the expression of a TA comprising administering to a subject in need thereof and of the above-described CD137 Binding Molecules, any of the above-described PD-L1 Binding Molecules, or any of the above-described pharmaceutical compositions.

The invention further concerns such a method, further comprising administering a tumor targeting agent.

The invention further concerns such a method, wherein the condition associated with a suppressed immune system or characterized by the expression of the tumor TA is cancer.

The invention further concerns all the above embodiments of such a method, wherein the tumor target agent is an antibody, an epitope binding fragment of an antibody, or an agent that mediates T-cell redirected killing of a target cell.

The invention additionally concerns the embodiment such methods, wherein the cancer is selected from the group consisting: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer.

The invention additionally concerns a nucleic acid encoding the CD137 Binding Molecule of any of the above embodiments, or the PD-L1 Binding Molecule of any of the above embodiments.

The invention further concerns an expression vector comprising such nucleic acid.

The invention additionally concerns a cell comprising a nucleic acid according to any of the above embodiments or an expression vector of any of the above embodiments.

The invention further concerns a such cell, wherein said cell is a mammalian cell.

### BRIEF DESCRIPTION OF THE DRAWINGS

FIG. 1A-1D provides schematics showing representative covalently bonded diabodies comprising Fc Regions. FIGS. 1A-1D show tetravalent diabodies having four epitope-binding sites composed of two pairs of polypeptide chains, (i.e., four polypeptide chains in all). One polypeptide of each pair possesses a CH2 and CH3 Domain, such that the associated chains form all or part of an Fc Region. VL and VH Domains that recognize the same epitope are shown using the same shading or fill pattern. The two pairs of polypeptide chains may be same. In such embodiments wherein the VL and VH Domains recognize different epitopes (as shown in FIGS. 1A-1B), the resulting molecule possesses four epitope-binding sites and is bispecific and bivalent with respect to each bound epitope. In such embodiments wherein the VL and VH Domains recognize the same epitope (e.g., the same VL Domain CDRs and the same VH Domain CDRs are used on both chains), the resulting molecule possesses four epitope-binding sites and is monospecific and tetravalent with respect to a single epitope. Alternatively, the two pairs of polypeptides may be different. In such embodiments wherein the VL and VH Domains of each pair of polypeptides recognize different epitopes (as shown in FIG. 1C), the resulting molecule possesses four epitope-binding sites and is tetraspecific and monovalent with respect to each bound epitope. FIG. 1A shows an Fc diabody which contains a peptide Heterodimer-Promoting Domain comprising a cysteine residue. FIG. 1B shows an Fc diabody composed of two pairs of polypeptide chains each having an E-coil or K-coil Heterodimer-Promoting Domain (i.e., four polypeptide chains in all). The wavy line (VWWV) in this and all of the Figures providing schematic presentations of binding molecule domains represents one or more optional Heterodimer-Promoting Domains, that is/are present. As shown, a cysteine residue may be present in a linker (main diagram) and/or in the Heterodimer-Promoting Domain (boxed). One polypeptide chain of each pair possesses a linker comprising a cysteine (which linker may comprise all or a portion of a hinge region) and a CH2 and CH3 Domain, such that the associated chains form all or part of an Fc Region. FIG. 1C, shows an Fc-Region-Containing diabody, which contains antibody CH1 and CL domains. FIG. 1D, shows a representative covalently bonded diabody molecule having two epitope-binding sites composed of three polypeptide chains. Two of the polypeptide chains possess a CH2 and CH3 Domain, such that the associated chains form all or part of an Fc Region. The polypeptide chains comprising the VL and VH Domain further comprise a Heterodimer-Promoting Domain, shown here comprising a cysteine residue.

FIG. 2 provides schematics of a representative covalently bonded binding molecule having four epitope-binding sites composed of five polypeptide chains. Two of the polypeptide chains possess a linker comprising a cysteine (which linker may comprise all or a portion of a hinge region) and a CH2 and CH3 Domain, such that the associated chains form an Fc Region that comprises all or part of an Fc Region. The polypeptide chains comprising the linked VL and VH Domains further comprise a linker and a Heterodimer-Promoting Domain (further described in FIG. 1B). VL and VH Domains that recognize the same epitope are shown using the same shading or fill pattern. The variable domains may be been selected to yield a resultant CD137×TA Binding Molecule that possesses two non-diabody type binding domains specific for a TA, and two diabody-type binding domains specific for CD137. Alternatively, the variable domains may be selected to yield a resultant CD137×TA Binding Molecule that possesses two non-diabody type binding domains specific for CD137 and two diabody-type binding domains specific for a TA. Such molecules are bispecific and have two binding sites for CD137, which may bind the same or different CD137 epitopes, and two binding sites for a TA which may bind the same or different TA epitopes.

FIGS. 3A-3C provide schematics of representative Fc Region-containing trivalent binding molecules having three epitope-binding sites. FIG. 3A illustrates schematically the domains of trivalent binding molecules comprising two diabody-type binding domains covalently bonded via linkers/Heterodimer-Promoting Domains (further described in FIG. 1B) and a Fab-type binding domain in which the binding domains are N-terminal to an Fc Region. The molecule in FIG. 3A comprises four chains. FIGS. 3B-3C, respectively, illustrate schematically the domains of trivalent binding molecules comprising two diabody-type binding domains, and a Fab-type binding domain in which the light chain and heavy chain are linked via a polypeptide spacer, or comprising an scFv-type binding domain. The trivalent binding molecules in FIGS. 3B-3C comprise three chains. VL and VH Domains that recognize the same epitope are shown using the same shading or fill pattern.

FIG. 4 shows the ability of CD137×TA Binding Molecules DART-A, TRIDENT-A, the comparator molecule TRIDENT-2, and the negative control hIgG1 to bind to CD137 expressed on the surface of engineered CHO cells.

FIGS. 5A-5B show the ability of CD137×TA Binding Molecules DART-A, TRIDENT-A, hPD-L1 MAB-2(1.1), and the negative control hIgG1 to bind to PD-L1 expressed on the cell surface of engineered CHO cells (FIG. 5A) and to block PD-L1/PD-1 interactions in a PD-L1 reporter assay (FIG. 5B).

FIG. 6 shows the ability of CD137×TA Binding Molecules DART-A, TRIDENT-A, the comparator molecules: DART-2, and TRIDENT-2, DART-3, r-urelumab, and the negative controls: DART-1 and hIgG1 to mediate target-dependent signal transduction in a CD137 Reporter Assay.

FIGS. 7A-7B show ability of CD137×TA Binding Molecules DART-A, TRIDENT-A, the comparator molecules: DART-2, and TRIDENT-2, DART-3, r-urelumab, and the negative controls: DART-1 and hIgG1 to mediate target-dependent release of cytokines INF-7 (FIG. 7A) and IL-2 (FIG. 7B) in a primary T cell cytokine release assay.

FIGS. 8A-8C show the serum levels and induction of immune cell proliferation by the CD137×TA Binding Molecule TRIDENT-A. Pharmacokinetics (serum clearance) (FIG. 8A), CD8⁺ T cell proliferation (FIG. 8A), NK cell proliferation (FIG. 8A) through day 20-24 in cynomolgus monkeys treated with TRIDENT-A at 1 mg/kg (solid circles) or 10 mg/kg (open circles) are plotted.

FIGS. 9A-9B show the binding activity of Fabs comprising deimmunized/optimized variants of hPD-L1 MAB-2(1.1). The ELISA binding curves of Fab variants hPD-L1 MAB-2B, hPD-L1 MAB-2D, and hPD-L1 MAB-2F (FIG. 9A) and hPD-L1 MAB-2A, hPD-L1 MAB-2C, and hPD-L1 MAB-2E (FIG. 9B) are plotted.

FIGS. 10A-10B show the ability of CD137×TA Binding Molecules comprising deimmunized or optimized PD-L1 binding domains to bind to PD-L1 expressed on the cell surface of engineered CHO cells. The binding curves of DART-A1, DART-A4, and the anti-PD-L1 antibody hPD-L1 MAB-2(1.1) (FIG. 10A), TRIDENT-A, TRIDENT-A4, and the negative control hIgG1 (FIG. 10B) are plotted.

FIGS. 11A-11C show ability of CD137×TA Binding Molecules comprising deimmunized and/or optimized PD-L1 binding domains to block PD-L1/PD-1 interactions in a PD-L1 reporter assay. The activity curves of DART-Ai, DART-A4, and the anti-PD-L1 antibody hPD-L1 MAB-2(1.1) (FIG. 11A), TRIDENT-A, TRIDENT-A4, and the negative control hIgG1 (FIG. 11B), DART-A4, DART-A7, DART-A8, DART-A9, and the negative control hIgG1 (FIG. 11C) are plotted.

FIGS. 12A-12B show the ability of CD137×TA Binding Molecules comprising deimmunized CD137 binding domains and/or deimmunized/optimized PD-L1 binding domains to bind to CD137 expressed on the surface of engineered CHO cells. The binding curves for DART-A4, DART-A5, DART-A6 (FIG. 12A), TRIDENT-A4, TRIDENT-A5, TRIDENT-A6 (FIG. 12B) are plotted. Also plotted on both figures are the comparator r-urelumab and the negative control hIgG1.

FIGS. 13A-13B show the ability of CD137×TA Binding Molecules comprising deimmunized CD137 binding domains, and/or deimmunized/optimized PD-L1 binding domains to mediate target-dependent signal transduction in a CD137 Reporter Assay performed with the low PD-L1 expressing N87 target cells (FIG. 13A), or the medium PD-L1 expressing JIMT-1 target cells (FIG. 13B). The activity of DART-A4, DART-A5, DART-A6, TRIDENT-A4, TRIDENT-A5, TRIDENT-A6, the comparator r-urelumab and the negative control hIgG1 are plotted.

FIGS. 14A-14B show the ability of CD137×TA Binding Molecules comprising deimmunized CD137 binding domains, and deimmunized/optimized PD-L1 binding domains to mediate target-dependent release of cytokines INF-7 (FIG. 14A) and IL-2 (FIG. 14B) in a primary T cell cytokine release assay. The activity of DART-A4, DART-A5, DART-A6, TRIDENT-A4, TRIDENT-A5, TRIDENT-A6, the comparator r-urelumab and the negative control hIgG1 are plotted.

FIGS. 15A-15B show the ability of CD137×TA Binding Molecules comprising parental, or deimmunized/optimized PD-L1 and/or CD137 binding domains to bind to PD-L1 (FIG. 15A) and CD137 (FIG. 15B) expressed on the cell surface of engineered CHO cells. The binding curves of DART-A, DART-A4, DART-A6, DART-A7, DART-A10, the anti-PD-L1 antibodies hPD-L1 MAB-2(1.1), and r-atezolizumab, and the negative control hIgG1 (FIG. 15A), DART-A, DART-A4, DART-A6, DART-A7, DART-A10, r-urelumab, and the negative control hIgG1 (FIG. 15B) are plotted.

FIGS. 16A-16B show the ability of CD137×TA Binding Molecules comprising parental, or deimmunized/optimized PD-L1 and/or CD137 binding domains to block PD-L1/PD-1 interactions in a PD-L1 reporter assay. The results for the tetravalent molecules DART-A, DART-A4, DART-A6, DART-A7, DART-A10, are plotted in FIG. 16A, and for the trivalent molecule TRIDENT-A, TRIDENT-A4, and TRIDENT-A6, are plotted in FIG. 16B. Also plotted on both figures are the anti-PD-L1 antibodies hPD-L1 MAB-2F and r-atezolizumab, and the negative control hIgG1.

FIGS. 17A-17B show the ability of CD137×TA Binding Molecules comprising parental, or deimmunized/optimized PD-L1 and/or CD137 binding domains to mediate target-dependent signal transduction in a CD137 Reporter Assay performed in the presence the medium PD-L1 expressing JIMT-1 target cells (FIG. 17A), or in the absence of target cells (FIG. 17B). The activity of DART-A, DART-A4, DART-A6, DART-A7, DART-A10, TRIDENT-A, TRIDENT-A4, TRIDENT-A6, the comparator r-urelumab and the negative control hIgG1 are plotted.

FIGS. 18A-18B show the ability of CD137×TA Binding Molecules comprising parental, or deimmunized/optimized PD-L1 and/or CD137 binding domains to mediate target-dependent release of cytokines INF-7 (FIG. 18A) and IL-2 (FIG. 18B). The activity of DART-A, DART-A4, DART-A6, DART-A7, DART-A10, TRIDENT-A, TRIDENT-A4, TRIDENT-A6, the combination of r-atezolizumab and r-urelumab (r-atezo+r-ure combo) and the negative control hIgG1 are plotted.

FIGS. 19A-19C show the ability of several representative PD-L1×CD137 bispecific molecules: DART-A (FIG. 19A), TRIDENT-A (FIG. 19B), or TRIDENT-A4 (FIG. 19C), in combination with a representative TA×CD3 bispecific molecules (5T4×CD3 diabody) to prevent or inhibit tumor growth or development of RKO colon carcinoma cells in vivo relative to a TA×CD3 bispecific molecule alone or a vehicle control in a murine PBMC-reconstituted xenograft model.

FIGS. 20A-20B show the ability of several representative PD-L1×CD137 bispecific molecules: DART-A6 (FIG. 20A), or TRIDENT-A6 (FIG. 20B), in combination with a representative TA×CD3 bispecific molecules (5T4×CD3 diabody), to prevent or inhibit tumor growth or development of RKO colon carcinoma cells in vivo relative to a TA×CD3 bispecific molecule alone or a vehicle control in a murine PBMC-reconstituted xenograft model.

FIGS. 21A-21B show the ability of several representative PD-L1×CD137 bispecific molecules: TRIDENT-A, TRIDENT-A6 comprising the VH/VL of CD137 MAB-6 binding domain, or comparator molecules: TRIDENT-2, DUO-1 comprising the VH/VL of different CD137 binding domains, in combination with a representative TA×CD3 bispecific molecules (5T4×CD3 diabody), to prevent or inhibit tumor growth or development of RKO colon carcinoma cells in vivo relative to a vehicle control in a murine PBMC-reconstituted xenograft model. Representative data from a first study are plotted in FIG. 21A, and from a second study in FIG. 21B.

FIGS. 22A-22B show the ability of CD137×TA Binding Molecules comprising CD137 binding domains, and HER2 binding domains, to mediate target-dependent signal transduction in a CD137 Reporter Assay performed with medium HER2 expressing JIMT-1 cells (FIG. 22A), or high HER2 expressing N87 target cells (FIG. 22B). The activity of DART-B1, DART-B2, TRIDENT-B1, TRIDENT-B2, the parental hHER2 MAB-1(1.3) and CD137 MAB-6(1.1) antibodies, and the negative controls, DART-4, DART-5, TRIDENT-3, TRIDENT-4, are plotted.

FIGS. 23A-23D show the ability of CD137×TA Binding Molecules comprising CD137 binding domains, and HER2 binding domains, to mediate target-dependent release of cytokines INF-7 (FIGS. 23A and 23B) and IL-2 (FIGS. 23C and 23D) in a primary T cell cytokine release assay performed with medium HER2 expressing JIMT-1 cells (FIGS. 22A and 23C), or high HER2 expressing N87 target cells (FIGS. 22B and 23D). The activity of DART-B1, DART-B2, TRIDENT-B1, TRIDENT-B2, the parental hHER2 MAB-1(1.3) and CD137 MAB-6(1.1) antibodies, and the negative controls, DART-4, DART-5, TRIDENT-3, TRIDENT-4, are plotted.

### DETAILED DESCRIPTION

The present invention is directed to CD137 Binding Molecules, such as monospecific antibodies, and molecules comprising epitope-binding fragments thereof, that are capable of binding to an epitope of CD137. The invention is further directed to multispecific CD137 Binding Molecules (e.g., bispecific antibodies, bispecific diabodies, BiTEs, trivalent binding molecules, etc.) that are capable of binding to both an epitope of CD137 and to an epitope of a second antigen, particularly a tumor antigen (“TA”) (e.g., a “CD137×TA Binding Molecule”). The invention also provides novel PD-L1 Binding Molecules, such as monospecific antibodies, and molecules comprising epitope-binding fragments thereof, that are capable of binding to an epitope of PD-L1, as well as derivatives thereof and uses thereof. The present invention is also directed to pharmaceutical compositions that comprise such molecules. The invention also includes the use of such molecules in the treatment of disease, especially cancer or a disease or condition associated with or characterized by the presence of a suppressed immune system.

### I. Antibodies and Other Binding Molecules

The CD137×TA Binding Molecules of the present invention may be antibodies, or be derivable from antibodies (e.g., by fragmentation, cleavage, etc. of antibody polypeptides, or from use of the amino acid sequences of antibody molecules or of polynucleotides (or their sequences) that encode such polynucleotides, etc.).

### A. Antibodies

Antibodies are immunoglobulin molecules capable of specific binding to a target region (“epitope”) of a molecule, such as a carbohydrate, polynucleotide, lipid, polypeptide, etc. (“antigen”), through at least one “epitope-binding site” located in the Variable Region of the immunoglobulin molecule. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies are capable of “immunospecifically binding” to a polypeptide or protein or a non-protein molecule due to the presence on such molecule of a particular domain or moiety or conformation (an “epitope”). As used herein, an “epitope-binding fragment of an antibody” is intended to denote a portion of an antibody capable of immunospecifically binding to an epitope. As used herein, such term encompasses fragments (such as Fab, Fab′, F(ab′)₂ Fv), and single chain (scFv), as well as the epitope-binding domain of a diabody. As used herein, an antibody or an epitope-binding fragment thereof is said to “immunospecifically” bind a region of another molecule (i.e., an epitope) if it reacts or associates more frequently, more rapidly, with greater duration and/or with greater affinity or avidity with that epitope relative to alternative epitopes. It is also understood by reading this definition that, for example, an antibody or an epitope-binding fragment thereof that immunospecifically binds to a first target may or may not specifically or preferentially bind to a second target. An epitope-containing molecule may have immunogenic activity, such that it elicits an antibody production response in an animal; such molecules are termed “antigens”. Natural antibodies are capable of binding to only one epitope species (i.e., they are “monospecific”), although they can bind multiple copies of that species (i.e., exhibiting “bivalency” or “multivalency”).

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Alternatively, existing monoclonal antibodies and any other equivalent antibodies that are immunospecific for a desired pathogenic epitope can be sequenced and produced recombinantly by any means known in the art. In one embodiment, such an antibody is sequenced, and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the epitope-binding portion of the antibody, while swapping the non-human remainder of the antibody with human antibody sequences.

The last few decades have seen a revival of interest in the therapeutic potential of antibodies, and antibodies have become one of the leading classes of biotechnology-derived drugs. Over 200 antibody-based drugs have been approved for use or are under development.

### 1. General Structural Attributes of Antibodies

The basic structural unit of naturally occurring immunoglobulins (e.g., IgG) is a tetramer composed of two shorter “Light Chains” complexed with two longer “Heavy Chains” and is usually expressed as a glycoprotein of about 150,000 Da. Each chain is composed of an amino-terminal (“N-terminal”) portion that comprises a “Variable Domain” and a carboxy-terminal (“C-terminal”) portion that comprises at least one “Constant Domain.” An IgG Light Chain is composed of a single “Light Chain Variable Domain” (“VL”) and a single “Light Chain Constant Domain” (“CL”). Thus, the structure of the light chains of an IgG molecule is n-VL-CL-c (where n and c represent, respectively, the N-terminus and the C-terminus of the polypeptide). An IgG Heavy Chain is composed of a single “Heavy Chain Variable Domain” (“VH1”), three “Heavy Chain Constant Domains” (“CH1,” “CH2” and “CH3”), and a “Hinge” Region (“H”), located between the CH1 and CH2 Domains. Thus, the structure of an IgG heavy chain is n-VH-CH1-H-CH2-CH3-c (where n and c represent, respectively, the N-terminus and the C-terminus of the polypeptide). The ability of an intact, unmodified antibody (e.g., an IgG antibody) to bind an epitope of an antigen depends upon the presence and sequences of the Variable Domains.

### a) Constant Domains

### (1) Light Chain Constant Domain

A representative CL Domain is a human IgG CL Kappa Domain. The amino acid sequence of an representative human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an representative CL Domain is a human IgG CL Lambda Domain. The amino acid sequence of an representative human CL Lambda Domain is SE ID NO:2):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

### (2) Heavy Chain CH1 Domains

An representative CH1 Domain is a human IgG1 CH1 Domain. The amino acid sequence of an representative human IgG1 CH1 Domain is (SEQ ID NO:3):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An representative CH1 Domain is a human IgG2 CH1 Domain. The amino acid sequence of an representative human IgG2 CH1 Domain is (SEQ ID NO:4):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An representative CH1 Domain is a human IgG3 CH1 Domain. The amino acid sequence of an representative human IgG3 CH1 Domain is (SEQ ID NO:5):

ASTKGPSVFP LAPCSRSTSG GTAALGCLVK DYFPEPVTVS
WNSGALTSGV HTFPAVLQSS GLYSLSSVVT VPSSSLGTQT
YTCNVNHKPS NTKVDKRV

An representative CH1 Domain is a human IgG4 CH1 Domain. The amino acid sequence of an representative human IgG4 CH1 Domain is (SEQ ID NO:6):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

### (3) Heavy Chain Hinge Regions

An representative Hinge Region is a human IgG1 Hinge Region. The amino acid sequence of an representative human IgG1 Hinge Region is (SEQ ID NO:7):

EPKSCDKTHT CPPCP

Another representative Hinge Region is a human IgG2 Hinge Region. The amino acid sequence of an representative human IgG2 Hinge Region is (SEQ ID NO:8):

ERKCCVECPP CP

Another representative Hinge Region is a human IgG3 Hinge Region. The amino acid sequence of an representative human IgG3 Hinge Region is (SEQ ID NO:9):

ELKTPLGDTT HTCPRCPEPK SCDTPPPCPR CPEPKSCDTP
PPCPRCPEPK SCDTPPPCPR CP

Another representative Hinge Region is a human IgG4 Hinge Region. The amino acid sequence of an representative human IgG4 Hinge Region is (SEQ ID NO:10):

ESKYGPPCPS CP

As described herein, an IgG4 Hinge Region may comprise a stabilizing mutation such as the S228P substitution (as numbered by the EU index as set forth in Kabat). The amino acid sequence of an representative stabilized IgG4 Hinge Region is (SEQ ID NO:11):

ESKYGPPCPP CP

### (4) Heavy Chain CH2 and CH3 Domains

The CH2 and CH3 Domains of the two heavy chains interact to form the “Fc Region” of IgG antibodies that is recognized by cellular Fc Receptors, including but not limited to Fc gamma Receptors (FcγRs). As used herein, the term “Fc Region” is used to define a C-terminal region of an IgG heavy chain. A portion of an Fc Region (including a portion that encompasses an entire Fc Region) is referred to herein as an “Fc Domain.” An Fc Region is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of an representative human IgG1 is SEQ ID NO:12).

```
231 240 250 260 270
APELLGGPSV FLFPPKPKDT LMISRTPEVT CVVVDVSHED
                  280 290 300 310
PEVKFNWYVD GVEVHNAKTK PREEQYNSTY RVVSVLTVLH
                  320 330 340 350
QDWLNGKEYK CKVSNKALPA PIEKTISKAK GQPREPQVYT
                  360 370 380 390
LPPSREEMTK NQVSLTCLVK GFYPSDIAVE WESNGQPENN
                  400 410 420 430
YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an representative human IgG2 is (SEQ ID NO:13):

```
231 240 250 260 270
APPVA-GPSV FLFPPKPKDT LMISRTPEVT CVVVDVSHED
                  280 290 300 310
PEVQFNWYVD GVEVHNAKTK PREEQFNSTF RVVSVLTVVH
                  320 330 340 350
QDWLNGKEYK CKVSNKGLPA PIEKTISKTK GQPREPQVYT
                  360 370 380 390
LPPSREEMTK NQVSLTCLVK GFYPSDISVE WESNGQPENN
                  400 410 420 430
YKTTPPMLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an representative human IgG3 is (SEQ ID NO:14):

```
231 240 250 260 270
APELLGGPSV FLFPPKPKDT LMISRTPEVT CVVVDVSHED
                  280 290 300 310
PEVQFKWYVD GVEVHNAKTK PREEQYNSTF RVVSVLTVLH
                  320 330 340 350
QDWLNGKEYK CKVSNKALPA PIEKTISKTK GQPREPQVYT
                  360 370 380 390
LPPSREEMTK NQVSLTCLVK GFYPSDIAVE WESSGQPENN
                  400 410 420 430
YNTTPPMLDS DGSFFLYSKL TVDKSRWQQG NIFSCSVMHE
                  440 447
ALHNRFTQKS LSLSPGX
```

as numbered by the EU index as set forth in Kabat, wherein X is a lysine (K) or is absent.

The amino acid sequence of the CH2-CH3 Domain of an representative human IgG4 is (SEQ ID NO:15):

```
231 240 250 260 270
APEFLGGPSV FLFPPKPKDT LMISRTPEVT CVVVDVSQED
                  280 290 300 310
PEVQFNWYVD GVEVHNAKTK PREEQFNSTY RVVSVLTVLH
                  320 330 340 350
QDWLNGKEYK CKVSNKGLPS SIEKTISKAK GQPREPQVYT
                  360 370 380 390
LPPSQEEMTK NQVSLTCLVK GFYPSDIAVE WESNGQPENN
                  400 410 420 430
YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by reference. The term “EU index as in Kabat” refers to the numbering of the constant domains of human IgG1 EU antibody.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the molecules of the invention. Specifically encompassed by the instant invention are molecules of the invention lacking the C-terminal residue of the CH3 Domain. Also specifically encompassed by the instant invention are such molecules comprising the C-terminal lysine residue of the CH3 Domain.

### b) Variable Domains

The Variable Domains of an IgG molecule consist of three “complementarity determining regions” (“CDRs”), which contain the amino acid residues of the antibody that will be in contact with the epitope, as well as intervening non-CDR segments, referred to as “framework regions” (“FR”), which, in general maintain the structure and determine the positioning of the CDR loops so as to permit such contacting (although certain framework residues may also contact the epitope). Thus, the VL and VH Domains have the structure n-FR1-CDR1-FR2-CDR2-FR3-CDR3-FR4-c. The amino acid sequences of the CDRs determine whether an antibody will be able to bind to a particular epitope. Interaction of an antibody light chain with an antibody heavy chain and, in particular, interaction of their VL and VH Domains, forms an epitope-binding site of the antibody.

Amino acids from the Variable Domains of the mature heavy and light chains of immunoglobulins are designated by the position of an amino acid in the chain. Kabat (SEQUENCES OF PROTEINS OF IMMUNOLOGICAL INTEREST, 5^{th} Ed. Public Health Service, NH1, MD (1991)) described numerous amino acid sequences for antibodies, identified an amino acid consensus sequence for each subgroup, and assigned a residue number to each amino acid, and the CDRs and FRs are identified as defined by Kabat (it will be understood that CDR_{H}1 as defined by Chothia, C. & Lesk, A. M. ((1987) “Canonical Structures For The Hypervariable Regions Of Immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain. The positions within the VL and VH Domains at which the their CDRs commence and end are thus well defined and can be ascertained by inspection of the sequences of the VL and VH Domains (see, e.g., Martin, C. R. (2010) “Protein Sequence and Structure Analysis of Antibody Variable Domains,” In: ANTIBODY ENGINEERING VOL. 2 (Kontermann, R. and Dübel, S. (eds.), Springer-Verlag Berlin Heidelberg, Chapter 3 (pages 33-51)).

Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDRHA Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe, etc.), or is another type of protein. Accordingly, as used herein, the term “Epitope-Binding Fragment” denotes a fragment of a molecule capable of immunospecifically binding to an epitope. An epitope-binding fragment may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Typically, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab2 fragment, etc.). Unless specifically noted, the order of domains of the protein molecules described herein is in the “N-Terminal to C-Terminal” direction.

The epitope-binding site may comprise either a complete Variable Domain fused onto Constant Domains or only the complementarity determining regions (CDRs) of such Variable Domain grafted to appropriate framework regions. Epitope-binding sites may be wild-type or modified by one or more amino acid substitutions.

### c) Humanization of Antibodies

The invention particularly encompasses binding molecules (including antibodies and diabodies) that comprise a VL and/or VH Domain of a humanized antibody. The term “humanized” antibody refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The polynucleotide sequence of the variable domains of such antibodies may be used for genetic manipulation to generate such derivatives and to improve the affinity, or other characteristics of such antibodies. It is known that the variable domains of both heavy and light chains contain three CDRs which vary in response to the antigens in question and determine binding capability, flanked by four FRs which are relatively conserved in a given species and which putatively provide a scaffolding for the CDRs. When non-human antibodies are prepared with respect to a particular antigen, the variable domains can be “reshaped” or “humanized.” The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

A number of humanized antibody molecules comprising an epitope-binding site derived from a non-human immunoglobulin have been described, including chimeric antibodies having rodent or modified rodent Variable Domain and their associated CDRs fused to human constant domains (see, for example, Lobuglio et al. (1989) “Mouse Human Chimeric Monoclonal Antibody In Man: Kinetics And Immune Response,” Proc. Natl. Acad. Sci. (U.S.A.) 86:4220-4224 (1989),). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; and 5,997,867. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

### 2. CD137 Binding Domains

The present invention is directed to CD137 Binding Molecules, such as monospecific antibodies, and molecules comprising epitope-binding fragments thereof, that are capable of binding to an epitope of CD137. The CD137 binding domains of the novel human monoclonal antibody “CD137 MAB-6” are provided below. The present invention specifically includes and encompasses CD137 Binding Molecules and multispecific CD137 Binding Molecules (e.g., bispecific antibodies, bispecific diabodies, BiTEs, trivalent binding molecules, etc.) such as CD137×TA Binding Molecules that comprise the VL and/or VH Domain, and/or 1, 2 or all 3 of the CDR_{L}s of the VL Region and/or 1, 2 or all 3 of the CDR_{H}S of the VH Domain of CD137 MAB-6, or any of the variants thereof provided below.

### a) Human CD137 MAB-6

CD137 MAB-6 is a novel human monoclonal antibody. The amino acid sequence of the VH Domain of CD137 MAB-6 (CD137 MAB-6 VH1) is (SEQ ID NO:46) (CDR_{H} residues are shown underlined):

QVQLQESGPG LVKPSETLSL TCTVSGGSIS
SYYWSWIRQP PGKGLEWIGR IYTSGSTNYN
PSLKSRVTMS VDTSKNQFSL KLSSVTAADT
AVYYCARDGW YDEDYNYYGM DVWGQGTTVT
VSS

The amino acid sequences of the CDR_{H}s of CD137 MAB-6 VH1 are:

| | |
|---|---|
| CDR_{H}1 (SEQ ID NO: 47): | SYYWS |
| CDR_{H}2 (SEQ ID NO: 48): | RIYTSGSTNYNPSLKS |
| CDR_{H}3 (SEQ ID NO: 49): | DGWYDEDYNYYGMDV |

The amino acid sequence of the VL Domain of CD137 MAB-6 (CD137 MAB-6 VL1) is (SEQ ID NO:50) (CDR_{L} residues are shown underlined):

EIVMTQSPAT LSLTPGERAT LSCRASQSVS
SNYLSWFQQI PGQAPRLLIY GASTRATGIP
ARFSGSGSGT DFTLTISSLQ PEDFAVYYCQ
QDYDLPWTFG QGTKVEIK

The amino acid sequences of the CDR_{L}s of CD137 MAB-6 VL1 are:

| | |
|---|---|
| CDR_{L}1 (SEQ ID NO: 51): | RASQSVSSNYLS |
| CDR_{L}2 (SEQ ID NO: 52): | GASTRAT |
| CDR_{L}3 (SEQ ID NO: 53): | QQDYDLPWT |

### b) Deimmunized CD137 MAB-6

As described in the examples below the VL Domain of CD137 MAB-6 was deimmunized to yield VL Domains designated “CD137 MAB-6 VLx” having the amino acid sequence of SEQ ID NO:54 (CDR_{L} residues are shown underlined):

EIVMTQSPAT LSLX₁PGERAT LSCRASQSVS
SNYLSWX₂QQX₃ PGQAPRLLIY GASTRATGIP
ARFSGSGSGT DFTLTISSLQ PEDFAVYYCQ
QDYDLPWTFG QGTKVEIK

wherein: X₁, X₂, and X₃ are independently selected, and
wherein: X₁ is S or T; X₂ is F or Y; and X₃ is I or K.

In particular embodiments
a) X₁ is S; X₂ is Y; and X₃ is K; or
b) X₁ is S; X₂ is F; and X₃ is K.

The amino acid sequences of variants of CD137 MAB-6 VL Domains designated CD137 MAB-6 VL2, and CD137 MAB-6 VL3 are presented below. Any of the variant VL Domains may be paired with the VH Domain. Molecules comprising particular combinations of CD137 MAB-6 VH/VL Domains are referred to by reference to the specific VH/VL Domains, for example, a molecule comprising the binding domains CD137 MAB-6 VH1 and CD137 MAB-6 VL3 is specifically referred to as “CD137 MAB-6(1.3).”

The amino acid sequence of the variant CD137 MAB-6 VL2 is (SEQ ID NO:55) (CDR_{L} residues are shown underlined):

EIVMTQSPAT LSLSPGERAT LSCRASQSVS
SNYLSWYQQK PGQAPRLLIY GASTRATGIP
ARFSGSGSGT DFTLTISSLQ PEDFAVYYCQ
QDYDLPWTFG QGTKVEIK

The amino acid sequence of the variant CD137 MAB-6 VL3 is (SEQ ID NO:56) (CDR_{L} residues are shown underlined):

EIVMTQSPAT LSLSPGERAT LSCRASQSVS
SNYLSWFQQK PGQAPRLLIY GASTRATGIP
ARFSGSGSGT DFTLTISSLQ PEDFAVYYCQ
QDYDLPWTFG QGTKVEIK

The CDRs, VL Domain, and/or VH Domain of any of such fully human, and/or variant VH and VL CD137 MAB-6 Domains, including any embraced within the generic sequence(s) of the CD137 MAB-6 VL Domains presented above may be used to form an antibody, diabody or binding molecule capable of binding CD137. In certain embodiments CD137 Binding Molecules of the invention, including CD137×TA Binding Molecules, comprise CD137 MAB-6 VH1 and CD137 MAB-6 VL3.

### B. Bispecific Antibodies, Multi-Specific Diabodies and Trivalent Molecules

As indicated above, natural antibodies are capable of binding to only one epitope species, although they can bind multiple copies of that species. The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two Epitope Binding Domains of a natural antibody, such as an IgG. Natural antibodies are capable of binding only one epitope species (i.e., they are mono-specific), although they can bind multiple copies of that species (i.e., exhibiting bi-valency or multi-valency).

The functionality of antibodies can be enhanced by generating multispecific antibody-based molecules that can simultaneously bind two separate and distinct antigens (or different epitopes of the same antigen) and/or by generating antibody-based molecule having higher valency (i.e., more than two Binding Domains) for the same epitope and/or antigen.

In order to provide molecules having greater capability than natural antibodies, a wide variety of recombinant bispecific antibody formats have been developed to produce such bispecific antibodies.

Most of such approaches use linker peptides to fuse a further binding domain (e.g. an scFv, VL, VH, etc.) to, or within the antibody core (IgA, IgD, IgE, IgG or IgM), or to fuse multiple antibody binding portions to one another (e.g. two Fab fragments or scFv). Alternative formats use linker peptides to fuse a binding protein (e.g., an scFv, VL, VH, etc.) to a dimerization domain such as the CH2-CH3 Domain or alternative polypeptides (WO 2005/070966, WO 2006/107786A WO 2006/107617A, WO 2007/046893). PCT Publication Nos. WO 2013/174873, WO 2011/133886 and WO 2010/136172 disclose mutispecific antibodies in which the CL and CH1 Domains are switched from their respective natural positions WO 2008/027236; and WO 2010/108127 disclose antibodies in which the VL and VH Domains have been diversified to allow them to bind to more than one antigen. PCT Publication Nos. WO 2010/028797, WO2010028796 and WO 2010/028795 disclose recombinant antibodies whose Fc Regions have been replaced with additional VL and VH Domains, so as to form trivalent binding molecules. PCT Publication Nos. WO 2003/025018 and WO 2003/012069 disclose recombinant diabodies whose individual chains contain scFv domains. PCT Publication No. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publication Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional Binding Domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another).

The art has additionally noted the capability of producing diabodies that differ from natural antibodies in being capable of binding two or more different epitope species (i.e., exhibiting bispecificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Baeuerle, P. A. et al. (2009) “Bispecific T cell Engaging Antibodies For Cancer Therapy,” Cancer Res. 69(12):4941-4944).

The provision of non-monospecific “diabodies” provides a significant advantage over antibodies: the capacity to co-ligate and co-localize cells that express different epitopes. Bispecific diabodies thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens.

The formation of such non-mono-specific diabodies requires the successful assembly of two or more distinct and different polypeptides (i.e., such formation requires that the diabodies be formed through the heterodimerization of different polypeptide chain species). In the face of this challenge, the art has succeeded in developing stable, covalently bonded heterodimeric non-mono-specific diabodies, (see, e.g., Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Veri, M. C. et al. (2010) “Therapeutic Control Of B Cell Activation Via Recruitment Of Fcgamma Receptor IIB (CD32B) Inhibitory Function With A Novel Bispecific Antibody Scaffold,” Arthritis Rheum. 62(7):1933-1943; Moore, P. A. et al. (2011) “Application OfDual Affinity Retargeting Molecules To Achieve Optimal Redirected T cell Killing Of B-Cell Lymphoma,” Blood 117(17):4542-4551; US Patent Publication Nos. 2007/0004909; 2009/0060910; 2010/0174053; 20130295121; 2014/0099318; 2015/0175697; 2016/0017038; 2016/0194396; 2016/0200827; and 2017/0247452). Such diabodies comprise two or more covalently complexed polypeptides and involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

### C. Components of the Representative CD137×TA Binding Molecules Of The Present Invention

The CD137×TA Binding Molecules of the present invention are composed of polypeptides, and may be composed of two, three, four or more than four polypeptide chains. As used herein, the term “composed of” is intended to be open-ended, such that a CD137×TA Binding Molecules of the present invention that is composed of two polypeptide chains may possess additional polypeptide chains. Such chains may have the same sequence as another polypeptide chain of the binding molecule, or may be different in sequence from any other polypeptide chain of the Binding Molecule.

### 1. Representative “Linker” Peptides

The polypeptides of the CD137×TA Binding Molecules of the present invention comprise domains that are preceded by, followed by, and/or linked to one another by “linker” peptides, such as Linker 1, Linker 2, Linker 3, etc. Although the invention utilizes certain specific “linker” peptides, in light of the teachings provided herein, alternative linkers could readily be identified and employed to achieve CD137×TA Binding Molecules.

The length of Linker 1, which separates the VL and VH domains of a polypeptide chain is selected to substantially or completely prevent such VL and VH domains from binding to one another (e.g., 12 or less amino acid residues in length). Thus, the VL1 and VH2 domains of the first polypeptide chain are substantially or completely incapable of binding to one another, and do not form an epitope-binding site that is capable of substantially binding to either the first or second antigen. Likewise, the VL2 and VH1 domains of the second polypeptide chain are substantially or completely incapable of binding to one another, and do not form an epitope-binding site that is capable of substantially binding to either the first or second antigen. A representative intervening linker peptide (Linker 1) has the amino acid sequence (SEQ ID NO:16): GGGSGGGG, which is too short to allow the VL and VH Domains of the same polypeptide chain to complex together (in contrast to the longer intervening linker peptide that is employed to produce scFv molecules (e.g., GGGGSGGGGSGGGGS (SEQ ID NO:17)).

One purpose of Linker 2 is to separate the VH Domain of a polypeptide chain from the optionally present Heterodimer-Promoting Domain of that polypeptide chain. Any of a variety of linkers can be used for the purpose of Linker 2. A representative sequence for such Linker 2 comprises the amino acid sequence: GGCGGG (SEQ ID NO:18), which possesses a cysteine residue that may be used to covalently bond the first and second polypeptide chains to one another via a disulfide bond, or AS TKG (SEQ ID NO:19), which is derived from the IgG CH1 domain. Since the Linker 2, AS TKG (SEQ ID NO:19) does not possess such a cysteine, the use of such Linker 2 is typically associated with the use of a cysteine-containing Heterodimer-Promoting Domain, such as the E-coil of SEQ ID NO:39 or the K-coil of SEQ ID NO:40 (see below).

One purpose of Linker 3 is to separate the Heterodimer-Promoting Domain of a polypeptide chain from the Fc Domain of that polypeptide chain. A second purpose is to provide a cysteine-containing polypeptide domain. Any of a variety of linkers can be used for the purpose of Linker 3. A representative sequence for such Linker 3 comprises the amino acid sequence: DKTHTCPPCP (SEQ ID NO:20). Another representative sequence for Linker 3 comprises the amino acid sequence: GGGDKTHTCPPCP (SEQ ID NO:21). Still other representative sequences for Linker 3 comprise the amino acid sequence: LEPKSADKTHTCPPCP (SEQ ID NO:30), or LEPKSSDKTHTCPPCP (SEQ ID NO:31).

One purpose of Linker 4 is to separate the C-terminus of the CH2-CH3 domains of an Fc Region (“Fc Domain”) from the N-terminus of a VL Domain. Any of a variety of linkers can be used for the purpose of Linker 4. A representative sequence for such Linker 4 comprises the amino acid sequence: APSSS (SEQ ID NO:22) or the amino acid sequence APSSSPME (SEQ ID NO:23), the amino acid sequence GGGSGGGSGGG (SEQ ID NO:24), or the amino acid sequence GGGGSGGGSGGG (SEQ ID NO:25).

The Fc Region-containing molecules of the present invention may include additional intervening linker peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Region (preferably a cysteine-containing portion of an IgG Hinge Region). Linkers that may be employed in the bispecific Fc Region-containing diabody molecules of the present invention include: GGC, GGG, ASTKG (SEQ ID NO:19), DKTHTCPPCP (SEQ ID NO:20), APSSS (SEQ ID NO:22), APSSSPME (SEQ ID NO:23), GGGSGGGSGGG (SEQ ID NO:24), GGGGSGGGSGGG (SEQ ID NO:25), LGGGSG (SEQ ID NO:26), GGGS (SEQ ID NO:27), LEPKSS (SEQ ID NO:28), VEPKSADKTHTCPPCP (SEQ ID NO:29), LEPKSADKTHTCPPCP (SEQ ID NO:30), and LEPKSSDKTHTCPPCP (SEQ ID NO:31). LEPKSS (SEQ ID NO:28) may be used in lieu of GGG or GGC for ease of cloning. Additionally, the amino acids GGG, or LEPKSS (SEQ ID NO:28) may be immediately followed by DKTHTCPPCP (SEQ ID NO:20) to form the alternate linkers: GGGDKTHTCPPCP (SEQ ID NO:21); and LEPKSSDKTHTCPPCP (SEQ ID NO:31). Bispecific Fc Region-containing molecules of the present invention may incorporate an IgG Hinge Region, such as the IgG Hinge Region of a human IgG1, IgG2, IgG3 or IgG4 antibody, or a portion thereof.

### 2. Representative Heterodimer-Promoting Domains

As indicated above, the formation of the CD137×TA Binding Molecules of the present invention involves the assembly of two or more different polypeptide chains (i.e., heterodimerization). The formation of heterodimers of the first and second polypeptide chains can be driven by the inclusion of “Heterodimer-Promoting Domains.” The Heterodimer-Promoting Domains may be a domain of a Hinge Region of an IgG (or a polypeptide derived from a Hinge Region, such as, for example, GVEPKSC (SEQ ID NO:32), VEPKSC (SEQ ID NO:33)) or AEPKSC (SEQ ID NO:34)) on one polypeptide chain, and a CL Domain (or a polypeptide derived from the CL Domain, such as, for example, GFNRGEC (SEQ ID NO:35) or FNRGEC (SEQ ID NO:36)) on the other polypeptide chain (US2007/0004909).

Alternatively, the Heterodimer-Promoting Domains of the present invention will comprise tandemly repeated coil domains of opposing charge, for example “E-coil” helical domains (SEQ ID NO:37: EVAALEK-EVAALEK-EVAALEK-EVAALEK), whose glutamate residues will form a negative charge at pH 7, while the other of the Heterodimer-Promoting Domains will comprise four tandem “K-coil” domains (SEQ ID NO:38: KVAALKE-KVAALKE-KVAALKE-KVAALKE), whose lysine residues will form a positive charge at pH 7. The presence of such charged domains promotes association between the first and second polypeptides, and thus fosters heterodimerization. In another embodiment, a Heterodimer-Promoting Domain in which one of the four tandem “E-coil” helical domains of SEQ ID NO:37 has been modified to contain a cysteine residue: EVAACEK-EVAALEK-EVAALEK-EVAALEK (SEQ ID NO:39) is utilized. Likewise, in another embodiment, a Heterodimer-Promoting Domain in which one of the four tandem “K-coil” helical domains of SEQ ID NO:38 has been modified to contain a cysteine residue: KVAACKE-KVAALKE-KVAALKE-KVAALKE (SEQ ID NO:40) is utilized. 3. Covalent Bonding of the Polypeptide Chains

The CD137×TA Binding Molecules of the present invention are engineered so that pairs of their polypeptide chains covalently bond to one another via one or more cysteine residues positioned along their length to produce a covalently associated molecular complex. Such cysteine residues may be introduced into the intervening linker that separates the VL and VH domains of the polypeptides. Optionally, or alternatively, Linker 2 or Linker 3, or an alternative linker may contain a cysteine residue. Optionally or alternatively, one or more coil domains of a coil-containing Heterodimer-Promoting Domain will comprise an amino acid substitution that incorporates a cysteine residue as in SEQ ID NO:39 or SEQ ID NO:40.

### 4. Representative Fc Domains

The Fc Domain of an Fc-bearing CD137×TA Binding Molecule of the present invention may comprise a complete Fc region (e.g., a complete IgG Fc region) or only a fragment of a complete Fc region. The Fc Domain of the Fc-bearing CD137×TA Binding Molecules of the present invention may thus include some or all of the CH2 Domain and/or some or all of the CH3 Domain of a complete Fc region, or may comprise a variant CH2 and/or a variant CH3 sequence (that may include, for example, one or more insertions and/or one or more deletions with respect to the CH2 or CH3 domains of a complete Fc region). The Fc Domain of the bispecific Fc diabodies of the present invention may comprise non-Fc polypeptide portions, or may comprise portions of non-naturally complete Fc regions, or may comprise non-naturally occurring orientations of CH2 and/or CH3 domains (such as, for example, two CH2 domains or two CH3 domains, or in the N-terminal to C-terminal direction, a CH3 Domain linked to a CH2 Domain, etc.).

Although the Fc Domain of an Fc-bearing CD137×TA Binding Molecule of the present invention may comprise the amino acid sequence of a naturally occurring Fc Domain, it may be desirable for the CH2-CH3 Domains that form such Fc Domain to comprise one or more substitutions such that the resultant Fc Domain exhibits decreased (e.g., less than 50%, less than 40%, less than 30%, less than 20%, or less than 10%, of the binding exhibited by such molecule if having an Fc Domain having the amino acid sequence of a naturally-occurring Fc Region), or substantially no detectable, binding to FcTRIA (CD64), FcTRIIA (CD32A), FcTRIIB (CD32B), FcTRIIIA (CD16a) or FcTRIIIB (CD16b) (relative to the binding exhibited by the wild-type Fc region). Fc variants and mutant forms capable of mediating such altered binding are well known in the art and include amino acid substitutions at one or more positions elected from the group consisting of. 234, 235, 265, and 297, wherein said numbering is that of the EU index as in Kabat (see, for example, U.S. Pat. No. 5,624,821). In one embodiment, the CH2-CH3 Domain of the first and/or third polypeptide chains of the Fc-bearing molecules of the invention include any 1, 2, 3, or 4 of the substitutions: L234A, L235A, D265A, N297Q, and N297G. Alternatively, a CH2-CH3 Domain of a naturally occurring Fc region that inherently exhibits decreased (or substantially no) binding to FcTRIIIA (CD16a) and/or reduced effector function (relative to the binding and effector function exhibited by the wild-type IgG1 Fc Region (SEQ ID NO:12)) is utilized. In a specific embodiment, the Fc-bearing molecules of the present invention comprise an IgG2 Fc Region (SEQ ID NO:13) or an IgG4 Fc Region (SEQ ID NO:15). When an IgG4 Fc Region is utilized, the instant invention also encompasses the introduction of a stabilizing mutation, such as the Hinge Region S228P substitution described above (see, e.g., SEQ ID NO:11).

In a representative embodiment, the employed IgG1 CH2-CH3 Domain of Fc-bearing CD137×TA Binding Molecules of the present invention include a substitution at position 234 with alanine and 235 with alanine, wherein said numbering is that of the EU index as

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

The serum half-life of proteins comprising Fc Regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered.

In some embodiments, the Fc-bearing CD137×TA Binding Molecules of the present invention comprise a variant Fc Region, wherein said variant Fc Region comprises at least one amino acid modification relative to a wild-type Fc Region, such that said molecule has an increased half-life (relative to a molecule comprising a wild-type Fc Region). In some embodiments, the Fc-bearing CD137×TA Binding Molecules of the present invention comprise a variant IgG Fc Region, wherein said variant Fc Region comprises a half-live extending amino acid substitution. Numerous amino acid substitutions capable of increasing the half-life of an Fc-bearing molecule are known in the art see for example the amino acid substitutions described in U.S. Pat. Nos. 6,277,375, 7,083,784; 7,217,797, 8,088,376; U.S. Publication Nos. 2002/0147311; 2007/0148164; and 2011/0081347. A Fc-bearing CD137×TA Binding Molecule having enhanced half-life may comprise two or more substitutions selected from: T250Q, M252Y, S254T, T256E, K288D, T307Q, V308P, A378V, M428L, N434A, H435K, and Y436I, wherein said numbering is that of the EU index as in Kabat.

In particular, the employed CH2-CH3 Domain may comprise the substitutions:
(A) M252Y, S254T and T256E;
(B) M252Y and S254T;
(C) M252Y and T256E;
(D) T250Q and M428L;
(E) T307Q and N434A;
(F) A378V and N434A;
(G) N434A and Y436I;
(H) V308P and N434A;
(I) K288D and H435K; or
(J) M428L and N434S,
wherein said numbering is that of the EU index as in Kabat.

A representative sequence for the CH2 and CH3 Domains comprises the triple amino acid substitution: M252Y/S254T/T256E (YTE), which significantly enhances serum-half life (Dall'Acqua, W. F. et al. (2006) “Properties of Human IgGs Engineered for Enhanced Binding to the Neonatal Fc Receptor (FcRn),” J. Biol. Chem. 281(33):23514-23524), as in SEQ ID NO:42 or SEQ ID NO:43, which are variants of the IgG1 CH2-CH3 domain, or as in SEQ ID NO:44, which is a variant of the IgG4 CH2-CH3 Domain:

SEQ ID NO: 42:
APELLGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

SEQ ID NO: 43:
APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

SEQ ID NO: 44:
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGX

wherein, X is a lysine (K) or is absent.

The invention also encompasses Fc-bearing CD137×TA Binding Molecules comprising variant Fc Domains that exhibit altered effector function, altered serum half-life, altered stability, altered susceptibility to cellular enzymes or altered effector function as assayed in an NK dependent or macrophage dependent assay, etc. Fc Domain modifications identified as altering effector function are known in the art, including modifications that increase binding to activating receptors (e.g., FcTRIIA (CD16A) and reduce binding to inhibitory receptors (e.g., FcTRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Representative variants of human IgG1 Fc Domains with reduced binding to CD32B and/or increased binding to CD16A contain L235V, F243L, R292P, Y300L, V305I or P296L substitutions. These amino acid substitutions may be present in a human IgG1 Fc Domain in any combination. In one embodiment, the human IgG1 Fc Domain variant contains a F243L, R292P and Y300L substitution, wherein said numbering is that of the EU index as in Kabat. In another embodiment, the human IgG1 Fc Domain variant contains a F243L, R292P, Y300L, V305I and P296L substitution, wherein said numbering is that of the EU index as in Kabat. In another embodiment, the human IgG1 Fc Domain variant contains a L235V, F243L, R292P, Y300L and P396L substitution, wherein said numbering is that of the EU index as in Kabat.

The CH2 and/or CH3 Domains of the CD137×TA Binding Molecules of the present invention need not be identical in sequence, and advantageously are modified to promote heterodimerization between the two CH2-CH3-bearing polypeptide chains. For example, an amino acid substitution (preferably a substitution with an amino acid comprising a bulky side group forming a “knob,” e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., a “hole” (e.g., a substitution with glycine). Such sets of mutations can be engineered into any pair of polypeptides comprising the bispecific Fc-bearing diabody molecule, and further, engineered into any portion of the polypeptides chains of said pair. Methods of protein engineering to favor heterodimerization over homodimerization are well known in the art, in particular with respect to the engineering of immunoglobulin-like molecules, and are encompassed herein (see e.g., Ridgway et al. (1996) “‘Knobs-Into-Holes' Engineering Of Antibody CH3 Domains For Heavy Chain Heterodimerization”, “Protein Engr. 9:617-621, Atwell et al. (1997) “Stable Heterodimers From Remodeling The Domain Interface Of A Homodimer Using A Phage Display Library,” J. Mol. Biol. 270: 26-3 5, and Xie et al. (2005) “A New Format Of Bispecific Antibody: Highly Efficient Heterodimerization, Expression And Tumor Cell Lysis,” J. Immunol. Methods 296:95-101; each of which documents is hereby incorporated herein by reference in its entirety). In one embodiment, the knob is engineered into the CH2-CH3 Domains of the first polypeptide chain and the hole is engineered into the CH2-CH3 Domains of the third polypeptide chain. Thus, the knob will help in preventing two molecules of the first polypeptide chain from homodimerizing via their CH2 and/or CH3 Domains. As the third polypeptide chain of this embodiment contains the hole substitution it will have the ability to heterodimerize with the first polypeptide chain as well as homodimerize with itself (however, such homodimerization does not form a molecule possessing epitope-binding sites). A representative knob is created by modifying a native IgG Fc Domain to contain the modification T366W, wherein said numbering is that of the EU index as in Kabat. A representative hole is created by modifying a native IgG Fc Domain to contain the modification T366S, L368A and Y407V, wherein said numbering is that of the EU index as in Kabat. To aid in purifying the third polypeptide chain homodimer from the final bispecific Fc-bearing diabody comprising heterodimers of the first and third polypeptide chains, the protein A binding site of the CH2 and CH3 Domains of the third polypeptide chain is preferably mutated by amino acid substitution at position 435 (H435R), wherein said numbering is that of the EU index as in Kabat. Thus, the third polypeptide chain homodimer will not bind to protein A, whereas the properly assembled bispecific Fc-bearing diabody will retain its ability to bind protein A via the protein A binding site on the first polypeptide chain.

SEQ ID NO:45, SEQ ID NO:146 and SEQ ID NO:147 provide representative sequences for “knob-bearing” CH2 and CH3 Domains that may be used in the CD137×TA Binding Molecules of the present invention:

SEQ ID NO: 45:
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent,

SEQ ID NO: 147:
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLWCLVK GFYPSDIAVE WESNGQPENN
YKTTPPVLDS DGSFFLYSRL TVDKSRWQEG NVFSCSVMHE
ALHNHYTQKS LSLSLGX

wherein X is a lysine (K) or is absent,

SEQ ID NO: 45:
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent,

SEQ ID NO:148, SEQ ID NO:149 and SEQ ID NO:150 provide representative sequences for “hole-bearing” CH2 and CH3 Domains that may be used in the CD137×TA Binding Molecules of the present invention:

SEQ ID NO: 148:
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 149:
APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 150:
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLSCAVK GFYPSDIAVE WESNGQPENN
YKTTPPVLDS DGSFFLVSRL TVDKSRWQEG NVFSCSVMHE
ALHNRYTQKS LSLSLGX
wherein X is a lysine (K) or is absent.

wherein X is a lysine (K) or is absent.

SEQ ID NO: 148:
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 149:
APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 150:
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLSCAVK GFYPSDIAVE WESNGQPENN
YKTTPPVLDS DGSFFLVSRL TVDKSRWQEG NVFSCSVMHE
ALHNRYTQKS LSLSLGX
wherein X is a lysine (K) or is absent.

wherein X is a lysine (K) or is absent.

SEQ ID NO: 148:
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 149:
APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX
wherein X is a lysine (K) or is absent.
SEQ ID NO: 150:
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLSCAVK GFYPSDIAVE WESNGQPENN
YKTTPPVLDS DGSFFLVSRL TVDKSRWQEG NVFSCSVMHE
ALHNRYTQKS LSLSLGX
wherein X is a lysine (K) or is absent.

wherein X is a lysine (K) or is absent.

As will be noted, the CH2-CH3 Domains of SEQ ID NOs:47 and 50 are IgG4 Domains, while the CH2-CH3 Domains of SEQ ID NOs:45, 146, 148 and 149 are IgG1 Domains. SEQ ID NOs:45, 146, 148 and 149 include a substitution at position 234 with alanine and 235 with alanine, and thus form an Fc Domain that exhibits decreased (or substantially no) binding to FcTRIA (CD64), FcTRIIA (CD32A), FcTRIIB (CD32B), FcTRIIIA (CD16a) or FcTRIIIB (CD16b) (relative to the binding exhibited by the wild-type Fc region (SEQ ID NO:12). The present invention specifically encompasses CD137×TA Binding Molecules comprising CH2-CH3 Domains from any class of human IgG comprising the substitutions described herein (e.g., M252Y/S254T/T256E; T366W; T366S/L368A/Y407V; and/or H435R). Furthermore, specifically encompassed by the instant invention are CD137×TA Binding Molecule constructs lacking the above-indicated C-terminal lysine residue.

In the embodiment described above, the first polypeptide chain will have a “knob-bearing” CH2-CH3 sequence, such as that of SEQ ID NOs:45, 146, and 147 and the third polypeptide chain will have a “hole-bearing” CH2-CH3 sequence, such as that of SEQ ID NO: 148, 149, and 150. However, as will be recognized, a “hole-bearing” CH2-CH3 Domain (e.g., SEQ ID NO:48) could be employed in the first polypeptide chain, in which case, a “knob-bearing” CH2-CH3 Domain (e.g., SEQ ID NO:45) would be employed in the third polypeptide chain.

### 5. Representative Tumor Antigens (TA) and Representative Variable Domains

The CD137×TA Binding Molecules of the present invention comprise at least one epitope-binding site specific for an epitope of a tumor antigen. Representative Tumor Antigens (“TAs”), which may be bound by the CD137×TA Binding Molecules of the present invention include, but are not limited to those presented in Table 1, and which may be referred to herein by a common name, short name, and/or a gene name.

TABLE 1
Representative Tumor Antigens

| | | see e.g., |
|---|---|---|
| Protein Tumor Antigen | Gene Name(s) | UniProtKB ID No. |
| Alpha-N-acetylgalactosaminide | ST6GALNAC6; | Q969X2 |
| alpha-2,6-sialyltransferase 6 | CA19-9 | |
| 5,6-dihydroxyindole-2- | TYRP1; gp75 | P17643 |
| carboxylic acid oxidase | | |
| Activated leukocyte cell adhesion | ALCAM; CD166 | Q13740 |
| molecule | | |
| Alpha-1,4-N- | A4GNT | Q9UNA3 |
| acetylglucosaminyltransferase | | |
| B melanoma antigen 1 | BAGE; CT2.1 | Q13072 |
| Basigin | BSG; CD147 | P35613 |
| B-cell antigen receptor complex- | CD79A | P11912 |
| associated protein alpha chain | | |
| B-cell antigen receptor complex- | CD79B | P40259 |
| associated protein beta chain | | |
| B-cell receptor CD22 | BL-CAM; CD22 | P20273 |
| B-lymphocyte antigen CD19 | CD19 | P15391 |
| B-lymphocyte antigen CD20 | MS4A1; CD20 | P11836 |
| Bone marrow stromal antigen 2 | BST2; CD317 | Q10589 |
| Campath-1 antigen | CD52 | P31358 |
| Carbonic anhydrase 14 | CA14 | Q9ULX7 |
| Carboxypeptidase M | CPM | P14384 |
| Carcinoembryonic antigen- | CEACAM5; | P06731 |
| related cell adhesion molecule 5 | CD66e | |
| Carcinoembryonic antigen- | CEACAM6; | P40199 |
| related cell adhesion molecule 6 | CD66c | |
| Catenin beta-1 | CTNNB1; beta- | P35222 |
| | catenin | |
| CD27 antigen | CD27 | P26842 |
| CD276 antigen | CD276; B7-H3 | Q5ZPR3 |
| CD40 ligand | CD40LG; CD154 | P29965 |
| Cell surface A33 antigen | GPA33 | Q99795 |
| Chondroitin sulfate proteoglycan | CSPG4 | Q6UVK1 |
| 4 | | |
| C-type lectin domain family | CLEC4C; | Q8WTT0 |
| member 4 C | BDCA2; CD303 | |
| Cyclin-dependent kinase 4 | CDK4 | P11802 |
| Cytotoxic T-lymphocyte protein | CTLA4 | P16410 |
| 4 | | |
| Disintegrin and metalloproteinase | ADAM-9 | Q13443 |
| domain-containing protein 9 | | |
| Ephrin type-A receptor 2 | EPHA2 | P29317 |
| Epidermal growth factor receptor | EGFR; ERBB1; | P00533 |
| | HER1 | |
| Epithelial cell adhesion molecule | EPCAM; CD326 | P16422 |
| G antigen 1 | GAGEl; CT4.1 | Q13065 |
| G antigen 2A | GAGE2A | Q6NT46 |
| G antigen 2B/C | GAGE2B | Q13066 |
| G antigen 2D | GAGE2D | Q9UEU5 |
| G antigen 2E | GAGE2E | Q4V326 |
| G2/mitotic-specific cyclin-B1 | CCNB1 | P14635 |
| GDP-L-fucose synthase | TSTA3 | Q13630 |
| Glutamate carboxypeptidase 2 | FOLH1; PSMA | Q04609 |
| Hyaluronidase-2 | HYLA2; LUCA2 | Q12891 |
| Inactive tyrosine-protein kinase | ROR1, NTRKR1 | Q01973 |
| transmembrane receptor ROR1 | | |
| Integrin alpha-E | ITGAE; CD103 | P38570 |
| Integrin beta-6 | ITGB6 | P18564 |
| Interleukin-13 receptor subunit | IL13RA2; | Q14627 |
| alpha-2 (subunit of CD 123, | CD213a2 | |
| interleukin-3 receptor) | | |
| Interleukin-2 receptor subunit | IL2RA: CD25 | P01589 |
| alpha | | |
| Junctional adhesion molecule C | JAM3 | Q9BX67 |
| Keratin, type II cytoskeletal 8 | CK-8; KRT8 | P05787 |
| Lactadherin | MFGE8 | Q08431 |
| Low affinity immunoglobulin | FCER2; CD23 | P06734 |
| epsilon Fc receptor | | |
| Melanocyte protein PMEL | PMEL; gp100 | P40967 |
| Melanoma antigen recognized by | MLANA; | Q16655 |
| T-cells 1 | MART1 | |
| Melanoma-associated antigen 1 | MAGEA1; | P43355 |
| | MAGE1 | |
| Melanoma-associated antigen 3 | MAGEA3; | P43357 |
| | MAGE3 | |
| Melanotransferrin | MELTF; | P08582 |
| | MAAp97; | |
| | CD228 | |
| Membrane cofactor protein | CD46 | P15529 |
| Mesothelin | MSLN | Q13421 |
| Mucin-1 | MUC1; PEM | P15941 |
| Mucin-16 | MUC16; CA-125 | Q8WXI7 |
| Myeloid cell surface antigen | CD33 | P20138 |
| CD33 | | |
| Neural cell adhesion molecule 1 | NCAM1; CD56 | P13591 |
| Oncostatin-M | OSM | P13725 |
| Oncostatin-M-specific receptor | OSMR; IL31RB | Q99650 |
| subunit beta | | |
| Platelet glycoprotein 4 | CD36 | P16671 |
| Programmed cell death 1 ligand 1 | CD274 | Q9NZQ7 |
| Prosaposin receptor GPR37 | GPR37 | O15354 |
| Prostate-specific antigen | KLK3; PSA | P07288 |
| Prostatic acid phosphatase | ACPP | P15309 |
| Protein PML | PML; TRIM19; | P29590 |
| | Myl | |
| PWWP domain-containing DNA | PWWP3A; | Q2TAK8 |
| repair factor 3A | MUM1 | |
| Receptor tyrosine-protein kinase | ERBB2; HER2; | P04626 |
| erbB-2 | CD340 | |
| Receptor tyrosine-protein kinase | ERBB3; HER3 | P21860 |
| erbB-3 | | |
| Receptor tyrosine-protein kinase | ERBB4; HER4 | Q15303 |
| erbB-4 | | |
| Receptor-type tyrosine-protein | PTPRC; CD45 | P08575 |
| phosphatase C | | |
| T-cell surface glycoprotein CD5 | CD5 | P06127 |
| T-cell-specific surface | CD28 | Pl0747 |
| glycoprotein CD28 | | |
| Transferrin receptor protein 1 | TFRC; CD71 | P02786 |
| Transmembrane 4 L6 family | TM4SF1; | P30408 |
| member 1 | TAAL6 | |
| Trophoblast glycoprotein | TPBG; 5T4 | Q13641 |
| Tumor necrosis factor receptor | TNFRSF10B; | 014763 |
| superfamily member 10B | DR5; CD262 | |
| Tumor necrosis factor receptor | TNFRSF1A; | P19438 |
| superfamily member 1A | TNFR1; CD120a | |
| Tumor necrosis factor receptor | TNFRSF1B; | P20333 |
| superfamily member 1B | TNFR2; CD120b | |
| Tumor necrosis factor receptor | LTBR; TNFR3 | P36941 |
| superfamily member 3 | | |
| Tumor necrosis factor receptor | CD40 | P25942 |
| superfamily member 5 | | |
| Tumor necrosis factor receptor | TNFR6; Apo-1; | P25445 |
| superfamily member 6 | Fas; CD95 | |
| Ubiquitin-conjugating enzyme | UBE2K | P61086 |
| E2 K | | |
| Ubiquitin-protein ligase E3A | UBE3A | Q05086 |
| Vascular endothelial growth | VEGFA | P15692 |
| factor A | | |
| Vascular endothelial growth | VEGFB | P49765 |
| factor B | | |
| Vascular endothelial growth | FLT1; VEGFR1 | P17948 |
| factor receptor 1 | | |
| Vascular endothelial growth | KDR; VEGFR2; | P35968 |
| factor receptor 2 | CD309 | |
| Vascular endothelial growth | FLT4; VEGFR3 | P35916 |
| factor receptor 3 | | |
| Zinc finger protein 354C | ZNF354C; KID3 | Q86Y25 |

| Other Tumor Antigen(s) | see e.g., Citation(s) |
|---|---|
| 3-fucosyl-N-acetyllactosamine | Gooi, H. C. (1983), “Marker of |
| | peripheral blood granulocytes and |
| | monocytes of man recognized by two |
| | monoclonal antibodies VEP8 and |
| | VEP9 involves the trisaccharide 3- |
| | fucosyl-N-acetyllactosamine,” Eur. J. |
| | Immuno. 13(4):306-12. |
| Blood group A antigen | Gooi, H. C., et al. (1983), |
| | “Monoclonal antibody reactive with |
| | the human epidermal-growth-factor |
| | receptor recognizes the blood-group- |
| | A antigen,” Biosci. Rep. 3(11):1045- |
| | 52. |
| Difucosyl type 1 chain (Aleb) | Dohi, T. et al. (1989), |
| Difucosyl type 2 chain (ALey) | “Immunohistochemical Study of |
| | carbohydrate antigen expression in |
| | gastric carcinoma,” Gastroenterol Jpn. |
| | 24(3): 239-45; Yazawa, S. et al. |
| | (1993), “Aberrant |
| | alpha1-->2Fucosyltransferases Found |
| | in Human Colorectal Carcinoma |
| | Involved in the Accumulation of Leb |
| | and Y Antigens in Colorectal |
| | Tumors,” Jpn. J. Cancer Res. 84:989- |
| | 995 |
| Ganglioside antigen 4.2 | Nudelman, E. et al. (1982), |
| | “Characterization of a human |
| | melanoma-associated ganglioside |
| | antigen defined by monoclonal |
| | antibody, 4.2,” J. Biol. Chern. |
| | 257(21): 12752-6 |
| Ganglioside antigen D1.1 | Levine, J. M., et al. (1984), “The |
| | D1.1 antigen: a cell surface marker for |
| | germinal cells of the central nervous |
| | system,” J. Neurosci. 4(3): 820-31 |
| Gangliosides | Krengel, U. and Bousquet P. A. |
| GD2/GD3/GM2/GM3 | (2014), “Molecular Recognition of |
| | Gangliosides and Their Potential for |
| | Cancer Immunotherapies,” Front. |
| | Immuno. 5(325): 1-11 |
| Lactosylceramide | Symington, F. W. (1984), |
| | “Monoclonal Antibody Specific for |
| | Lactosylceramide,” J. Biol. Chem. |
| | 259(9):6008-6012 |
| Rh antigens (D, C, c, E or e) | Avent, N. D. and Reid, M. E. (2000), |
| | “The Rh blood group system: a |
| | review,” Blood 95:375-387 |
| Sialyl-Tn | Holmberg, L. A. (2001) “Theratope |
| | Vaccine (STn-KLH),” Expert Opin. |
| | Biol. Ther. 1(5):881-91 |

Antibodies that recognize TAs are known in the art or can be generated using well-known methods, including those described herein. Representative antibodies that comprise VL and VH Domains capable of binding to a TA, and whose sequences or polypeptide chains may thus be employed in the construction of the CD137×TA Binding Molecules of the present invention, are listed in Table 2. Representative VH and VL Domains for antibodies binding to several Tumor Antigens are presented below.

TABLE 2

| Antibody Name | Tumor Antigen(s) | Therapeutic Target Application |
|---|---|---|
| Abagovomab | CA-125 | Ovarian Cancer |
| Adecatumumab | Epcam | Prostate And Breast Cancer |
| Afutuzumab | CD20 | Lymphoma |
| Alacizumab | VEGFR2 | Cancer |
| Altumomab | CEA | Colorectal Cancer |
| Amatuximab | Mesothelin | Cancer |
| Anatumomab | TAG-72 | Non-Small Cell Lung Carcinoma |
| Mafenatox | | |
| Anifrolumab | Interferon A/B | Systemic Lupus Erythematosus |
| | Receptor | |
| Anrukinzumab | IL-13 | Cancer |
| Apolizumab | HLA-DR | Hematological Cancers |
| Arcitumomab | CEA | Gastrointestinal Cancer |
| Atinumab | RTN4 | Cancer |
| Bectumomab | CD22 | Non-Hodgkin's Lymphoma |
| | | (Detection) |
| Belimumab | BAFF | Non-Hodgkin Lymphoma |
| Bevacizumab | VEGF-A | Metastatic Cancer, Retinopathy |
| | | Of Prematurity |
| Bivatuzumab | CD44 V6 | Squamous Cell Carcinoma |
| Blinatumomab | CD19 | Cancer |
| Brentuximab | CD30 (TNFRSF8) | Hematologic Cancers |
| Cantuzumab | MUC1 | Cancers |
| Cantuzumab | Mucin Canag | Colorectal Cancer |
| Mertansine | | |
| Caplacizumab | VWF | Cancers |
| Capromab | Prostatic | Prostate Cancer (Detection) |
| | Carcinoma Cells | |
| Carlumab | MCP-1 | Oncology/Immune Indications |
| Catumaxomab | Epcam, CD3 | Ovarian Cancer, Malignant |
| | | Ascites, Gastric Cancer |
| Cetuximab | EGFR | Metastatic Colorectal Cancer |
| | | And Head And Neck Cancer |
| Citatuzumab | Epcam | Ovarian Cancer And Other Solid |
| | | Tumors |
| Cixutumumab | IGF-1 Receptor | Solid Tumors |
| Clivatuzumab | MUC1 | Pancreatic Cancer |
| Conatumumab | TRAIL-R2 | Cancer |
| Dacetuzumab | CD40 | Hematologic Cancers |
| Dalotuzumab | Insulin-Like | Cancer |
| | Growth Factor 1 | |
| | Receptor | |
| Daratumumab | CD38 | Cancer |
| Demcizumab | DLL4 | Cancer |
| Denintuzumab | CD19 | Acute Lymphoblastic Leukemia |
| | | And B Cell Non-Hodgkin |
| | | Lymphoma |
| Detumomab | B-Lymphoma Cell | Lymphoma |
| Drozitumab | DR5 | Cancer |
| Duligotumab | HER3 | Cancer |
| Dusigitumab | ILGF2 | Cancer |
| Ecromeximab | GD3 Ganglioside | Malignant Melanoma |
| Edrecolomab | Epcam | Colorectal Carcinoma |
| Elotuzumab | SLAMF7 | Multiple Myeloma |
| Elsilimomab | IL-6 | Cancer |
| Enavatuzumab | TWEAK Receptor | Cancer |
| Enlimomab | ICAM-1 (CD54) | Cancer |
| Enoblituzumab | B7-H3 | Cancer |
| Enoticumab | DLL4 | Cancer |
| Ensituximab | 5AC | Cancer |
| Epitumomab | Episialin | Cancer |
| Cituxetan | | |
| Epratuzumab | CD22 | Cancer, SLE |
| Ertumaxomab | HER2, CD3 | Breast Cancer |
| Etaracizumab | Integrin Aᵥβ₃ | Melanoma, Prostate Cancer, |
| | | Ovarian Cancer |
| Faralimomab | Interferon | Cancer |
| | Receptor | |
| Farletuzumab | Folate Receptor 1 | Ovarian Cancer |
| Fasinumab | HNGF | Cancer |
| FbtaO5 (Bi20) | CD20 | Chronic Lymphocytic Leukemia |
| Ficlatuzumab | HGF | Cancer |
| Figitumumab | IGF-1 Receptor | Adrenocortical Carcinoma, Non- |
| | | Small Cell Lung Carcinoma |
| Flanvotumab | TYRP1 | Melanoma |
| | (Glycoprotein 75) | |
| Flotetuzumab | CD123 | Acute Myeloid Leukemia |
| Fresolimumab | TGF-B | Cancer |
| Futuximab | EGFR | Cancer |
| Galiximab | CD80 | B Cell Lymphoma |
| Ganitumab | IGF-I | Cancer |
| Gemtuzumab | CD33 | Acute Myelogenous Leukemia |
| Ozogamicin | | |
| Girentuximab | Carbonic | Clear Cell Renal Cell Carcinoma |
| | Anhydrase 9 (CA- | |
| | IX) | |
| Glembatumumab | GPNMB | Melanoma, Breast Cancer |
| Vedotin | | |
| Ibritumomab | CD20 | Non-Hodgkin's Lymphoma |
| Tiuxetan | | |
| Icrucumab | VEGFR-1 | Cancer |
| Imgatuzumab | EGFR | Cancer |
| Inclacumab | Selectin P | Cancer |
| Indatuximab | SDC1 | Cancer |
| Ravtansine | | |
| Inotuzumab | CD22 | Cancer |
| Ozogamicin | | |
| Intetumumab | CD51 | Solid Tumors (Prostate Cancer, |
| | | Melanoma) |
| Ipilimumab | CD 152 | Melanoma |
| Iratumumab | CD30 (TNFRSF8) | Hodgkin's Lymphoma |
| Itolizumab | CD6 | Cancer |
| Labetuzumab | CEA | Colorectal Cancer |
| Lampalizumab | CFD | Cancer |
| Lebrikizumab | Il-13 | Hodgkin's Lymphoma |
| Lexatumumab | TRAIL-R2 | Cancer |
| Ligelizumab | IGHE | Cancer |
| Lintuzumab | CD33 | Cancer |
| Lirilumab | KIR2D | Cancer |
| Lorvotuzumab | CD56 | Cancer |
| Lucatumumab | CD40 | Multiple Myeloma, Non- |
| | | Hodgkin's Lymphoma, |
| | | Hodgkin's Lymphoma |
| Lumiliximab | CD23 | Chronic Lymphocytic Leukemia |
| Mapatumumab | TRAIL-R1 | Cancer |
| Margetuximab | HER2 | HER2 positive Cancer |
| Matuzumab | EGFR | Colorectal, Lung And Stomach |
| | | Cancer |
| Milatuzumab | CD74 | Multiple Myeloma And Other |
| | | Hematological Malignancies |
| Minretumomab | TAG-72 | Cancer |
| Mitumomab | GD3 Ganglioside | Small Cell Lung Carcinoma |
| Mogamulizumab | CCR4 | Cancer |
| Morolimumab | Rhesus Factor | Cancer |
| Moxetumomab | CD22 | Cancer |
| Pasudotox | | |
| Nacolomab | C242 Antigen | Colorectal Cancer |
| Tafenatox | | |
| Namilumab | CSF2 | Cancer |
| Naptumomab | 5T4 | Non-Small Cell Lung |
| Estafenatox | | Carcinoma, Renal Cell |
| | | Carcinoma |
| Narnatumab | RON | Cancer |
| Naxitamab | GD2 | Neuroblastoma, Osteosarcoma |
| Necitumumab | EGFR | Non-Small Cell Lung Carcinoma |
| Nerelimomab | TNF-A | Cancer |
| Nesvacumab | Angiopoietin 2 | Cancer |
| Nimotuzumab | EGFR | Squamous Cell Carcinoma, Head |
| | | And Neck Cancer, |
| | | Nasopharyngeal Cancer, Glioma |
| Nivolumab | PD-1 | Cancer |
| Nofetumomab | Undetermined | Cancer |
| Merpentan | | |
| Ocaratuzumab | CD20 | Cancer |
| Ofatumumab | CD20 | Chronic Lymphocytic Leukemia |
| Olaratumab | PDGF-R A | Cancer |
| Olokizumab | IL6 | Cancer |
| Omburtamab | B7-H3 | Neuroblastoma, Sarcoma, |
| | | Metastatic Brain Cancers |
| Onartuzumab | Human Scatter | Cancer |
| | Factor Receptor | |
| | Kinase | |
| Ontuxizumab | TEM1 | Cancer |
| Oportuzumab | Epcam | Cancer |
| Monatox | | |
| Oregovomab | CA-125 | Ovarian Cancer |
| Orticumab | Oxldl | Cancer |
| Otlertuzumab | CD37 | Cancer |
| Panitumumab | EGFR | Colorectal Cancer |
| Pankomab | Tumor Specific | Ovarian Cancer |
| | Glycosylation Of | |
| | MUC1 | |
| Parsatuzumab | EGFL7 | Cancer |
| Patritumab | HER3 | Cancer |
| Pembrolizumab | PD-1 | Cancer |
| Pemtumomab | MUC1 | Cancer |
| Perakizumab | IL17A | Arthritis |
| Pertuzumab | HER2 | Cancer |
| Pidilizumab | PD-1 | Cancer |
| Pinatuzumab | CD22 | Cancer |
| Vedotin | | |
| Pintumomab | Adenocarcinoma | Adenocarcinoma |
| | Antigen | |
| Placulumab | Human TNF | Cancer |
| Polatuzumab | CD79B | Cancer |
| Vedotin | | |
| Pritoxaximab | E. Coli Shiga | Cancer |
| | Toxin Type-1 | |
| Pritumumab | Vimentin | Brain Cancer |
| Quilizumab | IGHE | Cancer |
| Racotumomab | N- | Cancer |
| | Glycolylneuraminic | |
| | Acid | |
| Radretumab | Fibronectin Extra | Cancer |
| | Domain-B | |
| Ramucirumab | VEGFR2 | Solid Tumors |
| Rilotumumab | HGF | Solid Tumors |
| Rituximab | CD20 | Lymphomas, Leukemias, Some |
| | | Autoimmune Disorders |
| Robatumumab | IGF-1 Receptor | Cancer |
| Roledumab | RHD | Cancer |
| Samalizumab | CD200 | Cancer |
| Satumomab | TAG-72 | Cancer |
| Pendetide | | |
| Seribantumab | ERBB3 | Cancer |
| Sibrotuzumab | FAP | Cancer |
| Siltuximab | IL-6 | Cancer |
| Solitomab | Epcam | Cancer |
| Sontuzumab | Episialin | Cancer |
| Tabalumab | BAFF | B Cell Cancers |
| Tacatuzumab | Alpha-F etoprotein | Cancer |
| Tetraxetan | | |
| Taplitumomab | CD19 | Cancer |
| Paptox | | |
| Telimomab | Undetermined | Cancer |
| Tenatumomab | Tenascin C | Cancer |
| Teneliximab | CD40 | Cancer |
| Teprotumumab | CD221 | Hematologic Tumors |
| Ticilimumab | CTLA-4 | Cancer |
| Tigatuzumab | TRAIL-R2 | Cancer |
| Tositumomab | CD20 | Follicular Lymphoma |
| Tovetumab | CD140a | Cancer |
| Trastuzumab | HER2 | Breast Cancer |
| Trbs07 | Gd2 | Melanoma |
| (Ektomab) | | |
| Tremelimumab | CTLA-4 | Cancer |
| Tucotuzumab | Epcam | Cancer |
| Celmoleukin | | |
| Ublituximab | MS4A1 | Cancer |
| Urelumab | 4-1BB | Cancer |
| Vadastuximab | CD33 | Acute Myeloid Leukemia |
| Vantictumab | Frizzled Receptor | Cancer |
| Vapaliximab | AOC3 (VAP-1) | Cancer |
| Vatelizumab | ITGA2 | Cancer |
| Veltuzumab | CD20 | Non-Hodgkin's Lymphoma |
| Vesencumab | NRP1 | Cancer |
| Volociximab | Integrin A5β1 | Solid Tumors |
| Vorsetuzumab | CD70 | Cancer |
| Votumumab | Tumor Antigen | Colorectal Tumors |
| | CTAA16.88 | |
| Zalutumumab | EGFR | Squamous Cell Carcinoma Of |
| | | The Head And Neck |
| Zatuximab | HER1 | Cancer |
| Ziralimumab | CD147 | Cancer |
| Zolbetuximab | Cldn18.2 | Gastrointestinal |
| | | Adenocarcinomas And |
| | | Pancreatic Tumor |

PD-L1 (also known as CD274 and B37-H1), is a 40 kDa transmembrane protein commonly expressed on the surface of T lymphocytes, B lymphocytes, DCs, macrophages and in non-blood cells. In addition, PD-L1 also shows abnormally high expression in tumor cells, which is considered the main factor responsible for promoting the ability of tumor immune escape. Engagement of PD-L1 with its receptor, PD-1 on T cells activates the down-stream signaling of PD-1 receptor delivering a signal that inhibits the proliferation, cytokine generation and release, and cytotoxicity of T cells. Antibodies which block PD-L1/PD-1 disrupt PD-1 axis thereby reverses T cell suppression and enhances endogenous antitumor immunity. CD137×TA Binding Molecules that bind PD-L1 can co-ligate tumor cells expressing PD-L1, and immune cells expressing CD137. Without being limited to any particular method, such co-localization can stimulate the immune cells, while also attenuating or blocking the immune system inhibition that occurs upon PD-L1-PD-1 binding.

The epitope-binding site of any anti-PD-L1 antibody may be used in accordance with the present invention, and the principles of the present invention are illustrated with respect to the PD-L1 tumor antigen. Representative antibodies that bind human PD-L1 include atezolizumab, avelumab, and durvalumab, each recently approved for use in humans. Atezolizumab (marketed as TECENTRIQ®; CAS Reg No. 1380723-44-3; see, U.S. Pat. No. 9,873,740) is a humanized monoclonal antibody having modified IgG1 and kappa constant regions. Avelumab (marketed as BAVENCIO®; CAS Reg No. 1537032-82-8; see, U.S. Pat. No. 9,873,740) is a fully human monoclonal antibody having IgG1/lambda constant regions. Durvalumab (marked as IMFINZI®; CAS Reg. No. 1428935-60-7; see U.S. Pat. No. 8,779,108) is a fully human monoclonal antibody having modified IgG1 and kappa constant regions. The amino acid sequence of the complete heavy and Light Chains of atezolizumab (WHO Drug Information, 2015, Recommended INN: List 74, 29(3):387), durvalumab (WHO Drug Information, 2015, Recommended INN: List 74, 29(3):393-394) and avelumab (WHO Drug Information, 2016, Recommended INN: List 74, 30(1):100-101) are known in the art. Additional anti-PD-L1 antibodies, including the humanized anti-PD-L1 antibody “hPD-L1 MAB-2” and optimized variants thereof, are also provided herein.

(1) hPD-L1 MAB-2

The amino acid sequence of the VH Domain of hPD-L1 MAB-2 (hPD-L1 MAB-2 VH1) is (SEQ ID NO:57) (CDR_{H} residues are shown underlined):

EVQLVESGGG LVQPGGSLRL SCAASGFTFS SYTMSWVRQA
PGKGLEWVAY ISIGGGTTYY PDTVKGRFTI SRDNAKNTLY
LQMNSLKTED TAVYYCARQG LPYYFDYWGQ GTLVTVSS

The amino acid sequence of the VL Domain of hPD-L1 MAB-2 (hPD-L1 MAB-2 VL1) is (SEQ ID NO:58) (CDR_{L} residues are shown underlined):

DIQMTQSPSS LSASVGDRVT ITCKASQDVN TAVAWYQQKP
GKAPKLLIYW ASTRHTGVPS RFSGSGSGTD FTLTISSLQP
EDFATYYCQQ HYNTPLTFGQ GTKVEIK

(2) Deimmunized and Optimized hPD-L1 MAB-2

As described in the examples below hPD-L1 MAB-2 was deimmunized and optimized for binding and expression to yield variant VH Domains designated “hPD-L1 MAB-2 VHx” and VL Domains designated “hPD-L1 MAB-2 VLx.” The amino acid sequences of particular deimmunized and optimized variant VH and VL Domains are presented below, additional variants are provided in the Examples.

The amino acid sequence of hPD-L1 MAB-2 VHx is (SEQ ID NO:59) (CDR_{H} residues are shown underlined):

EVQLVESGGG LVQPGGSLRL SCAASGFTFS SYTMSWVRQA
PGKGLEWVAY ISIX₄GGTTYY PDTVKGRFTI SRDNAKNX₅LY
LQMNSLX₆X₇ED TAVYYCARX₈G LPYYX₉DYWGQ GTLVTVSS

wherein: X₄, X₅, X₆, X₇, X₈, and X₉ are independently selected, and
wherein: X₄ is G or K; X₅ is S or T; X₆ is K or R; X₇ is A or T; X₈ is A or Q; and X₉ is F or G.

In specific embodiments:
a) X₄ is G; X₅ is S; X₆ is R; X₇ is A; X₈ is Q; and X₉ is F;
b) X₄ is K; X₅ is S; X₆ is R; X₇ is A; X₈ is Q; and X₉ is G;
c) X₄ is G; X₅ is S; X₆ is R; X₇ is A; X₈ is A; and X₉ is F;
d) X₄ is K; X₅ is S; X₆ is R; X₇ is A; X₈ is A; and X₉ is F;
e) X₄ is G; X₅ is S; X₆ is R; X₇ is A; X₈ is A; and X₉ is G; or
f) X₄ is K; X₅ is S; X₆ is R; X₇ is A; X₈ is Q; and X₉ is F.

The amino acid sequences of the CDR_{H}s of hPD-L1 MAB-2 VHx are:

CDR_{H}1 (SEQ ID NO: 60):
SYTMS
CDR_{H}2 (SEQ ID NO: 61):
YISIX₄GGTTYYPDTVKG
CDR_{H}3 (SEQ ID NO: 62):
X₈GLPYYX₉DY

wherein: X₄ is G or K; X₈ is A or Q; and X₉ is F or G

The amino acid sequence of hPD-L1 MAB-2 VLx is (SEQ ID NO:63) (CDR_{L} residues are shown underlined):

DIQMTQSPSS LSASVGDRVT ITCKASQDVN X₁₀AVAWYQQKP
GKAPKLLIYW ASTRHTGVPS RFSGSGSGTD FTLTISSLQP
EDFATYYCQQ HYNTPLTFGQ GTKVEIK

wherein: X₁₀ is E or T.

In a specific embodiment, X₁₀ is E.

The amino acid sequences of the CDR_{L}s of IPD-L1 MAB-2 VLx are:

CDR_{L}1 (SEQ ID NO: 64):
KASQDVNX₁₀AVA
CDR_{L}2 (SEQ ID NO: 65):
WASTRHT
CDR_{L}3 (SEQ ID NO: 66):
QQHYNTPLT

wherein: X₁₀ is E or T.

The amino acid sequences of five variant VH Domain designated herein as “hPD-L1 MAB-2 VH2,” “hPD-L1 MAB-2 VH3,” “hPD-L1 MAB-2 VH4,” “hPD-L1 MAB-2 VH5,” “hPD-L1 MAB-2 VH6,” and one variant VL Domain designated herein as “hPD-L1 MAB-2 VL2” are presented below. Any of the variant hPD-L1 MAB-2 VH Domains disclosed herein may be paired with any of the hPD-L1 MAB-2 VL domains. Molecules comprising particular combinations of PD-L1 MAB-2 VH/VL Domains are referred to by reference to the specific VH/VL Domains, for example, a molecule comprising the binding domains PD-L1 MAB-2 VH3 and hPD-L1 MAB-2 VL2 is specifically referred to as “PD-L1 MAB-2(3.2).” The amino acid sequences of the variant VH and VL Domains are provided below, substitutions in the CDRs relative to VH1 or VL1 are double underlined.

The amino acid sequence of hPD-L1 MAB-2 VH2 is (SEQ ID NO:67) (CDR_{H} residues are shown underlined):

EVQLVESGGG LVQPGGSLRL SCAASGFTFS SYTMSWVRQA
PGKGLEWVAY ISIGGGTTYY PDTVKGRFTI SRDNAKNSLY
LQMNSLRAED TAVYYCARQG LPYYFDYWGQ GTLVTVSS

The amino acid sequence of hPD-L1 MAB-2 VH3 is (SEQ ID NO:68) (CDR_{H} residues are shown underlined; substitutions are doubled underlined):

EVQLVESGGG LVQPGGSLRL SCAASGFTFS SYTMSWVRQA
PGKGLEWVAY ISIKGGTTYY PDTVKGRFTI SRDNAKNSLY
LQMNSLRAED TAVYYCARQG LPYYGDYWGQ GTLVTVSS

The amino acid sequence of hPD-L1 MAB-2 VH4 is (SEQ ID NO:69) (CDR_{H} residues are shown underlined; substitutions are doubled underlined):

EVQLVESGGG LVQPGGSLRL SCAASGFTFS SYTMSWVRQA
PGKGLEWVAY ISIGGGTTYY PDTVKGRFTI SRDNAKNSLY
LQMNSLRAED TAVYYCARAG LPYYFDYWGQ GTLVTVSS

The amino acid sequence of hPD-L1 MAB-2 VH5 is (SEQ ID NO:70) (CDR_{H} residues are shown underlined)

EVQLVESGGG LVQPGGSLRL SCAASGFTFS SYTMSWVRQA
PGKGLEWVAY ISIKGGTTYY PDTVKGRFTI SRDNAKNSLY
LQMNSLRAED TAVYYCARAG LPYYFDYWGQ GTLVTVSS

The amino acid sequence of hPD-L1 MAB-2 VH6 is (SEQ ID NO:71) (CDR_{H} residues are shown underlined):

EVQLVESGGG LVQPGGSLRL SCAASGFTFS SYTMSWVRQA
PGKGLEWVAY ISIGGGTTYY PDTVKGRFTI SRDNAKNSLY
LQMNSLRAED TAVYYCARAG LPYYGDYWGQ GTLVTVSS

The amino acid sequence of hPD-L1 MAB-2 VL2 is (SEQ ID NO:72) (CDR_{L} residues are shown underlined):

DIQMTQSPSS LSASVGDRVT ITCKASQDVN EAVAWYQQKP
GKAPKLLIYW ASTRHTGVPS RFSGSGSGTD FTLTISSLQP
EDFATYYCQQ HYNTPLTFGQ GTKVEIK

It will be noted that the amino acid sequences of CDR_{H}2, CDR_{H}3, and CDR_{L}1 of the optimized hPD-L1 MAB-2 variants differ from those present in the parental molecule. The different CDRs are summarized below with differences from VH1 and VL1 double underlined.

| | | hPD-L1 MAB-2 |
|---|---|---|
| CDR | Amino Acid Sequence | variant |
| CDR_{H}2 | YISIKGGTTYYPDTVKG | VH3/VH4 |
| | (SEQ ID NO: 73) | |
| CDR_{H}3 | QGLPYYGDY | VH3 |
| | (SEQ ID NO: 74) | |
| CDR_{H}3 | AGLPYYFDY | VH4/VH5 |
| | (SEQ ID NO: 75) | |
| CDR_{H}3 | AGLPYYGDY | VH6 |
| | (SEQ ID NO: 76) | |
| CDR_{L}1 | KASQDVNEAVA | VL2 |
| | (SEQ ID NO: 77) | |

The present invention specifically includes and encompasses CD137×PD-L1 Binding Molecules that comprise the VL and/or VH Domain, and/or 1, 2 or all 3 of the CDR_{L}s of the VL Region and/or 1, 2 or all 3 of the CDR_{H}s of the VH Domain of any of atezolizumab, avelumab, durvalumab, hPD-L1 MAB-2 and the variants there of, or any of the other anti-PD-L1 antibodies provided herein; and more typically possess 1, 2 or all 3 of the CDRLs of the VL Region and/or 1, 2 or all 3 of the CDR_{H}s of the VH Domain of such anti-PD-L1 monoclonal antibodies.

### b) HER2 Binding Domains

HER2 is a 185 kDa receptor protein that was originally identified as the product of the transforming gene from neuroblastomas of chemically treated rats. HER2 has been extensively investigated because of its role in many human carcinomas include breast and gastric cancers.

The epitope-binding site of any anti-HER2 antibody may be used in accordance with the present invention, and the principles of the present invention are illustrated with respect to the HER2 tumor antigen. Representative antibodies that bind human HER2 include margetuximab, trastuzumab and pertuzumab. Margetuximab (also known as MGAH22; CAS Reg No. 1350624-75-7, see, for example, U.S. Pat. No. 8,802,093) is an Fc-optimized monoclonal antibody that binds to HER2 and mediates enhanced ADCC activity. Trastuzumab (also known as rhuMAB4D5, and marketed as HERCEPTIN®; CAS Reg No 180288-69-1; see, U.S. Pat. No. 5,821,337) is the humanized version of antibody 4D5, having IgG1/kappa constant regions. Pertuzumab (also known as rhuMAB2C4, and marketed as PERJET®; CAS Reg No 380610-27-5; see for example, WO2001/000245) is a humanized version of antibody 2C4 having IgG1/kappa constant regions. The amino acid sequence of the complete heavy and Light Chains of margetuximab (WHO Drug Information, 2014, Recommended INN: List 70, 28(1):93-94), and trastuzumab (see WHO Drug Information, 2011, Recommended INN: List 65, 25(1):89-90 for trastuzumab emtansine), and the Fab Domain of pertuzumab (Protein Data Bank Accession No. 117i) are known in the art. Additional anti-HER2 antibodies, including HER2 MAB-1 and humanized variants thereof, are also provided herein.

(1) hHER2 MAB-1

Antibody hHER2 MAB-1 is a humanized anti-HER2 monoclonal antibody that binds an epitope of HER2 that is distinct from the epitope recognized by Margetuximab, Trastuzumab and Pertuzumab (see, e.g., WO 2018/156740).

The amino acid sequence of the VH Domain of humanized antibody (hHER2 MAB-1 VHx) is (SEQ ID NO:78) (CDR_{H} residues are shown underlined):

QVQLVQSGAE VKKPGASVKV SCKASGYTFT NYGMNWVRQA
PGQGLEWMGW INTNIGEPTY TEEFKGRVTM TRDTSISTAY
MELSRLRSDD TAVYYCARDX₁ X₂YGNRVSYWG QGTLVTVSS

wherein: X₁ is D or E and X₂ is G or I

The amino acid sequence of the VL Domain of such humanized antibody (hHER2 MAB-1 VLx) is (SEQ ID NO:79) (CDR_{L} residues are shown underlined):

DIQMTQSPSS LSASVGDRVT ITCKASQDIX₃ X₄YLSWFQQKP
GKAPKTLIYR ANRLX₅X₆GVPS RFSGSGSGTD FTLTISSLQP
EDFATYYCLQ HDEFPWTFGQ GTKLEIK

wherein: X₃ is N or S; X₄ is S, T or N; X₅ is V or Q and X₆ is D, E or S

Three variant hHER2 MAB-1 VH Domains were isolated: hHER2 MAB-1 VH1, hHER2 MAB-1 VH2, and hHER2 MAB-1 VH3. The amino acid sequences of such variant hHER2 MAB-1 VH Domains are presented below.

The amino acid sequence of hHER2 MAB-1 VH1 is (SEQ ID NO:80) (CDR_{H} residues are shown underlined; note that the second and third residues of CDR_{H}3 are D and G, respectively):

QVQLVQSGAE VKKPGASVKV SCKASGYTFT NYGMNWVRQA
PGQGLEWMGW INTNIGEPTY TEEFKGRVTM TRDTSISTAY
MELSRLRSDD TAVYYCARDD GYGNRVSYWG QGTLVTVSS

The amino acid sequence of hHER2 MAB-1 VH2 is (SEQ ID NO:81) (CDR_{H} residues are shown underlined; note that the second and third residues of CDR_{H}3 are E and G, respectively):

QVQLVQSGAE VKKPGASVKV SCKASGYTFT NYGMNWVRQA
PGQGLEWMGW INTNIGEPTY TEEFKGRVTM TRDTSISTAY
MELSRLRSDD TAVYYCARDE GYGNRVSYWG QGTLVTVSS

The amino acid sequence of hHER2 MAB-1 VH3 is (SEQ ID NO:82) (CDR_{H} residues are shown underlined; note that the second and third residues of CDR_{H}3 are D and I, respectively):

QVQLVQSGAE VKKPGASVKV SCKASGYTFT NYGMNWVRQA
PGQGLEWMGW INTNIGEPTY TEEFKGRVTM TRDTSISTAY
MELSRLRSDD TAVYYCARDD IYGNRVSYWG QGTLVTVSS

Three variant hHER2 MAB-1 VL Domains were isolated: hHER2 MAB-1 VL1, hHER2 MAB-1 VL2, and hHER2 MAB-1 VL3. The amino acid sequences of such variant hHER2 MAB-1 VL Domains are presented below.

The amino acid sequence of hHER2 MAB-1 VL1 is (SEQ ID NO:83) (CDR_{L} residues are shown underlined; note that the seventh and eighth residues of CDR_{L}1 are N and S, respectively, and that the sixth and seventh residues of CDR_{L}2 are V and E, respectively):

DIQMTQSPSS LSASVGDRVT ITCKASQDIN SYLSWFQQKP
GKAPKTLIYR ANRLVDGVPS RFSGSGSGTD FTLTISSLQP
EDFATYYCLQ HDEFPWTFGQ GTKLEIK

The amino acid sequence of hHER2 MAB-1 VL2 is (SEQ ID NO:84) (CDR_{L} residues are shown underlined; note that the seventh and eighth residues of CDR_{L}1 are N and T, respectively, and that the sixth and seventh residues of CDR_{L}2 are V and E, respectively):

DIQMTQSPSS LSASVGDRVT ITCKASQDIN TYLSWFQQKP
GKAPKTLIYR ANRLVEGVPS RFSGSGSGTD FTLTISSLQP
EDFATYYCLQ HDEFPWTFGQ GTKLEIK

The amino acid sequence of hHER2 MAB-1 VL3 is (SEQ ID NO:85) (CDR_{L} residues are shown underlined; note that the seventh and eighth residues of CDR_{L}1 are S and N, respectively, and that the sixth and seventh residues of CDR_{L}2 are Q and S, respectively):

DIQMTQSPSS LSASVGDRVT ITCKASQDIS NYLSWFQQKP
GKAPKTLIYR ANRLQSGVPS RFSGSGSGTD FTLTISSLQP
EDFATYYCLQ HDEFPWTFGQ GTKLEIK

Any of such humanized VH and VL hHER2 MAB-1 Domains, including any embraced within the generic sequence(s) of the hHER2 MAB-1 VH and/or VL Domains presented above may be used to form an antibody, diabody or binding molecule capable of binding Her2.

### (2) Other HER2 Binding Domains

In addition to the above-identified HER2 binding domains, the invention contemplates the use of any of the epitope-binding site of any of the following anti-Her-2 binding domains: 1.44.1; 1.140; 1.43; 1.14.1; 1.100.1; 1.96; 1.18.1; 1.20; 1.39; 1.24; and 1.71.3 (U.S. Pat. Nos. 8,350,011; 8,858,942; and PCT Patent Publication WO 2008/019290); F5 and C1 (U.S. Pat. Nos. 7,892,554; 8,173,424; 8,974,792; and PCT Patent Publication WO 99/55367); and also the binding domains of the anti-HER2 antibodies of US Patent Publication 2011/0097323, 2013/017114, 2014/0328836, 2016/0130360 and 2016/0257761, and PCT Patent Publication WO2011/147986).

The present invention specifically includes and encompasses CD137×HER2 Binding Molecules that comprise the VL and/or VH Domain, and/or 1, 2 or all 3 of the CDR_{L}s of the VL Region and/or 1, 2 or all 3 of the CDR_{H}s of the VH Domain of any of Margetuximab, Trastuzumab, Pertuzumab, hHER2 MAB-1, or any of the other anti-HER2 antibodies provided herein; and more typically possess 1, 2 or all 3 of the CDRLs of the VL Region and/or 1, 2 or all 3 of the CDRHs of the VH Domain of such anti-HER2 monoclonal antibodies.

### c) EphA2 Binding Domains

The receptor tyrosine kinase, ephrin type-A receptor 2 (EphA2) is normally expressed at sites of cell-to-cell contact in adult epithelial tissues, however, recent studies have shown that it is also overexpressed in various types of epithelial carcinomas, with the greatest level of EphA2 expression observed in metastatic lesions. High expression levels of EphA2 have been found in a wide range of cancers and in numerous tumor cell lines, including prostate cancer, breast cancer, non-small cell lung cancer and melanoma. EphA2 does not appear to be merely a marker for cancer, but rather appears to be persistently overexpressed and functionally changed in numerous human cancers. The epitope-binding site of any anti-EphA2 antibody may be used in accordance with the present invention. Presented below are several representative anti-EphA2 antibodies that may be used to generate the molecules of the present invention.

### (1) EphA2 MAB-1

Antibody EphA2 MAB-1 is a murine anti-EphA2 monoclonal antibody. The amino acid sequence of the VH Domain of EphA2 MAB-1 is (SEQ ID NO:86) (CDR residues are shown underlined):

QVQLKESGPG LVAPSQSLSI TCTVSGFSLS RYSVHWVRQP
PGKGLEWLGM IWGGGSTDYN SALKSRLSIS KDNSKSQVFL
KMNSLQTDDT AMYYCARKHG NYYTMDYWGQ GTSVTVSS

The amino acid sequence of the VL Domain of EphA2 MAB-1 is (SEQ ID NO:87) (CDR residues are shown underlined):

DIQMTQTTSS LSASLGDRIT ISCRASQDIS NYLNWYQQKP
DGTVKLLIYY TSRLHSGVPS RFSGSGSGTD YSLTISNLEQ
EDIATYFCQQ GYTLYTFGGG TKLEIK

### (2) EphA2 MAB-2

Antibody EphA2 MAB-2 is a murine anti-EphA2 monoclonal antibody. The amino acid sequence of the VH Domain of EphA2 MAB-2 is (SEQ ID NO:88) (CDR residues are shown underlined):

QIQLVQSGPE LKKPGETVKI SCKASGFTFT NYGMNWVKQA
PGKGLKWMGW INTYIGEPTY ADDFKGRFVF SLETSASTAY
LQINNLKNED MATYFCAREL GPYYFDYWGQ GTTLTVSS

The amino acid sequence of the VL Domain of EphA2 MAB-2 is (SEQ ID NO:89) (CDR residues are shown underlined):

DVVMTQTPLS LPVSLGDQAS ISCRSSQSLV HSSGNTYLHW
YLQKPGQSPK LLIYKVSNRF SGVPDRFSGS GSGTDFTLKI
SRVEAEDLGV YFCSQSTHVP TFGSGTKLEI K

### (3) EphA2 MAB-3

Antibody EphA2 MAB-3 is a murine anti-EphA2 monoclonal antibody. The amino acid sequence of the VH Domain of EphA2 MAB-3 is (SEQ ID NO:90) (CDR residues are shown underlined):

EVQLVESGGG SVKPGGSLKL SCAASGFTFT DHYMYWVRQT
PEKRLEWVAT ISDGGSFTSY PDSVKGRFTI SRDIAKNNLY
LQMSSLKSED TAMYYCTRDE SDRPFPYWGQ GTLVTVSS

The amino acid sequence of the VL Domain of EphA2 MAB-3 is (SEQ ID NO:91) (CDR residues are shown underlined):

DIVLTQSHRS MSTSVGDRVN ITCKASQDVT TAVAWYQQKP
GQSPKLLIFW ASTRHAGVPD RFTGSGSGTD FTLTISSVQA
GDLALYYCQQ HYSTPYTFGG GTKLEIK

### (4) Other EphA2 Binding Domains

In addition to the above-identified EphA2 binding domains, the invention contemplates the use of any of the epitope-binding site of any of the following anti-EphA2 antibodies: SPL1, LUCA19, SG5, or LUCA40 (see, PCT Patent Publication WO 2006/084226); B13 (see, U.S. Pat. No. 7,101,976); D7 (see, U.S. Pat. No. 7,192,698); B-233, and EA2 (see, PCT Patent Publication WO 2003/094859).

The present invention specifically includes and encompasses CD137×EphA2 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-EphA2 monoclonal antibodies EphA2 MAB-1, EphA2 MAB-2 or EphA2 MAB-3.

### d) 5T4 Binding Domains

The oncofetal protein, 5T4, is a tumor-associated protein displayed on the cell membrane of many carcinomas, including kidney, colon, prostate, lung, carcinoma and in acute lymphoblastic leukemia. The epitope-binding site of any anti-5T4 antibody may be used in accordance with the present invention. Presented below are two representative anti-5T4 antibodies, the humanized “5T4 MAB-1,” and the murine “5T4 MAB-2”. Additional ant-5T4 antibodies are described in the art (see, e.g., U.S. Pat. Nos. 8,084,249; 8,409,577; 8,759,495; 8,409,577; PCT Publication Nos: WO 2013/041687; WO 2014/137931; WO 2016/022939)

### (1) 5T4 MAB-1

The amino acid sequence of the VH Domain of 5T4 MAB-1 is (SEQ ID NO:92) (CDR residues are shown underlined):

QVQLVQSGAE VKKPGASVKV SCKASGYTFT SFWMHWVRQA
PGQGLEWMGR IDPNRGGTEY NEKAKSRVTM TADKSTSTAY
MELSSLRSED TAVYYCAGGN PYYPMDYWGQ GTTVTVSS

The amino acid sequence of the VL Domain of 5T4 MAB-1 is (SEQ ID NO:93) (CDR residues are shown underlined):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIK

### (2) 5T4 MAB-2

The amino acid sequence of the VH Domain of 5T4 MAB-2 is (SEQ ID NO:94) (CDR residues are shown underlined):

QVQLQQPGAE LVKPGASVKM SCKASGYTFT SYWITWVKQR
PGQGLEWIGD IYPGSGRANY NEKFKSKATL TVDTSSSTAY
MQLSSLTSED SAVYNCARYG PLFTTVVDPN
SYAMDYWGQG TSVTVSS

The amino acid sequence of the VL Domain of 5T4 MAB-2 is (SEQ ID NO:95) (CDR residues are shown underlined):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IK

The present invention specifically includes and encompasses CD137×5T4 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-5T4 monoclonal antibodies 5T4 MAB-1 or 5T4 MAB-2, or of any of the anti-5T4 antibodies provided in WO 2007/106744; WO 2013/041687 or WO 2015/184203.

### e) B7-H3 Binding Domains

B7-H3 is a Tumor Antigen that is over-expressed on a wide variety of solid tumor types and is a member of the B7 family of molecules that are involved in immune regulation. In particular, several independent studies have shown that human malignant tumor cells (e.g., tumor cells of neuroblastomas and gastric, ovarian and non-small cell lung cancers) exhibit a marked increase in expression of B7-H3 protein and that this increased expression was associated with increased disease severity, suggesting that B7-H3 is exploited by tumors as an immune evasion pathway.

The epitope-binding site of any anti-B7-H3 antibody may be used in accordance with the present invention. One representative humanized antibody that bind human B7-H3 is “Enoblituzumab.” Enoblituzumab (also known as MGA271; CAS Reg No. 1353485-38-7; see for example, U.S. Pat. No. 8,802,091) is an Fc-optimized monoclonal antibody that binds to B7-H3 and mediates enhanced ADCC activity. The amino acid sequence of the complete heavy and Light Chains of Enoblituzumab (WHO Drug Information, 2017, Recommended INN: List 77, 31(1):149) are known in the art. Additional representative anti-B7-H3 antibodies are presented.

(1) hBRCA69D

Representative VH and VL Domains of the humanized anti-B7-H3 antibody “hBRCA69D” are presented below. Two humanized VH Domains, hBRCA69D VH1 and hBRCA69D VH2; and two humanized VL Domains hBRCA69D VL1 and hBRCA69D VL2, which may be used in any combination of VH/VL to yield a functional humanized binding domain are provided below.

The amino acid sequence of the VH Domain of hBRCA69D VH1 is (SEQ ID NO:96) (CDR_{H} residues are shown underlined):

QVQLVQSGAE VKKPGASVKV SCKASGYTFT SYWMQWVRQA
PGQGLEWMGT IYPGDGDTRY TQKFKGRVTI TADKSTSTAY
MELSSLRSED TAVYYCARRG IPRLWYFDVW GQGTTVTVSS

The amino acid sequence of the VH Domain of hBRCA69D VH2 is (SEQ ID NO:97) (CDR_{H} residues are shown underlined):

QVQLVQSGAE VKKPGASVKV SCKASGYTFT SYWMQWVRQA
PGQGLEWMGT IYPGGGDTRY TQKFQGRVTI TADKSTSTAY
MELSSLRSED TAVYYCARRG IPRLWYFDVW GQGTTVTVSS

The amino acid sequence of the VL Domain of hBRCA69D VL1 is (SEQ ID NO:98) (CDR_{L} residues are shown underlined).

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIK

The amino acid sequence of the VL Domain of hBRCA69D VL2 is (SEQ ID NO:99) (CDR_{L} residues are shown underlined).

DIQMTQSPSS LSASVGDRVT ITCRASQSIS SYLNWYQQKP
GKAPKLLIYY TSRLQSGVPS RFSGSGSGTD FTLTISSLQP
EDIATYYCQQ GNTLPPTFGG GTKLEIK

(2) hPRCA157

Another representative humanized anti-B7-H3 antibody is “hPRCA157”. The amino acid sequence of the VH Domain of hPRCA157 VH1 is (SEQ ID NO:100) (CDR_{H} residues are shown underlined):

EVQLVESGGG LVKPGGSLRL SCAASGFTFS SYGMSWVRQA
PGKGLEWVAT INSGGSNTYY PDSLKGRFTI SRDNAKNSLY
LQMNSLRAED TAVYYCARHD GGAMDYWGQG TTVTVSS

The amino acid sequence of the VL Domain of hPRCA157 VL1 is (SEQ ID NO:101) (CDR_{L} residues are shown underlined):

DIQMTQSPSS LSASVGDRVT ITCRASESIY SYLAWYQQKP
GKAPKLLVYN TKTLPEGVPS RFSGSGSGTD FTLTISSLQP
EDFATYYCQH HYGTPPWTFG QGTRLEIK

### (3) Other B7-H3 Binding Domains

In addition to the above-identified B7-H3 binding domains, the invention contemplates the use of any of the epitope-binding site of any of the following anti-B7-H3 antibodies: LUCA1; BLA8; PA20; or SKN2 (see, U.S. Pat. Nos. 7,527,969; 8,779,098 and PCT Patent Publication WO 2004/001381); M30; cM30; M30-H1-L1; M30-H1-L2; M30-H1-L3; M30-H1-L4; M30-H1-L5; M30-H1-L6; M30-H1-L7; M30-H4-L1; M30-H4-L2; M30-H4-L3; and M30-H4-L4 (see, US Patent Publication 2013/0078234 and PCT Patent Publication WO 2012/147713; and 8H9 (see U.S. Pat. Nos. 7,666,424; 7,737,258; 7,740,845; 8,148,154; 8,414,892; 8,501,471; 9,062,110; US Patent Publication 2010/0143245 and PCT Patent Publication WO 2008/116219).

The present invention specifically includes and encompasses CD137×B7-H3 Binding Molecules that comprise the VL and/or VH Domain, and/or 1, 2 or all 3 of the CDR_{L}s of the VL Region and/or 1, 2 or all 3 of the CDR_{H}s of the VH Domain of any of, humanized BRCA69D, PRCA157, humanized PRCA157, or Enoblituzumab, or any of the other anti-B7-H3 antibodies provided herein; and more typically possess 1, 2 or all 3 of the CDRLs of the VL Region and/or 1, 2 or all 3 of the CDR_{H}s of the VH Domain of such anti-B7-H3 monoclonal antibodies.

### f) GpA33 Binding Domains

The 43 kD transmembrane glycoprotein A33 (gpA33) is expressed in >95% of all colorectal carcinomas. The epitope-binding site of any anti-gpA33 antibody may be used in accordance with the present invention. An representative humanized anti-gpA33 antibody (“gpA33 MAB-1”) is presented below.

The amino acid sequence of the VH Domain o gpA33 MAB-1 is (SEQ ID NO:102) (CDR residues are shown underlined):

QVQLVQSGAE VKKPGASVKV SCKASGYTFT GSWMNWVRQA
PGQGLEWIGR IYPGDGETNY NGKFKDRVTI TADKSTSTAY
MELSSLRSED TAVYYCARIY GNNVYFDVWG QGTTVTVSS

The amino acid sequence of the VL Domain of gpA33 MAB-1 is (SEQ ID NO:103 (CDR residues are shown underlined

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIK

The present invention specifically includes and encompasses CD137×gpA33 Binding Molecules that comprise the VL and/or VH Domain, and/or 1, 2 or all 3 of the CDR_{L}s of the VL Region and/or 1, 2 or all 3 of the CDR_{H}s of the VH Domain of anti-gpA33 monoclonal antibodies gpA33 MAB-1, or of any of the anti-gpA33 monoclonal antibodies provided in WO 2015/026894.

### g) CEACAM5 and CEACAM6 Binding Domains

Carcinoembryonic Antigen-Related Cell Adhesion Molecules 5 (CEACAM5) and 6 (CEACAM6) have been found to be associated with various types of cancers including medullary thyroid cancer, colorectal cancer, pancreatic cancer, hepatocellular carcinoma, gastric cancer, lung cancer, head and neck cancers, urinary bladder cancer, prostate cancer, uterine cancer, endometrial cancer, breast cancer, hematopoietic cancer, leukemia and ovarian cancer, and particularly colorectal, gastrointestinal, pancreatic, non-small cell lung cancer (NSCL), breast, thyroid, stomach, ovarian and uterine carcinomas. The epitope-binding site of any anti-CEACAM5/CEACAM6 antibody may be used in accordance with the present invention. Representative anti-CEACAM5/CEACAM6 antibodies are provided below.

### (1) 16C3

The amino acid sequence of the VH Domain of the humanized anti-CEACAM5/CEACAM6 antibody 16C3 (EP 2585476) is (SEQ ID NO:104) (CDR residues are shown underlined):

QVQLQQSGPE VVRPGVSVKI SCKGSGYTFT DYAMHWVKQS
HAKSLEWIGL ISTYSGDTKY NQNFKGKATM TVDKSASTAY
MELSSLRSED TAVYYCARGD YSGSRYWFAY WGQGTLVTVS S

The amino acid sequence of the VL Domain of the humanized anti-CEACAM5/CEACAM6 antibody 16C3 (EP 2585476) is (SEQ ID NO:105) (CDR residues are shown underlined):

DIQMTQSPSS LSASVGDRVT ITCGASENIY GALNWYQRKP
GKSPKLLIWG ASNLADGMPS RFSGSGSGRQ YTLTISSLQP
EDVATYYCQN VLSSPYTFGG GTKLEIK

(2) hMN15

The amino acid sequence of the VH Domain of the humanized anti-CEACAM5/CEACAM6 antibody hMN15 (U.S. Pat. No. 8,287,865) is (SEQ ID NO:106) (CDR residues are shown underlined):

QVQLVESGGG VVQPGRSLRL SCSSSGFALT DYYMSWVRQA
PGKGLEWLGF IANKANGHTT DYSPSVKGRF TISRDNSKNT
LFLQMDSLRP EDTGVYFCAR DMGIRWNFDV WGQGTPVTVS
S

The amino acid sequence of the VL Domain of the humanized anti-CEACAM5/CEACAM6 antibody hMN15 (U.S. Pat. No. 8,287,865) is (SEQ ID NO:107) (CDR residues are shown underlined)

DIQLTQSPSS LSASVGDRVT MTCSASSRVS YIHWYQQKPG
KAPKRWIYGT STLASGVPAR FSGSGSGTDF TFTISSLQPE
DIATYYCQQW SYNPPTFGQG TKVEIKR

The present invention specifically includes and encompasses CD137×CEACAM5/CEACAM6 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-CEACAM5/CEACAM6 monoclonal antibodies 16C3 or hMN15.

### h) CD19 Binding Domains

CD19 (B lymphocyte surface antigen B4, Genbank accession number M28170) is a component of the B cell-receptor (BCR) complex, and is a positive regulator of B cell signaling that modulates the threshold for B cell activation and humoral immunity. CD19 is one of the most ubiquitously expressed antigens in the B cell lineage and is expressed on >95% of B cell malignancies, including acute lymphoblastic leukemia (ALL), chronic lymphocytic leukemia (CLL), and non-Hodgkin's Lymphoma (NHL). Notably, CD19 expression is maintained on B cell lymphomas that become resistant to anti-CD20 therapy. CD19 has also been suggested as a target to treat autoimmune diseases.

The epitope-binding site of any anti-CD19 antibody may be used in accordance with the present invention. An representative humanized antibody that binds to human CD19, and that may be employed in the present invention, is the humanized anti-CD19 antibody disclosed in WO 2016/048938 (referred to herein as “CD19 MAB-1”).

The amino acid sequence of the VH Domain of CD19 MAB-1 is (SEQ ID NO:108) (CDR_{H} residues are shown underlined):

QVTLRESGPA LVKPTQTLTL TCTFSGFSLS TSGMGVGWIR
QPPGKALEWL AHIWWDDDKR YNPALKSRLT ISKDTSKNQV
FLTMTNMDPV DTATYYCARM ELWSYYFDYW GQGTTVTVSS

The amino acid sequence of the VL Domain of CD19 MAB-1 is (SEQ ID NO:109) (CDR_{L} residues are shown underlined):

ENVLTQSPAT LSVTPGEKAT ITCRASQSVS YMHWYQQKPG
QAPRLLIYDA SNRASGVPSR FSGSGSGTDH TLTISSLEAE
DAATYYCFQG SVYPFTFGQG TKLEIK

The present invention specifically includes and encompasses CD137×CD19 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-CD19 monoclonal antibody CD19 MAB-1, or any of the anti-CD19 antibodies disclosed in U.S. Pat. No. 7,112,324, or present in blinatumomab (BLINCYTO®; amino acid sequence found in WHO Drug Information, 2009, Recommended INN: List 62, 23(3):240-241) and duvortuxizumab (aka MGD011; amino acid sequence found in WHO Drug Information, 2016, Proposed INN: List 116, 30(4):627-629).

### i) CD123 Binding Domains

CD123 (interleukin 3 receptor) comprises a unique alpha chain, IL-3Ra that is a 40 kDa molecule. Interleukin 3 (IL-3) drives early differentiation of multipotent stem cells into cells of the erythroid, myeloid and lymphoid progenitors. CD123 has been reported to be overexpressed on malignant cells in a wide range of hematologic malignancies including acute myeloid leukemia (AML) and myelodysplastic syndrome (MDS). Overexpression of CD123 is associated with poorer prognosis in AIL.

The epitope-binding site of any anti-CD123 antibody may be used in accordance with the present invention. An representative humanized antibody that binds to human CD123, and that may be employed in the present invention, is “CD123 MAB-1” (see, e.g., PCT Patent Publication WO 2015/026892).

The amino acid sequence of the VH Domain of CD123 MAB-1 is (SEQ ID NO:110) (CDR_{H} residues are shown underlined):

EVQLVQSGAE LKKPGASVKV SCKASGYTFT DYYMKWVRQA
PGQGLEWIGD IIPSNGATFY NQKFKGRVTI TVDKSTSTAY
MELSSLRSED TAVYYCARSH LLRASWFAYW GQGTLVTVSS

The amino acid sequence of the VL Domain of CD123 MAB-1 is (SEQ ID NO:111) (CDR_{L} residues are shown underlined):

DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIK

The present invention specifically includes and encompasses CD137×CD123 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-CD123 monoclonal antibody CD123 MAB-1, or any of the anti-CD123 antibodies disclosed in US 2017/081424 and WO 2016/036937, or present in JNJ-63709178 (Johnson & Johnson, also see, WO 2016/036937) and XmAbl4045 (Xencor, also see, US 2017/081424).

### j) IL13Rα 2

Interleukin-13 Receptor α2 (IL13Rα2) is overexpressed in a variety of cancers, including glioblastoma, colorectal cancer, cervical cancer, pancreatic cancer, multiple melanoma, osteosarcoma, leukemia, lymphoma, prostate cancer and lung cancer Antibodies that immunospecifically bind to IL13Ra2 are commercially available and have been described in the art (see, e.g., WO 2008/146911). Representative humanized antibodies that bind to human IL13Ra2 include “hu08” (see, e.g., WO 2014/072888).

The amino acid sequence of the VH Domain of hu08 (SEQ ID NO:112) is shown below (CDR residues are shown underlined):

EVQLVESGGG LVQPGGSLRL SCAASGFTFS RNGMSWVRQA
PGKGLEWVAT VSSGGSYIYY ADSVKGRFTI SRDNAKNSLY
LQMNSLRAED TAVYYCARQG TTALATRFFD VWGQGTLVTV
SS

The amino acid sequence of the VL Domain of hu08 (SEQ ID NO:113) is shown below (CDR residues are shown underlined):

DIQMTQSPSS LSASVGDRVT ITCKASQDVG TAVAWYQQKP
GKAPKLLIYS ASYRSTGVPS RFSGSGSGTD FTLTISSLQP
EDFATYYCQH HYSAPWTFGG GTKVEIK

The present invention specifically includes and encompasses CD137×IL13Ra2 Binding Molecules that comprise the VL and/or VH Domain, and/or 1, 2 or all 3 of the CDR_{L}s of the VL Region and/or 1, 2 or all 3 of the CDR_{H}s of the VH Domain of the anti-IL13Ra2 monoclonal antibody hu08.

### k) ROR1

Receptor Tyrosine Kinase-Like Orphan Receptor 1 (“ROR1”) is a type I membrane protein belonging to the ROR subfamily of cell surface receptors. ROR1 is an onco-embryonic antigen that is expressed by many tissues during embryogenesis, is absent from most mature tissues and is expressed in numerous blood and solid malignancies including ovarian, colon, lung, lymphoma, skin, pancreatic, testicular, bladder, uterus, prostate, adrenal, breast, and B-cell malignancies, as well as in some cancer stem cells. ROR1 expression is associated with high-grade tumors exhibiting a less-differentiated morphology and is correlated with poor clinical outcomes. The epitope-binding site of any anti-RORi antibody may be used in accordance with the present invention. Presented below in an representative humanized/optimized anti-RORi antibody that may be used to generate the molecules of the present invention, variations of this antibody are described in WO 2017/142928.

### (1) Anti-ROR1

The amino acid sequence of an representative VH of the anti-RORi antibody is (SE ID NO:114 CDR residues are shown underlined):

QEQLVESGGG LVQPGGSLRL SCAASGFTFS DYYMSWXRQA
PGKGLEWVAT IYPSSGKTYY ADSAKGRLTI SSDNAKDSLY
LQMNSLRAED TAVYYCTRDS YADDAALFDI WGQGTTVTVS
S

wherein X is I or V

The amino acid sequence of an representative VL of the anti-RORi antibody is (SEQ ID NO:115) (CDR residues are shown underlined):

QLVLTQSPSA SASLGSSVKL TCTLSSGHKT DTIDWYQQQP
GKAPRYLMKL EGSGSYNKGS GVPDRFSGSS SGADWYLTIS
SLQSEDEADY YCGTDYPGNY LFGGGTQLTV LG

### (2) Other ROR1 Binding Domains

In addition to the above-identified ROR1 binding domains, the invention contemplates the use of any of the epitope-binding site of any of the following anti-RORi antibodies: 4A5 (see, U.S. Pat. No. 8,212,009); R11, R12, and Y31 (see, U.S. Pat. No. 9,758,586); and A1-A14 (see, e.g., U.S. Pat. No. 9,228,023).

The present invention specifically includes and encompasses CD137×ROR1 Binding Molecules that comprise the VL and/or VH Domain, and/or 1, 2 or all 3 of the CDRLs of the VL Region and/or 1, 2 or all 3 of the CDRHs of the VH Domain of any of the anti-ROR1 monoclonal antibodies provided herein.

### D. CD137×TA Binding Molecules of the Present Invention

The present invention is particularly directed to Fc-bearing tetravalent and trivalent CD137×TA Binding Molecules capable of simultaneous binding to CD137 and a TA, and other Fc-bearing CD137×TA Binding Molecules capable of simultaneous binding to CD137 and a TA. The present invention is further directed to the use of such molecules in the treatment of cancer and other diseases and conditions.

### 1. Tetravalent CD137×TA Fc-Bearing Diabodies

The present invention particularly encompasses a wide variety of Fc-bearing diabodies capable of simultaneous binding to CD137 and a TA. Representative CD137×TA Fc-bearing Diabodies are described below.

Such tetravalent Fc-bearing diabodies will comprise two polypeptide chains. The first of such polypeptide chains may contain, in the N-terminal to C-terminal direction, an N-terminus, a Light Chain Variable Domain (VL) of an antibody capable of binding to an epitope of a “first” antigen (VL1) (either CD137 or TA), a Heavy Chain Variable Domain (VII) of an antibody capable of binding to an epitope of a “second” antigen (VH2) (TA, if VL1 was selected to bind to an epitope of CD137; CD137 if VL1 was selected to bind to an epitope of TA), a cysteine-containing domain, one or more additional domains as provided in more detail below, and a C-terminus. The second of such polypeptide chains may contain, in the N-terminal to C-terminal direction, an N-terminus, a Light Chain Variable Domain (VL) of an antibody capable of binding to an epitope of the “second” antigen (VL2) (TA, if the first antigen was CD137; CD137, if the first antigen was TA)), a Heavy Chain Variable Domain (VII) of an antibody capable of binding to an epitope of the “second” antigen (VH2) (TA, if VL2 was selected to bind to an epitope of CD137; CD137 if VL2 was selected to bind to an epitope of TA, a cysteine-containing domain, one or more additional domains as provided in more detail below, and a C-terminus. An intervening linker peptide (Linker 1) separates the Light Chain Variable Domain (VL1 or VL2) from the Heavy Chain Variable Domain (VH1 or VH2).

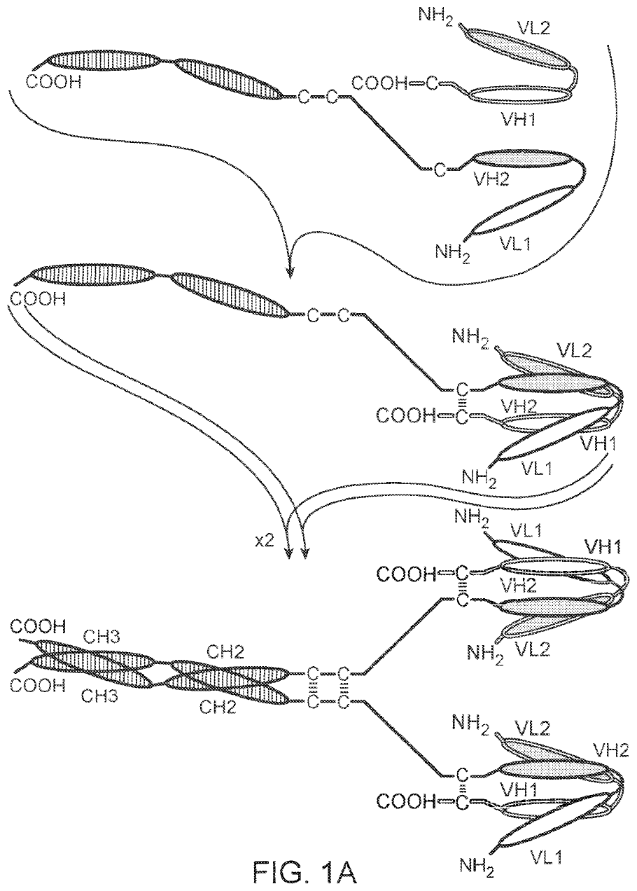

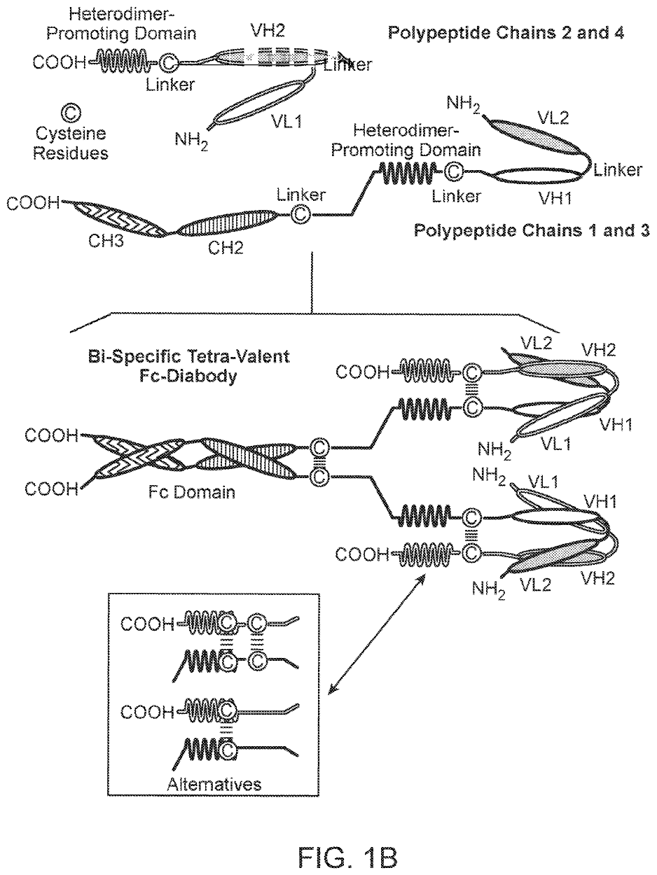

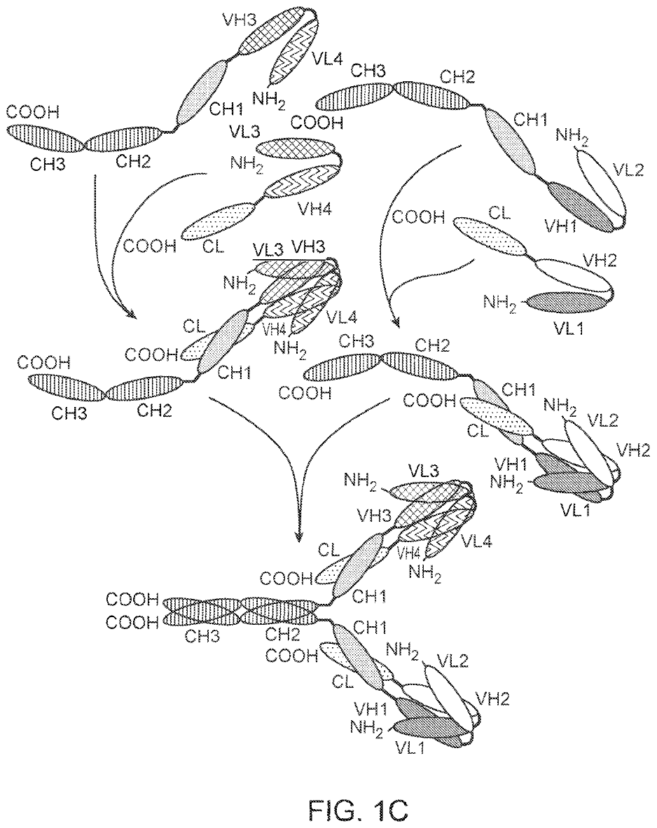

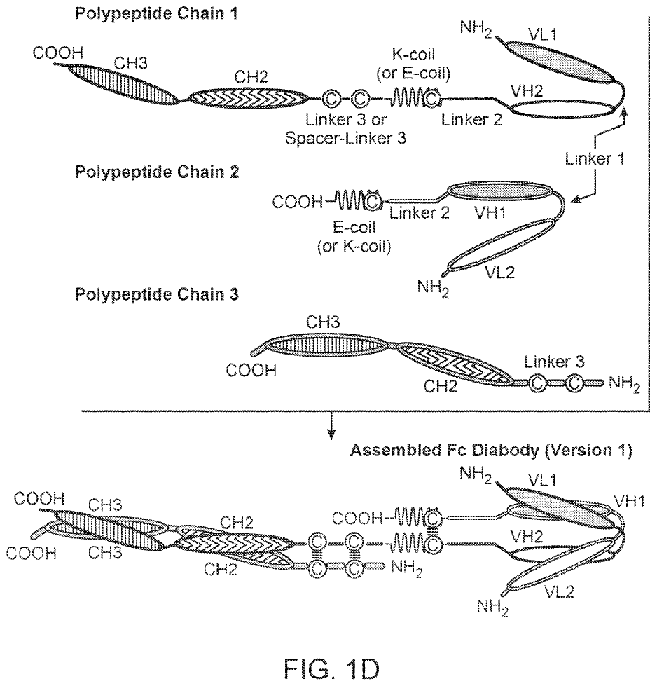

In certain embodiments, Fc-bearing diabodies of the present invention are covalently bonded tetravalent diabodies having four epitope-binding sites that comprise four polypeptide chains, and have the general structure depicted in FIG. 1A. The first and third polypeptide chains of such a diabody contain in the N-terminal to C-terminal direction: (i) a VL1-containing Domain, (ii) a VH2-containing Domain, (iii) Heterodimer-Promoting Domain and (iv) a Domain containing a CH2-CH3 sequence. The second and fourth polypeptide chains contain: (i) a VL2-containing Domain, (ii) a VH1-containing Domain and (iii) a Heterodimer-Promoting Domain, where the Heterodimer-Promoting Domains promote the dimerization and covalent bonding of the first/third polypeptide chains with the second/fourth polypeptide chains. The VH Domains are linked to the Heterodimer-Promoting Domains by intervening linker peptides (Linker 2), which may comprise a cysteine residue. Optionally, or additionally the Heterodimer Promoting Domains may comprise a cysteine residues. In a representative CD137×TA bispecific Fc-bearing diabody embodiment, the C-terminus of the Heterodimer-Promoting Domain of the first polypeptide chain is linked to CH2-CH3 domains by an intervening linker peptide (Linker 3). The VL and/or VH Domains of the third and fourth polypeptide chains, and VL and/or VH Domains of the first and second polypeptide chains may be the same or different so as to permit tetravalent binding that is either monospecific, bispecific or tetraspecific. In Table 3 below, notations “VL3” and “VH3” denote, respectively, the Light Chain Variable Domain and Variable Heavy Chain Domain that bind a “third” epitope of such diabody. Similarly, the notations “VL4” and “VH4” denote, respectively, the Light Chain Variable Domain and Variable Heavy Chain Domain that bind a “fourth” epitope of such diabody. The general structure of the polypeptide chains of a representative four-chain bispecific Fc Region-containing diabodies of invention is provided in Table 3:

TABLE 3

| | | |
|---|---|---|
| Bispecific | 2^{nd} Chain | NH₂-VL2-VH1-\textcircled{c}-HPD-COOH |
| | 1^{st} Chain | NH₂-VL1-VH2-\textcircled{c}-HPD-\textcircled{c}-CH2—CH3—COOH |
| | 1^{st} Chain | NH₂-VL1-VH2-\textcircled{c}-HPD-\textcircled{c}-CH2—CH3—COOH |
| | 2^{nd} Chain | NH₂-VL2-VH1-\textcircled{c}-HPD-COOH |
| Tetraspecific | 2^{nd} Chain | NH₂-VL2-VH1-\textcircled{c}-HPD-COOH |
| | 1^{st} Chain | NH₂-VL1-VH2-\textcircled{c}-HPD-\textcircled{c}-CH2—CH3—COOH |
| | 3^{rd} Chain | NH₂-VL3-VH4-\textcircled{c}-HPD-\textcircled{c}-CH2—CH3—COOH |
| | 4^{th} Chain | NH₂-VL4-VH3-\textcircled{c}-HPD-COOH |

(-\textcircled{c}-denotes a cysteine-containing polypeptide domain that possesses one, two, or more than two cysteine residues. The representation is intended to be illustrative and non-limiting. Cysteine residues may be present in additional or alternative domains, such as within the Heterodimer-Promoting Domain (HPD))

In certain embodiments, CD137×TA Binding Molecules of the present invention are bispecific, tetravalent (i.e., possess four epitope-binding sites), Fc-bearing diabodies that are composed of four total polypeptide chains (FIGS. 1A-1C). The CD137×TA Binding Molecules of the invention are bispecific, tetravalent, Fc-bearing diabodies that comprise two epitope-binding sites immunospecific for CD137 (which may be capable of binding to the same epitope of CD137 or to different epitopes of CD137), and two epitope-binding sites immunospecific for a tumor antigen (which may be capable of binding to the same epitope of a TA or to different epitopes of a TA or different epitopes of different TAs).

In a further embodiment, the Fc Domain-containing diabodies of the present invention may comprise three polypeptide chains. The first polypeptide of such a diabody often contains three domains: (i) a VL1-containing Domain, (ii) a VH2-containing Domain and (iii) a Domain containing a CH2-CH3 sequence. The second polypeptide of such a diabody often contains: (i) a VL2-containing Domain, (ii) a VH1-containing Domain and (iii) a Domain that promotes heterodimerization and covalent bonding with the diabody's first polypeptide chain. The third polypeptide of such a diabody often comprises a CH2-CH3 sequence. Thus, the first and second polypeptide chains of such a diabody often associate together to form a VL1/VH1 Epitope-Binding Domain that is capable of binding either the first or second epitope, as well as a VL2/VH2 Epitope-Binding Domain that is capable of binding the other of such epitopes. The first and second polypeptides often are bonded to one another through a disulfide bond involving cysteine residues in their respective Third Domains. Notably, the first and third polypeptide chains often complex with one another to form an Fc Domain that is stabilized via a disulfide bond. FIG. 1D illustrates a representative structure of such diabodies.

In each of the above embodiments, the Light Chain Variable Domain of the first polypeptide chain (VL1) is coordinately selected so as to permit it to interact with the Heavy Chain Variable Domain of the second polypeptide chain (VH1) to thereby form a functional epitope-binding site that is capable of immunospecifically binding an epitope of the first antigen (i.e., either TA or CD137). Likewise, the Light Chain Variable Domain of the second polypeptide chain (VL2) is coordinately selected so as to permit it to interact with the Heavy Chain Variable Domain of the first polypeptide chain (VH2) to thereby form a functional epitope-binding site that is capable of immunospecifically binding an epitope of the second antigen (i.e., either a TA or CD137). Thus, the selection of the Light Chain Variable Domains and the Heavy Chain Variable Domains are coordinated, such that the two polypeptide chains collectively comprise epitope-binding sites capable of binding to CD137 and a TA.

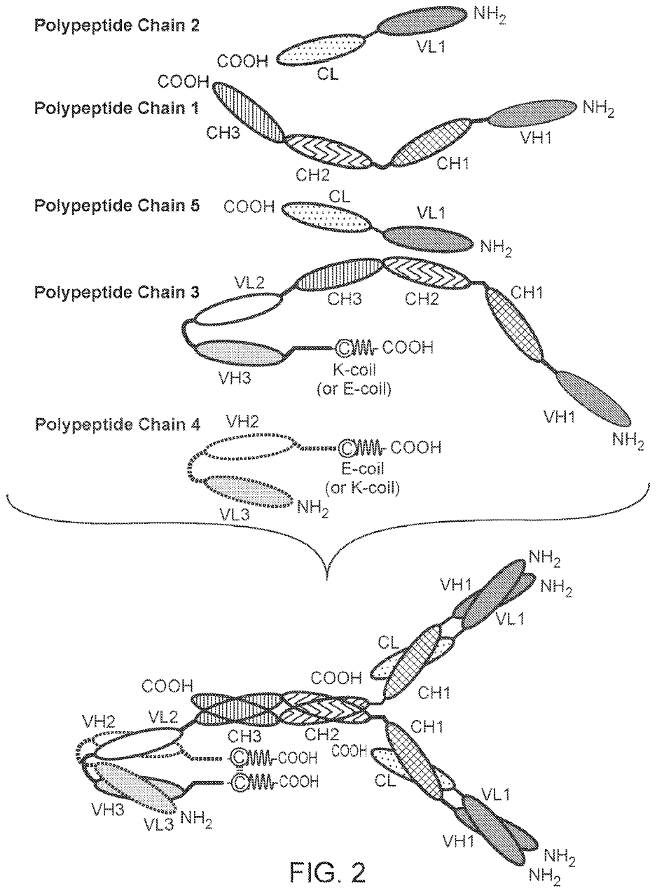

Additional Fc-bearing diabodies of the present invention comprise five polypeptide chains, and are depicted in FIG. 2. The first polypeptide chain of such a diabody contains: (i) a VH1-containing domain, (ii) a CH1-containing domain, and (iii) a Domain containing a CH2-CH3 sequence. The first polypeptide chain may be the heavy chain of an antibody that contains a VH1 and a heavy chain constant region. The second and fifth polypeptide chains of such a diabody contain: (i) a VL1-containing domain, and (ii) a CL-containing domain. The second and/or fifth polypeptide chains of such a diabody may be light chains of an antibody that contains a VL1 complementary to the VH1 of the first/third polypeptide chain. The first, second and/or fifth polypeptide chains may be isolated from a naturally occurring antibody. Alternatively, they may be constructed recombinantly. In one embodiment, the second and fifth polypeptide chains have the same amino acid sequence. The third polypeptide chain of such a diabody contains: (i) a VH1-containing domain, (ii) a CH1-containing domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain. The C-terminus of the VH3- and VH2-containing domains of the third and fourth polypeptide chains are linked to a Heterodimer-Promoting Domain by an intervening linker peptide (Linker 2), and the C-terminus of the CH2-CH3 domains of the third polypeptide chain is linked to the VL2-containing Domain by an intervening linker peptide (Linker 4).

Thus, the first and second, and the third and fifth, polypeptide chains of such diabodies associate together to form two VL1/VH1 binding sites capable of binding a first epitope. The third and fourth polypeptide chains of such diabodies associate together to form one diabody binding domain comprising a VL2/VH2 binding site that is capable of binding to a second epitope, as well as a VL3/VH3 binding site that is capable of binding to a third epitope. The first and third polypeptides are bonded to one another through a disulfide bond involving cysteine residues in their respective constant regions. Notably, the first and third polypeptide chains complex with one another to form an Fc Region. Such bispecific diabodies have enhanced potency. FIG. 2 illustrates the structure of such diabodies. It will be understood that the VL1/VH1, VL2/VH2, and VL3/VH3 Domains may be the same or different so as to permit binding that is monospecific, bispecific or trispecific. However, as provided herein, these domains are selected so as to bind CD137 and a TA.

The VL and VH Domains of the polypeptide chains are selected so as to form VL/VH binding sites specific for a desired epitope. The VL/VH binding sites formed by the association of the polypeptide chains may be the same or different so as to permit tetravalent binding that is monospecific, bispecific, trispecific or tetraspecific. In particular, the VL and VH Domains may be selected such that a bispecific diabody may comprise two binding sites for a first epitope and two binding sites for a second epitope, or three binding sites for a first epitope and one binding site for a second epitope, or two binding sites for a first epitope, one binding site for a second epitope and one binding site for a third epitope (as depicted in FIG. 2). The general structure of the polypeptide chains of representative five-chain Fc Region-containing diabodies of invention is provided in Table 4:

TABLE 4

| | | |
|---|---|---|
| Bispecific | 2^{nd} Chain | NH₂-VL1-CL-\textcircled{c}-COOH |
| (2 × 2) | 1^{st} Chain | NH₂-VH1-CH1-\textcircled{c}-\textcircled{c}-CH2—CH3—COOH |
| | 3^{rd} Chain | NH₂-VH1-CH1-\textcircled{c}-\textcircled{c}-CH2—CH3-VL2-VH2-\textcircled{c}-HPD-COOH |
| | 5^{nd} Chain | NH₂-VL1-CL-\textcircled{c}-COOH |
| | 4^{th} Chain | NH₂-VL2-VH2-\textcircled{c}-HPD-COOH |
| Bispecific | 2^{nd} Chain | NH₂-VL1-CL-\textcircled{c}-COOH |
| (3 × 1) | 1^{st} Chain | NH₂-VH1-CH1-\textcircled{c}-\textcircled{c}-CH2—CH3—COOH |
| | 3^{rd} Chain | NH₂-VH1-CH1-\textcircled{c}-\textcircled{c}-CH2—CH3-VL1-VH2-\textcircled{c}-HPD-COOH |
| | 5^{nd} Chain | NH₂-VL1-CL-\textcircled{c}-COOH |
| | 4^{th} Chain | NH₂-VL2-VH1-\textcircled{c}-HPD-COOH |
| Trispecific | 2^{nd} Chain | NH₂-VL1-CL-\textcircled{c}-COOH |
| (2 × 1 × 1) | 1^{st} Chain | NH₂-VH1-CH1-\textcircled{c}-\textcircled{c}-CH2—CH3—COOH |
| | 3^{rd} Chain | NH₂-VH1-CH1-\textcircled{c}-\textcircled{c}-CH2—CH3-VL2-VH3-\textcircled{c}-HPD-COOH |
| | 5^{nd} Chain | NH₂-VL1-CL-\textcircled{c}-COOH |
| | 4^{th} Chain | NH₂-VL3-VH2-\textcircled{c}-HPD-COOH |

(-\textcircled{c}-denotes a cysteine-containing polypeptide domain that possesses one, two, or more than two cysteine residues. The representation is intended to be illustrative and non-limiting. Cysteine residues may be present in additional or alternative domains, such as within the Heterodimer-Promoting Domain (HPD))

In certain embodiments, CD137×TA Binding Molecules of the present invention are bispecific, tetravalent (i.e., possess four epitope-binding sites), Fc-bearing diabodies that are composed of five total polypeptide chains having two epitope-binding sites immunospecific for CD137 (which may be capable of binding to the same epitope of CD137 or to different epitopes of CD137), and two epitope-binding sites immunospecific for a TA (which may be capable of binding to the same epitope of a TA or to different epitopes of a TA or different epitopes of different TAs). In another embodiment, the CD137×TA Binding Molecules of the invention are bispecific, tetravalent, Fc-bearing diabodies that comprise three epitope-binding sites immunospecific for CD137 (which may be capable of binding to the same epitope of CD137 or to two or three different epitopes of CD137), and one epitope-binding site specific for a TA.

### 2. Trivalent CD137×TA Binding Molecules

In one embodiment, the CD137×TA Binding Molecules of the present invention are trivalent and will comprise a first epitope-binding site (e.g., a VL1 and VH1), a second epitope-binding site (e.g., a VL2 and VH2), and a third epitope-binding site (e.g., a VL3 and VH3), and will thus be able to bind to an epitope of TA, an epitope of CD137, and a third epitope, which third epitope may be:
(a) the same or a different epitope of the TA;
(b) the same or a different epitope of CD137; or
(c) an epitope of a different TA.

In certain embodiments, such “Trivalent CD137×TA Binding Molecules” of the present invention will comprise two epitope-binding sites for an epitope of CD137 (which epitopes may be the same or different), and one epitope-binding site for an epitope of a TA.

In general, such Trivalent CD137×TA Binding Molecules of the present invention are composed of three, four, five or more than five polypeptide chains that, by virtue of one or more disulfide bonds between pairs of such polypeptides, form a covalently bonded molecular complex that comprises a “Diabody-Type Binding Domain” and a “Non-Diabody-Type Binding Domain.”

A “Diabody-Type Binding Domain” is the Epitope-Binding Domain of a diabody, and especially, a DART® diabody. The terms “diabody” and “DART® diabody” have been discussed above. A “Non-Diabody-Type” Binding Domain is intended to denote a Binding Domain that does not have the structure of a Diabody-Type Binding Domain. Typically, a Non-Diabody-Type Binding Domain is a Fab-Type Binding Domain or an ScFv-Type Binding Domain. As used herein, the term “Fab-Type Binding Domain” refers to an Epitope-Binding Domain that is formed by the interaction of the VL Domain of an immunoglobulin Light Chain and a complementing VH Domain of an immunoglobulin heavy chain. Fab-Type Binding Domains differ from Diabody-Type Binding Domain in that the two polypeptide chains that form a Fab-Type Binding Domain comprise only a single Epitope-Binding Domain, whereas the two polypeptide chains that form a Diabody-Type Binding Domain comprise at least two Epitope-Binding Domains. ScFv-Type Binding Domains differ from Diabody-Type Binding Domain in that VL and VH Domains of the same polypeptide chain interact to form an Epitope-Binding Domain. Thus, as used herein, Fab-Type Binding Domains and ScFv-Type Binding Domains are distinct from Diabody-Type Binding Domain.

Thus, the Trivalent CD137×TA Binding Molecules of the present invention comprise:
(I) a “first” Epitope-Binding Domain that is capable of immunospecifically binding to a “first” epitope;
(II) a “second” Epitope-Binding Domain that is capable of immunospecifically binding to a “second” epitope;
(III) a “third” Epitope-Binding Domain that is capable of immunospecifically binding to a “third” epitope; and
(IV) an Fc Domain that is formed by the association of two CH2-CH3 Domains to one another;
wherein:
   (A) the “first” Epitope-Binding Domain and the “second” Epitope-Binding Domain are both “Diabody-Type Binding Domains;
   (B) the “third” Epitope-Binding Domain is a Non-Diabody-Type Binding Domain; and
   (C) one of such “first,” “second,” or “third” Epitope-Binding Domains binds an epitope of TA, and another of such “first,” “second,” or “third” Epitope-Binding Domains binds an epitope of CD137;

The epitope that is bound by the remaining Epitope-Binding Domain may be any desired epitope, for example, an epitope of CD137. Such epitope which may be the same or different from the CD137 epitope that is bound by other Epitope-Binding Domains of the molecule.

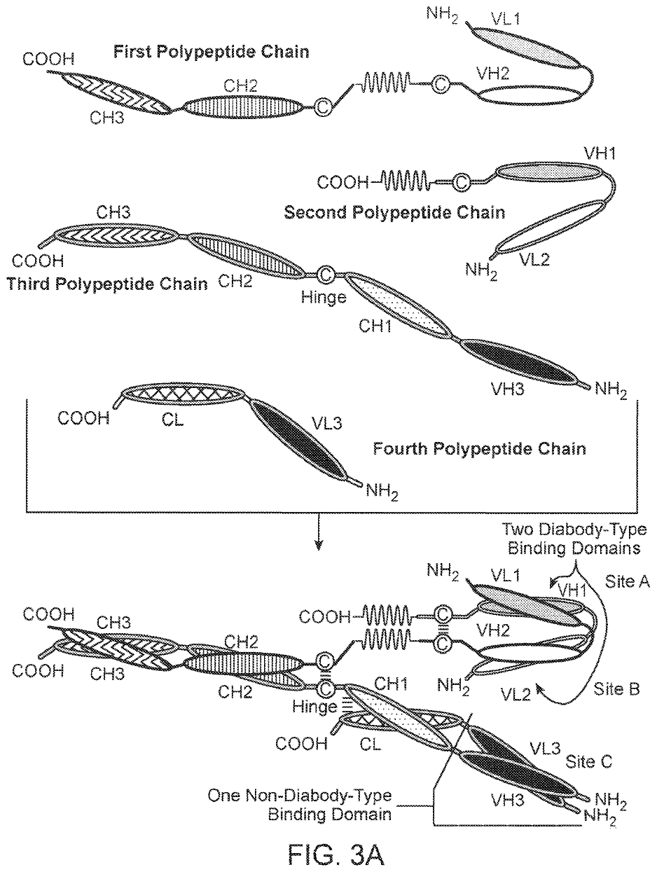

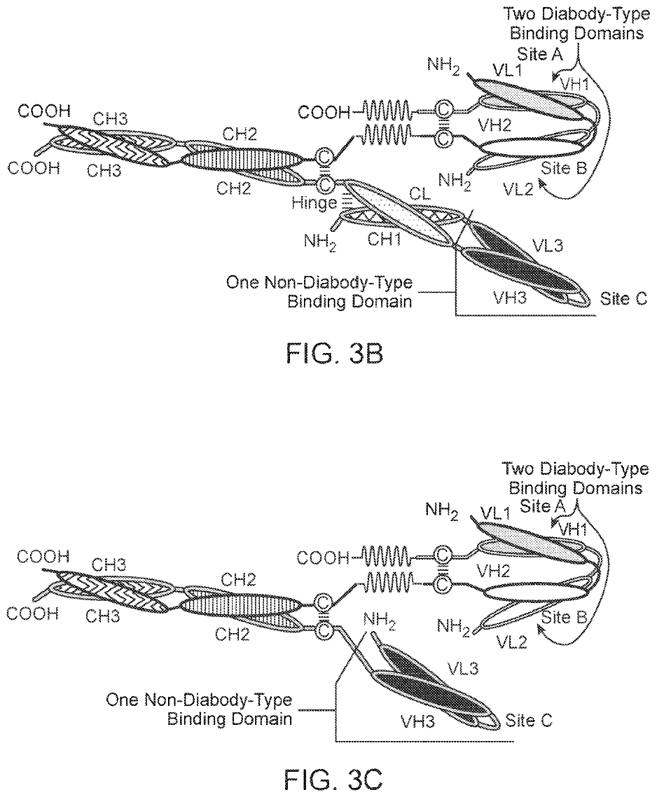

FIGS. 3A-3C provide a diagrammatic representation of the Domains of representative Trivalent CD137×TA Binding Molecules. FIG. 3A illustrates schematically the Domains of representative Trivalent CD137×TA Binding Molecules that are composed from the covalent complexing of four polypeptide chains and possess one Non-Diabody-Type Binding Site (VL3/VH3 and thus being monovalent for such epitope), and two Diabody-Type Binding Sites (VL1/VH1 and VL2/VH2, and thus being monovalent for each of such epitopes). FIGS. 3B-3C illustrate schematically the Domains of representative Trivalent CD137×TA Binding Molecules that are composed from the covalent complexing of three polypeptide chains and possess one Non-Diabody-Type Binding Site (VL3/VH3 and thus being monovalent for such epitope), and two Diabody-Type Binding Sites (VL1/VH1 and VL2/VH2, and thus being monovalent for each of such epitopes). The Non-Diabody-Type Binding Site is a Fab-Type Binding Domain in FIGS. 3A-3B and is an scFv-Type Binding Domain in FIG. 3C. As provided below, VL/VH binding sites formed by the association of the polypeptide chains may be the same or different so as to permit trivalent binding that is monospecific, bispecific, or trispecific.

### II. Representative CD137×TA Binding Molecules

The invention provides CD137×TA Binding Molecules that are bispecific tetravalent Fc diabodies capable of simultaneously and specifically binding to CD137 and to a TA. As indicated above, the CD137×TA Binding Molecules of the present invention may comprise three, four or five polypeptide chains. The polypeptide chains of representativeCD137×TA Binding Molecules capable of binding to CD137 and to the TA, PD-L1 or HER2 are provided below (designated “DART-A,” “DART-A1,” “DART-A2,” “DART-A3,” “DART-A4,” “DART-A5,” “DART-A6,” “DART-A7,” “DART-A8,” “DART-A9,” “DART-A10,” “DART-B1,” and “DART-B2”). The invention further provides CD137×TA Binding Molecule that are bispecific trivalent binding molecules capable of simultaneously and specifically binding to CD137 and to a TA. As indicated above, the trivalent CD137×TA Binding Molecules of the present invention may comprise four polypeptide chains. The polypeptide chains of representative trivalent CD137×TA Binding Molecules capable of binding to CD137 and to the TA, PD-L1, or HER2, (designated “TRIDENT-A,” “TRIDENT-A4,” “TRIDENT-A5,” “TRIDENT-A6,” “TRIDENT-B1,” “TRIDENT-B1,”).

### A. Tetravalent CD137×TA Binding Molecules

### 1. DART-A

DART-A is a tetravalent CD137×CD137×TA×TA Binding Molecule having two CD137 binding sites and two binding site for the representative TA, PD-L1. DART-A is composed of four polypeptide chains, in which the first and third polypeptide chains are the same and the second and fourth polypeptide chains are the same (see FIG. 1B). DART-A comprises the binding domains of CD137 MAB-6(1.1) and hPD-L1 MAB-2(1.1).

The first and third polypeptide chains of DART-A comprise, in the N-terminal to C-terminal direction, an N-terminus, a VL domain of a monoclonal antibody capable of binding to PD-L1 (V_{LPD-L1}) (hPD-L1 MAB-2 VL1 (SEQ ID NO:58)), an intervening linker peptide (Linker 1; GGGSGGGG (SEQ ID NO:16)), a VH domain of a monoclonal antibody capable of binding to CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAACEK-EVAALEK-EVAALEK-EVAALEK (SEQ ID NO:39)), a linker (LEPKSADKTHTCPPCP (SEQ ID NO:30)), the CH2-CH3 Domain of a representative human IgG1 comprising the L234A/L235A/M252Y/S254T/T256E substitutions (SEQ ID NO:43, wherein X is absent), and a C-terminus.

Thus, the first and third polypeptide chain of DART-A are composed of: SEQ ID NO:58-SEQ ID NO:16-SEQ ID NO:46-SEQ ID NO:18-SEQ ID NO:39-SEQ ID NO:30-SEQ ID NO: 43.

The amino acid sequence of the first and third polypeptide chains of DART-A is (SEQ ID NO:116):

DIQMTQSPSS LSASVGDRVT ITCKASQDVN TAVAWYQQKP
GKAPKLLIYW ASTRHTGVPS RFSGSGSGTD FTLTISSLQP
EDFATYYCQQ HYNTPLTFGQ GTKVEIKGGG SGGGGQVQLQ
ESGPGLVKPS ETLSLTCTVS GGSISSYYWS WIRQPPGKGL
EWIGRIYTSG STNYNPSLKS RVTMSVDTSK NQFSLKLSSV
TAADTAVYYC ARDGWYDEDY NYYGMDVWGQ GTTVTVSSGG
CGGGEVAACE KEVAALEKEV AALEKEVAAL EKLEPKSADK
THTCPPCPAP EAAGGPSVFL FPPKPKDTLY ITREPEVTCV
VVDVSHEDPE VKFNWYVDGV EVHNAKTKPR EEQYNSTYRV
VSVLTVLHQD WLNGKEYKCK VSNKALPAPI EKTISKAKGQ
PREPQVYTLP PSREEMTKNQ VSLTCLVKGF YPSDIAVEWE
SNGQPENNYK TTPPVLDSDG SFFLYSKLTV DKSRWQQGNV
FSCSVMHEAL HNHYTQKSLS LSPG

Alternative DART-A first and third polypeptide chains may be employed that comprise Heterodimer-Promoting (E-coil) Domain lacking a cysteine residue (EVAALEK-EVAALEK-EVAALEK-EVAALEK (SEQ ID NO:37)). Such alternative DART-A first and third polypeptide chains are composed of: SEQ ID NO:58-SEQ ID NO:16-SEQ ID NO:46-SEQ ID NO:18-SEQ ID NO:37-SEQ ID NO:30-SEQ ID NO:43. Additional alternative DART-A first and third polypeptide chains may be employed in which the amino acid residues SEQ ID NO:58 (hPD-L1 MAB-2 VL1) are replaced with the amino acid residues of SEQ ID NO:72 (hPD-L1 MAB-2 VL2). Alternative molecules comprising many of such polypeptide chains are described below.

The second and fourth polypeptide chain of DART-A comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH1, SEQ ID NO:57)), an intervening linker peptide (Linker 2; GGCGGG (SEQ ID NO:18)), a Heterodimer-Promoting (K-coil) Domain (_KVAACKE-KVAALKE-KVAALKE-KVAALKE (SEQ ID NO:40), and a C-terminus.

Thus, the second and fourth polypeptide chain of DART-A are composed of: SEQ ID NO:50-SEQ ID NO:16-SEQ ID NO:57-SEQ ID NO:18-SEQ ID NO:40.

The amino acid sequence of the second and fourth polypeptide chain of DART-A is (SEQ ID NO:117):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIG GGTTYYPDTV KGRFTISRDN AKNTLYLQMN
SLKTEDTAVY YCARQGLPYY FDYWGQGTLV TVSSGGCGGG
KVAACKEKVA ALKEKVAALK EKVAALKE

Alternative DART-A second and fourth polypeptide chains may be employed that comprise a Heterodimer-Promoting (K-coil) Domain lacking a cysteine residue (e.g., KVAALKE-KVAALKE-KVAALKE-KVAALKE (SEQ ID NO:38)). Such alternative second and fourth polypeptide chains of DART-A sometimes are composed of: SEQ ID NO:50-SEQ ID NO:16-SEQ ID NO:57-SEQ ID NO:18-SEQ ID NO:38. Additional alternative DART-A first and third polypeptide chains may be employed in which the amino acid residues of SEQ ID NO:50 (CD137 MAB-6 VL1) are replaced with the amino acid residues of SEQ ID NO:55 (CD137 MAB-6 VL2), or SEQ ID NO:56 (CD137 MAB-6 VL3), and/or the amino acid residues of SEQ ID NO:57 (hPD-L1 MAB-2 VH1) are replaced with the amino acid residues of SEQ ID NO:67 (hPD-L1 MAB-2 VH2), SEQ ID NO:68 (hPD-L1 MAB-2 VH3), SEQ ID NO:69 (hPD-L1 MAB-2 VH4), SEQ ID NO:70 (hPD-L1 MAB-2 VH5), or SEQ ID NO:72 (hPD-L1 MAB-2 VH6). Alternatively, the PD-L1 VL/VH domains may be replaced with the VL/VH domains of a TA binding molecule that binds a different epitope of PD-L1 or that binds a different TA. Alternative molecules comprising many of such polypeptide chains are described below.

### 2. DART-A1

DART-A1 is a tetravalent CD137×CD137×TA×TA Binding Molecule having two CD137 binding sites and two binding sites for the representative TA, PD-L1. DART-A1 is composed of four polypeptide chains, in which the first and third polypeptide chains are the same and the second and fourth polypeptide chains are the same (see FIG. 3B). DART-A1 comprises the binding domains of CD137 MAB-6(1.1) and hPD-L1 MAB-2(2.1).

The first and third polypeptide chain of DART-A1 comprise, in the N-terminal to C-terminal direction, an N-terminus, a VL domain of a monoclonal antibody capable of binding to PD-L1 (VL_{PD-L1}) (hPD-L1 MAB-2 VL1 (SEQ ID NO:58)), an intervening linker peptide (Linker 1; GGGSGGGG (SEQ ID NO:16)), a VH domain of a monoclonal antibody capable of binding to CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAAQEK-EVAALEK-EVAALEK-EVAALEK (SEQ ID NO:39)), a linker (LEPKSADKTHTCPPCP (SEQ ID NO:30)), the CH2-CH3 Domain of an representative human IgG1 comprising the L234A/L235A/M252Y/S254T/T256E substitutions (SEQ ID NO:43), and a C-terminus.

Thus, the first and third polypeptide chain of DART-A1 are composed of: SEQ ID NO:58-SEQ ID NO:16-SEQ ID NO:46-SEQ ID NO:18-SEQ ID NO:39-SEQ ID NO:30-SEQ ID NO43.

The amino acid sequence of the first and third polypeptide chains of DART-A1 is (SEQ ID NO:118):

DIQMTQSPSS LSASVGDRVT ITCKASQDVN TAVAWYQQKP
GKAPKLLIYW ASTRHTGVPS RFSGSGSGTD FTLTISSLQP
EDFATYYCQQ HYNTPLTFGQ GTKVEIKGGG SGGGGQVQLQ
ESGPGLVKPS ETLSLTCTVS GGSISSYYWS WIRQPPGKGL
EWIGRIYTSG STNYNPSLKS RVTMSVDTSK NQFSLKLSSV
TAADTAVYYC ARDGWYDEDY NYYGMDVWGQ GTTVTVSSGG
CGGGEVAACE KEVAALEKEV AALEKEVAAL EKLEPKSADK
THTCPPCPAP EAAGGPSVFL FPPKPKDTLY ITREPEVTCV
VVDVSHEDPE VKFNWYVDGV EVHNAKTKPR EEQYNSTYRV
VSVLTVLHQD WLNGKEYKCK VSNKALPAPI EKTISKAKGQ
PREPQVYTLP PSREEMTKNQ VSLTCLVKGF YPSDIAVEWE
SNGQPENNYK TTPPVLDSDG SFFLYSKLTV DKSRWQQGNV
FSCSVMHEAL HNHYTQKSLS LSPGK

The second and fourth polypeptide chain of DART-A1 comprise, in the N-terminal to C-terminal direction, an N-terminus, a VL domain of a monoclonal antibody capable of binding to CD137 (V_{LCD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH2, SEQ ID NO:67)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A1 are composed of: SEQ ID NO:50-SEQ ID NO:16-SEQ ID NO:67-SEQ ID NO:18-SEQ ID NO:40.

The amino acid sequence of the second and fourth polypeptide chain of DART-A1 is (SEQ ID NO:119):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIG GGTTYYPDTV KGRFTISRDN AKNSLYLQMN
SLRAEDTAVY YCARQGLPYY FDYWGQGTLV TVSSGGCGGG
KVAACKEKVA ALKEKVAALK EKVAALKE

It is specifically contemplated that alternative DART-A1 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A1 first/third polypeptide chains sometimes are composed of: SEQ ID NO:58-SEQ ID NO:16-SEQ ID NO:46-SEQ ID NO:18-SEQ ID NO:37-SEQ ID NO:30-SEQ ID NO:43, and such alternative DART-A1 second/fourth chains sometimes are composed of: SEQ ID NO:50-SEQ ID NO:16-SEQ ID NO:67-SEQ ID NO:18-SEQ ID NO:38.

### 3. DART-A2

DART-A2 is a tetravalent CD137×CD137×TA×TA Binding Molecule having two CD137 binding sites and two binding sites for the representative TA, PD-L1. DART-A2 is composed of four polypeptide chains, in which the first and third polypeptide chains are the same and the second and fourth polypeptide chains are the same (see FIG. 3B). DART-A2 comprises the binding domains of CD137 MAB-6(1.1) and hPD-L1 MAB-2(2.2).

The first and third polypeptide chain of DART-A2 comprise, in the N-terminal to C-terminal direction, an N-terminus, a VL domain of a monoclonal antibody capable of binding to PD-L1 (VL_{PD-L1}) (hPD-L1 MAB-2 VL2 (SEQ ID NO:72)), an intervening linker peptide (Linker 1; GGGSGGGG (SEQ ID NO:16)), a VH domain of a monoclonal antibody capable of binding to CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAACEK-EVAALEK-EVAALEK-EVAALEK (SEQ ID NO:39)), a linker (LEPKSADKTHTCPPCP (SEQ ID NO:30)), the CH2-CH3 Domain of an representative human IgG1 comprising the L234A/L235A/M252Y/S254T/T256E substitutions (SEQ ID NO:43), and a C-terminus.

Thus, the first and third polypeptide chain of DART-A2 are composed of: SEQ ID NO:72-SEQ ID NO:16-SEQ ID NO:46-SEQ ID NO:18-SEQ ID NO:39-SEQ ID NO:30-SEQ ID NO:43.

The amino acid sequence of the first and third polypeptide chains of DART-A2 is (SEQ ID NO:120):

DIQMTQSPSS LSASVGDRVT ITCKASQDVN EAVAWYQQKP
GKAPKLLIYW ASTRHTGVPS RFSGSGSGTD FTLTISSLQP
EDFATYYCQQ HYNTPLTFGQ GTKVEIKGGG SGGGGQVQLQ
ESGPGLVKPS ETLSLTCTVS GGSISSYYWS WIRQPPGKGL
EWIGRIYTSG STNYNPSLKS RVTMSVDTSK NQFSLKLSSV
TAADTAVYYC ARDGWYDEDY NYYGMDVWGQ GTTVTVSSGG
CGGGEVAACE KEVAALEKEV AALEKEVAAL EKLEPKSADK
THTCPPCPAP EAAGGPSVFL FPPKPKDTLY ITREPEVTCV
VVDVSHEDPE VKFNWYVDGV EVHNAKTKPR EEQYNSTYRV
VSVLTVLHQD WLNGKEYKCK VSNKALPAPI EKTISKAKGQ
PREPQVYTLP PSREEMTKNQ VSLTCLVKGF YPSDIAVEWE
SNGQPENNYK TTPPVLDSDG SFFLYSKLTV DKSRWQQGNV
FSCSVMHEAL HNHYTQKSLS LSPGK

The second and fourth polypeptide chain of DART-A2 are the same as the second and fourth polypeptide chain of DART-A1 (SEQ ID NO:119).

It is specifically contemplated that alternative DART-A2 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A2 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A2 second/fourth chains sometimes are composed of: SEQ ID NO:50-SEQ ID NO:16-SEQ ID NO:67-SEQ ID NO:18-SEQ ID NO:38.

### 4. DART-A3

DART-A3 is a tetravalent CD137×CD137×TA×TA Binding Molecule having two CD137 binding sites and two binding sites for the representative TA, PD-L1. DART-A3 is composed of four polypeptide chains, in which the first and third polypeptide chains are the same and the second and fourth polypeptide chains are the same (see FIG. 3B). DART-A3 comprises the binding domains of CD137 MAB-6(1.1) and hPD-L1 MAB-2(3.1).

The first and third polypeptide chain of DART-A3 are the same as the first and third polypeptide chain of DART-A1 (SEQ ID NO:118).

The second and fourth polypeptide chain of DART-A3 comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH3, SEQ ID NO:68)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A3 are composed of: SEQ ID NO:50-SEQ ID NO:16-SEQ ID NO:68-SEQ ID NO:18-SEQ ID NO:40.

The amino acid sequence of the second and fourth polypeptide chain of DART-A3 is (SEQ ID NO:121):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIK GGTTYYPDTV KGRFTISRDN AKNSLYLQMN
SLRAEDTAVY YCARQGLPYY GDYWGQGTLV TVSSGGCGGG
KVAACKEKVA ALKEKVAALK EKVAALKE

It is specifically contemplated that alternative DART-A3 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A3 first/third polypeptide chains sometimes are composed of: SEQ ID NO:58-SEQ ID NO:16-SEQ ID NO:46-SEQ ID NO:18-SEQ ID NO:37-SEQ ID NO:30-SEQ ID NO:43, and such alternative DART-A3 second/fourth chains sometimes are composed of: SEQ ID NO:50-SEQ ID NO:16-SEQ ID NO:68-SEQ ID NO:18-SEQ ID NO:38.

### 5. DART-A4

DART-A4 is a tetravalent CD137×CD137×TA×TA Binding Molecule having two CD137 binding sites and two binding sites for the representative TA, PD-L1. DART-A4 is composed of four polypeptide chains, in which the first and third polypeptide chains are the same and the second and fourth polypeptide chains are the same (see FIG. 3B). DART-A4 comprises the binding domains of CD137 MAB-6(1.1) and hPD-L1 MAB-2(3.2).

The first and third polypeptide chain of DART-A4 are the same as the first and third polypeptide chain of DART-A2 (SEQ ID NO:120).

The second and fourth polypeptide chain of DART-A4 are the same as the second and fourth polypeptide chain of DART-A3 (SEQ ID NO:121).

It is specifically contemplated that alternative DART-A4 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A4 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A4 second/fourth chains sometimes are composed of: SEQ ID NO:50-SEQ ID NO:16-SEQ ID NO:68-SEQ ID NO:18-SEQ ID NO:38.

### 6. DART-A5

DART-A5 is a tetravalent CD137×CD137×TA×TA Binding Molecule having two CD137 binding sites and two binding sites for the representative TA, PD-L1. DART-A5 is composed of four polypeptide chains, in which the first and third polypeptide chains are the same and the second and fourth polypeptide chains are the same (see FIG. 3B). DART-A5 comprises the binding domains of CD137 MAB-6(1.2) and hPD-L1 MAB-2(3.2).

The first and third polypeptide chain of DART-A5 are the same as the first and third polypeptide chain of DART-A2 (SEQ ID NO:120).

The second and fourth polypeptide chain of DART-A5 comprise, in the N-terminal to C-terminal direction, an N-terminus, a VL domain of a monoclonal antibody capable of binding to CD137 (VL_{CD137} (CD137 MAB-6 VL2 (SEQ ID NO:55)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH2, SEQ ID NO:68)), an intervening linker peptide (Linker 2; GGCGGG (SEQ ID NO:18)), a Heterodimer-Promoting (K-coil) Domain (_KVAACKE-KVAALKE-KVAALKE-KVAALKE (SEQ ID NO:40), and a C-terminus.

Thus, the second and fourth polypeptide chain of DART-A5 are composed of: SEQ ID NO:55-SEQ ID NO:16-SEQ ID NO:68-SEQ ID NO:18-SEQ ID NO:40.

The amino acid sequence of the second and fourth polypeptide chain of DART-A5 is (SEQ ID NO:122):

EIVMTQSPAT LSLSPGERAT LSCRASQSVS SNYLSWYQQK
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIK GGTTYYPDTV KGRFTISRDN AKNSLYLQMN
SLRAEDTAVY YCARQGLPYY GDYWGQGTLV TVSSGGCGGG
KVAACKEKVA ALKEKVAALK EKVAALKE

It is specifically contemplated that alternative DART-A5 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A5 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A5 second/fourth chains sometimes are composed of: SEQ ID NO:55-SEQ ID NO:16-SEQ ID NO:68-SEQ ID NO:18-SEQ ID NO:38.

### 7. DART-A6

DART-A6 is a tetravalent CD137×CD137×TA×TA Binding Molecule having two CD137 binding sites and two binding sites for the representative TA, PD-L1. DART-A6 is composed of four polypeptide chains, in which the first and third polypeptide chains are the same and the second and fourth polypeptide chains are the same (see FIG. 3B). DART-A6 comprises the binding domains of CD137 MAB-6(1.3) and hPD-L1 MAB-2(3.2).

The first and third polypeptide chain of DART-A6 are the same as the first and third polypeptide chain of DART-A2 (SEQ ID NO:120).

The second and fourth polypeptide chain of DART-A6 comprise, in the N-terminal to C-terminal direction, an N-terminus, a VL domain of a monoclonal antibody capable of binding to CD137 (VL_{CD137} (CD137 MAB-6 VL3 (SEQ ID NO:56)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH3, SEQ ID NO:68)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A6 are composed of: SEQ ID NO:56-SEQ ID NO:16-SEQ ID NO:68-SEQ ID NO:18-SEQ ID NO:40.

The amino acid sequence of the second and fourth polypeptide chain of DART-A6 is (SEQ ID NO:123):

EIVMTQSPAT LSLSPGERAT LSCRASQSVS SNYLSWFQQK
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIK GGTTYYPDTV KGRFTISRDN AKNSLYLQMN
SLRAEDTAVY YCARQGLPYY GDYWGQGTLV TVSSGGCGGG
KVAACKEKVA ALKEKVAALK EKVAALKE

It is specifically contemplated that alternative DART-A6 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A6 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A6 second/fourth chains sometimes are composed of: SEQ ID NO:56-SEQ ID NO:16-SEQ ID NO:68-SEQ ID NO:18-SEQ ID NO:38.

### 8. DART-A7

DART-A7 is a tetravalent CD137×CD137×TA×TA Binding Molecule having two CD137 binding sites and two binding sites for the representative TA, PD-L1. DART-A7 is composed of four polypeptide chains, in which the first and third polypeptide chains are the same and the second and fourth polypeptide chains are the same (see FIG. 3B). DART-A7 comprises the binding domains of CD137 MAB-6(1.1) and hPD-L1 MAB-2(4.2).

The first and third polypeptide chain of DART-A7 are the same as the first and third polypeptide chain of DART-A2 (SEQ ID NO:120).

The second and fourth polypeptide chain of DART-A7 comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH4, SEQ ID NO:69)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A7 are composed of: SEQ ID NO:50-SEQ ID NO:16-SEQ ID NO:69-SEQ ID NO:18-SEQ ID NO:40.

The amino acid sequence of the second and fourth polypeptide chain of DART-A7 is (SEQ ID NO:124):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIG GGTTYYPDTV KGRFTISRDN AKNSLYLQMN
SLRAEDTAVY YCARAGLPYY FDYWGQGTLV TVSSGGCGGG
KVAACKEKVA ALKEKVAALK EKVAALKE

It is specifically contemplated that alternative DART-A7 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A7 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A7 second/fourth chains sometimes are composed of: SEQ ID NO:50-SEQ ID NO:16-SEQ ID NO:69-SEQ ID NO:18-SEQ ID NO:38.

### 9. DART-A8

DART-A8 is a tetravalent CD137×CD137×TA×TA Binding Molecule having two CD137 binding sites and two binding sites for the representative TA, PD-L1. DART-A8 is composed of four polypeptide chains, in which the first and third polypeptide chains are the same and the second and fourth polypeptide chains are the same (see FIG. 3B). DART-A8 comprises the binding domains of CD137 MAB-6(1.1) and hPD-L1 MAB-2(5.2).

The first and third polypeptide chain of DART-A8 are the same as the first and third polypeptide chain of DART-A2 (SEQ ID NO:120).

The second and fourth polypeptide chain of DART-A8 comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH5, SEQ ID NO:70)), an intervening linker peptide (Linker 2; GGCGGG (SEQ ID NO:18)), a Heterodimer-Promoting (K-coil) Domain (_KVAACKE-KVAALKE-KVAALKE-KVAALKE (SEQ ID NO:40), and a C-terminus.

Thus, the second and fourth polypeptide chain of DART-A8 are composed of: SEQ ID NO:50-SEQ ID NO:16-SEQ ID NO:70-SEQ ID NO:18-SEQ ID NO:40.

The amino acid sequence of the second and fourth polypeptide chain of DART-A8 is (SEQ ID NO:125):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIK GGTTYYPDTV KGRFTISRDN AKNSLYLQMN
SLRAEDTAVY YCARAGLPYY FDYWGQGTLV TVSSGGCGGG
KVAACKEKVA ALKEKVAALK EKVAALKE

It is specifically contemplated that alternative DART-A8 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A8 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A8 second/fourth chains sometimes are composed of: SEQ ID NO:50-SEQ ID NO:16-SEQ ID NO:70-SEQ ID NO:18-SEQ ID NO:38.

### 10. DART-A9

DART-A9 is a tetravalent CD137×CD137×TA×TA Binding Molecule having two CD137 binding sites and two binding sites for the representative TA, PD-L1. DART-A9 is composed of four polypeptide chains, in which the first and third polypeptide chains are the same and the second and fourth polypeptide chains are the same (see FIG. 3B). DART-A9 comprises the binding domains of CD137 MAB-6(1.1) and hPD-L1 MAB-2(6.2).

The first and third polypeptide chain of DART-A9 are the same as the first and third polypeptide chain of DART-A2 (SEQ ID NO:120).

The second and fourth polypeptide chain of DART-A9 comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH6, SEQ ID NO:71)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A9 are composed of: SEQ ID NO:50-SEQ ID NO:16-SEQ ID NO:71-SEQ ID NO:18-SEQID NO:40.

The amino acid sequence of the second and fourth polypeptide chain of DART-A9 is (SEQ ID NO:126):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIG GGTTYYPDTV KGRFTISRDN AKNSLYLQMN
SLRAEDTAVY YCARAGLPYY GDYWGQGTLV TVSSGGCGGG
KVAACKEKVA ALKEKVAALK EKVAALKE

It is specifically contemplated that alternative DART-A9 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A9 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A9 second/fourth chains sometimes are composed of: SEQ ID NO:50-SEQ ID NO:16-SEQ ID NO:71-SEQ ID NO:18-SEQ ID NO:38.

### 11. DART-A10

DART-A10 is a tetravalent CD137×CD137×TA×TA Binding Molecule having two CD137 binding sites and two binding sites for the representative TA, PD-L1. DART-A10 is composed of four polypeptide chains, in which the first and third polypeptide chains are the same and the second and fourth polypeptide chains are the same (see FIG. 3B). DART-A10 comprises the binding domains of CD137 MAB-6(1.3) and hPD-L1 MAB-2(4.2).

The first and third polypeptide chain of DART-A10 are the same as the first and third polypeptide chain of DART-A2 (SEQ ID NO:120).

The second and fourth polypeptide chain of DART-A10 comprise, in the N-terminal to C-terminal direction, an N-terminus, a VL domain of a monoclonal antibody capable of binding to CD137 (VL_{CD137} (CD137 MAB-6 VL3 (SEQ ID NO:56)), an intervening linker peptide (Linker 1; GGGSGGGG (SEQ ID NO:16)), a VH domain of a monoclonal antibody capable of binding to PD-L1 (VH_{PD-L1} (hPD-L1 MAB-2 VH4, SEQ ID NO:69)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-A10 are composed of: SEQ ID NO:56-SEQ ID NO:16-SEQ ID NO:69-SEQ ID NO:18-SEQ ID NO:40.

The amino acid sequence of the second and fourth polypeptide chain of DART-A10 is (SEQ ID NO:139):

EIVMTQSPAT LSLSPGERAT LSCRASQSVS SNYLSWFQQK
PGQAPRLLIY GASTRATGIP ARFSGSGSGT DFTLTISSLQ
PEDFAVYYCQ QDYDLPWTFG QGTKVEIKGG GSGGGGEVQL
VESGGGLVQP GGSLRLSCAA SGFTFSSYTM SWVRQAPGKG
LEWVAYISIG GGTTYYPDTV KGRFTISRDN AKNSLYLQMN
SLRAEDTAVY YCARAGLPYY FDYWGQGTLV TVSSGGCGGG
KVAACKEKVA ALKEKVAALK EKVAALKE

It is specifically contemplated that alternative DART-A10 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-A10 first/third polypeptide chains sometimes are composed of: SEQ ID NO:72-SEQ ID NO:16-SEQ ID NO:46-SEQ ID NO:18-SEQ ID NO:37-SEQ ID NO:30-SEQ ID NO:43, and such alternative DART-A10 second/fourth chains sometimes are composed of: SEQ ID NO:56-SEQ ID NO:16-SEQ ID NO:69-SEQ ID NO:18-SEQ ID NO:38.

### 12. DART-B1

DART-B1 is a bivalent CD137×TA Binding Molecule having one CD137 binding site and one binding site for the representative TA, HER2. DART-B1 is composed of three polypeptide chains, in which the first, second, and third polypeptide chains are different (see FIG. 1D). DART-B1 comprises the binding domains of CD137 MAB-6(1.1) and hHER2 MAB-1(1.3).

The first polypeptide chain of DART-B1 comprises, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to HER2 (VHHER2 (hHER2 MAB-1 VH1, SEQ ID NO:80)), an intervening linker peptide (Linker 2; GGCGGG (SEQ ID NO:18)), a Heterodimer-Promoting (E-coil) Domain (EVAALEK-EVAALEK-EVAALEK-EVAALEK (SEQ ID NO:37)), an intervening linker peptide (GGGDKTHTCPPCP (SEQ ID NO:21)), a “knob-bearing” CH2 and CH3 Domain comprising the L234A/L235A/M252Y/S254T/T256E substitutions (SEQ ID NO:146), and a C-terminus.

Thus, the first polypeptide chain of DART-B1 is composed of: SEQ ID NO:50-SEQ ID NO:16-SEQ ID NO:80-SEQ ID NO:18-SEQ ID NO:37-SEQ ID NO:21-SEQ ID NO:146.

The amino acid sequence of the first polypeptide chain of DART-B1 is (SEQ ID NO:143):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGQVQL
VQSGAEVKKP GASVKVSCKA SGYTFTNYGM NWVRQAPGQG
LEWMGWINTN IGEPTYTEEF KGRVTMTRDT SISTAYMELS
RLRSDDTAVY YCARDDGYGN RVSYWGQGTL VTVSSGGCGG
GEVAALEKEV AALEKEVAAL EKEVAALEKG GGDKTHTCPP
CPAPEAAGGP SVFLFPPKPK DTLYITREPE VTCVVVDVSH
EDPEVKFNWY VDGVEVHNAK TKPREEQYNS TYRVVSVLTV
LHQDWLNGKE YKCKVSNKAL PAPIEKTISK AKGQPREPQV
YTLPPSREEM TKNQVSLWCL VKGFYPSDIA VEWESNGQPE
NNYKTTPPVL DSDGSFFLYS KLTVDKSRWQ QGNVFSCSVM
HEALHNHYTQ KSLSLSPGK

The second polypeptide chain of DART-B1 comprises, in the N-terminal to C-terminal direction, an N-terminus, a VL domain of a monoclonal antibody capable of binding to HER2 (VL_{HER2} (hHER2 MAB-1 VL3, SEQ ID NO:85)), an intervening linker peptide (Linker 1; GGGSGGGG (SEQ ID NO:16)), a VH domain of a monoclonal antibody capable of binding to CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (K-coil) Domain (_KVAALKE-KVAALKE-KVAALKE-KVAALKE (SEQ ID NO:38)), and a C-terminus.

Thus, the second polypeptide chain of DART-B1 is composed of: SEQ ID NO:85-SEQ ID NO:16-SEQ ID NO:46-SEQ ID NO:18-SEQ ID NO:38.

The amino acid sequence of the second polypeptide chain of DART-B1 is (SEQ ID NO:144):

DIQMTQSPSS LSASVGDRVT ITCKASQDIS NYLSWFQQKP
GKAPKTLIYR ANRLQSGVPS RFSGSGSGTD FTLTISSLQP
EDFATYYCLQ HDEFPWTFGQ GTKLEIKGGG SGGGGQVQLQ
ESGPGLVKPS ETLSLTCTVS GGSISSYYWS WIRQPPGKGL
EWIGRIYTSG STNYNPSLKS RVTMSVDTSK NQFSLKLSSV
TAADTAVYYC ARDGWYDEDY NYYGMDVWGQ GTTVTVSSGG
CGGGKVAALK EKVAALKEKV AALKEKVAAL KE

The third polypeptide chain of DART-B1 comprises, in the N-terminal to C-terminal direction, a Linker DKTHTCPPCP (SEQ ID NO:20) and a “hole-bearing” CH2 and CH3 Domain comprising the L234A/L235A/M252Y/S254T/T256E/H435R substitutions (SEQ ID NO:149).

Thus the third polypeptide chain of DART-B1 is composed of: SEQ ID NO:20-SEQ ID NO:149.

As will be recognized, the third polypeptide chain of DART-B does not contain any Epitope-Binding Domains and may thus be employed in various CD137×TA Binding Molecules having the diabody structure shown in FIG. 1D.

The third polypeptide chain of DART-B1 has the amino acid sequence of SEQ ID NO:145:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

### 13. DART-B2

DART-B2 is a tetravalent CD137×CD137×TA×TA Binding Molecule having two CD137 binding sites and two binding site for the representative TA, HER2. DART-B1 is composed of four polypeptide chains, in which the first and third polypeptide chains are the same and the second and fourth polypeptide chains are the same (see FIG. 1B). DART-B1 comprises the binding domains of CD137 MAB-6(1.1) and hHER2 MAB-1(1.3).

The first and third polypeptide chains of DART-B2 comprises, in the N-terminal to C-terminal direction, an N-terminus, a VL domain of a monoclonal antibody capable of binding to HER2 (VL_{HER2} (hHER2 MAB-1 VL3, SEQ ID NO:85)), an intervening linker peptide (Linker 1; GGGSGGGG (SEQ ID NO:16)), a VH domain of a monoclonal antibody capable of binding to CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAACEK-EVAALEK-EVAALEK-EVAALEK (SEQ ID NO:39)), a linker (LEPKSADKTHTCPPCP (SEQ ID NO:30)), the CH2-CH3 Domain of a representative human IgG1 comprising the L234A/L235A/M252Y/S254T/T256E substitutions (SEQ ID NO:43, wherein X is absent), and a C-terminus.

Thus, the first and third polypeptide chain of DART-B2 are composed of: SEQ ID NO:85-SEQ ID NO:16-SEQ ID NO:46-SEQ ID NO:18-SEQ ID NO:39-SEQ ID NO:30-SEQ ID NO: 43.

The amino acid sequence of the first and third polypeptide chains of DART-B2 is (SEQ ID NO:151):

DIQMTQSPSS LSASVGDRVT ITCKASQDIS NYLSWFQQKP
GKAPKTLIYR ANRLQSGVPS RFSGSGSGTD FTLTISSLQP
EDFATYYCLQ HDEFPWTFGQ GTKLEIKGGG SGGGGQVQLQ
ESGPGLVKPS ETLSLTCTVS GGSISSYYWS WIRQPPGKGL
EWIGRIYTSG STNYNPSLKS RVTMSVDTSK NQFSLKLSSV
TAADTAVYYC ARDGWYDEDY NYYGMDVWGQ GTTVTVSSGG
CGGGEVAACE KEVAALEKEV AALEKEVAAL EKLEPKSADK
THTCPPCPAP EAAGGPSVFL FPPKPKDTLY ITREPEVTCV
VVDVSHEDPE VKFNWYVDGV EVHNAKTKPR EEQYNSTYRV
VSVLTVLHQD WLNGKEYKCK VSNKALPAPI EKTISKAKGQ
PREPQVYTLP PSREEMTKNQ VSLTCLVKGF YPSDIAVEWE
SNGQPENNYK TTPPVLDSDG SFFLYSKLTV DKSRWQQGNV
FSCSVMHEAL HNHYTQKSLS LSPGK

The second and fourth polypeptide chain of DART-B2 comprise, in the N-terminal to C-terminal direction, an N-terminus, a VL domain of a monoclonal antibody capable of binding to CD137 (VL_{CD137} (CD137 MAB-6 VL1 (SEQ ID NO:50)), an intervening linker peptide (Linker 1; GGGSGGGG (SEQ ID NO:16)), a VH domain of a monoclonal antibody capable of binding to HER2 (VHHER2 (hHER2 MAB-1 VH1, SEQ ID NO:80)), an intervening linker peptide (Linker 2; GGCGGG (SEQ ID NO:18)), a Heterodimer-Promoting (K-coil) Domain (KVAACKE-KVAALKE-KVAALKE-KVAALKE (SEQ ID NO:40), and a C-terminus.

Thus, the second and fourth polypeptide chain of DART-B2 are composed of: SEQ ID NO:50-SEQ ID NO:16-SEQ ID NO:80-SEQ ID NO:18-SEQ ID NO:40.

The amino acid sequence of the second and fourth polypeptide chains of DART-B7 is (SEQ ID NO-157):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGQVQL
VQSGAEVKKP GASVKVSCKA SGYTFTNYGM NWVRQAPGQG
LEWMGWINTN IGEPTYTEEF KGRVTMTRDT SISTAYMELS
RLRSDDTAVY YCARDDGYGN RVSYWGQGTL VTVSSGGCGG
GKVAACKEKV AALKEKVAAL KEKVAALKE

It is specifically contemplated that alternative DART-B2 first/third and second/forth polypeptide chains may be employed that comprise Heterodimer-Promoting (E-coil and K-coil) Domains lacking a cysteine residue (e.g., EVAALEK-EVAALEK-EVAALEK-EVAALEK (SEQ ID NO:37) and KVAALKE-KVAALKE-KVAALKE-KVAALKE (SEQ ID NO:38)). Such alternative DART-B2 first/third polypeptide chains sometimes are composed of: SEQ ID NO:85-SEQ ID NO:16-SEQ ID NO:46-SEQ ID NO:18-SEQ ID NO:37-SEQ ID NO:30-SEQ ID NO:43, and such alternative DART-B2 second/fourth chains sometimes are composed of: SEQ ID NO:50-SEQ ID NO:16-SEQ ID NO:80-SEQ ID NO:18-SEQ ID NO:38.

### B. Trivalent CD137×TA Binding Molecules

### 1. TRIDENT-A

TRIDENT-A is a trivalent CD137×CD137×TA Binding Molecule having two CD137 binding sites and one binding site for the representative TA, PD-L1. TRIDENT-A is composed of four polypeptide chains (see, FIG. 3A, wherein VL1/VH1 (Site A) are the same as VL2/VH2 (Site B) and bind CD137, and VL3/VH3 (Site C) bind PD-L1). TRIDENT-A comprises the binding domains of CD137 MAB-6(1.1) and hPD-L1 MAB-2(1.1).

The first polypeptide chain of TRIDENT-A comprises, in the N-terminal to C-terminal direction, an N-terminus, a VL domain of a monoclonal antibody capable of binding CD137 (VL_{CD137}) (CD137 MAB-6 VL1 (SEQ ID NO:50)), an intervening linker peptide (Linker 1; GGGSGGGG (SEQ ID NO:16)), a VH domain of a monoclonal antibody capable of binding CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAALEK-EVAALEK-EVAALEK-EVAALEK (SEQ ID NO:37)), an intervening linker peptide (GGGDKTHTCPPCP (SEQ ID NO:21)), a “knob-bearing” CH2 and CH3 Domain comprising the L234A/L235A/M252Y/S254T/T256E substitutions (SEQ ID NO:146), and a C-terminus.

Thus, the first polypeptide chain of TRIDENT-A is composed of: SEQ ID NO:50-SEQID NO:16-SEQID NO:46-SEQID NO:18-SEQID NO:37-SEQID NO:21-SEQ ID NO:146.

The amino acid sequence of the first polypeptide chain of TRIDENT-A is (SEQ ID NO:127):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGEVAAL EKEVAALEKE VAALEKEVAA LEKGGGDKTH
TCPPCPAPEA AGGPSVFLFP PKPKDTLYIT REPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The second polypeptide chain of TRIDENT-A comprises, in the N-terminal to C-terminal direction, an N-terminus, a VL domain of a monoclonal antibody capable of binding CD137 (VL_{CD137}) (CD137 MAB-6 VL1 (SEQ ID NO:50)), an intervening linker peptide (Linker 1; GGGSGGGG (SEQ ID NO:16)), a VH domain of a monoclonal antibody capable of binding CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (K-coil) Domain (KVAALKE-KVAALKE-KVAALKE-KVAALKE (SEQ ID NO:38)), and a C-terminus.

Thus, the second polypeptide chain of TRIDENT-A is composed of: SEQ ID NO:50-SEQ ID NO:16-SEQ ID NO:46-SEQ ID NO:18-SEQ ID NO:38.

The amino acid sequence of the second polypeptide chain of TRIDENT-A is (SEQ ID NO:128):

EIVMTQSPAT LSLTPGERAT LSCRASQSVS SNYLSWFQQI
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGKVAAL KEKVAALKEK VAALKEKVAA LKE

Alternative TRIDENT-A first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-A molecules, the first polypeptide chain sometimes is composed of: SEQ ID NO:50-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:50-SEQ ID NO:16-SEQ ID NO:46-SEQ ID NO:18-SEQ ID NO:40. Additional alternative TRIDENT-A first and second polypeptide chains may be employed in which the amino acid residues of SEQ ID NO:50 (CD137 MAB-6 VL1) are replaced with the amino acid residues of SEQ ID NO:55 (CD137 MAB-6 VL2), or SEQ ID NO:56 (CD137 MAB-6 VL3). It is also specifically contemplated that one CD137 VL/VH domain pair may be replaced with the VL/VH pair of a TA binding molecule. Alternative molecules comprising many of such polypeptide chains are described below.

The third polypeptide chain of TRIDENT-A comprises, in the N-terminal to C-terminal direction, an N-terminus, a VH domain of a monoclonal antibody capable of binding to PD-L1 (VH_{PD-L1}) (hPD-L1 MAB-2 VH1 (SEQ ID NO:57)), a human IgG1 CHI Domain (SEQ ID NO:3), a human IgG1 Hinge Region (SEQ ID NO:7), and a “hole-bearing” CH2 and CH3 Domain comprising the L234A/L235A/M252Y/S254T/T256E/H435R substitutions (SEQ ID NO:149).

Thus, the third polypeptide chain of TRIDENT-A is composed of: SEQ ID NO:57-SEQ ID NO:3-SEQ ID NO:7-SEQ ID NO:149.

The amino acid sequence of the third polypeptide chain of TRIDENT-A is (SEQ ID NO:129):

EVQLVESGGG LVQPGGSLRL SCAASGFTFS SYTMSWVRQA
PGKGLEWVAY ISIGGGTTYY PDTVKGRFTI SRDNAKNTLY
LQMNSLKTED TAVYYCARQG LPYYFDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLYITREPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The fourth polypeptide chain of TRIDENT-A comprises, in the N-terminal to C-terminal direction, an N-terminus, a VL domain of a monoclonal antibody capable of binding to PD-L1 (VL_{PD-L1}) (hPD-L1 MAB-1 VL1 (SEQ ID NO:58)), a human IgG CL Kappa Domain (SEQ ID NO:1), and a C-terminus.

Thus, the fourth polypeptide chain of TRIDENT-A is composed of: SEQ ID NO:69-SEQ ID NO:1.

DIQMTQSPSS LSASVGDRVT ITCKASQDVN TAVAWYQQKP
GKAPKLLIYW ASTRHTGVPS RFSGSGSGTD FTLTISSLQP
EDFATYYCQQ HYNTPLTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

Alternative TRIDENT-A third and fourth polypeptide chains may be employed in which the amino acid residues of SEQ ID NO:57 (hPD-L1 MAB-2 VH1) are replaced with the amino acid residues of SEQ ID NO:67 (hPD-L1 MAB-2 VH2), SEQ ID NO:68 (hPD-L1 MAB-2 VH3), SEQ ID NO:69 (hPD-L1 MAB-2 VH4), SEQ ID NO:70 (hPD-L1 MAB-2 VH5), or SEQ ID NO:72 (hPD-L1 MAB-2 VH6), and/or the amino acid residues of SEQ ID NO:58 (hPD-L1 MAB-2 VL1) are replaced with the amino acid residues of SEQ ID NO:72 (hPD-L1 MAB-2 VL2). Alternatively, the PD-L1 VL/VH domains may be replaced with the VL/VH domains of a TA binding molecule that binds a different epitope of PD-L1 or that binds a different TA. It is also specifically contemplated that where a TA binding site is formed by the association of the first and second polypeptide chains, the VL/VH Domains of the third and fourth polypeptide chains may be replaced with any of the CD137 MAB-6 VL/VH domains provided herein. Alternative molecules comprising several of such polypeptide chains are described below.

### 2. TRIDENT-A4

TRIDENT-A4 is a trivalent CD137×CD137×TA Binding Molecule having two CD137 binding sites and one binding site for the representative TA, PD-L1. TRIDENT-A4 is composed of four polypeptide chains (see, FIG. 3A, wherein VL1/VH1 (Site A) are the same as VL2/VH2 (Site B) and bind CD137, and VL3/VH3 (Site C) bind PD-L1). TRIDENT-A4 comprises the binding domains of CD137 MAB-6(1.1) and hPD-L1 MAB-2(3.2).

The first polypeptide chain of TRIDENT-A4 is the same as the first polypeptide chain of TRIDENT-A (SEQ ID NO:127).

The second polypeptide chain of TRIDENT-A4 is the same as the second polypeptide chain of TRIDENT-A (SEQ ID NO:128).

It is specifically contemplated that alternative TRIDENT-A4 first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-A4 molecules, the first polypeptide chain sometimes is composed of: SEQ ID NO:50-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:50-SEQ ID NO:16-SEQ ID NO:46-SEQ ID NO:18-SEQ ID NO:40.

The third polypeptide chain of TRIDENT-A4 comprises, in the N-terminal to C-terminal direction, an N-terminus, a VH domain of a monoclonal antibody capable of binding to PD-L1 (VH_{PD-L1}) (hPD-L1 MAB-2 VH3 (SEQ ID NO:68)), a human IgG1 CH1 Domain (SEQ ID NO:3), a human IgG1 Hinge Region (SEQ ID NO:7), and a “hole-bearing” CH2 and CH3 Domain comprising the L234A/L235A/M252Y/S254T/T256E/H435R substitutions (SEQ ID NO:149).

Thus, the third polypeptide chain of TRIDENT-A4 is composed of: SEQ ID NO:68-SEQ ID NO:3-SEQ ID NO:7-SEQ ID NO: 149.

The amino acid sequence of the third polypeptide chain of TRIDENT-A4 is (SEQ ID NO:131):

EVQLVESGGG LVQPGGSLRL SCAASGFTFS SYTMSWVRQA
PGKGLEWVAY ISIKGGTTYY PDTVKGRFTI SRDNAKNSLY
LQMNSLRAED TAVYYCARQG LPYYGDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLYITREPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The fourth polypeptide chain of TRIDENT-A4 comprises, in the N-terminal to C-terminal direction, an N-terminus, a VL domain of a monoclonal antibody capable of binding to PD-L1 (VL_{PD-L1}) (hPD-L1 MAB-1 VL2 (SEQ ID NO:72)), a human IgG CL Kappa Domain (SEQ ID NO:1), and a C-terminus.

Thus, the fourth polypeptide chain of TRIDENT-A4 is composed of: SEQ ID NO:72-SEQ ID NO:1.

The amino acid sequence of the fourth polypeptide chain of TRIDENT-A is (SEQ ID NO:132):

DIQMTQSPSS LSASVGDRVT ITCKASQDVN EAVAWYQQKP
GKAPKLLIYW ASTRHTGVPS RFSGSGSGTD FTLTISSLQP
EDFATYYCQQ HYNTPLTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

### 3. TRIDENT-A5

TRIDENT-A5 is a trivalent CD137×CD137×TA Binding Molecule having two CD137 binding sites and one binding site for the representative TA, PD-L1. TRIDENT-A5 is composed of four polypeptide chains (see, FIG. 3A, wherein VL1/VH1 (Site A) are the same as VL2/VH2 (Site B) and bind CD137, and VL3/VH3 (Site C) bind PD-L1). TRIDENT-A5 comprises the binding domains of CD137 MAB-6(1.2) and hPD-L1 MAB-2(3.2).

The first polypeptide chain of TRIDENT-A5 comprises, in the N-terminal to C-terminal direction, an N-terminus, a VL domain of a monoclonal antibody capable of binding CD137 (VL_{CD137}) (CD137 MAB-6 VL2 (SEQ ID NO:55)), an intervening linker peptide (Linker 1; GGGSGGGG (SEQ ID NO:16)), a VH domain of a monoclonal antibody capable of binding CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAALEK-EVAALEK-EVAALEK-EVAALEK (SEQ ID NO:37)), an intervening linker peptide (GGGDKTHTCPPCP (SEQ ID NO:21)), a “knob-bearing” CH2 and CH3 Domain comprising the L234A/L235A/M252Y/S254T/T256E substitutions (SEQ ID NO:146), and a C-terminus.

Thus, the first polypeptide chain of TRIDENT-A5 is composed of: SEQ ID NO:55-SEQID NO:16-SEQID NO:46-SEQID NO:18-SEQID NO:37-SEQID NO:21-SEQ ID NO:146.

The amino acid sequence of the first polypeptide chain of TRIDENT-A5 is (SEQ ID NO:133):

EIVMTQSPAT LSLSPGERAT LSCRASQSVS SNYLSWYQQK
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGEVAAL EKEVAALEKE VAALEKEVAA LEKGGGDKTH
TCPPCPAPEA AGGPSVFLFP PKPKDTLYIT REPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The second polypeptide chain of TRIDENT-A5 comprises, in the N-terminal to C-terminal direction, an N-terminus, a VL domain of a monoclonal antibody capable of binding CD137 (VL_{CD137}) (CD137 MAB-6 VL2 (SEQ ID NO:55)), an intervening linker peptide (Linker 1; GGGSGGGG (SEQ ID NO:16)), a VH domain of a monoclonal antibody capable of binding CD137 (VHcD137) (CD137 MAB-6 VH1 (SEQ ID NO:46)), an intervening linker peptide (Linker 2; GGCGGG (SEQ ID NO:18)), a Heterodimer-Promoting (K-coil) Domain (KVAALKE-KVAALKE-KVAALKE-KVAALKE (SEQ ID NO:38)), and a C-terminus.

Thus, the second polypeptide chain of TRIDENT-A5 is composed of: SEQ ID NO:55-SEQ ID NO:16-SEQ ID NO:46-SEQ ID NO:18-SEQ ID NO:38.

The amino acid sequence of the second polypeptide chain of TRIDENT-A5 is (SEQ ID NO:134):

EIVMTQSPAT LSLSPGERAT LSCRASQSVS SNYLSWYQQK
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGKVAAL KEKVAALKEK VAALKEKVAA LKE

It is specifically contemplated that alternative TRIDENT-A5 first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-A5 molecules, the first polypeptide chain sometimes is composed of: SEQ ID NO:55-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:55-SEQ ID NO:16-SEQ ID NO:46-SEQ ID NO:18-SEQ ID NO:40.

The third polypeptide chain of TRIDENT-A5 is the same as the third polypeptide chain of TRIDENT-A4 (SEQ ID NO:131).

The fourth polypeptide chain of TRIDENT-A5 is the same as the fourth polypeptide chain of TRIDENT-A4 (SEQ ID NO:132).

### 4. TRIDENT-A6

TRIDENT-A6 is a trivalent CD137×CD137×TA Binding Molecule having two CD137 binding sites and one binding site for the representative TA, PD-L1. TRIDENT-A6 is composed of four polypeptide chains (see, FIG. 3A, wherein VL1/VH1 (Site A) are the same as VL2/VH2 (Site B) and bind CD137, and VL3/VH3 (Site C) bind PD-L1). TRIDENT-A6 comprises the binding domains of CD137 MAB-6(1.3) and hPD-L1 MAB-2(3.2).

The first polypeptide chain of TRIDENT-A6 comprises, in the N-terminal to C-terminal direction, an N-terminus, a VL domain of a monoclonal antibody capable of binding CD137 (VL_{CD137}) (CD137 MAB-6 VL3 (SEQ ID NO:56)), an intervening linker peptide (Linker 1; GGGSGGGG (SEQ ID NO:16)), a VH domain of a monoclonal antibody capable of binding CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (E-coil) Domain (EVAALEK-EVAALEK-EVAALEK-EVAALEK (SEQ ID NO:37)), an intervening linker peptide (GGGDKTHTCPPCP (SEQ ID NO:21)), a “knob-bearing” CH2 and CH3 Domain comprising the L234A/L235A/M252Y/S254T/T256E substitutions (SEQ ID NO:146), and a C-terminus.

Thus, the first polypeptide chain of TRIDENT-A6 is composed of: SEQ ID NO:56-SEQ ID NO:16-SEQ ID NO:46-SEQ ID NO:18-SEQ ID NO:37-SEQ ID NO:21-SEQ ID NO:146.

The amino acid sequence of the first polypeptide chain of TRIDENT-A6 is (SEQ ID NO:135):

EIVMTQSPAT LSLSPGERAT LSCRASQSVS SNYLSWFQQK
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGEVAAL EKEVAALEKE VAALEKEVAA LEKGGGDKTH
TCPPCPAPEA AGGPSVFLFP PKPKDTLYIT REPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The second polypeptide chain of TRIDENT-A6 comprises, in the N-terminal to C-terminal direction, an N-terminus, a VL domain of a monoclonal antibody capable of binding CD137 (VL_{CD137}) (CD137 MAB-6 VL3 (SEQ ID NO:56)), an intervening linker peptide (Linker 1; GGGSGGGG (SEQ ID NO:16)), a VH domain of a monoclonal antibody capable of binding CD137 (VH_{CD137}) (CD137 MAB-6 VH1 (SEQ ID NO:46)), an intervening linker peptide (Linker 2; GGCGGG (SEQ ID NO:18)), a Heterodimer-Promoting (K-coil) Domain (_KVAALKE-KVAALKE-KVAALKE-KVAALKE (SEQ ID NO:38)), and a C-terminus.

Thus, the second polypeptide chain of TRIDENT-A6 is composed of: SEQ ID NO:56-SEQ ID NO:16-SEQ ID NO:46-SEQ ID NO:18-SEQ ID NO:38.

The amino acid sequence of the second polypeptide chain of TRIDENT-A6 is (SEQ ID NO:136):

EIVMTQSPAT LSLSPGERAT LSCRASQSVS SNYLSWFQQK
PGQAPRLLIY GASTRATGIP ARFSGSGSGT DFTLTISSLQ
PEDFAVYYCQ QDYDLPWTFG QGTKVEIKGG GSGGGGQVQL
QESGPGLVKP SETLSLTCTV SGGSISSYYW SWIRQPPGKG
LEWIGRIYTS GSTNYNPSLK SRVTMSVDTS KNQFSLKLSS
VTAADTAVYY CARDGWYDED YNYYGMDVWG QGTTVTVSSG
GCGGGKVAAL KEKVAALKEK VAALKEKVAA LKE

It is specifically contemplated that alternative TRIDENT-A6 first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-A6 molecules, the first polypeptide chain sometimes is composed of: SEQ ID NO:56-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:56-SEQ ID NO:16-SEQ ID NO:46-SEQ ID NO:18-SEQ ID NO:40.

The third polypeptide chain of TRIDENT-A6 is the same as the third polypeptide chain of TRIDENT-A4 (SEQ ID NO:131).

The fourth polypeptide chain of TRIDENT-A6 is the same as the fourth polypeptide chain of TRIDENT-A4 (SEQ ID NO:132).

### 5. TRIDENT-B1

TRIDENT-B1 is a trivalent CD137×CD137×TA Binding Molecule having two CD137 binding sites and one binding site for the representative TA, HER2. TRIDENT-B1 is composed of four polypeptide chains (see, FIG. 3A, wherein VL1/VH1 (Site A) are the same as VL2/VH2 (Site B) and bind CD137, and VL3/VH3 (Site C) bind HER2). TRIDENT-B1 comprises the binding domains of CD137 MAB-6(1.1) and hHER2 MAB-1(1.3).

The first polypeptide chain of TRIDENT-B1 is the same as the first polypeptide chain of TRIDENT-A (SEQ ID NO:127).

The second polypeptide chain of TRIDENT-B1 is the same as the second polypeptide chain of TRIDENT-A (SEQ ID NO:128).

It is specifically contemplated that alternative TRIDENT-B1 first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-B1 molecules, the first polypeptide chain sometimes is composed of: SEQ ID NO:85-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:85-SEQ ID NO:16-SEQ ID NO:46-SEQ ID NO:18-SEQ ID NO:40.

The third polypeptide chain of TRIDENT-B1 comprises, in the N-terminal to C-terminal direction, an N-terminus, a VH domain of a monoclonal antibody capable of binding to HER2 (VHHER2 (hHER2 MAB-1 VH1, SEQ ID NO:80)), a human IgG1 CH1 Domain (SEQ ID NO:3), a human IgG1 Hinge Region (SEQ ID NO:7), and a “hole-bearing” CH2 and CH3 Domain comprising the L234A/L235A/M252Y/S254T/T256E/H435R substitutions (SEQ ID NO:149).

Thus, the third polypeptide chain of TRIDENT-B1 is composed of: SEQ ID NO:80-SEQ ID NO:3-SEQ ID NO:7-SEQ ID NO: 149.

The amino acid sequence of the third polypeptide chain of TRIDENT-B1 is (SEQ ID NO:153):

QVQLVQSGAE VKKPGASVKV SCKASGYTFT NYGMNWVRQA
PGQGLEWMGW INTNIGEPTY TEEFKGRVTM TRDTSISTAY
MELSRLRSDD TAVYYCARDD GYGNRVSYWG QGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLYITREPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

The fourth polypeptide chain of TRIDENT-BI comprises, in the N-terminal to C-terminal direction, an N-terminus, a VL domain of a monoclonal antibody capable of binding to HER2 (VL_{HER2} (hHER2 MAB-1 VL3, SEQ ID NO:85)), a human IgG CL Kappa Domain (SEQ ID NO:1), and a C-terminus.

Thus, the fourth polypeptide chain of TRIDENT-B1 is composed of: SEQ ID NO:85-SEQ ID NO:1.

The amino acid sequence of the fourth polypeptide chain of TRIDENT-B1 is (SEQ ID NO:154):

DIQMTQSPSS LSASVGDRVT ITCKASQDIS NYLSWFQQKP
GKAPKTLIYR ANRLQSGVPS RFSGSGSGTD FTLTISSLQP
EDFATYYCLQ HDEFPWTFGQ GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

### 6. TRIDENT-B2

TRIDENT-B2 is a trivalent CD137×CD137×TA Binding Molecule having two CD137 binding sites and one binding site for the representative TA, HER2. TRIDENT-B1 is composed of four polypeptide chains (see, FIG. 3A, wherein VL1/VH1 (Site A) are the same as VL3/VH3 (Site C) and bind CD137, and VL2/VH2 (Site B) bind HER2). TRIDENT-B1 comprises the binding domains of CD137 MAB-6(1.1) and hHER2 MAB-1(1.3).

The first polypeptide chain of TRIDENT-B2 is the same as the first polypeptide chain of DART-B1 (SEQ ID NO:143).

The second polypeptide chain of TRIDENT-B2 is the same as the second polypeptide chain of DART-B1 (SEQ ID NO:144).

It is specifically contemplated that alternative TRIDENT-B2 first and second polypeptide chains may be employed that comprise Heterodimer-Promoting (E-coil and K-coil) Domains comprising a cysteine residue (e.g., EVAACEK-EVAALEK-EVAALEK-EVAALEK (SEQ ID NO:39) and KVAACKE-KVAALKE-KVAALKE-KVAALKE (SEQ ID NO:40)). In such alternative TRIDENT-B2 molecules, the first polypeptide chain sometimes is composed of: SEQ ID NO:50-SEQ ID NO:16-SEQ ID NO:46-SEQ ID NO:18-SEQ ID NO:39-SEQ ID NO:21-SEQ ID NO:146 and the second polypeptide chain sometimes is composed of SEQ ID NO:50-SEQ ID NO:16-SEQ ID NO:46-SEQ ID NO:18-SEQ ID NO:40.

The third polypeptide chain of TRIDENT-B2 comprises, in the N-terminal to C-terminal direction, an N-terminus, a VH domain of a monoclonal antibody capable of binding a VH domain of a monoclonal antibody capable of binding CD137 (VHC_{D137}) (CD137 MAB-6 VH1 (SEQ ID NO:46)), a human IgG1 CH1 Domain (SEQ ID NO:3), a human IgG1 Hinge Region (SEQ ID NO:7), and a “hole-bearing” CH2 and CH3 Domain comprising the L234A/L235A/M252Y/S254T/T256E/H435R substitutions (SEQ ID NO:149).

Thus, the third polypeptide chain of TRIDENT-B2 is composed of: SEQ ID NO:46-SEQ ID NO:3-SEQ ID NO:7-SEQ ID NO: 149.

The amino acid sequence of the third polypeptide chain of TRIDENT-B2 is (SEQ ID NO:155):

QVQLQESGPG LVKPSETLSL TCTVSGGSIS SYYWSWIRQP
PGKGLEWIGR IYTSGSTNYN PSLKSRVTMS VDTSKNQFSL
KLSSVTAADT AVYYCARDGW YDEDYNYYGM DVWGQGTMVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEA
AGGPSVFLFP PKPKDTLYIT REPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LSCAVKGFYP SDIAVEWESN GQPENNYKTT
PPVLDSDGSF FLVSKLTVDK SRWQQGNVFS CSVMHEALHN
RYTQKSLSLS PGK

The fourth polypeptide chain of TRIDENT-B2 comprises, in the N-terminal to C-terminal direction, an N-terminus, a VL domain of a monoclonal antibody capable of binding CD137 (VL_{CD137}) (CD137 MAB-6 VL1 (SEQ ID NO:50)), a human IgG CL Kappa Domain (SEQ ID NO:1), and a C-terminus.

Thus, the fourth polypeptide chain of TRIDENT-B2 is composed of: SEQ ID NO:50-SEQ ID NO:1.

The amino acid sequence of the fourth polypeptide chain of TRIDENTB2 is (SEQ ID NO:156):

EDFAVYYCQQ DYDLPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

### C. Alternative CD137×TA Binding Molecules

As will be recognized in view of the instant disclosure, additional CD137×TA Binding Molecules having the general structure of any of the above representative molecules and comprising a binding site for an alternative TA may be constructed by employing the VL and VH domains of alternative Tumor Antigen antibodies in lieu of the VL and VH domains of the anti-PD-L1 or anti-HER2. Similarly, as provided herein, alternative CD137×TA Binding Molecules may likewise be constructed incorporating alternative linkers and/or heterodimer promoting domains and/or antibody constant regions (e.g., CL, CH2-CH3 Domain).

### D. Control Molecules

In order to more meaningfully demonstrate the properties of the CD137×TA Binding Molecules of the present invention, comparator and control antibodies, whose VL and VH domains may be used to produce control Fc-bearing diabodies and other comparator and control binding molecules, are described herein.

Palivizumab (see, e.g., Protein Data Bank (PDB) ID No. 2 HWZ) is a humanized monoclonal antibody (IgG) directed against an epitope in the A antigenic site of the F protein of RSV, and is a suitable control antibody, whose VL and VH domains may be used to produce control diabodies and other control binding molecules. Alternative anti-RSV glycoprotein F antibodies include motavizumab (see, e.g., PDB ID No. 3JXT) and a variant of palivizumab engineered to remove a cysteine residues from CDR 1 of the light chain. The variant of palivizumab was used for generation of the negative control molecule described below.

The amino acid sequence of the VH Domain of the variant of palivizumab is (SEQ ID NO:137) (CDR_{H} residues are shown underlined):

QVTLRESGPA LVKPTQTLTL TCTFSGFSLS TSGMSVGWIR
QPPGKALEWL ADIWWDDKKD YNPSLKSRLT ISKDTSKNQV
VLKVTNMDPA DTATYYCARS MITNWYFDVW GAGTTVTVSS

The amino acid sequence of the VL Domain of the variant of palivizumab is (SEQ ID NO:138) (CDR_{L} residues are shown underlined):

DIQMTQSPST LSASVGDRVT ITCRASQSVG YMHWYQQKPG
KAPKLLIYDT SKLASGVPSR FSGSGSGTEF TLTISSLQPD
DFATYYCFQG SGYPFTFGGG TKLEIK

Several molecules comprising the epitope-binding site ofpreviously described anti-CD137 antibodies including urelumab (also known as BMS-663513, see, U.S. Pat. No. 8,137,667) and utomilumab (also known as PF-05082566, see, U.S. Pat. No. 8,337,850) and murine and humanized hCD137 MAB-3 (see, WO 2018/156740) are used herein for comparison purposes. The amino acid sequence of the complete Heavy and Light Chains of urelumab (WHO Drug Information, 2011, Recommended INN: List 66, 25(3):334) and utomilumab (WHO Drug Information, 2017, Recommended INN: List 77, 31(1):140-141) are known in the art. The amino acid sequence of the VH and VL Domains of the humanized hCD137 MAB-3(1B.3) used as a comparator herein are provided in WO 2018/156740, see paragraphs [00254] and [00261].

### E. Summary of CD137×TA Binding and Control Molecules

Table 5 summarizes the domain attributes of DART-A-DART-A9, TRIDENT-A, and TRMDENT-A4-A6:

TABLE 5

| Name | | | | SEQ | |
|---|---|---|---|---|---|
| (No. of | Parental | Fc | Chain | ID | Other |
| Chains) | mAbs | Domain | No. | NOs. | Components |
| DART-A | hPD-L1 | IgG1 | 1 | 116 | E/K Coils |
| (4 Chains) | MAB-2 (1.1) | (AA/YTE) | 2 | 117 | |
| | CD137 | | 3 | 116 | |
| | MAB-6 (1.1) | | 4 | 117 | |
| DART-A1 | hPD-L1 | IgG1 | 1 | 118 | E/K Coils |
| (4 Chains) | MAB-2 (2.1) | (AA/YTE) | 2 | 119 | |
| | CD137 | | 3 | 118 | |
| | MAB-6 (1.1) | | 4 | 119 | |
| DART-A2 | hPD-L1 | IgG1 | 1 | 120 | E/K Coils |
| (4 Chains) | MAB-2 (2.2) | (AA/YTE) | 2 | 119 | |
| | CD137 | | 3 | 120 | |
| | MAB-6 (1.1) | | 4 | 119 | |
| DART-A3 | hPD-L1 | IgG1 | 1 | 118 | E/K Coils |
| (4 Chains) | MAB-2 (3.1) | (AA/YTE) | 2 | 121 | |
| | CD137 | | 3 | 118 | |
| | MAB-6 (1.1) | | 4 | 121 | |
| DART-A4 | hPD-L1 | IgG1 | 1 | 120 | E/K Coils |
| (4 Chains) | MAB-2 (3.2) | (AA/YTE) | 2 | 121 | |
| | CD137 | | 3 | 120 | |
| | MAB-6 (1.1) | | 4 | 121 | |
| DART-A5 | hPD-L1 | IgG1 | 1 | 120 | E/K Coils |
| (4 Chains) | MAB-2 (3.2) | (AA/YTE) | 2 | 122 | |
| | CD137 | | 3 | 120 | |
| | MAB-6 (1.2) | | 4 | 122 | |
| DART-A6 | hPD-L1 | IgG1 | 1 | 120 | E/K Coils |
| (4 Chains) | MAB-2 (3.2) | (AA/YTE) | 2 | 123 | |
| | CD137 | | 3 | 120 | |
| | MAB-6 (1.3) | | 4 | 123 | |
| DART-A7 | hPD-L1 | IgG1 | 1 | 120 | E/K Coils |
| (4 Chains) | MAB-2 (4.2) | (AA/YTE) | 2 | 124 | |
| | CD137 | | 3 | 120 | |
| | MAB-6 (1.1) | | 4 | 124 | |
| DART-A8 | hPD-L1 | IgG1 | 1 | 120 | E/K Coils |
| (4 Chains) | MAB-2 (5.2) | (AA/YTE) | 2 | 125 | |
| | CD137 | | 3 | 120 | |
| | MAB-6 (1.1) | | 4 | 125 | |
| DART-A9 | hPD-L1 | IgG1 | 1 | 120 | E/K Coils |
| (4 Chains) | MAB-2 (6.2) | (AA/YTE) | 2 | 126 | |
| | CD137 | | 3 | 120 | |
| | MAB-6 (1.1) | | 4 | 126 | |
| DART-A10 | hPD-L1 | IgG1 | 1 | 120 | E/K Coils |
| (4 Chains) | MAB-2 (4.2) | (AA/YTE) | 2 | 139 | |
| | CD137 | | 3 | 120 | |
| | MAB-6 (1.3) | | 4 | 139 | |
| DART-B1 | hHER2 | IgG1 | 1 | 143 | E/K Coils |
| | MAB-1 (1.3) | (AA/TYE) | 2 | 144 | |
| | CD137 | (knob/hole) | 3 | 145 | |
| | MAB-6(1.1) | | | | |
| DART-B2 | hHER2 | IgG1 | 1 | 151 | E/K Coils |
| | MAB-1 (1.3) | (AA/YTE) | 2 | 152 | |
| | CD137 | | 3 | 151 | |
| | MAB-6(1.1) | | 4 | 152 | |
| TRIDENT- | hPD-L1 | IgG1 | 1 | 127 | CL/CH1 and |
| A | MAB-2 (1.1) | (knob/hole) | 2 | 128 | E/K Coils |
| (4 Chains) | CD137 | (AA/TYE) | 3 | 129 | |
| | MAB-6 (1.1) | | 4 | 130 | |
| TRIDENT- | hPD-L1 | IgG1 (AA) | 1 | 127 | CL/CH1 and |
| A4 | MAB-2 (3.2) | (knob/hole) | 2 | 128 | E/K Coils |
| (4 Chains) | CD137 | | 3 | 131 | |
| | MAB-6 (1.1) | | 4 | 132 | |
| TRIDENT | hPD-L1 | IgG1 (AA) | 1 | 133 | CL/CH1 and |
| A5 | MAB-2 (3.2) | (knob/hole) | 2 | 134 | E/K Coils |
| (4 chains) | CD137 | | 3 | 131 | |
| | MAB-6 (1.2) | | 4 | 132 | |
| TRIDENT- | hPD-L1 | IgG1 (AA) | 1 | 135 | CL/CH1 and |
| A6 | MAB-2 (3.2) | (knob/hole) | 2 | 136 | E/K Coils |
| | CD137 | | 3 | 131 | |
| | MAB-6 (1.3) | | 4 | 132 | |
| TRIDENT- | hHER2 | IgG1 (AA) | 1 | 127 | CL/CH1 and |
| B1 | MAB-1 (1.3) | (knob/hole) | 2 | 128 | E/K Coils |
| | CD137 | | 3 | 153 | |
| | MAB-6(1.1) | | 4 | 154 | |
| TRIDENT- | hHER2 | IgG1 (AA) | 1 | 143 | CL/CH1 and |
| B2 | MAB-1 (1.3) | (knob/hole) | 2 | 144 | E/K Coils |
| | CD137 | | 3 | 155 | |
| | MAB-6(1.1) | | 4 | 156 | |

Table 6 shows the attributes of additional DART and TRIDENT molecules that were prepared as comparators and negative controls:

TABLE 6

| Name | | | |
|---|---|---|---|
| (No. of | | Fc | |
| Chains) | Parental mAbs | Domain | Other Components |
| DART-1 | hPD-L1 MAB- | IgG1 | Same as DART-A except |
| (4 Chains) | 2 (1.1) variant | (AA/YTE) | comprising VH/VL of variant |
| | palivizumab | | palivizumab in place of the |
| | | | VH/VL of CD137 MAB-6(1.1) |
| DART-2 | hPD-L1 MAB- | IgG1 (AA) | Same as DART-A except |
| (4 Chains) | 2 (1.1) CD137 | | comprising VH/VL of CD137 |
| | MAB-2 | | MAB-2 in place of the VH/VL |
| | | | of hCD137 MAB-3(1B.3) |
| DART-3 | hPD-L1 MAB- | IgG1 (AA) | Same as DART-A except |
| (4 Chains) | 2 (1.1) VL/VL | | comprising VH/VL of |
| | utomilumab | | utomilumab in place of the |
| | | | VH/VL of hCD137 MAB- |
| | | | 6(1.1) |
| DART-4 | variant | IgG1 (AA) | Same as DART-B1 except |
| (3 Chains) | palivizumab | (knob/hole) | comprising the VH/VL of |
| | CD137 MAB- | | variant palivizumab in place of |
| | 6(1.1) | | the VH/VL of hHER2 MAB-1 |
| | | | (1.3) |
| DART-5 | variant | IgG1 (AA) | Same as DART-B2 except |
| (4 Chains) | palivizumab | | comprising the VH/VL of |
| | CD137 MAB- | | variant palivizumab in place of |
| | 6(1.1) | | the VH/VL of hHER2 MAB-1 |
| | | | (1.3) |
| TRIDENT- | hPD-L1 MAB- | IgG1 (AA) | Same as TRIDENT-A except |
| 2 | 2 (1.1) | (knob/hole) | comprising the VH/VL of |
| (4 Chains) | hCD137 MAB- | | hCD137 MAB-3(1B.3) in place |
| | 3(1B.3) | | of the VH/VL of, andCD137 |
| | | | MAB6(1.1), SEQ ID NO: 19 |
| | | | for Linker 2 and cysteine- |
| | | | containing Heterodimer- |
| | | | Promoting Domains (see, |
| | | | paragraphs [00306]-[00311] of |
| | | | WO 2020/041404 for full |
| | | | sequences) |
| TRIDENT- | variant | IgG1 (AA) | Same as TRIDENT-B1 except |
| 3 | palivizumab | (knob/hole) | comprising the VH/VL of |
| | CD137 MAB- | | VH/VL of variant palivizumab |
| | 6(1.1) | | in place of the VH/VL of |
| | | | hHER2 MAB-1 (1.3) |
| TRIDENT- | variant | IgG1 (AA) | Same as TRIDENT-B2 except |
| 4 | palivizumab | (knob/hole) | comprising the VH/VL of |
| | CD137 | | VH/VL of variant palivizumab |
| | MAB-6(1.1) | | in place of the VH/VL of |
| | | | hHER2 MAB-1 (1.3) |

### III. Methods of Production PP12I

The binding molecules of the invention may be made recombinantly and expressed using any method known in the art. Such molecules may be made recombinantly by, obtaining the nucleic acids encoding the binding molecules, and using the nucleic acids to generate vectors useful for recombinant expression of the molecules in host cells (e.g., CHO cells). Another method that may be employed is to express the molecules in plants (e.g., tobacco) or transgenic milk.

Vectors containing polynucleotides of interest (e.g., polynucleotides encoding the polypeptide chains of the binding molecules of the present invention) can be introduced into the host cell by any of a number of appropriate means, including electroporation; microprojectile bombardment; lipofection; and infection (e.g., where the vector is an infectious agent such as vaccinia virus). Techniques for the introduction of nucleic acid or vectors into host cells are well established in the art and any suitable technique may be employed.

Any host cell capable of overexpressing heterologous DNAs can be used for the purpose of expressing a binding molecule (e.g., antibody, diabody, trivalent binding molecule) of interest. Non-limiting examples of suitable mammalian host cells include but are not limited to COS, NSO, HEK-293, HeLa, and CHO cells. Methods for culturing host cells are well-known in the art.

The binding molecules are typically isolated and/or purified from the host cell, culture media, etc. Techniques for the purification of recombinant binding molecules comprising antibody domains (e.g., Fc Domains) are well-known in the art and include, for example the use of HPLC, FPLC, or affinity chromatography, (e.g., using Protein A or Protein G). Following purification, the binding molecules of the invention may be formulated into a pharmaceutical composition, optionally with a pharmaceutically acceptable excipient or other substance as described below.

### IV. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a CD137×TA Binding Molecule of the present invention, or a combination of such agents and a pharmaceutically acceptable carrier. As provided herein, compositions of the invention comprise a prophylactically or therapeutically effective amount of the CD137×TA bispecific Fc-bearing diabody of the invention and a pharmaceutically acceptable carrier.

The invention also encompasses pharmaceutical compositions comprising a CD137×TA Binding Molecules of the invention and one or more additional molecules that are effective in stimulating an immune response (e.g., an immune checkpoint inhibitor) and/or in combination with one or more additional molecules that specifically bind a tumor antigen (e.g., a tumor-specific monoclonal antibody or diabody) that is specific for at least one particular TA, as described above, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids. Aqueous carriers, such as saline solutions, aqueous dextrose and glycerol solutions are preferred when the pharmaceutical composition is administered intravenously.

Generally, the ingredients of compositions of the invention are supplied either separately or mixed together in unit dosage form, for example, as a dry lyophilized powder or water free concentrate, or in liquid form in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers containing a CD137×TA Binding Molecule of the present invention alone or with other agents, for example, with a pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

A kit can comprise a CD137×TA Binding Molecule of the invention. The kit can further comprise one or more other prophylactic and/or therapeutic agents useful for the treatment of cancer, in one or more containers; and/or the kit can further comprise one or more cytotoxic antibodies that bind one or more tumor antigens (TAs). In certain embodiments, the other prophylactic or therapeutic agent is a chemotherapeutic. In other embodiments, the prophylactic or therapeutic agent is a biological or hormonal therapeutic.

### V. Methods of Administration

The compositions of the present invention may be provided for the treatment, prophylaxis, and amelioration of one or more symptoms associated with cancer or other disease, or disorder by administering to a subject an effective amount of a molecule of the invention, or a pharmaceutical composition comprising a molecule of the invention. In one aspect, such compositions are substantially purified (i.e., substantially free from substances that limit its effect or produce undesired side effects). In a specific embodiment, the subject is an animal. In another specific embodiment, the subject is a mammal such as non-primate (e.g., bovine, equine, feline, canine, rodent, etc.) or a primate (e.g., monkey such as, a cynomolgus monkey, human, etc.). In another embodiment, the subject is a human.

Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the CD137×TA Binding Molecules of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

The invention also provides that the CD137×TA Binding Molecules of the invention are packaged in a hermetically sealed container such as an ampoule or sachette indicating the quantity of the molecule. In one embodiment, the CD137×TA Binding Molecules of the invention are supplied as a dry sterilized lyophilized powder or water free concentrate in a hermetically sealed container and can be reconstituted, e.g., with water or saline to the appropriate concentration for administration to a subject. The lyophilized CD137×TA Binding Molecules of the present invention should be stored at between 2 and 8° C. in their original container and the molecules should be administered within 12 hours, within 6 hours, within 5 hours, within 3 hours, or within 1 hour after being reconstituted.

In an alternative embodiment, CD137×TA Binding Molecules of the invention are supplied in liquid form in a hermetically sealed container indicating the quantity and concentration of the molecule, fusion protein, or conjugated molecule. In certain embodiments, the liquid form of the CD137×TA Binding Molecules of the invention are supplied in a hermetically sealed container and do not require reconstitution.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

As used herein, an “effective amount” of a pharmaceutical composition, in one embodiment, is an amount sufficient to effect beneficial or desired results including, without limitation, clinical results such as decreasing symptoms resulting from the disease attenuating a symptom of disease (e.g., the proliferation of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

Such effective amount can be administered in one or more administrations. For purposes of this invention, an effective amount of drug, compound, or pharmaceutical composition is an amount sufficient to reduce the proliferation of (or the effect of) viral presence and to reduce and/or delay the development of the disease (e.g., cancer) either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more additional agents (e.g., chemotherapeutic agents, or other agents considered standard of care for the particular condition), and a single agent may be considered to be given in an effective amount if, in conjunction with one or more other agents, a desirable result may be or is achieved. While individual needs vary, determination of optimal ranges of effective amounts of each component is within the skill of the art.

For the CD137×TA Binding Molecules encompassed by the invention, the dosage administered to a patient may be determined based upon the body weight (kg) of the recipient subject, or alternatively may be based on a fixed dose.

The dosage and frequency of administration of the CD137×TA Binding Molecules of the present invention may be reduced or altered by enhancing uptake and tissue penetration of the CD137×TA Binding Molecules by modifications such as, for example, lipidation.

The dosage of the CD137×TA Binding Molecules of the invention administered to a patient may be calculated for use as a single agent therapy. Alternatively, the CD137×TA Binding Molecules of the invention are used in combination with other therapeutic compositions such that the dosage administered to a patient is lower than when said molecules are used as a single agent therapy.

Treatment of a subject with a therapeutically or prophylactically effective amount of a CD137×TA Binding Molecules of the invention can include a single treatment or, can include a series of treatments. In one example, a subject is treated with a molecule of the invention one time per week, one time bi-weekly (i.e., once every other week), or one time every three weeks, for between about 1 to 52 weeks. The pharmaceutical compositions of the invention can be administered once a day, twice a day, or three times a day. Alternatively, the pharmaceutical compositions can be administered once a week, twice a week, once every two weeks, once a month, once every six weeks, once every two months, twice a year or once per year. It will also be appreciated that the effective dosage of the molecules used for treatment may increase or decrease over the course of a particular treatment.

### VI. Uses of the Compositions of the Invention

The CD137×TA Binding Molecules of the present invention have the ability to bind T cells (APCs) (for example, by binding to CD137 expressed on the surfaces of such T cells) and the ability to bind TA-expressing tumor cells (for example, by binding to a TA expressed on the surfaces of such tumor cells). Thus, the CD137×TA Binding Molecules of the present invention have the ability to co-localize T cells to TA-expressing tumor cells, and thus may be used to treat any disease or condition associated with or characterized by the expression of a TA. Thus, without limitation, pharmaceutical compositions comprising such molecules may be employed in the diagnosis or treatment of cancers that express a TA, including, but not limited to: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer. Particularly, such cancers which highly express TAs.

The CD137×TA Binding Molecules of the present invention may additionally be used in the manufacture of medicaments for the treatment of the above-described conditions.

In certain embodiments a CD137×TA Binding Molecule of the invention is used in combination with one or more other prophylactic and/or therapeutic agents useful for the treatment of cancer. In certain embodiments, the other prophylactic or therapeutic agent is a chemotherapeutic. In other embodiments, the prophylactic or therapeutic agent is a biological or hormonal therapeutic. In other embodiments, biological therapeutic is a cytotoxic antibody-based molecule, including but not limited to, an antibody, an antigen binding fragment of an antibody (e.g., an scFv, a Fab, a F(ab)₂, etc.), a TandAb, etc.), a multispecific binding molecule (e.g., a diabody, a bispecific antibody, a trivalent binding molecule, etc.), that binds one or more tumor antigens (TAs).

The CD137×TA Binding Molecules of the present invention can enhance the activity of tumor targeting agents. According, the CD137×TA Binding Molecules of the present invention may additionally be used in combination with other a tumor targeting agents, including but not limited to an antibody, an antigen binding fragment of an antibody (e.g., an scFv, a Fab, a F(ab)₂, etc.), a TandAb, etc.), a multispecific binding molecule (e.g., a diabody, a bispecific antibody, a trivalent binding molecule, etc.), capable of binding a desired TA. It is specifically contemplated that the tumor targeting agent may bind the same or a different TA as the CD137×TA Binding Molecule used in such combinations. In particular embodiments, the tumor targeting agent is a multispecific molecule that binds to a TA and to an epitope expressed on T-cells including, for example, CD3, and/or CD8, and mediate T cell redirected killing. Representative tumor targeting agents include, but are not limited to, molecules that bind to a TA and CD3 (“TA×CD3”). Representative TA×CD3 Binding Molecules (e.g., bispecific antibodies, DART® molecules, BiTe® molecules, TandAbs, etc. and trivalent molecules), and methods for making the same, which may be used in such combinations are well known in the art. (see for e.g., WO 2013/026835, WO 2013/158856, WO 2014/047231; WO 2014/110601; WO 2014/131711; WO 2015/026894; WO 2015/026892; WO 2015/184203; WO 2015/184207; WO 2016/036937; WO 2016/182751; WO 2017091656; WO 2017/142928; WO 2017/118675).

The use of CD137×TA Binding Molecules of the present invention in combination with a tumor targeting agent (e.g., a TA×CD3 Binding Molecule) can lead to up-regulation of the inhibitory immune modulator Programmed Death-1 (“PD-1,” also known as “CD279”). PD-1 mediates its inhibition of the immune system by binding PD-L1 and PD-L2 (also known as B7-H1 and B7-DC) (Flies, D. B. et al. (2007) “The New B7s: Playing a Pivotal Role in Tumor Immunity,” J. Immunother. 30(3):251-260; U.S. Pat. Nos. 6,803,192; 7,794,710). Thus, the further addition of an agent that inhibits the inhibitory activity of PD-1 (“PD-1/PD-L1 Checkpoint Inhibitor”) down regulates the expression of PD-1 can further enhances the activity of the CD137×TA and tumor targeting agents such as TA×CD3 Binding Molecules. The invention particularly encompasses PD-1/PD-L1 Checkpoint Inhibitors comprising an epitope-binding site of an antibody that binds PD-1.

Accordingly, the CD137×TA Binding Molecules of the present invention may additionally be used in combination with other tumor targeting agents, in further combination with a PD-1/PD-L1 checkpoint inhibitor. PD-1/PD-L1 Checkpoint Inhibitors include, but not limited to, an antibody, an antigen binding fragment of an antibody (e.g., an scFv, a Fab, a F(ab)₂, etc.), a TandAb, etc.), a multispecific binding molecule (e.g., a diabody, a bispecific antibody, a trivalent binding molecule, etc.), capable of binding to PD-1 and/or PD-L1. Representative PD-1/PD-L1 Checkpoint Inhibitors and methods for making the same, which may be used in such combinations are well known in the art. PD-1 Binding Molecules useful in the methods of the instant invention include: nivolumab (CAS Reg. No.: 946414-94-4, also known as 5C4, BMS-936558, ONO-4538, MDX-1106, and marketed as OPDIVO® by Bristol-Myers Squibb); pembrolizumab (formerly known as lambrolizumab), CAS Reg. No.: 1374853-91-4, also known as MK-3475, SCH-900475, and marketed as KEYTRUDA® by Merck); cemiplimab (CAS Reg. No.: 1801342-60-8, also known as REGN-2810, SAR-439684, and marketed as LIBTAYO®). The amino acid sequences of the complete Heavy and Light Chains of nivolumab (WHO Drug Information, 2013, Recommended INN: List 69, 27(1):68-69), pembrolizumab (WHO Drug Information, 2014, Recommended INN: List 75, 28(3):407), and cemiplimab (WHO Drug Information 2018, Proposed INN: List 119) are known in the art. Additional anti-PD-1 antibodies (e.g., hPD-1 mAb 7(1.2); possessing unique binding characteristics useful in the methods and compositions of the instant inventions have recently been identified (see, PCT Publication No. WO 2017/019846).

Where such combinations are employed, it is specifically contemplated that, one or more of the molecules may be administered to a subject “concurrently” (e.g., a CD137×TA Binding Molecule may be administered at the same time as a TA×CD3 Binding Molecule and/or a PD-1/PD-L1 Checkpoint Inhibitor is administered) and/or that one or more of the molecules may be administered “sequentially” (e.g., a CD137×TA Binding Molecule is administered and, at a later time, a TA×CD3 Binding Molecule and/or a PD-1/PD-L1 Checkpoint Inhibitor is administered, or vice versa).

### VII. Embodiments of the Invention

Having now generally described the invention, the same will be more readily understood through reference to the following numbered Embodiments (“E”), which are provided by way of illustration and are not intended to be limiting of the present invention unless specified:
E1. A CD137 Binding Molecule comprising a first binding site that immunospecifically binds to an epitope of CD137, wherein said first binding site comprises a first Light Chain Variable Domain that comprises a CDR_{L}1, CDR_{L}2 and CDR_{L}3, and a first Heavy Chain Variable Domain that comprises a CDR_{H}1, CDR_{H}2 and CDR_{H}3; and wherein:
   (A) said first Light Chain Variable Domain CDR_{L}1, CDR_{L}2, and CDR_{L}3 are the Light Chain CDRs of CD137 MAB-6 VL1 (SEQ ID NO:50); and
   (B) said first Heavy Chain Variable Domain CDR_{H}1, CDR_{H}2, and CDR_{H}3 are the Heavy Chain CDRs of CD137 MAB-6 VII (SEQ ID NO:46).
E2. The CD137 Binding Molecule of E1, wherein said first Heavy Chain Variable Domain comprises the amino acid sequence of: hCD137 MAB-6 VH1 (SEQ ID NO:46).
E3. The CD137 Binding Molecule of any one of E1-E2, wherein said first Light Chain Variable Domain comprises the amino acid sequence of:
   (A) hCD137 MAB-6 VLx (SEQ ID NO:54);
   (B) hCD137 MAB-6 VL1 (SEQ ID NO:50);
   (C) hCD137 MAB-6 VL2 (SEQ ID NO:55); or
   (D) hCD137 MAB-6 VL3 (SEQ ID NO:56).
E4. The CD137 Binding Molecule of any one of E1-E3, wherein:
   (A) said first Heavy Chain Variable Domain comprises the amino acid sequence of: hCD137 MAB-6 VH1 (SEQ ID NO:46); and
   (B) said first Light Chain Variable Domain comprises the amino acid sequence of: hCD137 MAB-6 VL1 (SEQ ID NO:50).
E5. The CD137 Binding Molecule of any one of E1-E3, wherein:
   (A) said first Heavy Chain Variable Domain comprises the amino acid sequence of: hCD137 MAB-6 VH1 (SEQ ID NO:46); and
   (B) said first Light Chain Variable Domain comprises the amino acid sequence of: hCD137 MAB-6 VL3 (SEQ ID NO:56).
E6. The CD137 Binding Molecule of any one of E1-E5, wherein said molecule is a bispecific molecule comprising a second binding site that immunospecifically binds a tumor antigen (TA), and wherein said second binding site comprises a second Light Chain Variable Domain that comprises a CDR_{L}1, CDR_{L}2 and CDR_{L}3, and a second Heavy Chain Variable Domain that comprises a CDR_{H}1, CDR_{H}2 and CDR_{H}3.
E7. The CD137 Binding Molecule of E6, wherein said TA is selected from the antigens presented in Tables 1-2.
E8. The CD137 Binding Molecule of E6, wherein said TA is PD-L1 and wherein:
   (A) said second Light Chain Variable Domain CDR_{L}1, CDR_{L}2, and CDR_{L}3 are the Light Chain CDRs of hPD-L1 MAB-2 VLx (SEQ ID NO:63); and
   (B) said second Heavy Chain Variable Domain CDR_{H}1, CDR_{H}2, and CDR_{H}3 are the Heavy Chain CDRs of hPD-L1 MAB-2 VHx (SEQ ID NO:59).
E9. The CD137 Binding Molecule of E8, wherein:
   (A) (1) said second Light Chain Variable Domain CDR_{L}1, CDR_{L}2, and CDR_{L}3 are the Light Chain CDRs of hPD-L1 MAB-2 VL1 (SEQ ID NO:63); or
   (2) said second Light Chain Variable Domain CDR_{L}1, CDR_{L}2, and CDR_{L}3 are the Light Chain CDRs of hPD-L1 MAB-2 VLx (SEQ ID NO:58); or
   (3) said second Light Chain Variable Domain CDR_{L}1, CDR_{L}2, and CDR_{L}3 are the Light Chain CDRs of hPD-L1 MAB-2 VL2 (SEQ ID NO:72);
and
(B) (1) said second Heavy Chain Variable Domain CDR_{H}1, CDR_{H}2, and CDR_{H}3 are the Heavy Chain CDRs of hPD-L1 MAB-2 VH1 (SEQ ID NO:59);
   (2) said second Heavy Chain Variable Domain CDR_{H}1, CDR_{H}2, and CDR_{H}3 are the Heavy Chain CDRs of hPD-L1 MAB-2 VHx (SEQ ID NO:57);
   (3) said second Heavy Chain Variable Domain CDR_{H}1, CDR_{H}2, and CDR_{H}3 are the Heavy Chain CDRs of hPD-L1 MAB-2 VH2 (SEQ ID NO:67);
   (4) said second Heavy Chain Variable Domain CDR_{H}1, CDR_{H}2, and CDR_{H}3 are the Heavy Chain CDRs of hPD-L1 MAB-2 VH3 (SEQ ID NO:68);
   (5) said second Heavy Chain Variable Domain CDR_{H}1, CDR_{H}2, and CDR_{H}3 are the Heavy Chain CDRs of hPD-L1 MAB-2 VH2 (SEQ ID NO:69);
   (6) said second Heavy Chain Variable Domain CDR_{H}1, CDR_{H}2, and CDR_{H}3 are the Heavy Chain CDRs of hPD-L1 MAB-2 VH2 (SEQ ID NO:70); or
   (7) said second Heavy Chain Variable Domain CDR_{H}1, CDR_{H}2, and CDR_{H}3 are the Heavy Chain CDRs of hPD-L1 MAB-2 VH2 (SEQ ID NO:71).
E10. The CD137 Binding Molecule of E9, wherein said second Heavy Chain Variable Domain comprises the amino acid sequence of:
   (A) hPD-L1 MAB-2 VHx (SEQ ID NO:59);
   (B) hPD-L1 MAB-2 VH1 (SEQ ID NO:57);
   (C) hPD-L1 MAB-2 VH2 (SEQ ID NO:67);
   (D) hPD-L1 MAB-2 VH3 (SEQ ID NO:68);
   (E) hPD-L1 MAB-2 VH4 (SEQ ID NO:69);
   (F) hPD-L1 MAB-2 VH5 (SEQ ID NO:70); or
   (G) hPD-L1 MAB-2 VH6 (SEQ ID NO:71).
E11. The CD137 Binding Molecule of any one of E9 or E10, wherein said second Light Chain Variable Domain comprises the amino acid sequence of:
   (A) hPD-L1 MAB-2 VLx (SEQ ID NO:63);
   (B) hPD-L1 MAB-2 VL1 (SEQ ID NO:58); or
   (B) hPD-L1 MAB-2 VL2 (SEQ ID NO:72).
E12. The CD137 Binding Molecule of any one of E10-E11, wherein:
   (A) said second Heavy Chain Variable Domain comprises the amino acid sequence of: hPD-L1 MAB-2 VHx (SEQ ID NO:59); and
   (B) said second Light Chain Variable Domain comprises the amino acid sequence of: hPD-L1 MAB-2 VLx (SEQ ID NO:63).
E13. The CD137 Binding Molecule of any one of E10-E11, wherein:
   (A) said second Heavy Chain Variable Domain comprises the amino acid sequence of: hPD-L1 MAB-2 VH1 (SEQ ID NO:57); and
   (B) said second Light Chain Variable Domain comprises the amino acid sequence of: hPD-L1 MAB-2 VLx (SEQ ID NO:63).
E14. The CD137 Binding Molecule of any one of E10-E11, wherein:
   (A) said second Heavy Chain Variable Domain comprises the amino acid sequence of: hPD-L1 MAB-2 VH2 (SEQ ID NO:67); and
   (B) said second Light Chain Variable Domain comprises the amino acid sequence of: hPD-L1 MAB-2 VLx (SEQ ID NO:63).
E15. The CD137 Binding Molecule of any one of E10-E11, wherein:
   (A) said second Heavy Chain Variable Domain comprises the amino acid sequence of: hPD-L1 MAB-2 VH3 (SEQ ID NO:68); and
   (B) said second Light Chain Variable Domain comprises the amino acid sequence of: hPD-L1 MAB-2 VLx (SEQ ID NO:63).
E16. The CD137 Binding Molecule of any one of E10-E11, wherein:
   (A) said second Heavy Chain Variable Domain comprises the amino acid sequence of: hPD-L1 MAB-2 VH4 (SEQ ID NO:69); and
   (B) said second Light Chain Variable Domain comprises the amino acid sequence of: hPD-L1 MAB-2 VLx (SEQ ID NO:63).
E17. The CD137 Binding Molecule of any one of E10-E11, wherein:
   (A) said second Heavy Chain Variable Domain comprises the amino acid sequence of: hPD-L1 MAB-2 VH5 (SEQ ID NO:70); and
   (B) said second Light Chain Variable Domain comprises the amino acid sequence of: hPD-L1 MAB-2 VLx (SEQ ID NO:63).
E18. The CD137 Binding Molecule of any one of E10-E11, wherein:
   (A) said second Heavy Chain Variable Domain comprises the amino acid sequence of: hPD-L1 MAB-2 VH6 (SEQ ID NO:71); and
   (B) said second Light Chain Variable Domain comprises the amino acid sequence of: hPD-L1 MAB-2 VLx (SEQ ID NO:63).
E19. The CD137 Binding Molecule of any one of E10-E11, wherein:
   (A) said second Heavy Chain Variable Domain comprises the amino acid sequence of: hPD-L1 MAB-2 VH1 (SEQ ID NO:57); and
   (B) said second Light Chain Variable Domain comprises the amino acid sequence of: hPD-L1 MAB-2 VL1 (SEQ ID NO:58).
E20. The CD137 Binding Molecule of any one of E10-E11, wherein:
   (A) said second Heavy Chain Variable Domain comprises the amino acid sequence of: hPD-L1 MAB-2 VH3 (SEQ ID NO:68); and
   (B) said second Light Chain Variable Domain comprises the amino acid sequence of: hPD-L1 MAB-2 VL2 (SEQ ID NO:72).
E21. The CD137 Binding Molecule of E6, wherein the TA is 5T4 and wherein:
   (A) (1) said second Light Chain Variable Domain CDR_{L}1, CDR_{L}2, and CDR_{L}3 are the Light Chain CDRs of 5T4 MAB-1 VL (SEQ ID NO:93); and
      (2) said second Heavy Chain Variable Domain CDR_{H}1, CDR_{H}2, and CDR_{H}3 are the Heavy Chain CDRs of 5T4 MAB-1 VH (SEQ ID NO:92); or
   (B) (1) said second Light Chain Variable Domain CDR_{L}1, CDR_{L}2, and CDR_{L}3 are the Light Chain CDRs of 5T4 MAB-2 VL (SEQ ID NO:95); and
      (2) said second Heavy Chain Variable Domain CDR_{H}1, CDR_{H}2, and CDR_{H}3 are the Heavy Chain CDRs of 5T4 MAB-2 VH (SEQ ID NO:96).
E22. The CD137×TA Binding Molecule of E21, wherein the second Heavy Chain Variable Domain comprises the amino acid sequence of: 5T4 MAB-1 VH (SEQ ID NO:92).
E23. The CD137×TA Binding Molecule of E21 or E22 wherein the second Light Chain Variable Domain comprises the amino acid sequence of: 5T4 MAB-1 VL (SEQ ID NO:93).
E24. The CD137 Binding Molecule of E6, wherein the TA is HER2 and wherein:
   (A) said second Light Chain Variable Domain CDR_{L}1, CDR_{L}2, and CDR_{L}3 are the Light Chain CDRs of hHER2-MAB-1 VLx (SEQ ID NO:79); and
   (B) said second Heavy Chain Variable Domain CDR_{H}1, CDR_{H}2, and CDR_{H}3 are the Heavy Chain CDRs of hHER2-MAB-1 VHx (SEQ ID NO:78);
E25. The CD137 Binding Molecule of E24, wherein:
   (A) (1) said second Light Chain Variable Domain CDR_{L}1, CDR_{L}2, and CDR_{L}3 are the Light Chain CDRs of hHER2-MAB-1 VL1 (SEQ ID NO:83);
      (2) said second Light Chain Variable Domain CDR_{L}1, CDR_{L}2, and CDR_{L}3 are the Light Chain CDRs of hHER2-MAB-1 VL2 (SEQ ID NO:84); or
      (3) said second Light Chain Variable Domain CDR_{L}1, CDR_{L}2, and CDR_{L}3 are the Light Chain CDRs of hHER2-MAB-1 VL3 (SEQ ID NO:85);
and
(B) (1) said second Heavy Chain Variable Domain CDR_{H}1, CDR_{H}2, and CDR_{H}3 are the Heavy Chain CDRs of hHER2-MAB-1 VH1 (SEQ ID NO:80);
   (2) said second Heavy Chain Variable Domain CDR_{H}1, CDR_{H}2, and CDR_{H}3 are the Heavy Chain CDRs of hHER2-MAB-1 VH2 (SEQ ID NO:81); or
   (3) said second Heavy Chain Variable Domain CDR_{H}1, CDR_{H}2, and CDR_{H}3 are the Heavy Chain CDRs of hHER2-MAB-1 VH3 (SEQ ID NO:82).
E26. The CD137 Binding Molecule of E25, wherein said second Heavy Chain Variable Domain comprises the amino acid sequence of:
   (A) hHER2-MAB-1 VHx (SEQ ID NO:78);
   (B) hHER2-MAB-1 VH1 (SEQ ID NO:80);
   (C) hHER2-MAB-1 VH2 (SEQ ID NO:81); or
   (D) hHER2-MAB-1 VH3 (SEQ ID NO:82).
E27. The CD137 Binding Molecule of any one of E25 or 26, wherein said second Light Chain Variable Domain comprises the amino acid sequence of:
   (A) hHER2-MAB-1 VLx (SEQ ID NO:79);
   (B) hHER2-MAB-1 VL1 (SEQ ID NO:83);
   (C) hHER2-MAB-1 VL2 (SEQ ID NO:84); or
   (D) hHER2-MAB-1 VL3 (SEQ ID NO:85).
E28. The CD137 Binding Molecule of any one of E24-E26, wherein:
   (A) (1) said second Heavy Chain Variable Domain comprises the amino acid sequence of: hHER2-MAB-1 VHx (SEQ ID NO:78); and
      (2) said second Light Chain Variable Domain comprises the amino acid sequence of: hHER2-MAB-1 VLx (SEQ ID NO:79);
or
(B) (1) said second Heavy Chain Variable Domain comprises the amino acid sequence of: hHER2-MAB-1 VH1 (SEQ ID NO:80); and
   (2) said second Light Chain Variable Domain comprises the amino acid sequence of: hHER2-MAB-1 VL3 (SEQ ID NO:85).
E29. The CD137 Binding Molecule of any one of E1-E29, which is an antibody, a bispecific antibody, a bispecific bivalent Fc-bearing diabody, or a bispecific tetravalent Fc-bearing diabody, or a bispecific trivalent molecule.
E30 The CD137 Binding Molecule of any one of E1-E29, wherein said molecule is bispecific and bivalent, and comprises a first, a second, and a third polypeptide chain, wherein said polypeptide chains form a covalently bonded complex.
E31. The CD137 Binding Molecule of any one of E1-E29, wherein said molecule is bispecific and tetravalent, and comprises a first, a second, a third, and a fourth polypeptide chain, wherein said polypeptide chains form a covalently bonded complex.
E32. The CD137 Binding Molecule of any one of E1-E29, wherein said molecule is bispecific and trivalent, and comprises a first, a second, a third, and a fourth, polypeptide chain, wherein said polypeptide chains form a covalently bonded complex.
E33. The CD137 Binding Molecule of E31, wherein said TA is PD-L1 and wherein:
   (A) said first and third polypeptide chains comprise in the N-terminal to C-terminal direction:
      (i) SEQ ID NO:63-SEQ ID NO:16-SEQ ID NO:46-SEQ ID NO:18-SEQ ID NO:39-SEQ ID NO:30-SEQ ID NO:43;
      (ii) SEQ ID NO:63-SEQ ID NO:16-SEQ ID NO:46-SEQ ID NO:18-SEQ ID NO:37-SEQ ID NO:30-SEQ ID NO:43;
      (iii) SEQ ID NO:58-SEQ ID NO:16-SEQ ID NO:46-SEQ ID NO:18-SEQ ID NO:39-SEQ ID NO:30-SEQ ID NO:43;
      (iv) SEQ ID NO:58-SEQ ID NO:16-SEQ ID NO:46-SEQ ID NO:18-SEQ ID NO:37-SEQ ID NO:30-SEQ ID NO:43;
      (v) SEQ ID NO:72-SEQ ID NO:16-SEQ ID NO:46-SEQ ID NO:18-SEQ ID NO:39-SEQ ID NO:30-SEQ ID NO:43; or
      (vi) SEQ ID NO:72-SEQ ID NO:16-SEQ ID NO:46-SEQ ID NO:18-SEQ ID NO:37-SEQ ID NO:30-SEQ ID NO:43;
   and
   (B) said second and fourth polypeptide chains comprise in the N-terminal to C-terminal direction:
      (i) SEQ ID NO:54-SEQ ID NO:16-SEQ ID NO:59-SEQ ID NO:18-SEQ ID NO:40;
      (ii) SEQ ID NO:54-SEQ ID NO:16-SEQ ID NO:59-SEQ ID NO:18-SEQ ID NO:38;
      (iii) SEQ ID NO:50-SEQ ID NO:16-SEQ ID NO:57-SEQ ID NO:18-SEQ ID NO:40;
      (iv) SEQ ID NO:50-SEQ ID NO:16-SEQ ID NO:57-SEQ ID NO:18-SEQ ID NO:38;
      (v) SEQ ID NO:50-SEQ ID NO:16-SEQ ID NO:67-SEQ ID NO:18-SEQ ID NO:40;
      (vi) SEQ ID NO:50-SEQ ID NO:16-SEQ ID NO:67-SEQ ID NO:18-SEQ ID NO:38;
      (vii) SEQ ID NO:50-SEQ ID NO:16-SEQ ID NO:68-SEQ ID NO:18-SEQ ID NO:40;
      (viii) SEQ ID NO:50-SEQ ID NO:16-SEQ ID NO:68-SEQ ID NO:18-SEQ ID NO:38;
      (ix) SEQ ID NO:55-SEQ ID NO:16-SEQ ID NO:68-SEQ ID NO:18-SEQ ID NO:40;
      (x) SEQ ID NO:55-SEQ ID NO:16-SEQ ID NO:68-SEQ ID NO:18-SEQ ID NO:38;
      (xi) SEQ ID NO:56-SEQ ID NO:16-SEQ ID NO:68-SEQ ID NO:18-SEQ ID NO:40;
      (xii) SEQ ID NO:56-SEQ ID NO:16-SEQ ID NO:68-SEQ ID NO:18-SEQ ID NO:38;
      (xiii) SEQ ID NO:50-SEQ ID NO:16-SEQ ID NO:69-SEQ ID NO:18-SEQ ID NO:40;
      (xiv) SEQ ID NO:50-SEQ ID NO:16-SEQ ID NO:69-SEQ ID NO:18-SEQ ID NO:38;
      (xv) SEQ ID NO:50-SEQ ID NO:16-SEQ ID NO:70-SEQ ID NO:18-SEQ ID NO:40;
      (xvi) SEQ ID NO:50-SEQ ID NO:16-SEQ ID NO:70-SEQ ID NO:18-SEQ ID NO:38;
      (xvii) SEQ ID NO:50-SEQ ID NO:16-SEQ ID NO:71-SEQ ID NO:18-SEQ ID NO:40;
      (xviii) SEQ ID NO:50-SEQ ID NO:16-SEQ ID NO:71-SEQ ID NO:18-SEQ ID NO:38;
      (xix) SEQ ID NO:56-SEQ ID NO:16-SEQ ID NO:69-SEQ ID NO:18-SEQ ID NO:40; or
      (xx) SEQ ID NO:56-SEQ ID NO:16-SEQ ID NO:69-SEQ ID NO:18-SEQ ID NO:38.
E34. The CD137 Binding Molecule of E31 or E33, wherein said TA is PD-L1 and wherein:
   (A) said first and third polypeptide chains comprise the amino acid sequence of SEQ ID NO:116, SEQ ID NO:118, or SEQ ID NO:120; and
   (B) said second and fourth polypeptide chains comprise the amino acid sequence of SEQ ID NO:117, SEQ ID NO:119, SEQ ID NO:121, SEQ ID NO:122, SEQ ID NO:123, SEQ ID NO:124, SEQ ID NO:125, SEQ ID NO:126, or SEQ ID NO:139.
E35. The CD137 Binding Molecule of E34, wherein said molecule comprises:
   (A) SEQ ID NO:116 and SEQ ID NO:117;
   (B) SEQ ID NO:118 and SEQ ID NO:119;
   (C) SEQ ID NO:120 and SEQ ID NO:119;
   (D) SEQ ID NO:118 and SEQ ID NO:121;
   (E) SEQ ID NO:120 and SEQ ID NO:121;
   (F) SEQ ID NO:120 and SEQ ID NO:122;
   (G) SEQ ID NO:120 and SEQ ID NO:123;
   (H) SEQ ID NO:120 and SEQ ID NO:124;
   (I) SEQ ID NO:120 and SEQ ID NO:125;
   (J) SEQ ID NO:120 and SEQ ID NO:126; or
   (K) SEQ ID NO:120 and SEQ ID NO:139.
E36. The CD137 Binding Molecule of E32, wherein said TA is PD-L1 and wherein:
   (A) said first polypeptide chain comprises in the N-terminal to C-terminal direction:
      (i) SEQ ID NO:54-SEQ ID NO:16-SEQ ID NO:46-SEQ ID NO:18-SEQ ID NO:37-SEQ ID NO:21-SEQ ID NO:146;
      (ii) SEQ ID NO:54-SEQ ID NO:16-SEQ ID NO:46-SEQ ID NO:18-SEQ ID NO:39-SEQ ID NO:21-SEQ ID NO:146;
      (iii) SEQ ID NO:50-SEQ ID NO:16-SEQ ID NO:46-SEQ ID NO:18-SEQ ID NO:37-SEQ ID NO:21-SEQ ID NO:146;
      (iv) SEQ ID NO:50-SEQ ID NO:16-SEQ ID NO:46-SEQ ID NO:18-SEQ ID NO:39-SEQ ID NO:21-SEQ ID NO:146;
      (v) SEQ ID NO:55-SEQ ID NO:16-SEQ ID NO:46-SEQ ID NO:18-SEQ ID NO:37-SEQ ID NO:21-SEQ ID NO:146;
      (vi) SEQ ID NO:55-SEQ ID NO:16-SEQ ID NO:46-SEQ ID NO:18-SEQ ID NO:39-SEQ ID NO:21-SEQ ID NO:146;
      (vi) SEQ ID NO:56-SEQ ID NO:16-SEQ ID NO:46-SEQ ID NO:18-SEQ ID NO:37-SEQ ID NO:21-SEQ ID NO:146; or
      (vii) SEQ ID NO:56-SEQ ID NO:16-SEQ ID NO:46-SEQ ID NO:18-SEQ ID NO:39-SEQ ID NO:21-SEQ ID NO:146;
   (B) said second polypeptide chain comprises in the N-terminal to C-terminal direction:
      (i) SEQ ID NO:54-SEQ ID NO:16-SEQ ID NO:46-SEQ ID NO:18-SEQ ID NO:38;
      (ii) SEQ ID NO:54-SEQ ID NO:16-SEQ ID NO:46-SEQ ID NO:18-SEQ ID NO:40;
      (iii) SEQ ID NO:50-SEQ ID NO:16-SEQ ID NO:46-SEQ ID NO:18-SEQ ID NO:38;
      (iv) SEQ ID NO:50-SEQ ID NO:16-SEQ ID NO:46-SEQ ID NO:18-SEQ ID NO:40;
      (v) SEQ ID NO:55-SEQ ID NO:16-SEQ ID NO:46-SEQ ID NO:18-SEQ ID NO:38;
      (vi) SEQ ID NO:55-SEQ ID NO:16-SEQ ID NO:46-SEQ ID NO:18-SEQ ID NO:40;
      (vi) SEQ ID NO:56-SEQ ID NO:16-SEQ ID NO:46-SEQ ID NO:18-SEQ ID NO:38; or
      (vii) SEQ ID NO:56-SEQ ID NO:16-SEQ ID NO:46-SEQ ID NO:18-SEQ ID NO:40;
   (C) said third polypeptide chain comprises in the N-terminal to C-terminal direction:
      (i) SEQ ID NO:59-SEQ ID NO:3-SEQ ID NO:7-SEQ ID NO:149;
      (ii) SEQ ID NO:57-SEQ ID NO:3-SEQ ID NO:7-SEQ ID NO:149;
      (iii) SEQ ID NO:67-SEQ ID NO:3-SEQ ID NO:7-SEQ ID NO:149;
      (iv) SEQ ID NO:68-SEQ ID NO:3-SEQ ID NO:7-SEQ ID NO:149;
      (v) SEQ ID NO:69-SEQ ID NO:3-SEQ ID NO:7-SEQ ID NO:149;
      (vi) SEQ ID NO:70-SEQ ID NO:3-SEQ ID NO:7-SEQ ID NO:149; or
      (vii) SEQ ID NO:71-SEQ ID NO:3-SEQ ID NO:7-SEQ ID NO:149;
   and
   (D) said fourth polypeptide chain comprises in the N-terminal to C-terminal direction:
      (i) SEQ ID NO:63-SEQ ID NO:1; or
      (ii) SEQ ID NO:72-SEQ ID NO:1.
E37. The CD137 Binding Molecule of E32, wherein said TA is PD-L1 and wherein:
   (A) said first polypeptide chain comprises in the N-terminal to C-terminal direction:
      (i) SEQ ID NO:54-SEQ ID NO:16-SEQ ID NO:59-SEQ ID NO:18-SEQ ID NO:37-SEQ ID NO:21-SEQ ID NO:146; or
      (ii) SEQ ID NO:54-SEQ ID NO:16-SEQ ID NO:59-SEQ ID NO:18-SEQ ID NO:39-SEQ ID NO:21-SEQ ID NO:146;
   (B) said second polypeptide chain comprises in the N-terminal to C-terminal direction:
      (i) SEQ ID NO:63-SEQ ID NO:16-SEQ ID NO:46-SEQ ID NO:18-SEQ ID NO:38; or
      (ii) SEQ ID NO:63-SEQ ID NO:16-SEQ ID NO:46-SEQ ID NO:18-SEQ ID NO:40;
   (C) said third polypeptide chain comprises in the N-terminal to C-terminal direction:
      (i) SEQ ID NO:46-SEQ ID NO:3-SEQ ID NO:7-SEQ ID NO:149;
   and
   (D) said fourth polypeptide chain comprises in the N-terminal to C-terminal direction:
      (i) SEQ ID NO:54-SEQ ID NO:1; or
      (ii) SEQ ID NO:50-SEQ ID NO:1.
E38. The CD137 Binding Molecule of E32 or E36, wherein said TA is PD-L1 and wherein:
   (A) said first polypeptide chain comprises the amino acid sequence of SEQ ID NO:127, SEQ ID NO:133, or SEQ ID NO:135;
   (B) said second polypeptide chain comprises the amino acid sequence of SEQ ID NO:128, SEQ ID NO:134, or SEQ ID NO:136;
   (C) said third polypeptide chain comprises the amino acid sequence of SEQ ID NO:129, or SEQ ID NO:131; and
   (D) said fourth polypeptide chain comprises the amino acid sequence of SEQ ID NO:130, SEQ ID NO:132.
E39. The CD137 Binding Molecule of E38, wherein said molecule comprises:
   (A) SEQ ID NO:127, SEQ ID NO:128, SEQ ID NO:129, and SEQ ID NO:130;
   (B) SEQ ID NO:127, SEQ ID NO:128, SEQ ID NO:131, and SEQ ID NO:132;
   (C) SEQ ID NO:133, SEQ ID NO:134, SEQ ID NO:131, and SEQ ID NO:132; or
   (D) SEQ ID NO:135, SEQ ID NO:136, SEQ ID NO:131, and SEQ ID NO:132.
E40. The CD137 Binding Molecule of E31, wherein said TA is HER2 and wherein:
   (A) said first and third polypeptide chains comprise in the N-terminal to C-terminal direction:
      (i) SEQ ID NO:79-SEQ ID NO:16-SEQ ID NO:46-SEQ ID NO:18-SEQ ID NO:39-SEQ ID NO:30-SEQ ID NO:43; or
      (ii) SEQ ID NO:79-SEQ ID NO:16-SEQ ID NO:46-SEQ ID NO:18-SEQ ID NO:37-SEQ ID NO:30-SEQ ID NO:43;
      (iii) SEQ ID NO:85-SEQ ID NO:16-SEQ ID NO:46-SEQ ID NO:18-SEQ ID NO:39-SEQ ID NO:30-SEQ ID NO:43; or
      (iv) SEQ ID NO:85-SEQ ID NO:16-SEQ ID NO:46-SEQ ID NO:18-SEQ ID NO:37-SEQ ID NO:30-SEQ ID NO:43;
   and
   (B) said second and fourth polypeptide chains comprise in the N-terminal to C-terminal direction:
      (i) SEQ ID NO:54-SEQ ID NO:16-SEQ ID NO:78-SEQ ID NO:18-SEQ ID NO:40;
      (ii) SEQ ID NO:54-SEQ ID NO:16-SEQ ID NO:78-SEQ ID NO:18-SEQ ID NO:38.
      (iii) SEQ ID NO:50-SEQ ID NO:16-SEQ ID NO:80-SEQ ID NO:18-SEQ ID NO:40; or
      (iv) SEQ ID NO:50-SEQ ID NO:16-SEQ ID NO:80-SEQ ID NO:18-SEQ ID NO:38.
E41. The CD137 Binding Molecule of E32, wherein said TA is HER2 and wherein:
   (A) said first polypeptide chain comprises in the N-terminal to C-terminal direction:
      (i) SEQ ID NO:54-SEQ ID NO:16-SEQ ID NO:46-SEQ ID NO:18-SEQ ID NO:37-SEQ ID NO:21-SEQ ID NO:146;
      (ii) SEQ ID NO:54-SEQ ID NO:16-SEQ ID NO:46-SEQ ID NO:18-SEQ ID NO:39-SEQ ID NO:21-SEQ ID NO:146;
      (iii) SEQ ID NO:50-SEQ ID NO:16-SEQ ID NO:46-SEQ ID NO:18-SEQ ID NO:37-SEQ ID NO:21-SEQ ID NO:146; or
      (iv) SEQ ID NO:50-SEQ ID NO:16-SEQ ID NO:46-SEQ ID NO:18-SEQ ID NO:39-SEQ ID NO:21-SEQ ID NO:146;
   (B) said second polypeptide chain comprises in the N-terminal to C-terminal direction:
      (i) SEQ ID NO:54-SEQ ID NO:16-SEQ ID NO:46-SEQ ID NO:18-SEQ ID NO:38;
      (ii) SEQ ID NO:54-SEQ ID NO:16-SEQ ID NO:46-SEQ ID NO:18-SEQ ID NO:40;
      (iii) SEQ ID NO:50-SEQ ID NO:16-SEQ ID NO:46-SEQ ID NO:18-SEQ ID NO:38; or
      (iv) SEQ ID NO:50-SEQ ID NO:16-SEQ ID NO:46-SEQ ID NO:18-SEQ ID NO:40;
   (C) said third polypeptide chain comprises in the N-terminal to C-terminal direction:
      (i) SEQ ID NO:78-SEQ ID NO:3-SEQ ID NO:7-SEQ ID NO:149; or
      (ii) SEQ ID NO:80-SEQ ID NO:3-SEQ ID NO:7-SEQ ID NO:149; and
   (D) said fourth polypeptide chain comprises in the N-terminal to C-terminal direction:
      (i) SEQ ID NO:79-SEQ ID NO:1; or
      (ii) SEQ ID NO:85-SEQ ID NO:1.
E42. The CD137 Binding Molecule of E32, wherein said TA is HER2 and wherein:
   (A) said first polypeptide chain comprises in the N-terminal to C-terminal direction:
      (i) SEQ ID NO:54-SEQ ID NO:16-SEQ ID NO:78-SEQ ID NO:18-SEQ ID NO:37-SEQ ID NO:21-SEQ ID NO:146;
      (ii) SEQ ID NO:54-SEQ ID NO:16-SEQ ID NO:78-SEQ ID NO:18-SEQ ID NO:39-SEQ ID NO:21-SEQ ID NO:146;
      (iii) SEQ ID NO:50-SEQ ID NO:16-SEQ ID NO:80-SEQ ID NO:18-SEQ ID NO:37-SEQ ID NO:21-SEQ ID NO:146; or
      (iv) SEQ ID NO:50-SEQ ID NO:16-SEQ ID NO:46-SEQ ID NO:18-SEQ ID NO:39-SEQ ID NO:21-SEQ ID NO:146;
   (B) said second polypeptide chain comprises in the N-terminal to C-terminal direction:
      (i) SEQ ID NO:79-SEQ ID NO:16-SEQ ID NO:46-SEQ ID NO:18-SEQ ID NO:38;
      (ii) SEQ ID NO:79-SEQ ID NO:16-SEQ ID NO:46-SEQ ID NO:18-SEQ ID NO:40;
      (iii) SEQ ID NO:85-SEQ ID NO:16-SEQ ID NO:46-SEQ ID NO:18-SEQ ID NO:38; or
      (iv) SEQ ID NO:85-SEQ ID NO:16-SEQ ID NO:46-SEQ ID NO:18-SEQ ID NO:40;
   (C) said third polypeptide chain comprises in the N-terminal to C-terminal direction:
      (i) SEQ ID NO:46-SEQ ID NO:3-SEQ ID NO:7-SEQ ID NO:149; and
   (D) said fourth polypeptide chain comprises in the N-terminal to C-terminal direction:
      (i) SEQ ID NO:54-SEQ ID NO:1; or
      (ii) SEQ ID NO:50-SEQ ID NO:1.
E43. The CD137 Binding Molecule of any one of E40-E42, wherein said molecule comprises:
   (A) SEQ ID NO:151, and SEQ ID NO:152;
   (B) SEQ ID NO:127, SEQ ID NO:128, SEQ ID NO:153, and SEQ ID NO:154; or
   (C) SEQ ID NO:143, SEQ ID NO:144, SEQ ID NO:155, and SEQ ID NO:165.
E44. A pharmaceutical composition comprising the CD137 Binding Molecule of any one E1-43, and a physiologically acceptable carrier.
E45. Use of the CD137 Binding Molecule of any one of E6-E43, or the pharmaceutical composition of E44, in the treatment of a disease or condition associated with or characterized by the expression of said TA.
E46. A PD-L1 Binding Molecule comprising a Light Chain Variable Domain that comprises a CDR_{L}1, CDR_{L}2 and CDR_{L}3, and a Heavy Chain Variable Domain that comprises a CDR_{H}1, CDR_{H}2 and CDR_{H}3; wherein:
   (A) said Light Chain Variable Domain CDR_{L}1, CDR_{L}2, and CDR_{L}3 are the Light Chain CDRs of hPD-L1 MAB-2 VL2 (SEQ ID NO:72);
   and
   (B) (1) said Heavy Chain Variable Domain CDR_{H}1, CDR_{H}2, and CDR_{H}3 are the Heavy Chain CDRs of hPD-L1 MAB-2 VH2 (SEQ ID NO:67);
      (2) said Heavy Chain Variable Domain CDR_{H}1, CDR_{H}2, and CDR_{H}3 are the Heavy Chain CDRs of hPD-L1 MAB-2 VH3 (SEQ ID NO:68)
      (3) said second Heavy Chain Variable Domain CDR_{H}1, CDR_{H}2, and CDR_{H}3 are the Heavy Chain CDRs of hPD-L1 MAB-2 VH4 (SEQ ID NO:69)
      (4) said second Heavy Chain Variable Domain CDR_{H}1, CDR_{H}2, and CDR_{H}3 are the Heavy Chain CDRs of hPD-L1 MAB-2 VH5 (SEQ ID NO:70); or
      (5) said second Heavy Chain Variable Domain CDR_{H}1, CDR_{H}2, and CDR_{H}3 are the Heavy Chain CDRs of hPD-L1 MAB-2 VH6 (SEQ ID NO:71).
E47. The PD-L1 Binding Molecule of E46, wherein said Heavy Chain Variable Domain comprises the amino acid sequence of:
   (A) hPD-L1 MAB-2 VH2 (SEQ ID NO:67);
   (B) hPD-L1 MAB-2 VH3 (SEQ ID NO:68);
   (C) hPD-L1 MAB-2 VH4 (SEQ ID NO:69);
   (D) hPD-L1 MAB-2 VH5 (SEQ ID NO:70); or
   (E) hPD-L1 MAB-2 VH6 (SEQ ID NO:71).
E48. The PD-L1 Binding Molecule of any one of E46-E47, wherein said Light Chain Variable Domain comprises the amino acid sequence of hPD-L1 MAB-2 VL2 (SEQ ID NO:72).
E49. The PD-L1 Binding Molecule of any one E46-E48, wherein said molecule is an antibody or an antigen binding fragment thereof.
E50. The PD-L1 Binding Molecule of any one E46-E48, wherein said molecule is a multispecific binding molecule.
E51. The PD-L1 Binding Molecule of any one E50, wherein said molecule is a bispecific diabody, a bispecific antibody, or a trivalent binding molecule.
E52. A pharmaceutical composition comprising the PD-L1 Binding Molecule of any one E46-E51, and a physiologically acceptable carrier.
E53. Use of the PD-L1 Binding Molecule of any one of E46-E51, or the pharmaceutical composition of E52, in the treatment of a disease or condition associated with a suppressed immune system or characterized by the expression of PD-L1.
E54. The use of E53, wherein said disease or condition associated with a suppressed immune system or characterized by the expression of PD-L1 is cancer.
E55. The use of any one of E45, or E54, wherein said cancer is selected from the group consisting: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer.
E56. A method of enhancing the activity of a tumor targeting agent comprising administering said tumor target agent in combination with the CD137 Binding Molecule of any one of E1-E43, the PD-L1 Binding Molecule of any one of E46-E51, or the pharmaceutical composition of any one of E44 or E52.
E57. A method of treating a disease or condition associated with a suppressed immune system or characterized by the expression of a TA comprising administering to a subject in need thereof of the CD137 Binding Molecule of any one of E1-E43, the PD-L1 Binding Molecule of any one of E46-E53, or the pharmaceutical composition of E44 or E53.
E58. The method of E57, wherein the condition associated with a suppressed immune system or characterized by the expression of the TA is cancer.
E59. The method of E57 or E58, further comprising administering a tumor targeting agent.
E60. The method of E56 or E59, wherein said tumor target agent is an antibody, an epitope binding fragment of an antibody, or an agent that mediates T-cell redirected killing of a target cell.
E61. The method of any one of E57-E60, wherein the cancer is selected from the group consisting: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer.
E62. A nucleic acid encoding the CD137 Binding Molecule of any one of E1-E43, or the PD-L1 Binding Molecule of any one of E46-E51.
E63. An expression vector comprising a nucleic acid according to E62.
E64. A cell comprising a nucleic acid according to E62 or an expression vector according to E63.
E65. The cell according to E64, wherein said cell is a mammalian cell.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### Methods

The ability of a test article (e.g., antibody, diabody, or trivalent molecule) to mediate target-dependent signal transduction of NF/kB pathway was evaluated using a CD137 expressing reporter cell line (Jurkat-NF—KB-Luc) in a CD137 reporter assay performed essentially as follows: for bispecific molecules target cells (type and number indicated in figures and below) in 100 μL of assay media (RPMI-1640, 10% FBS)) were plated into sterile assay microplates and incubated at 37° C. overnight. On day 2, Jurkat-NF—KB-Luc reporter cells overexpressing CD137 (4×10⁴ to 7.5×10⁴ cells in 50 μL of assay media) and 50 μL of serially diluted test articles were added to each well containing target cells and to control wells without target cells in quick succession. The plates were incubated for 4-5 hours at 37° C. BioGlo Substrate (Promega) was then added to each well (50 μL) and the plates incubated for an additional 5-10 minutes at room temperature (“RT”), the signal transduction was measured by detecting luminescence for example using Perkin Elmer Envision device) with luminescence relative light unit (RLU) as the read-out. For mono-specific anti-CD137 antibodies, no target cells were used, instead a four-fold excess of goat anti-mouse or goat anti-human antibody was added to cross link the antibodies.

ELISA assays to evaluate the test articles for CD137 binding were performed essentially as follows: flat bottom maxisorb 96-well plates were coated with soluble human or cynomolgus monkey CD137 (the extracellular domain of human or cynomolgus monkey CD137 fused to a His tag (shCD137 His or scyCD137 His) or to a human Fc Region (shCD137 hFc or scyCD137 hFc)), each at 0.5 or 1 g/mL. The plates were washed, blocked with PBS buffer containing 0.5% bovine serum albumin and 0.1% Tween20, and incubated with a test article (e.g., cell supernatants or purified mAb). For the hybridoma supernatants anti-CD137 antibody was utilized at 1.0 g/mL and six three-fold serial dilutions. The amount of test article binding to the immobilized CD137 (human or cynomolgus monkey) was assessed using a goat anti-mouse IgG-HRP secondary antibody. All samples were analyzed on a plate reader (Victor 2 Wallac, Perkin Elmer) and EC50 values were calculated from dose-response curves by nonlinear regression analysis.

The T cell cytokine release assays (using suboptimal stimulated primary T cells in the absence of target cells) were performed essentially as follows: 50 μL of serially diluted test article (antibodies (+/−cross-linking with anti-human Fc (Fab)′₂)), 50 μL of prewashed Dynabeads aCD3 (REF 11151D; Invitrogen by Thermo Fisher Scientific, or similar) at 2×10⁶ beads/mL, and 100 μL/well of human pan T cells (purified from donor PBMC using Dynabeads Untouched Human T Cells Kit (Invitrogen Cat #11344D) or similar, per manufacture's protocol) at 10⁶ cells/mL were added to each well of the assay plate. The final volume of each well on the plate was 200 μL. For those control wells that did not contain test article or aCD3 beads, assay media was added to bring up the total volume to 200 μL and the plates were incubated for 72 hours in a tissue culture incubator. The supernatants were then collected from each well and the released cytokines of IL-2, IL-10, TNF-α, and IFN-7, were measured using a Cytokine ELISA Kit (e.g., R&D System Human IL-2 DuoSet ELISA (Cat: DY202), Human IFN-gamma DuoSet ELISA (Cat: DY285) and Human TNF-alpha DuoSet ELISA (Cat: DY210) or similar commercial reagents) according to the manufacturer's instructions. Microsoft Excel and SoftMax Pro were used for data analysis to extrapolate cytokine levels, which were plotted with Prism.

FACS analysis to evaluate the test articles for binding to cell surface CD137 was performed essentially as follows: 100 L of CHO cells expressing CD137 (CHO/CD137) (1.0×10⁵ to 1×10⁶ cells/well) and 100 L of serially diluted test article or control was added to each well of microtiter assay plate(s), mixed and incubated at RT for about 30 min. The cells were washed with FACS Buffer and secondary antibody (goat anti-human-APC, PE, or FITC) was then added to each well (1:1000), after which, the components were mixed and the wells were incubated at RT for about 30 min. Cells were washed and resuspended in 250 L FACS Buffer and analyzed by flow cytometry (BD LSR Fortessa or FACSCanto II) for cell events collection. Data analysis were performed via FloJo v10.

Competition between anti-CD137 antibodies and CD137 ligand for binding to CD137 was determined using a real-time, label-free biolayer interferometry assay on an Octet biosensor (Pall ForteBio) essentially as follows: Anti-CD137 antibodies were immobilized on an anti-human Fc biosensor and then associated with the extracellular domain of human CD137 fused to a murine Fc domain (shCD137 mFc; 10 μg/mL) for 30 seconds. The sensors were then dipped into recombinant human CD137 ligand (R&D Systems; 5 μg/mL) for 30 seconds to monitor if the ligand could bind to the preformed CD137/antibody complex or if its binding was blocked.

The binding kinetics of the anti-CD137 antibodies (having human Fc regions) were investigated using BIACORE™ SPR analysis. The anti-CD137 antibodies were captured on a Fab′2 goat-anti-human Fc surface. The association and dissociation of shCD137 His or scyCD137 His (12.5 nM, 50 nM, 200 nM) were monitored and sensograms were fitted using a 1:1 binding model to calculate association and dissociation rate constants and the KD determined.

FACS analysis to evaluate the test articles for binding to cell surface PD-L1 was performed essentially as follows: 100 L of CHO cells expressing PD-L1 (CHO/PD-L1) (1.0×10⁵ to 1.0×10⁶ cells/well) and 100 L of serially diluted test article was added to each well of microtiter assay plate(s), mixed and incubated at RT for about 30 min. The cells were washed with FACS Buffer and secondary antibody (goat anti-human-FITC, PE, or APC) was then added to each well, after which, the components were mixed and the wells were incubated at RT for about 30 min. Cells were washed and resuspended in 250 L FACS Buffer and analyzed by flow cytometry (BD LSR Fortessa or FACSCanto II) for cell events collection. Data analysis were performed via FloJo v10.

The ability of a test article to antagonize the PD-1/PD-L1 axis (i.e., block the PD-1/PD-L1 interaction and prevent down-regulation of T-cell responses) was evaluated in a Jurkat-luc-NFAT/CHO/PD-L1 luciferase PD-L1 reporter assay, performed essentially as follows: CHO/PD-L1 cells were plated at 40,000/well in 100 L of culture medium (DMEM/F12+10% FBS+200 g/mL Hygromycin B+250 g/mL G418) and incubated overnight. The next day the media was removed and NFAT-luc2/PD-1 Jurkat cells (Promega) at 125,000 cells/well in 50 L assay buffer (RPMI+2% FBS), and 50 μL of serial diluted test articles were added to each well and incubated for 6 hours at 37° C. 80 L of BioGlo Substrate (Promega) was then added to each well and the plate was incubated for an additional 5-10 minutes at RT, PD-1/PD-L1 blockade was measured by detecting luminescence (for example using Perkin Elmer Envision device) with luminescence relative light unit (RLU) as the read-out.

The T cell cytokine release assay (using suboptimal stimulated primary T cells in the presence of target cells) were performed essentially as follows: human pan T cells (purified from donor PBMC, see above) were resuspended in assay media and placed in a tissue culture incubator overnight. TA positive target cells (e.g., CHO/PD-L1 cells, JIMT-1 cells, N87 cells), and control TA negative cells (e.g., CHO cells) were obtained from culture. After washing, target cells (number indicated in figures and below) were pre-seeded in flat-bottom bright 96-well plates and placed in a tissue culture incubator overnight. The next day, rested human pan T cells were measured for density and viability by trypan blue exclusion using a Beckman Coulter Vi-Cell counter and adjusted to a density of 2×10⁶ cells/mL. The next day the supernatants were discarded and 50 μL of serially diluted test article (antibodies, diabodies, trivalent molecules, etc.), 50 μL of prewashed Dynabeads aCD3 (REF 11151D; Invitrogen by Thermo Fisher Scientific) at 2.0×10⁶ beads/mL, 50 μL/well of human pan T cells at 2.0×10⁶ cells/mL, and 50 μL of assay media were added to each well of the assay plate. The final volume of each well on the plate was 200 μL. For control wells that did not contain test article or aCD3 beads, assay media was added to bring up the total volume to 200 μL and the plates were incubated for 72 hours in a tissue culture incubator. The supernatants were then collected from each well and the released cytokines of IFN-7 and IL-2 were measured using a Cytokine ELISA Kit (e.g., R&D System (Human IL-2 DuoSet ELISA (Cat: DY202), Human IFN-gamma DuoSet ELISA (Cat: DY285) or similar commercial regents) according to the manufacturer's instructions. Microsoft Excel and SoftMax Pro were used for data analysis to extrapolate cytokine levels, which were plotted with Prism.

Quantification of CD137×TA Binding Molecules in cynomolgus monkey serum was performed essentially as follows: Assay Plates were coated with 2.0 μg/mL of a His-tagged soluble human CD137 fusion protein (huCD137) (containing an extracellular portion of human CD137 fused to a histidine-containing peptide) overnight. After blocking the non-specific sites with 0.5% bovine serum albumin (BSA) in phosphate buffered saline (PBS) with 0.1% Tween-20 (PBST), the plate was incubated with CD137×TA Binding Molecule standard calibrators, quality controls and test samples. The immobilized huCD137-His captures the CD137×TA Binding Molecules present in the standard calibrators, quality controls and test samples. The captured CD137×TA Binding Molecule was detected by the addition of 0.05 μg/mL goat anti-human IgG (Fc)-HRP. The bound HRP activity was quantified by the luminescence light generation using Thermo Scientific SuperSignal ELISA Pico Chemiluminescent Substrate. The luminescence light intensity, expressed as relative light unit (RLU), was measured by the Victor X4 plate reader. The standard curve was generated by fitting RLU signal from AEX3370 standards with a five-parameter logistic model. The concentration of the CD137×TA Binding Molecule in the serum samples was then interpolated from the samples' RLU signal and the equation describing the standard curve. Lower limit of quantification (LLOQ) for the assay was 6.1 ng/mL.

T cell and NK cell proliferation assays were performed essentially as follows: A panel T cell and NK cell marker antibodies including CD3, CD4, CD8, CD56 and CD159a was added into sample plate wells containing well-mixed anticoagulated whole blood samples obtained from the cynomolgus monkey studies, mixed thoroughly using a pipette, and incubated in the dark for 25-35 minutes at ambient temperature. 1×BD FACS lysing solution was then added to each well and mixed using a pipette; each plate was then incubated in the dark for an additional 10-20 minutes at ambient temperature. Each plate was centrifuged at 400×g for 5 minutes and the supernatant was discarded. FACS buffer was added in each well and mixed as a washing step. Each plate was then centrifuged at 400×g for 5 minutes and the supernatant was discarded. The cell pellet was resuspended with BD Cytofix/Cytoperm solution and incubated for 20-40 minutes at 2-8° C. At the end of incubation, each plate was washed as in previous wash steps and the cell pellet was resuspended with Ki67 antibody or isotype control and incubated for 30-60 minutes at 2-8° C. Each plate was again washed as in previous wash steps and the cell pellet was finally resuspended in FACS buffer and the samples were analyzed with a BD FACSCanto II cell analyzer. The proliferation of T-cells and NK cells were quantified by monitoring CD3+CD8+Ki67+ and CD56+CD159a+Ki67+ populations, respectively.

### Example 2

### The Isolation and Characterization of A Human Non-Blocking Anti-CD137 mAb

To identify a CD137 binding domain having improved characteristics, particularly when incorporated into different CD137×TA Binding Molecules, a panel of monoclonal antibodies having fully human variable domains specific for human CD137 were generated using the TRIANNI MOUSE® platform by immunizing mice with a His-tagged soluble human CD137 fusion protein (huCD137) (containing an extracellular portion of human CD137 fused to a histidine-containing peptide). The supernatants from the resulting hybridomas were evaluated for CD137 binding, ability to mediate dose dependent T-cell signal transduction in a CD137 reporter assay, and for the ability to induce cytokine (e.g., IFN-7, TNF-α) release from T cells. The VH and VL Domains of several hybridomas were cloned and expressed in CHO cells as human IgG1 (L234A/L235A) antibodies and evaluated for binding affinity, ligand blocking activity, binding to CD137 on the cell surface and again in both the CD137 reporter and cytokine release assays. An irrelevant negative control antibody and/or the previously described chimeric anti-CD137 antibody chCD137 MAB-3 (see, WO 2018/156740 and above) were included in these evaluations. The methods used for such evaluations are provided above. One antibody designated CD137 MAB-6 (1.1), was selected for further study. The amino acid sequences of the VH and VL Domains CD137 MAB-6 (1.1) are provide above and representative results from these evaluations are summarized in Tables 7A-D.

TABLE 7A
Hybridoma Supernatants

| | Reporter Assay (RLU) | | ELISA (EC50 | |
|---|---|---|---|---|
| | IgG conc | | ng/mL) | |
| Antibody (conc.) | 0.02 μg/mL | 0.1 μg/mL | huCD137 | cyCD137 |
| CD137MAB-6(1.1) | 3280 | 11670 | 38 | 49 |
| chCD137MAB-3 | 5540 | 18650 | 34 | 44 |
| non-specific mAb | 1020 | 1240 | — | — |

TABLE 7B
Hybridoma Supernatants
T cell assay-cytokine release

| | IL-2 | | | | IL-10 | | | |
|---|---|---|---|---|---|---|---|---|
| | Donor 1 | | Donor 2 | | Donor 1 | | Donor 2 | |
| (conc.) | 0.1 | 1 | 0.1 | 1 | 0.1 | 1 | 0.1 | 1 |
| Antibody | μg/mL | μg/mL | μg/mL | μg/mL | μg/mL | μg/mL | μg/mL | μg/mL |
| CD137 MAB-6 (1.1) | 25 | 307 | 172 | 402 | 35 | 393 | 204 | 973 |
| chCD137 MAB-3 | 124 | 223 | 313 | 628 | 52 | 63 | 225 | 391 |
| non-specific mAb | 16 | 29 | 27 | 29 | 33 | 35 | 45 | 54 |
| | TNF-α | | | | IFN-γ | | | |
| | Donor 1 | | Donor 2 | | Donor 1 | | Donor 2 | |
| (conc.) | 0.1 | 1 | 0.1 | 1 | 0.1 | 1 | 0.1 | 1 |
| Antibody | μg/mL | μg/mL | μg/mL | μg/mL | μg/mL | μg/mL | μg/mL | μg/mL |
| CD137 MAB-6 (1.1) | 76 | 908 | 355 | 702 | 116 | 2901 | 546 | 2573 |
| chCD137 MAB-3 | 275 | 368 | 370 | 508 | 194 | 303 | 287 | 455 |
| non-specific mAb | 35 | 113 | 68 | 102 | 33 | 92 | 56 | 99 |

TABLE 7C
CHO cell Supernatants

| | | SPR | | | Surface Binding | |
|---|---|---|---|---|---|---|
| | Ligand | kon, | koff, | KD, | | EC50 |
| Antibody | Blocking | 1/(Ms) | 1/s | nM | MFI max | ng/mL |
| CD137 MAB-6 (1.1) | no | 4.2E+5 | 6.7E−3 | 16 | 5745 | 21 |
| chCD137 MAB-3 | yes | 6.6E+5 | 2.5E−2 | 38 | 6046 | 10 |
| non-specific mAb | — | | — | — | 149 | — |

TABLE 7D
CHO cell Supernatants

| | Reporter | Primary T cell assay | |
|---|---|---|---|
| | Assay | Donor 1 | |
| | (RLU) | INF γ | TNF-α |
| (conc.) | 0.08 | 0.1 | 0.1 |
| Antibody | μg/mL | μg/mL | μg/mL |
| CD137MAB-6(1.1) | 8530 | 174 | 101 |
| chCD137 MAB-3 | 9170 | 63 | 45 |
| non-specific mAb | 1250 | 0 | 6 |

Epitope binning was performed by cross-competition studies. The results of these and ligand binding competition studies indicate that CD137 MAB-6 binds an epitope distinct from a comparator antibody comprising the variable domains of utomilumab, a comparator antibody comprising the variable domains of urelumab, and all of the anti-CD137 antibodies described in WO 2018/156740 including chCD137 MAB-3. In sum, these studies demonstrate that CD137 MAB-6 binds a unique non-blocking epitope and exhibits better binding affinity than the previously described chCD137 MAB-3 as determined by ELISA, FACS and BIACORE™ assays. CD137 MAB-6 also exhibits higher activity in T-cell cytokine release assay.

### Example 3

### Characterization of CD137×TA Binding Molecules

CD137×TA Binding Molecules capable of binding to CD137 and to the representative TA, PD-L1, were generated incorporating the VH and VL Domains of CD137 MAB-6(1.1) and hPD-1 MAB-2(1.1). In particular, a tetravalent bispecific diabody designated “DART-A,” comprising two identical bispecific diabody binding domains and having the antibody-like Y structure shown in FIG. 1B, and a trivalent binding molecule designated “TRIDENT-A,” comprising one mono-specific diabody-type binding domain, and a non-diabody-type binding domain having the structure shown in FIG. 3A, were generated. The domain attributes of these molecules, and certain bispecific control and comparator molecules having the same structures are discussed above (see e.g., Tables 5-6).

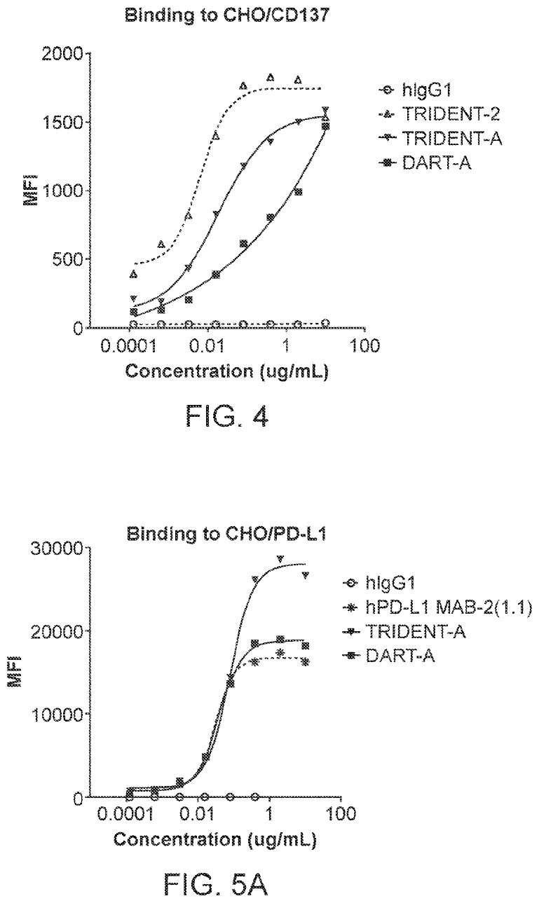

DART-A, TRIDENT-A, a comparator molecule TRIDENT-2 (comprising the binding domain of hCD137 MAB-3(1B.3)), and a negative control (hIgG1—an irrelevant antibody as an isotype control) were evaluated for their ability to bind to cell surface CD137 by FACS analysis performed essentially as described-above, test articles were used at 10 μg/mL and five-fold serial dilutions. The results of a representative assay shown in FIG. 4 demonstrate that all the CD137×PD-L1 bispecific molecules were capable of efficiently binding to CD137 expressed on the cell surface. In this assay the comparator molecules appear to exhibit better binding.

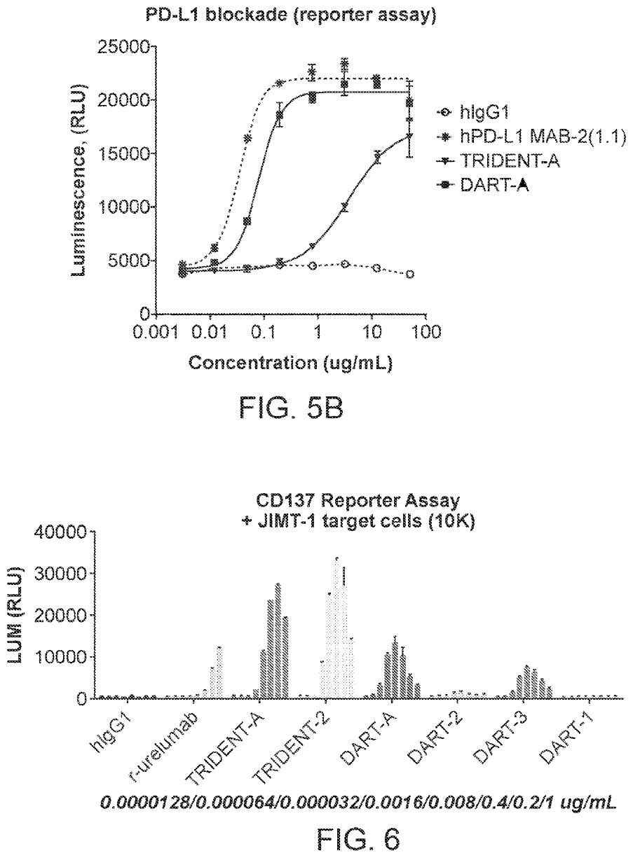

DART-A, TRIDENT-A, hPD-1 MAB-2(1.1), and the negative control, hIgG1, were evaluated for their ability to bind to the surface of CHO cells expressing PD-L1 (CHO/PD-L1) by FACS analysis, and for their ability to antagonize the PD-1/PD-L1 axis (i.e., block the PD-1/PD-L1 interaction and prevent down-regulation of T-cell responses) in the Jurkat-luc-NFAT/CHO/PD-L1 luciferase reporter assay, both assays were performed essentially as described above. Test articles were used at 10 μg/mL and five-fold serial dilutions for the FACS analysis and at 50 μg/mL and five-fold serial dilutions for the PD-L1 reporter assay. The results of a representative assay shown in FIGS. 5A-5B demonstrate that DART-A and TRIDENT-A were capable of efficiently binding to PD-L1 expressed on the cell surface (FIG. 5A), and of blocking PD-1/PD-L1 interactions (FIG. 5B). It will be noted that the binding curves of molecules having two PD-L1 binding sites (DART-A, and hPD-1 MAB-2(1.1)) reach saturation sooner indicating that some of the molecules are exhibiting bivalent binding (i.e., binding two PD-L1 molecules on the surface). Bivalent bonding is more likely in the presence of high concentrations of target ligand expressed on the CHO/PD-L1 cells. It was also observed that the molecules, possessing two PD-L1 binding sites, exhibited greater PD-L1 blocking activity relative to the trivalent molecules.

The functional activity of DART-A, TRIDENT-A, the comparator molecules: DART-2, and TRIDENT-2 (each comprising the binding domain of hCD137 MAB-3(1B.3)), DART-3 (comprising the binding domain of utomilumab), a replica of the agonistic anti-CD137 mAb urelumab (r-urelumab), and the negative controls: DART-1 (an RSV×PD-L1 binding molecule) and hIgG1 was evaluated in a CD137 reporter assay performed essentially as described above in the presence and absence of PD-L1 expressing JIMT-1 cells (10,000 cells per well), test articles were used at 1 μg/mL and five-fold serial dilutions. The results of a representative assay shown in FIG. 6 demonstrate that both the tetravalent and trivalent CD137×PD-L1 bispecific molecules comprising the binding domain CD137 MAB-6 (1.1) (DART-A and TRIDENT-A, respectively) mediated target-dependent signal transduction, in contrast only the trivalent molecule comprising the binding domain of hCD137 MAB-3(1B.3) (TRIDENT-2) exhibited activity while the corresponding tetravalent molecule DART-2, did not exhibit any activity in the presence of target cells. Additionally, DART-A exhibited the highest activity of all the tetravalent molecules tested. None of the CD137×TA Bispecific Molecules exhibited activity in the absence of target cells. As expected, the agonist r-urelumab exhibited activity in the presence and absence of PD-L1 expressing target cells, and the negative controls did not exhibit activity at all.

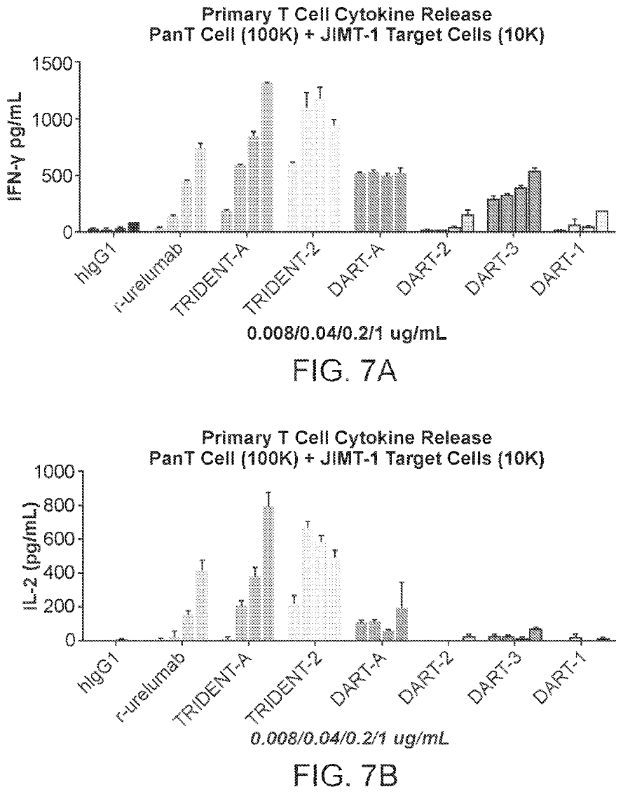

The functional activity of DART-A, TRIDENT-A, comparator molecules: DART-2, TRIDENT-2, DART-3, r-urelumab, and the negative controls: DART-1 and hIgG1 was also evaluated in the primary T cell cytokine release assay presence of PD-L1 expressing JIMT-1 cells (10,000 cells per well) performed essentially as described above, test articles were used at 1 μg/mL and five-fold serial dilutions. The results of a representative assay for the representative cytokines INF-7 and IL-2 are shown in FIGS. 7A and 7B, respectively. As was seen in the CD137 reporter assay, both the tetravalent and trivalent CD137×PD-L1 bispecific molecules comprising the binding domain CD137 MAB-6(1.1) (DART-A and TRIDENT-A, respectively) mediated cytokine release, in contrast only the trivalent molecule comprising the binding domain of hCD137 MAB-3(1B.3) (TRIDENT-2) exhibited activity while the tetravalent molecule DART-2, did not exhibit any activity. Again DART-A exhibited the highest activity of all the tetravalent molecules tested and the negative controls did not exhibit activity.

These studies demonstrate that the fully human binding domain CD137 MAB-6(1.1) is active in both tetravalent and trivalent CD137×TA bispecific molecules, and does not exhibit agonist activity in the absence of a PD-L1 expressing target cell. Although TRIDENT-2 exhibited higher binding to CHO/CD137 cells, the activity of TRIDENT-A and TRIDENT-2 were comparable while DART-2 exhibited no activity in either of the functional assays. Indeed, CD137 MAB-6(1.1) is more active in the tetravalent antibody-like structure shown in FIG. 1B than molecules comprising the binding domains of hCD137 MAB-3(1B.3) or utomilumab.

### Example 4

### Pharmacokinetics of CD137×TA Molecules

The pharmacokinetics of the trivalent CD137×TA bispecific molecules TRIDENT-A (comprising the binding domain of CD137 MAB-6(1.1)) and TRIDENT-2 (comprising the binding domain of hCD137 MAB-3(1B.3)) were evaluated in Cynomolgus monkeys. Briefly, two cynomolgus monkeys (females) were infused with a single dose of each test article at 1 mg/kg or 10 mg/kg (four groups) and the animals were monitored for 22 days, no necropsies were performed. Animals were monitored for food consumption, body weight, and full hematology, and clinical chemistry were performed during the study. Transient increases in liver enzymes (ALT, AST, and bilirubin) were observed. The test articles were well tolerated and no adverse effects were observed.

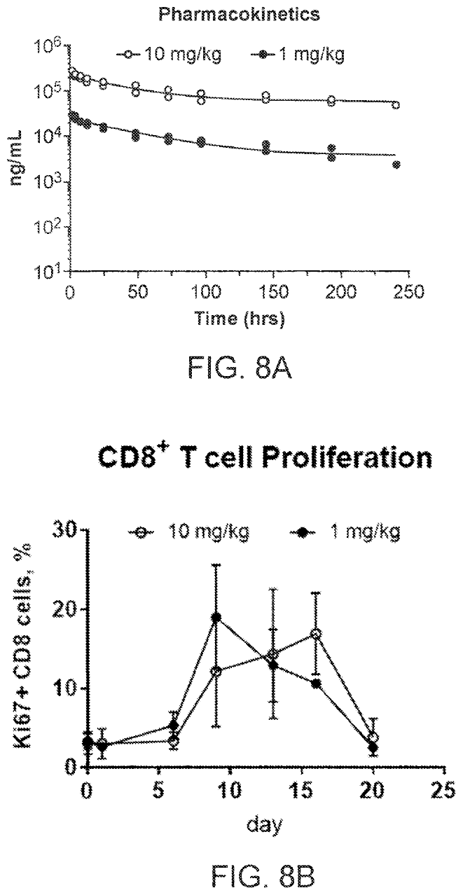

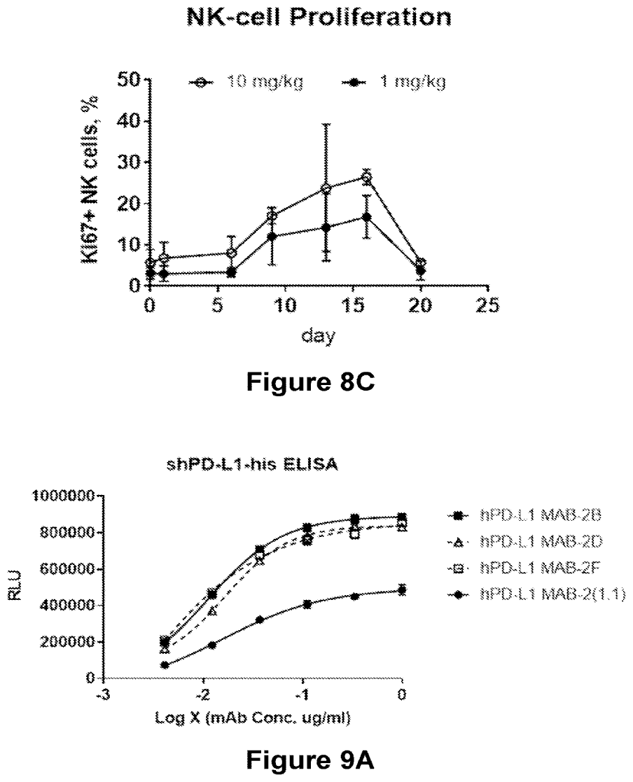

The serum concentration of the molecules was monitored over time, essentially as described above. The Cmax, AUC, t1/2beta and CL values are presented in Table 8 and show that the trivalent CD137×TA bispecific molecule comprising the binding domain of CD137 MAB-6(1.1) exhibits a serum half-life about two-fold longer than the one comprising hCD137 MAB-3(1B.3). The serum concentration of TRIDENT-A over the first 10 days (240 hours) is plotted in FIG. 8A. In addition, the proliferation of CD8+ T cells (FIG. 8B), and NK cells (FIG. 8C) was examined essentially as described above. This study shows that the representative CD137×TA bispecific molecule TRIDENT-A, comprising CD137 binding domains of the novel anti-CD137 antibody CD137 MAB-6 exhibited slow clearance and administration correlated with a temporary induction in the proliferation of both CD8+ T cells and NK cells indicating a stimulation of these immune cells. In addition, as noted above CD137 MAB-6 is more active in the tetravalent antibody-like structures. Thus, the CD137 MAB-6 binding domain provides several advantages as compared to previously described CD137 binding domains.

TABLE 8
Pharmacokinetics

| | Dose | Animal | Cmax | AUC | t½ Beta | CL |
|---|---|---|---|---|---|---|
| Molecule | (mg/kg) | ID | (ug/mL) | (hr*ug/mL) | (hr) | (mL/hr/kg) |
| TRIDENT-A | 1 | 1F001 | 28 | 1598 | 98 | 0.48 |
| | | 1F002 | 32 | 1968 | 148 | 0.32 |
| TRIDENT-A | 10 | 2F003 | 242 | 18752 | 217 | 0.30 |
| | | 2F004 | 293 | 21333 | 167 | 0.27 |
| TRIDENT-2 | 1 | 5F009 | 31 | 1227 | 29 | 0.81 |
| | | 5F010 | 26 | 945 | 42 | 0.97 |
| TRIDENT-2 | 10 | 6F011 | 282 | 10313 | 100 | 0.78 |
| | | 6F012 | 293 | 11486 | 99 | 0.69 |

### Example 5

### Deimmunization and Optimization of hPD-1 MAB-2 (1.1) Variable Domains

Intact hPD-1 MAB-2(1.1) antibody was analyzed using an MAPPs assay (performed by Abzena), to identify peptide clusters that could be presented by antigen presenting cells. This was followed by iTope™ in silico analysis of the amino acid sequence of the VH and VL Domains of hPD-1 MAB-2(1.1), in which peptide clusters were divided into overlapping 9-mer peptides (8 aa overlap between adjacent peptides) and binding affinity of the 9-mer peptides to HLA-DR proteins was predicted and the 9-mer peptides were crosschecked against a database of peptides which have been experimentally shown to stimulate T cell responses. A potential T cell epitope within Kabat residues 72-88 of the framework region 2 of hPD-1 MAB-2 VH1 (corresponding to residues 73-92 of SEQ ID NO:57) was identified. Further analysis identified three non-germline amino acids: T77, K83, and T84, numbered according to Kabat (corresponding to residues T78, K87, and T88 of SEQ ID NO:57) in this region and the following substitutions were introduced: T77S; K83R and T84A; or T77S, K83R and T84A, numbered according to Kabat. The binding of antibodies comprising these substitutions were evaluated for binding to a soluble PD-L1-fused to a His tag (shPD-L1) using an ELISA assay essentially as described above for CD137 binding except the plates were coated with shPD-L1 at 0.5 μg/mL and a goat anti-human IgG-HRP secondary antibody was used. The results are summarized in Table 9.

TABLE 9
Summary of shPD-L1 ELISA

| Antibody | Binding |
|---|---|
| hPD-1 MAB-2 VH1 | ++++ |
| hPD-1 MAB-2 VH1 + T77S | ++ |
| hPD-1 MAB-2 VH1 + K83R and T84A | +++ |
| hPD-1 MAB-2 VH1 + T77S, K83R and | ++++ |
| T84A | |

All the deimmunized variants bound to shPD-L1, with the variant comprising the T77S, K83R and T84A substitutions exhibiting binding that was indistinguishable from the parental antibody. The VH comprising these mutations was designated hPD-L1 MAB-2 VH2.

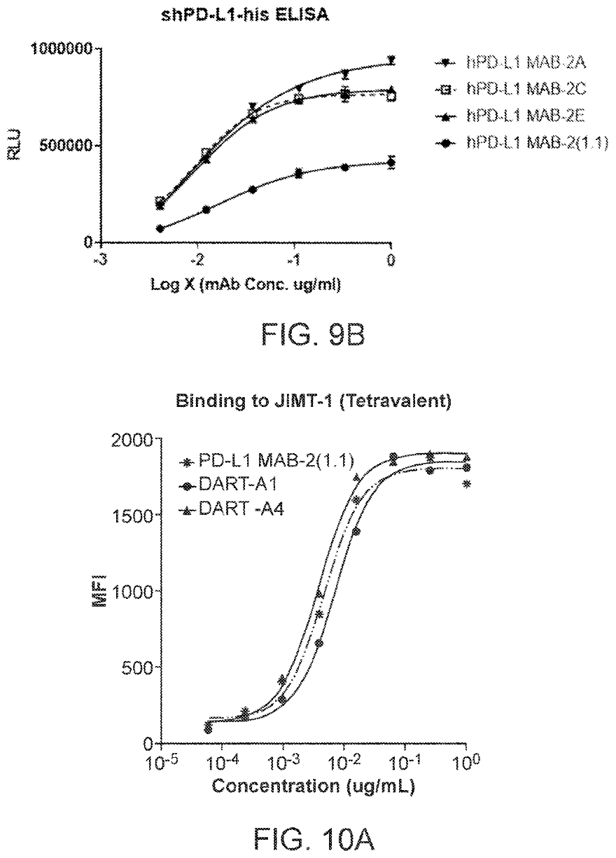

In addition, a mutational analysis was undertaken to identify amino acid substitutions in the CDRs of the VH and/or VL domains of hPD-L1 MAB-2(1.1) that enhanced binding to shPD-L1. A number of substitutions that resulted in improved binding were identified and are provided in Table 10 with the substitution presented using the Kabat numbering system and the corresponding amino acid residue in the sequences presented above indicated. The binding of Fab fragments comprising different combinations of these substitutions to shPD-L1-his was evaluated by ELISA essentially as described above except a goat anti-human kappa-HRP secondary antibody was used. Representative variants evaluated are summarized in Table 11, and the binding curves are presented in FIGS. 9A-9B. These studies show that Fabs comprising each of the variants (hPD-L1 MAB-2B, hPD-L1 MAB-2D, and hPD-L1 MAB-2F plotted in FIG. 9A; hPD-L1 MAB-2A, hPD-L1 MAB-2C, and hPD-L1 MAB-2E plotted in FIG. 9B) exhibited higher affinity than the Fab comprising the parental hPD-L1 MAB-2(1.1).

TABLE 10
CDR Substitutions

| VH Domain | | VL Domain | |
|---|---|---|---|
| | SEQ ID | | SEQ ID |
| Kabat* | NO: 57\hat{ } | Kabat* | NO: 58\hat{ } |
| S31G | 31 | T31E | 31 |
| G53K | 54 | D28H or D28V or D28L | 28 |
| Q95A or Q95G | 99 | S52E | 52 |
| F100aG | 105 | | |

*substitution numbering according to Kabat
\hat{ }corresponding amino acid residue number of the indicated sequence identification number

TABLE 11
hPD-L1 MAB-2 Variants Comprising CDR Substitutions

| Variant | Substitutions* |
|---|---|
| hPD-L1 MAB-2A | VH(S31G/G53K/Q95A) & VL(D28H) |
| hPD-L1 MAB-2B | VH(S31G/G53K/Q95G) & VL(D28V) |
| hPD-L1 MAB-2C | VH(S31G/G53K/F100aG) & VL(D28L) |
| hPD-L1 MAB-2D | VH(F100aG) & VL(D28L/S52E) |
| hPD-L1 MAB-2E | VH(G53K/Q95A) & VL(D28L) |
| hPD-L1 MAB-2F | VH(G53K/F100aG) & VL(T31E) |

*substitution numbering according to Kabat

A number of variant VH and/or VL Domains of hPD-L1 MAB-2 were used to generate CD137×TA bispecific molecules (all comprising two CD137 MAB-6(1.1) binding sites). The particular hPD-L1 MAB-2 VL and VH variants used are summarized in Table 12, the amino sequences of these variants and the bispecific molecules comprising them are provided above. As noted above molecules comprising PD-L1 MAB-2 VH/VL Domains are referred to by reference to the specific VH/VL Domains, for example, a molecule comprising the binding domains PD-L1 MAB-2 VH3 and hPD-L1 MAB-2 VL2 is specifically referred to as “PD-L1 MAB-2(3.2).”

TABLE 12
hPD-L1 MAB-2 Variant VH and VL Domains

| Variant | Substitutions* |
|---|---|
| hPD-L1 MAB-2 VH2 | T77S/K83R/T84A |
| hPD-L1 MAB-2 VH3 | T77S/K83R/T84A + G53K/F100aG |
| hPD-L1 MAB-2 VH4 | T77S/K83R/T84A + Q95A |
| hPD-L1 MAB-2 VH5 | T77S/K83R/T84A + G53K/Q95A |
| hPD-L1 MAB-2 VH6 | T77S/K83R/T84A + Q95A/F100aG |
| hPD-L1 MAB-2 VL2 | VL(T31E) |

*substitution numbering according to Kabat

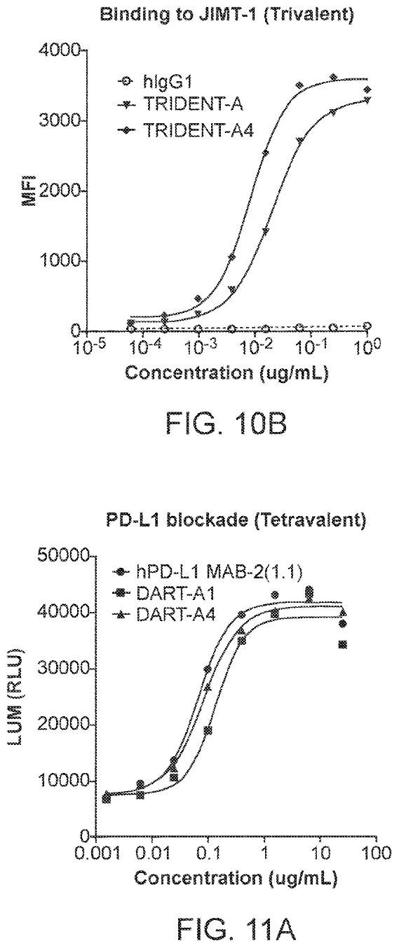

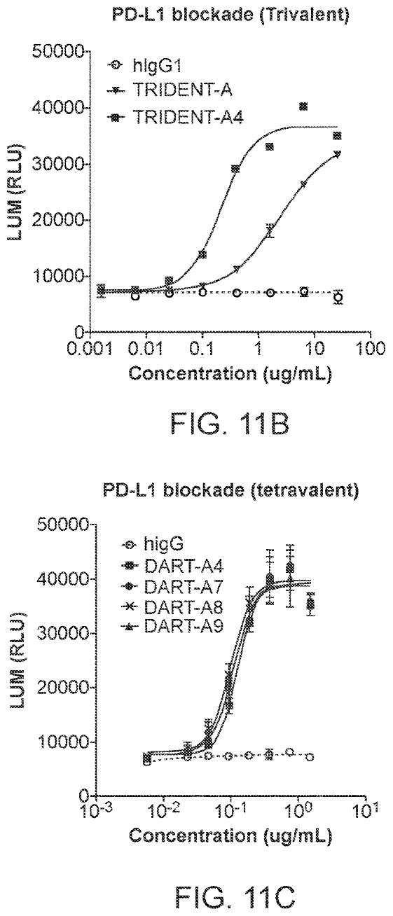

The following molecules comprising two PD-L1 binding sites: DART-A1 (comprising hPD-L1 MAB-2(2.1)); DART-A4 (comprising hPD-L1 MAB-2(3.2)); and hPD-L1 MAB-2(1.1), and the following molecules comprising one PD-L1 binding site: TRIDENT-A (detailed above); and TRIDENT-A4 (comprising hPD-L1 MAB-2(3.2)), were evaluated for PD-L1 binding on the surface of JIMT-1 cells by FACS analysis essentially as described above, test articles were used at 1 μg/mL and four-fold serial dilutions. The results of representative assays are shown in FIGS. 10A-10B. These molecules were also examined for their ability to block the PD-1/PD-L1 interaction in a PD-L1 reporter assay essentially as described above test articles at 25 μg/mL and four-fold serial dilutions. The results of representative assays are shown in FIGS. 11A-11B. These data show that the CD137×TA bispecific molecules comprising the binding domains of the deimmunized/optimized hPD-L1 MAB-2(3.2) exhibited improved binding (FIGS. 10A-10B) and more efficient blocking of PD-1/PD-L1 interactions (FIGS. 11A-11B). A modest improvement was seen in the tetravalent molecules comprising two PD-L1 binding domains (DART-A4 in FIGS. 10A and 11A), a larger improvement in activity was seen in the trivalent molecules comprising only one PD-L1 binding domain (TRIDENT-A4 in FIGS. 10B and 11B). Additional molecules comprising two PD-L1 binding sites: DART-A4 (comprising hPD-L1 MAB-2(3.2)); DART-A7 (comprising hPD-L1 MAB-2(4.2)); DART-A8 (comprising hPD-L1 MAB-2(5.2)); and DART-A9 (comprising hPD-L1 MAB-2(6.2)); were evaluated for their ability to block the PD-1/PD-L1 interaction in a PD-L1 reporter assay essentially as described above with the test articles at 1.5 μg/mL and two-fold serial dilutions. The results of a representative assay is shown in FIG. 11C. This study shows that CD137×TA bispecific molecules comprising the alternative deimmunized/optimized hPD-L1 MAB-2(4.2), hPD-L1 MAB-2(5.2), and hPD-L1 MAB-2(6.2) exhibit a blocking activity similar or improved as compared to that of DART-A4 comprising hPD-L1 MAB-2(3.2).

### Example 6

### Deimmunization of CD137 MAB-6

To minimize the likelihood of immunogenicity substitutions were introduced into the framework regions of the VL Domain of CD137 MAB-6 to replace non-germline residues with those present in the human germline. In particular, combinations of the following substitutions were introduced T14S, F36Y and I39K, numbered according to Kabat (corresponding to residues 14, 37, and 40 of SEQ ID NO:50) the resulting CD137 MAB-6 VL Domains are summarized in Table 13 below and were used to generate CD137×TA bispecific molecules capable of binding to CD137 and to the representative TA, PD-L1, the amino sequences of these variants and the bispecific molecules comprising them are provided above.

TABLE 13
CD137 MAB-6 Variant VL Domains

| Variant | Substitutions* |
|---|---|
| CD137 MAB-6 VL2 | L14S/F36Y/I39K |
| CD137 MAB-6 VL3 | F14S/I39K |

*substitution numbering according to Kabat

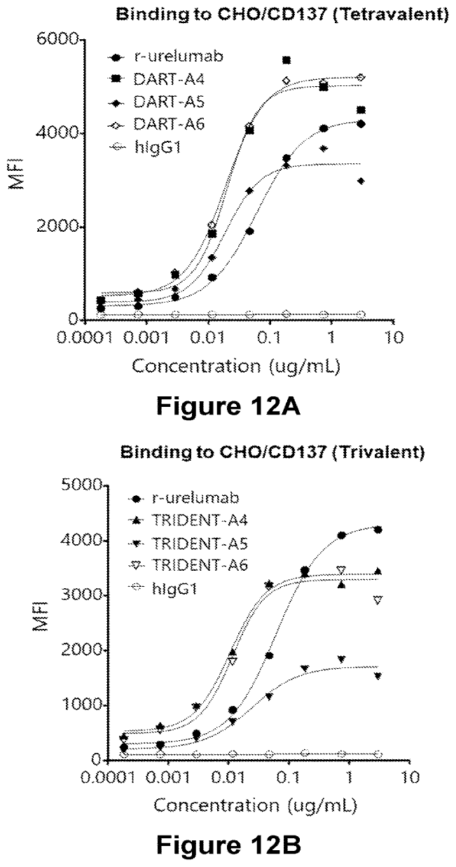

The following molecules comprising two hPD-L1 MAB-2(3.2) binding sites: DART-A4 (comprising CD137 MAB-6(1.1)); DART-A5 (comprising CD137 MAB-6(1.2)); DART-A6 (comprising CD137 MAB-6(1.3)); and the following molecules comprising one hPD-L1 MAB-2(3.2) binding site: TRIDENT-A4 (comprising CD137 MAB-6(1.1)); TRIDENT-A5 (comprising CD137 MAB-6(1.2)); and TRIDENT-A6 (comprising CD137 MAB-6(1.3), a replica of urelumab (r-urelumab), and the negative control hIgG1, were evaluated for their ability to bind to cell surface CD137 by FACS analysis performed essentially as described-above, test articles were used at 3 μg/mL and four-fold serial dilutions. The results of a representative assay shown in FIGS. 12A-12B demonstrate that the tetravalent (FIG. 12A) and trivalent (FIG. 12B) CD137×PD-L1 bispecific molecules comprising the binding domains of CD137 MAB-6(1.3) exhibit binding profiles nearly identical to the same molecules comprising the binding domains of CD137 MAB-6(1.1), while those comprising CD137 MAB-6(1.2) exhibited reduced binding.

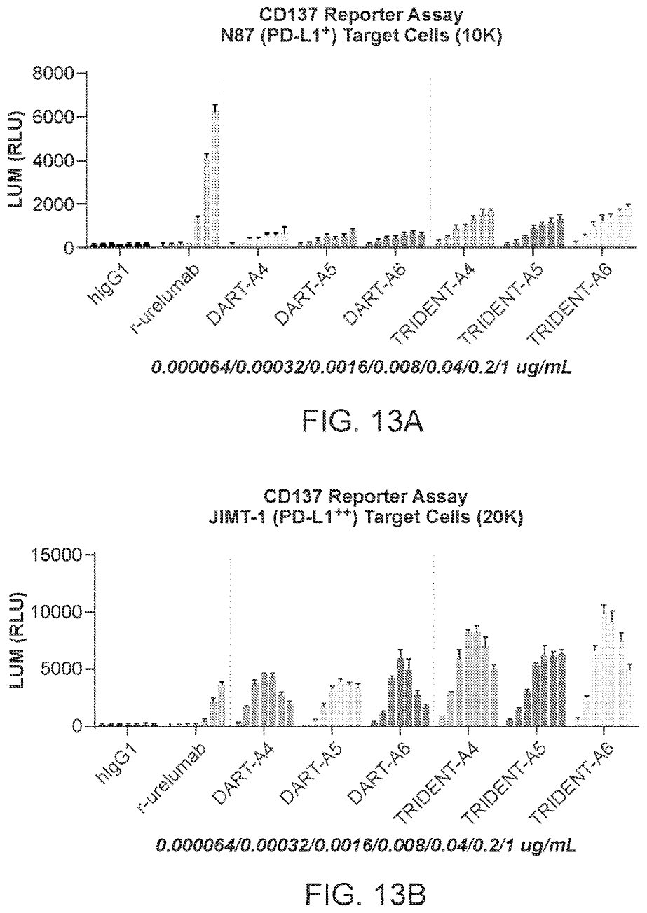

The functional activity of DART-A4, DART-A5, DART-A6, r-urelumab, TRIDENT-A4, TRIDENT-A5, and TRIDENT-A6, and the negative control hIgG1, was examined in a CD137 reporter assay performed essentially as described above in the presence and absence of PD-L1 expressing N87 Target cells (10,000 cells per well,) or JIMT-1 cells (20,000 cells per well), test articles were used at 1 μg/mL and five-fold serial dilutions. The results of a representative assay shown in FIGS. 13A-13B demonstrate that all the molecules exhibiting activity in a target dependent manner with higher activity in the presence of JIMT-1 cells (PD-L1⁺⁺, FIG. 13B) than in the presence ofN87 cells (PD-L1⁺, FIG. 13A). CD137×PD-L1 bispecific molecules comprising the binding domains of CD137 MAB-6(1.3) exhibit activity profiles nearly identical to the same molecules comprising the binding domains of CD137 MAB-6(1.1), while those comprising CD137 MAB-6(1.2) exhibited reduced activity. None of the CD137×PD-L1 bispecific molecules exhibited activity in the absence of target cells. As expected, the agonist r-urelumab exhibited activity in the presence and absence of PD-L1 expressing target cells, and the negative controls did not exhibit activity at all.

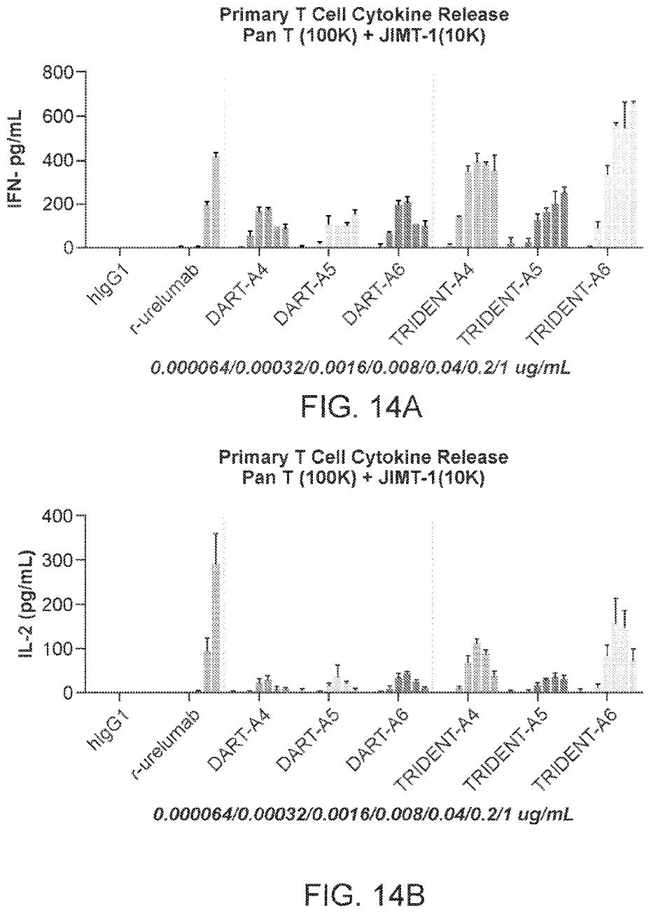

The functional activity of DART-A4, DART-A5, DART-A6, r-urelumab, TRIDENT-A4, TRIDENT-A5, and TRIDENT-A6, and the negative control hIgG1, was examined in the primary T cell cytokine release assay presence of PD-L1 expressing JIMT-1 cells (10,000 cells per well) performed essentially as described above, test articles were used at 1 μg/mL and five-fold serial dilutions. The results of a representative assay for the representative cytokines INF-7 and IL-2 are shown in FIGS. 14A and 14B, respectively. As was seen in the CD137 reporter assay, CD137×PD-L1 bispecific molecules comprising the binding domains of CD137 MAB-6(1.3) exhibit activity profiles nearly identical to, or slightly better than the same molecules comprising the binding domains of CD137 MAB-6(1.1), while those comprising CD137 MAB-6(1.2) exhibited reduced activity.

### Example 7

### Additional Characterization of CD137×TA Molecules

Additional in-vitro studies were undertaken to evaluate the activity of the following representative CD137×PD-L1 bispecific molecules comprising the PD-L1 and CD137 binding domains of PD-L1 MAB-2, the novel CD137 MAB-6, or the deimmunized/optimized variants thereof: DART-A; DART-A4; DART-A6; DART-A7; TRIDENT-A; TRIDENT-A4; TRIDENT-A6; and an additional tetravalent molecule: DART-A10 (comprising two hPD-L1 MAB-2(4.2) binding sites and two CD137 MAB-6(1.3) binding sites). The CD137 and PD-L1 binding domains of these molecule are summarized in Table 5 above.

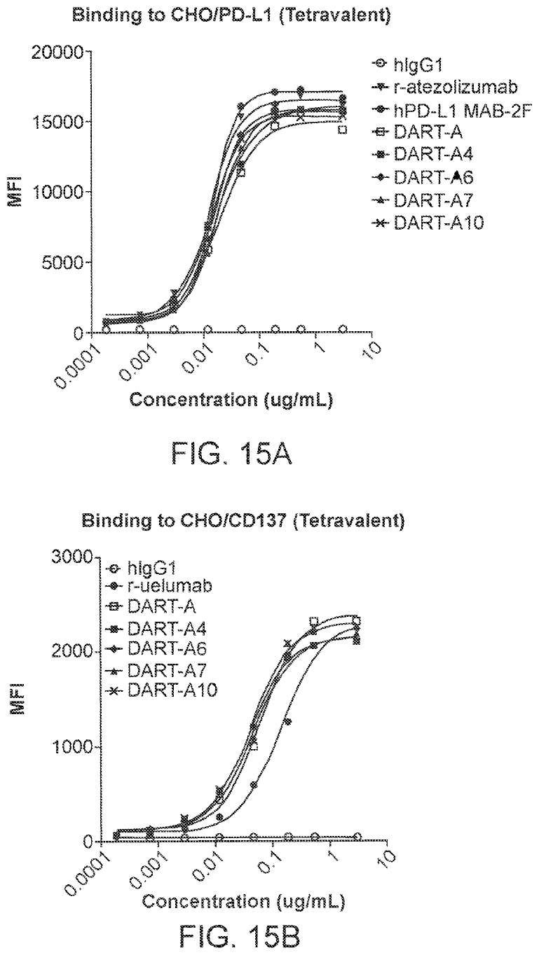

DART-A, DART-A4, DART-A6, DART-A7, DART-A10, the negative control hIgG1, and a replica of the anti-PD-L1 antibody atezolizumab (r-atezolizumab), hPD-L1 MAB-2F or r-urelumab, were evaluated for their ability to bind to the surface of CHO cells expressing PD-L1 (CHO/PD-L1) or expressing CD137 (CHO/CD137) by FACS analysis essentially as described above with the test articles used at a starting concentration of 3 g/mL and 3 to 4-fold dilutions. The results of representative assays are shown in FIGS. 15A-15B. These binding studies demonstrate that the CD137×TA Binding Molecules comprising optimized PD-L1 binding domains (DART-A4, DART-A6, DART-A7, DART-A10) exhibit improved binding to PD-L1 as compared to DART-A (FIG. 15A). Similar PD-L1 binding profiles were observed for binding to JIMT-1 (PD-L1+) cells. These studies also demonstrate that CD137×TA Binding Molecules comprising binding domains of CD137 MAB-6(1.3) and CD137 MAB-6(1.1) exhibit similar binding that is improved as compared to r-uelumab (FIG. 15B).

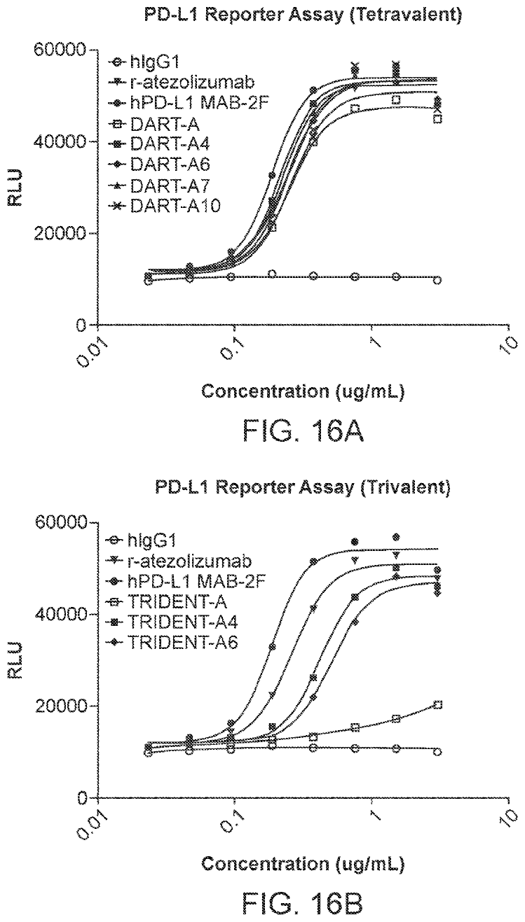

DART-A, DART-A4, DART-A6, DART-A7, DART-A10, TRIDENT-A, TRIDENT-A4, TRIDENT-A6, hPD-L1 MAB-2F, r-atezolizumab and the negative control hIgG1, were evaluated for their ability to block the PD-1/PD-L1 interaction in a PD-L1 reporter assay essentially as described above test articles at 3 μg/mL and two-fold serial dilutions. The results of representative assays are shown in FIGS. 16A-16B. These studies again show that tetravalent (FIG. 16A) and trivalent (FIG. 16B) CD137×TA bispecific molecules comprising the binding domains of the deimmunized/optimized hPD-L1 MAB-2(3.2) or hPD-L1 MAB-2(4.2), exhibited more efficient blocking of PD-1/PD-L1 interactions as compared to molecules comprising the parental binding domain, hPD-L1 MAB-2(1.1), the enhanced activity was independent of the CD137 binding domain. As noted above, in this assay a larger improvement in activity was observed in the trivalent molecules comprising one PD-L1 binding domain, with activity approaching that observed with the anti-PD-L1 antibodies hPD-L1 MAB-2F and r-atezolizumab (each having two PD-L1 binding domains).

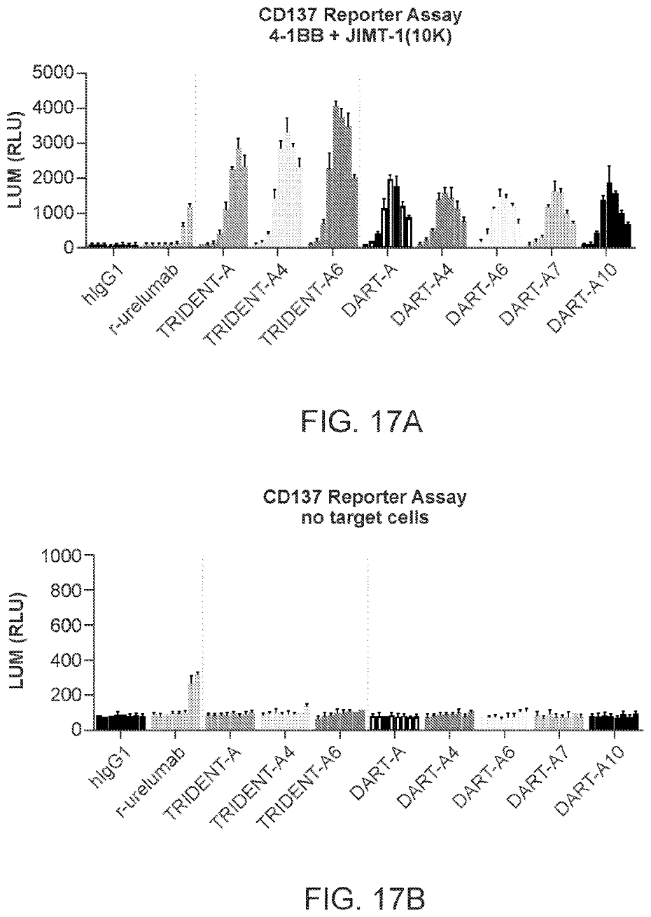

The functional activity of DART-A, DART-A4, DART-A5, DART-A6, TRIDENT-A4, TRIDENT-A5, and TRIDENT-A6, r-urelumab, and the negative control hIgG1, was examined in a CD137 reporter assay performed essentially as described above in the presence and absence of PD-L1 expressing JIMT-1 cells (10,000 cells per well), test articles were used at 1 μg/mL and five-fold serial dilutions (amounts are depicted at the bottom of FIG. 14A, for example). The results of a representative assay are shown in FIGS. 17A-17B and demonstrate that all the bivalent molecules exhibit activity in a target dependent manner with high levels of activity in the presence of JIMT-1 cells (FIG. 17A) and no activity in the absence of target cells (FIG. 17B). Molecules comprising the deimmunized/optimized PD-L1 and CD137 binding domains exhibited higher activity (e.g., TRIDENT-A6 and DART-A10) as compared to those comprising the parental domains. In this assay, the trivalent molecules exhibit higher activity and the increase in activity was greater for the trivalent molecules.

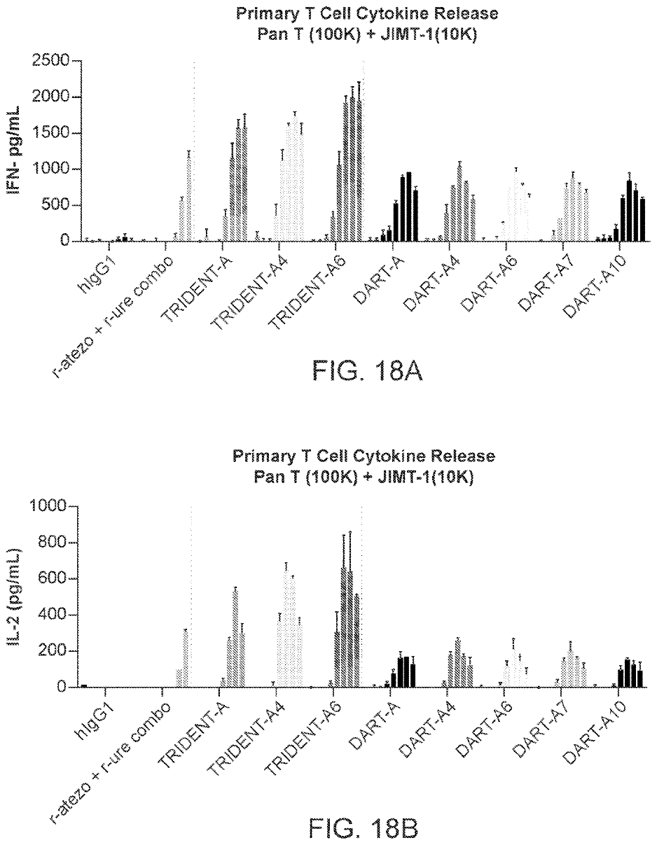

The functional activity of DART-A, DART-A4, DART-A5, DART-A6, r-urelumab, TRIDENT-A4, TRIDENT-A5, and TRIDENT-A6, a combination of r-urelumab and r-atezolizumab, and the negative control hIgG1, was also examined in the primary T cell cytokine release assay presence of PD-L1 expressing JIMT-1 cells (10,000 cells per well) performed essentially as described above, test articles were used at 1 μg/mL and five-fold serial dilutions (amounts are depicted at the bottom of FIG. 14A, for example). The results of a representative assay for the representative cytokines INF-7 and IL-2 are shown in FIGS. 18A and 18B, respectively. As was seen in the CD137 reporter assay, the trivalent CD137×PD-L1 bispecific molecules comprising the deimmunized/optimized PD-L1 and CD137 binding domains exhibited higher activity (e.g., TRIDENT-A6).

### Example 8

### Murine Xenograft Models

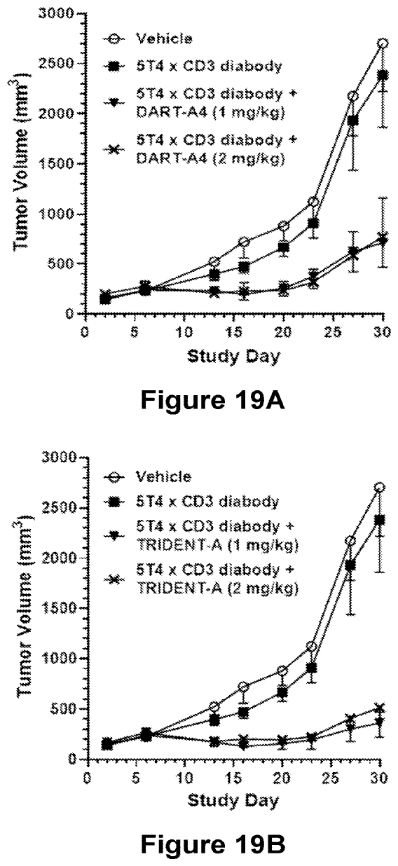

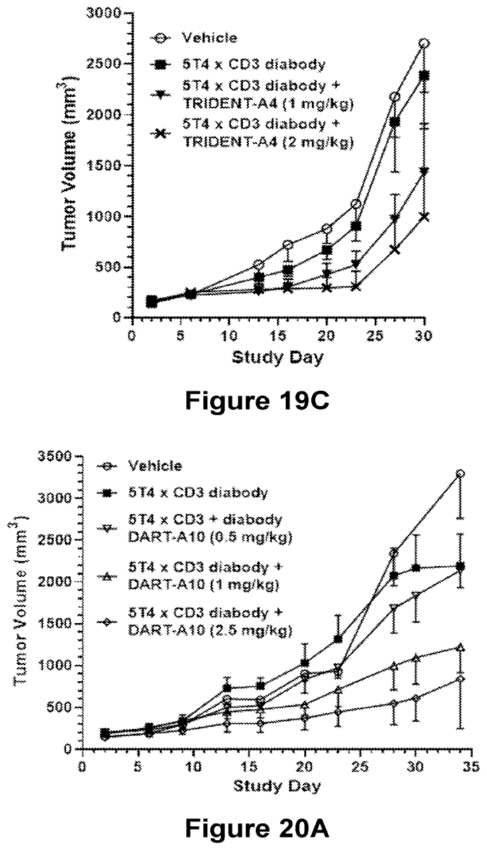

As provided herein, the CD137×TA Binding Molecules of the present invention may be used in combination with other tumor targeting agents. The ability of the representative PD-L1×CD137 bispecific molecules DART-A4, TRIDENT-A, and TRIDENT-A4 to enhance the anti-tumor active of a representative TA×CD3 bispecific molecule, 5T4×CD3 diabody (sequence provided below), was tested in vivo in a human PBMC-reconstituted murine xenograft model. Briefly, freshly isolated PBMCs (8×10⁶) were injected retro-orbitally on Study Day (SD) 0 into MHCI−/− mice. On SD7, RKO colon carcinoma cells (5×10⁶) were injected subcutaneously in a 1:1 mixture with Matrigel. On SD7 the mice were treated with OKT4. On SD14 treatment (IV) with the 5T4×CD3 diabody (twice weekly at 0.025 mg/kg), and treatment with the PD-L1×CD137 bispecific molecule (weekly at 1 or 2 mg/kg) was initiated. Tumor growth was measured twice weekly with calipers (N=7/group). As shown in FIGS. 19A-19C the TA×CD3 exhibited only minimal inhibition on tumor growth at the concentration tested. However, the combination of a PD-L1×CD137 bispecific molecule and the 5T4×CD3 diabody dramatically inhibited tumor growth. This study demonstrates that PD-L1×CD137 bispecific molecules comprising the binding domain of the novel CD137 MAB-6 antibody can inhibit tumor growth in-combination with a TA×CD3 bispecific molecule in vivo.

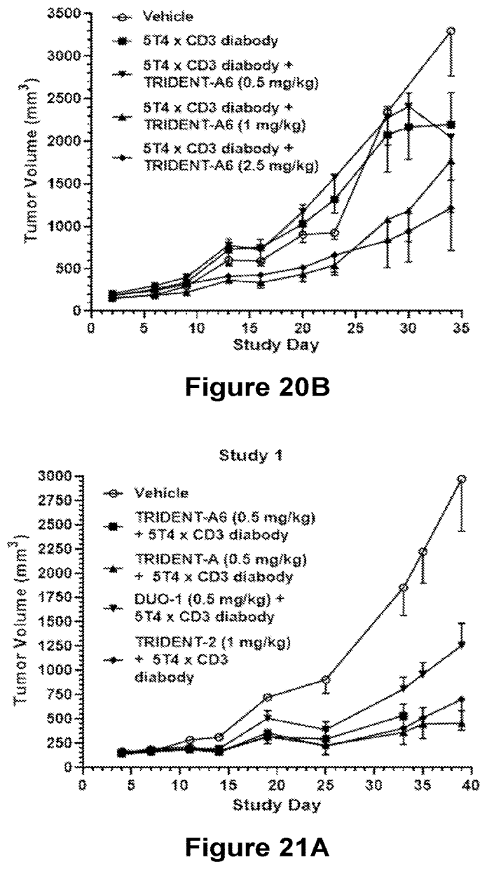

In another study, the ability of the representative PD-L1×CD137 bispecific molecules DART-A10, and TRIDENT-A6, each comprising two CD137 MAB-6(1.3) binding sites, to enhance the anti-tumor active of the 5T4×CD3 diabody, was examined. The study was performed essentially as described above except that the PD-L1×CD137 bispecific molecules were administered every 5 days (4 if over a weekend) at 0.5, 1, or 2.5 mg/kg. Tumor growth was measured twice weekly with calipers (N=8/group). As shown in FIGS. 20A-20B the DART-A10, and TRIDENT-A6 also inhibited tumor growth in combination with the 5T4×CD3 diabody. This study demonstrates that PD-L1×CD137 bispecific molecules comprising the binding domain of the deimmunized CD137 MAB-6 antibody can inhibit tumor growth in-combination with a TA×CD3 bispecific molecule in vivo.

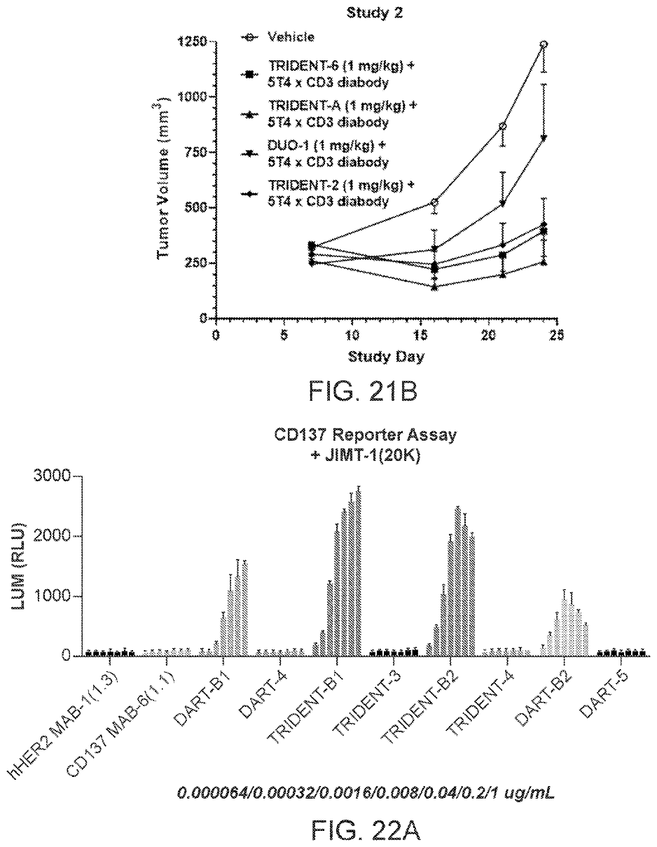

In additional combination treatment studies, the activity of the PD-L1×CD137 bispecific molecules TRIDENT-A6, TRIDENT-A (each comprising the VH/VL of a CD137 MAB-6 binding domain), was compared to that of TRIDENT-2, and a PD-L1×CD137 DUOBODY® bispecific molecule designated “PD-L1-547-FEALxCD137-009-HC7LC2-FEAR” described in WO 2019/025545, abbreviated herein as DUO-1 (amino acid sequence provided below). The studies were conducted essentially as described above except that the PD-L1×CD137 bispecific molecules were administered every 5 days (4 if over a weekend) at concentrations ranging between 0.1 mg/kg and 5 mg/kg in the different experiments. Tumor growth was measured twice weekly with calipers (N=8/group). Representative data from two studies (note—TRIDENT-2 was only dosed at 1 mg/kg in study 1) are plotted in FIGS. 21A and 21B and show that TRIDENT-A and TRIDENT-A6 exhibit anti-tumor activity that is comparable or slightly better than that of TRIDENT-2, and are more active than DUO-1.

The representative TA×CD3 bispecific molecule, the 5T4×CD3 diabody used in the above murine xenograft studies, is a bivalent diabody having one binding site for the 5T4 tumor antigen and one binding site for CD3. The molecule has the general structure shown in FIG. 1D and comprises the following three polypeptide chains:

| | |
|---|---|
| 5T4 x CD3 | DIQMTQSPSS LSASVGDRVT ITCRASQGIS NYLAWFQQKP GKAPKSLIYR |
| diabody | ANRLQSGVPS RFSGSGSGTD FTLTISSLQP EDVATYYCLQ YDDFPWTFGQ |
| Chain 1 | GTKLEIKGGG SGGGGEVQLV ESGGGLVQPG GSLRLSCAAS GFTFSTYAMN |
| | WVRQAPGKGL EWVGRIRSKY NNYATYYADS VKGRFTISRD DSKNSLYLQM |
| | NSLKTEDTAV YYCVRHGNFG NSYVSWFAYW GQGTLVTVSS GGCGGGEVAA |
| | LEKEVAALEK EVAALEKEVA ALEKGGGDKT HTCPPCPAPE AAGGPSVFLF |
| | PPKPKDTLMI SRTPEVTCVV VDVSHEDPEV KFNWYVDGVE VHNAKTKPRE |
| | EQYNSTYRVV SVLTVLHQDW LNGKEYKCKV SNKALPAPIE KTISKAKGQP |
| | REPQVYTLPP SREEMTKNQV SLWCLVKGFY PSDIAVEWES NGQPENNYKT |
| | TPPVLDSDGS FFLYSKLTVD KSRWQQGNVF SCSVMHEALH NHYTQKSLSL |
| | SPGK (SEQ ID NO: 140) |
| 5T4 x CD3 | QAVVTQEPSL TVSPGGTVTL TCRSSTGAVT TSNYANWVQQ KPGQAPRGLI |
| diabody | GGTNKRAPWT PARFSGSLLG GKAALTITGA QAEDEADYYC ALWYSNLWVF |
| Chain 2 | GGGTKLTVLG GGGSGGGGQV QLVQSGAEVK KPGASVKVSC KASGYTFTSF |
| | WMHWVRQAPG QGLEWMGRID PNRGGTEYNE KAKSRVTMTA DKSTSTAYME |
| | LSSLRSEDTA VYYCAGGNPY YPMDYWGQGT TVTVSSGGCG GGKVAALKEK |
| | VAALKEKVAA LKEKVAALKE (SEQ ID NO: 141) |
| 5T4 x CD3 | DKTHTCPPCP APEAAGGPSV FLFPPKPKDT LMISRTPEVT CVVVDVSHED |
| diabody | PEVKFNWYVD GVEVHNAKTK PREEQYNSTY RVVSVLTVLH QDWLNGKEYK |
| Chain 3 | CKVSNKALPA PIEKTISKAK GQPREPQVYT LPPSREEMTK NQVSLSCAVK |
| | GFYPSDIAVE WESNGQPENN YKTTPPVLDS DGSFFLVSKL TVDKSRWQQG |
| | NVFSCSVMHE ALHNRYTQKS LSLSPGK (SEQ ID NO: 142) |

The PD-L1-547-FEALxCD137-009-HC7LC2-FEALR bispecific molecule used in the above murine xenograft studies is described in WO 2019/025545. The molecule comprises different PD-L1 and CD137 binding specificities from those provided herein and comprises the followings four polypeptide chains:

| | |
|---|---|
| VH_CD137- | EVQLVESGGG LVQPGRSLRL SCTASGFSLN DYWMSWVRQA PGKGLEWVGY |
| 009-H7 | IDVGGSLYYA ASVKGRFTIS RDDSKSIAYL QMNSLKTEDT AVYYCARGGL |
| IgG1- | TYGFDLWGQG TLVTVSSAST KGPSVFPLAP SSKSTSGGTA ALGCLVKDYF |
| FEAR-Fc | PEPVTVSWNS GALTSGVHTF PAVLQSSGLY SLSSVVTVPS SSLGTQTYIC |
| | NVNHKPSNTK VDKRVEPKSC DKTHTCPPCP APEFEGGPSV FLFPPKPKDT |
| | LMISRTPEVT CVVVAVSHED PEVKFNWYVD GVEVHNAKTK PREEQYNSTY |
| | RVVSVLTVLH QDWLNGKEYK CKVSNKALPA PIEKTISKAK GQPREPQVYT |
| | LPPSREEMTK NQVSLTCLVK GFYPSDIAVE WESNGQPENN YKTTPPVLDS |
| | DGSFFLYSRL TVDKSRWQQG NVFSCSVMHE ALHNHYTQKS LSLSPGK |
| | (SEQ ID NO: 157) |
| VL_CD137- | DIVMTQSPSS LSASVGDRVT ITCQASEDIS SYLAWYQQKP GKAPKRLIYG |
| 009-L2 | ASDLASGVPS RFSASGSGTD YTFTISSLQP EDIATYYCHY YATISGLGVA |
| Kappa-C | FGGGTKVEIK RTVAAPSVFI FPPSDEQLKS GTASVVCLLN NFYPREAKVQ |
| | WKVDNALQSG NSQESVTEQD SKDSTYSLSS TLTLSKADYE KHKVYACEVT |
| | HQGLSSPVTK SFNRGEC |
| | (SEQ ID NO: 158) |
| VH-PD-L1- | EVQLLEPGGG LVQPGGSLRL SCEASGSTFS TYAMSWVRQA PGKGLEWVSG |
| 547 IgG1- | FSGSGGFTFY ADSVRGRFTI SRDSSKNTLF LQMSSLRAED TAVYYCAIPA |
| FEAL-Fc | RGYNYGSFQH WGQGTLVTVS SASTKGPSVF PLAPSSKSTS GGTAALGCLV |
| | KDYFPEPVTV SWNSGALTSG VHTFPAVLQS SGLYSLSSVV TVPSSSLGTQ |
| | TYICNVNHKP SNTKVDKRVE PKSCDKTHTC PPCPAPEFEG GPSVFLFPPK |
| | PKDTLMISRT PEVTCVVVAV SHEDPEVKFN WYVDGVEVHN AKTKPREEQY |
| | NSTYRVVSVL TVLHQDWLNG KEYKCKVSNK ALPAPIEKTI SKAKGQPREP |
| | QVYTLPPSRE EMTKNQVSLT CLVKGFYPSD IAVEWESNGQ PENNYKTTPP |
| | VLDSDGSFLL YSKLTVDKSR WQQGNVFSCS VMHEALHNHY TQKSLSLSPGK |
| | (SEQ ID NO: 159) |
| VL-PD-L1- | SYVLTQPPSV SVAPGQTARI TCGGNNIGSK SVHWYQQKPG QAPVLVVYDD |
| 547 | NDRPSGLPER FSGSNSGNTA TLTISRVEAG DEADYYCQVW DSSSDHVVFG |
| Lambda-C | GGTKLTVLGQ PKAAPSVTLF PPSSEELQAN KATLVCLISD FYPGAVTVAW |
| | KADSSPVKAG VETTTPSKQS NNKYAASSYL SLTPEQWKSH RSYSCQVTHE |
| | GSTVEKTVAP TECS |
| | (SEQ ID NO: 160) |

### Example 9

### Characterization of Additional CD137×TA Binding Molecules

CD137×TA Binding Molecules capable of binding to CD137 and to the representative TA, ITER2, were generated incorporating the VH and VL Domains of CD137 MAB-6(1.1) and the VH1 and VL. Domains of hHER2 MAB-1(1.3). In this study a number of additional bispecific configurations were examined. In particular, a bivalent bispecific diabody designated “DART-B1,” comprising bispecific diabody domains and having the asymmetric structure shown in FIG. 1D, and a trivalent binding molecule designated “TRIDENT-B2,” comprising bispecific diabody-type binding domains (where Site A binds CD137, and site B binds the TA), and a non-diabody-type binding domain (Site C binding CD137), having the structure shown in FIG. 3A, were generated. In addition, molecules having the same general configurations as those previous characterized were generated. In particular, a tetravalent bispecific diabody designated “DART-B2,” comprising identical bispecific diabody binding domains and having the antibody-like Y structure shown in FIG. 1B, and a trivalent binding molecule designated “TRIDENT-B1,” comprising mono-specific diabody-type binding domains (where Sites A and B bind CD137), and a non-diabody-type binding domain (Site C binding the TA), were generated. The domain attributes of these molecules, and certain bispecific control and comparator molecules having the same structures are discussed above (see e.g., Tables 5-6).

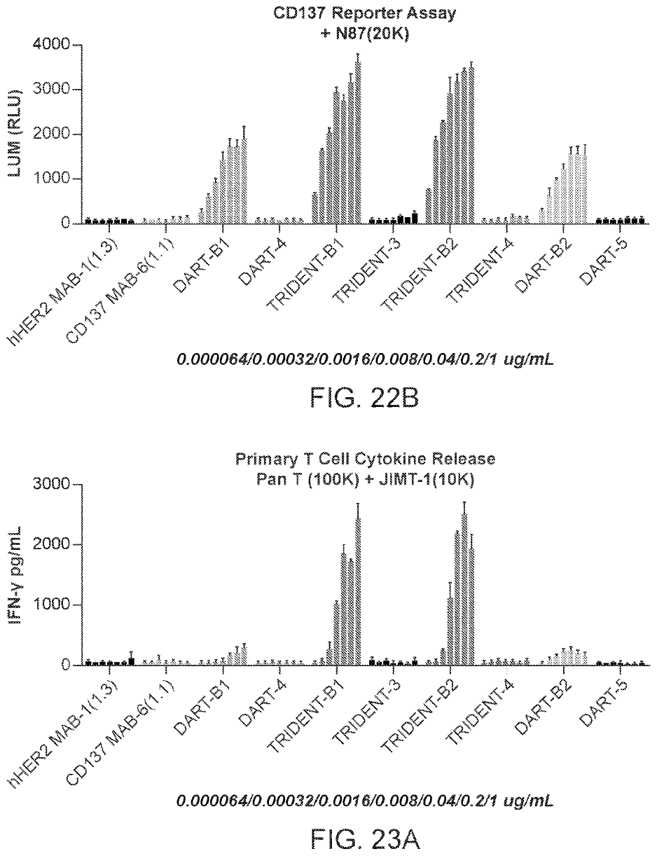

The functional activity of DART-B1, DART-B2, TRIDENT-B1, TRIDENT-B2, the parental CD137 MAB-6 (1.1) and HER2 MAB-1(1.3) antibodies, and the negative controls: DART-4, DART-5, TRIDENT-3, and TRIDENT-4 (CD137×RSV binding molecules having structures comparable to each test article), was evaluated in a CD137 reporter assay performed essentially as described above in the presence and absence of HER2 expressing cells (JIMT-1 (HER2++) or N87 (HER2+**) 20,000 cells per well). Test articles were used at 1 μg/mL and five-fold serial dilutions. The results of a representative assay using JIMT-1 and N87 cells are shown in FIGS. 22A and 22B, respectively, and demonstrate that all of CD137×HER2 bispecific molecules comprising the binding domain CD137 MAB-6 (1.1) mediated target-dependent signal transduction while the parental antibodies and the negative controls did not exhibit activity.

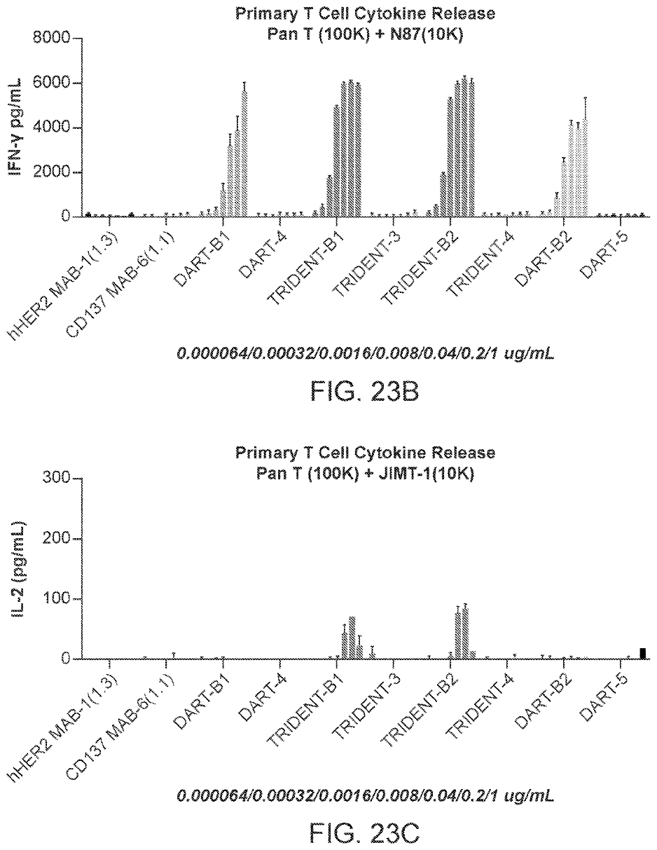

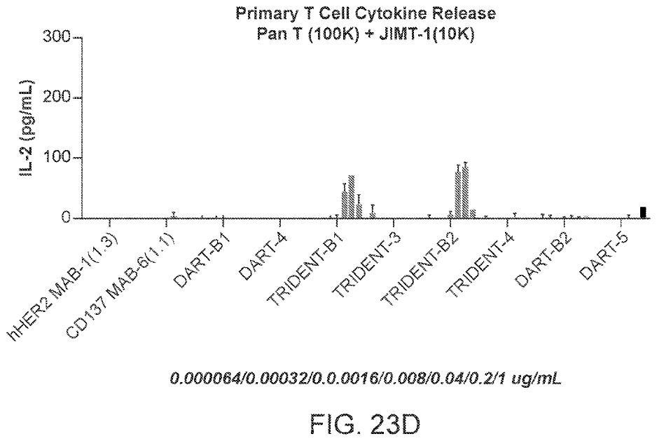

The functional activity of DART-B1, DART-B2, TRIDENT-B1, TRIDENT-B2, the parental CD137 MAB-6 (1.1) and HER2 MAB-1(1.3) antibodies, and the negative controls: DART-4, DART-5, TRIDENT-3, and TRIDENT-4, was also evaluated in the primary T cell cytokine release assay presence of HER2 expressing cells (JIMT-1 and N87 10,000 cells per well) performed essentially as described above. Test articles were used at 1 μg/mL and five-fold serial dilutions. The results of a representative assay using JIMT-1 cells are shown in FIGS. 23A (INF-γ) and 23C (IL-2), and using N87 cells are shown in FIG. 23B (INF-γ) and 23D (IL-2). As was seen in the CD137 reporter assay, all of CD137×HER2 bispecific molecules comprising the binding domain CD137 MAB-6 (1.1) mediated target-dependent cytokine release, particularly with high HER2 expressing N87 cells, while the parental antibodies and the negative controls did not exhibit activity.

These studies demonstrate that bivalent, tetravalent, and trivalent CD137×TA bispecific molecules comprising the binding domain CD137 MAB-6 (1.1) (DART-B1, DART-B2, TRIDENT-B1/B2, respectively) mediated target-dependent signal transduction. In will be noted that the position of the CD137 binding domains is shifted in TRIDENT-B2 relative to TRIDENT-B1. This study demonstrates that CD137 MAB-6 is functional when paired with additional tumor antigens, in numerous configurations, and even when present as a single binding site.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-44. (canceled)

45. A method of treating a cancer comprising administering to a subject in need thereof a therapeutically effective amount of a CD137 Binding Molecule comprising a first binding site that immunospecifically binds to an epitope of CD137, wherein said first binding site comprises a first Light Chain Variable Domain that comprises a CDR_{L}1, CDR_{L}2 and CDR_{L}3 of CD137 MAB-6 VL3 (SEQ ID NO:56), and a first Heavy Chain Variable Domain that comprises a CDR_{H}1, CDR_{H}2 and CDR_{H}3 of CD137 MAB-6 VH1 (SEQ ID NO:46).

46. The method of claim 45, wherein:
(A) said first Heavy Chain Variable Domain comprises the amino acid sequence of: hCD137 MAB-6 VH1 (SEQ ID NO:46); and
(B) said first Light Chain Variable Domain comprises the amino acid sequence of: hCD137 MAB-6 VL3 (SEQ ID NO:56).

47. The method of claim 45, wherein said CD137 Binding Molecule comprises a second binding site that immunospecifically binds to an epitope of PD-L1, wherein said second binding site comprises a second Light Chain Variable Domain that comprises a CDRL1, CDRL2 and CDRL3, and a second Heavy Chain Variable Domain that comprises a CDRH1, CDRH2 and CDRH3; and wherein:
(A) said second Light Chain Variable Domain CDR_{L}1, CDR_{L}2, and CDR_{L}3 are the Light Chain CDRs of hPD-L1 MAB-2 VLx (SEQ ID NO:63); and
(B) said second Heavy Chain Variable Domain CDR_{H}1, CDR_{H}2, and CDR_{H}3 are the Heavy Chain CDRs of hPD-L1 MAB-2 VHx (SEQ ID NO:59).

48. The method of claim 47, wherein:
(A) said second Heavy Chain Variable Domain comprises the amino acid sequence of:
(1) hPD-L1 MAB-2 VH1 (SEQ ID NO:57);
(2) hPD-L1 MAB-2 VH2 (SEQ ID NO:67);
(3) hPD-L1 MAB-2 VH3 (SEQ ID NO:68);
(4) hPD-L1 MAB-2 VH4 (SEQ ID NO:69);
(5) hPD-L1 MAB-2 VH5 (SEQ ID NO:70); or
(6) hPD-L1 MAB-2 VH6 (SEQ ID NO:71); and
(B) said second Light Chain Variable Domain comprises the amino acid sequence of:
(1) hPD-L1 MAB-2 VL1 (SEQ ID NO:58); or
(2) hPD-L1 MAB-2 VL2 (SEQ ID NO:72).

49. The method of claim 47, wherein said second Heavy Chain Variable Domain comprises the amino acid sequence of hPD-L1 MAB-2 VH4 (SEQ ID NO:69) and said second Light Chain Variable Domain comprises the amino acid sequence of hPD-L1 MAB-2 VL2 (SEQ ID NO:72).

50. The method of claim 47, wherein said first Heavy Chain Variable Domain comprises the amino acid sequence of hCD137 MAB-6 VH1 (SEQ ID NO:46), said first Light Chain Variable Domain comprises the amino acid sequence of hCD137 MAB-6 VL3 (SEQ ID NO:56), said second Heavy Chain Variable Domain comprises the amino acid sequence of hPD-L1 MAB-2 VH4 (SEQ ID NO:69) and said second Light Chain Variable Domain comprises the amino acid sequence of hPD-L1 MAB-2 VL2 (SEQ ID NO:72).

51. The method of claim 45, wherein said CD137 Binding Molecule comprises a second binding site that immunospecifically binds to an epitope of HER2, wherein said second binding site comprises a second Light Chain Variable Domain that comprises a CDRL1, CDRL2 and CDRL3, and a second Heavy Chain Variable Domain that comprises a CDRH1, CDRH2 and CDRH3; and wherein:
(A) (1) said second Light Chain Variable Domain CDR_{L}1, CDR_{L}2, and CDR_{L}3 are the Light Chain CDRs of hHER2-MAB-1 VLx (SEQ ID NO:79); and
(2) said second Heavy Chain Variable Domain CDR_{H}1, CDR_{H}2, and CDR_{H}3 are the Heavy Chain CDRs of hHER2-MAB-1 VHx (SEQ ID NO:78).

52. The method of claim 51, wherein:
(A) said second Heavy Chain Variable Domain comprises the amino acid sequence of
(1) hHER2-MAB-1 VH1 (SEQ ID NO:80);
(2) hHER2-MAB-1 VH2 (SEQ ID NO:81); or
(3) hHER2-MAB-1 VH3 (SEQ ID NO:82); and
(B) said second Light Chain Variable Domain comprises the amino acid sequence of:
(1) hHER2-MAB-1 VL1 (SEQ ID NO:83);
(2) hHER2-MAB-1 VL2 (SEQ ID NO:84); or
(3) hHER2-MAB-1 VL3 (SEQ ID NO:85).

53. The method of claim 51, wherein said second Heavy Chain Variable Domain comprises the amino acid sequence of hHER2-MAB-1 VH1 (SEQ ID NO:80) and said second Light Chain Variable Domain comprises the amino acid sequence of hHER2-MAB-1 VL3 (SEQ ID NO:85).

54. The method of claim 45, wherein said CD137 Binding Molecule is an antibody or a bispecific tetravalent Fc-bearing diabody, or a bispecific trivalent molecule.

55. The method of claim 45, wherein said CD137 Binding Molecule is bispecific and tetravalent, and comprises a first polypeptide chain, a second polypeptide chain, a third polypeptide chain, and a fourth polypeptide chain, and said polypeptide chains form a covalently bonded complex.

56. The method of claim 55, wherein said first polypeptide chain comprises the amino acid sequence of SEQ ID NO:120; the second polypeptide chain comprises the amino acid sequence of SEQ ID NO:139; the third polypeptide chain comprises the amino acid sequence of SEQ ID NO:120 and the fourth polypeptide chain comprises the amino acid sequence of SEQ ID NO:139.

57. The method of claim 45, wherein said CD137 Binding Molecule is bispecific and trivalent, and comprises a first polypeptide chain, a second polypeptide chain, a third polypeptide chain, and a fourth polypeptide chain, and said polypeptide chains form a covalently bonded complex

58. The method of claim 57, wherein said first polypeptide chain comprises the amino acid sequence of SEQ ID NO:135; the second polypeptide chain comprises the amino acid sequence of SEQ ID NO:136; the third polypeptide chain comprises the amino acid sequence of SEQ ID NO:131 and the fourth polypeptide chain comprises the amino acid sequence of SEQ ID NO:132.

59. The method of claim 57, wherein said first polypeptide chain comprises the amino acid sequence of SEQ ID NO:127; the second polypeptide chain comprises the amino acid sequence of SEQ ID NO:128; the third polypeptide chain comprises the amino acid sequence of SEQ ID NO:129 and the fourth polypeptide chain comprises the amino acid sequence of SEQ ID NO:130.

60. The method of claim 45, wherein the cancer is selected from the group consisting: bladder cancer, bone cancer, a brain and spinal cord cancer, breast cancer, cervical cancer, colorectal cancer, gallbladder or bile duct cancer, gastric cancer, glioblastoma, head and neck cancer, hepatocellular carcinoma, kidney cancer, leukemia, liver cancer, lung cancer, melanoma, neuroblastoma, non-small cell lung cancer (NSCLC), ovarian cancer, pancreatic cancer, pharyngeal cancer, prostate cancer, renal cell carcinoma, rhabdomyosarcoma, skin cancer, squamous cell cancer of the head and neck (SCCHN), stomach cancer, testicular cancer, thymic carcinoma, and uterine cancer.


SEQUENCE LISTING (160 sequences)

SEQ ID NO: 1 - protein, 107 aa, Homo sapiens [sequence omitted]

SEQ ID NO: 2 - protein, 104 aa, Homo sapiens
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 5 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 6 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 7 - protein, 15 aa, Homo sapiens
EPKSCDKTHTCPPCP

SEQ ID NO: 8 - protein, 12 aa, Homo sapiens
ERKCCVECPPCP

SEQ ID NO: 9 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 10 - protein, 12 aa, Homo sapiens
ESKYGPPCPSCP

SEQ ID NO: 11 - protein, 12 aa, synthetic construct
  REGION 1..12: note = Description of Artificial Sequence: Synthetic construct
ESKYGPPCPPCP

SEQ ID NO: 12 - protein, 217 aa, Homo sapiens
  VARIANT 217: note = Lysine (K) or is absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 13 - protein, 216 aa, Homo sapiens
  VARIANT 216: note = Lysine (K) or is absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 14 - protein, 217 aa, Homo sapiens
  VARIANT 217: note = Lysine (K) or is absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 15 - protein, 217 aa, Homo sapiens
  VARIANT 217: note = Lysine (K) or is absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 16 - protein, 8 aa, Homo sapiens
GGGSGGGG

SEQ ID NO: 17 - protein, 15 aa, synthetic construct
  REGION 1..15: note = Description of Artificial Sequence: Synthetic construct
GGGGSGGGGSGGGGS

SEQ ID NO: 18 - protein, 6 aa, synthetic construct
  REGION 1..6: note = Description of Artificial Sequence: Synthetic construct
GGCGGG

SEQ ID NO: 19 - protein, 5 aa, synthetic construct
  REGION 1..5: note = Description of Artificial Sequence: Synthetic construct
ASTKG

SEQ ID NO: 20 - protein, 10 aa, synthetic construct
  REGION 1..10: note = Description of Artificial Sequence: Synthetic construct
DKTHTCPPCP

SEQ ID NO: 21 - protein, 13 aa, synthetic construct
  REGION 1..13: note = Description of Artificial Sequence: Synthetic construct
GGGDKTHTCPPCP

SEQ ID NO: 22 - protein, 5 aa, synthetic construct
  REGION 1..5: note = Description of Artificial Sequence: Synthetic construct
APSSS

SEQ ID NO: 23 - protein, 8 aa, synthetic construct
  REGION 1..8: note = Description of Artificial Sequence: Synthetic construct
APSSSPME

SEQ ID NO: 24 - protein, 11 aa, synthetic construct
  REGION 1..11: note = Description of Artificial Sequence: Synthetic construct
GGGSGGGSGGG

SEQ ID NO: 25 - protein, 12 aa, synthetic construct
  REGION 1..12: note = Description of Artificial Sequence: Synthetic construct
GGGGSGGGSGGG

SEQ ID NO: 26 - protein, 6 aa, synthetic construct
  REGION 1..6: note = Description of Artificial Sequence: Synthetic construct
LGGGSG

SEQ ID NO: 27 - protein, 4 aa, synthetic construct
  REGION 1..4: note = Description of Artificial Sequence: Synthetic construct
GGGS

SEQ ID NO: 28 - protein, 6 aa, synthetic construct
  REGION 1..6: note = Description of Artificial Sequence: Synthetic construct
LEPKSS

SEQ ID NO: 29 - protein, 16 aa, synthetic construct
  REGION 1..16: note = Description of Artificial Sequence: Synthetic construct
VEPKSADKTHTCPPCP

SEQ ID NO: 30 - protein, 16 aa, synthetic construct
  REGION 1..16: note = Description of Artificial Sequence: Synthetic construct
LEPKSADKTHTCPPCP

SEQ ID NO: 31 - protein, 16 aa, synthetic construct
  REGION 1..16: note = Description of Artificial Sequence: Synthetic construct
LEPKSSDKTHTCPPCP

SEQ ID NO: 32 - protein, 7 aa, synthetic construct
  REGION 1..7: note = Description of Artificial Sequence: Synthetic construct
GVEPKSC

SEQ ID NO: 33 - protein, 6 aa, synthetic construct
  REGION 1..6: note = Description of Artificial Sequence: Synthetic construct
VEPKSC

SEQ ID NO: 34 - protein, 6 aa, synthetic construct
  REGION 1..6: note = Description of Artificial Sequence: Synthetic construct
AEPKSC

SEQ ID NO: 35 - protein, 7 aa, synthetic construct
  REGION 1..7: note = Description of Artificial Sequence: Synthetic construct
GFNRGEC

SEQ ID NO: 36 - protein, 6 aa, synthetic construct
  REGION 1..6: note = Description of Artificial Sequence: Synthetic construct
FNRGEC

SEQ ID NO: 37 - protein, 28 aa, synthetic construct
  REGION 1..28: note = Description of Artificial Sequence: Synthetic construct
EVAALEKEVAALEKEVAALEKEVAALEK

SEQ ID NO: 38 - protein, 28 aa, synthetic construct
  REGION 1..28: note = Description of Artificial Sequence: Synthetic construct
KVAALKEKVAALKEKVAALKEKVAALKE

SEQ ID NO: 39 - protein, 28 aa, synthetic construct
  REGION 1..28: note = Description of Artificial Sequence: Synthetic construct
EVAACEKEVAALEKEVAALEKEVAALEK

SEQ ID NO: 40 - protein, 28 aa, synthetic construct
  REGION 1..28: note = Description of Artificial Sequence: Synthetic construct
KVAACKEKVAALKEKVAALKEKVAALKE

SEQ ID NO: 41 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 42 - protein, 217 aa, synthetic construct
  REGION 1..217: note = Description of Artificial Sequence: Synthetic construct
  VARIANT 217: note = Lysine (K) or is absent
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 43 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 44 - protein, 217 aa, synthetic construct
  REGION 1..217: note = Description of Artificial Sequence: Synthetic construct
  VARIANT 217: note = Lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 45 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 46 - protein, 123 aa, Homo sapiens
QVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSS

SEQ ID NO: 47 - protein, 5 aa, Homo sapiens
SYYWS

SEQ ID NO: 48 - protein, 16 aa, Homo sapiens
RIYTSGSTNYNPSLKS

SEQ ID NO: 49 - protein, 15 aa, Homo sapiens
DGWYDEDYNYYGMDV

SEQ ID NO: 50 - protein, 108 aa, Homo sapiens
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIK

SEQ ID NO: 51 - protein, 12 aa, Homo sapiens
RASQSVSSNYLS

SEQ ID NO: 52 - protein, 7 aa, Homo sapiens
GASTRAT

SEQ ID NO: 53 - protein, 9 aa, Homo sapiens
QQDYDLPWT

SEQ ID NO: 54 - protein, 108 aa, synthetic construct
  REGION 1..108: note = Description of Artificial Sequence: Synthetic construct
  VARIANT 14: note = Serine (S) or Threonine (T)
  VARIANT 37: note = Phenylalanine (F) or Tyrosine (Y)
  VARIANT 40: note = Isoleucine (I) or Lysine (K)
EIVMTQSPATLSLXPGERATLSCRASQSVSSNYLSWXQQXPGQAPRLLIYGASTRATGIPARFSGSGSGTDFTLTISSLQPEDFAVYYCQQDYDLPWTFGQGTKVEIK

SEQ ID NO: 55 - protein, 108 aa, synthetic construct
  REGION 1..108: note = Description of Artificial Sequence: Synthetic construct
EIVMTQSPATLSLSPGERATLSCRASQSVSSNYLSWYQQKPGQAPRLLIYGASTRATGIPARFSGSGSGTDFTLTISSLQPEDFAVYYCQQDYDLPWTFGQGTKVEIK

SEQ ID NO: 56 - protein, 108 aa, synthetic construct
  REGION 1..108: note = Description of Artificial Sequence: Synthetic construct
EIVMTQSPATLSLSPGERATLSCRASQSVSSNYLSWFQQKPGQAPRLLIYGASTRATGIPARFSGSGSGTDFTLTISSLQPEDFAVYYCQQDYDLPWTFGQGTKVEIK

SEQ ID NO: 57 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIGGGTTYYPDTVKGRFTISRDNAKNTLYLQMNSLKTEDTAVYYCARQGLPYYFDYWGQGTLVTVSS

SEQ ID NO: 58 - protein, 107 aa, synthetic construct
  REGION 1..107: note = Description of Artificial Sequence: Synthetic construct
DIQMTQSPSSLSASVGDRVTITCKASQDVNTAVAWYQQKPGKAPKLLIYWASTRHTGVPSRFSGSGSGTDFTLTISSLQPEDFATYYCQQHYNTPLTFGQGTKVEIK

SEQ ID NO: 59 - protein, 118 aa, synthetic construct
  REGION 1..118: note = Description of Artificial Sequence: Synthetic construct
  VARIANT 54: note = Glycine (G) or Lysine (K)
  VARIANT 78: note = Threonine (T) or Serine (S)
  VARIANT 87: note = Lysine (K) or Arginine (R)
  VARIANT 88: note = Alanine (A) or Threonine (T)
  VARIANT 99: note = Alanine (A) or Glutamine (Q)
  VARIANT 105: note = Phenylalanine (F) or Glycine (G)
EVQLVESGGGLVQPGGSLRLSCAASGFTFSSYTMSWVRQAPGKGLEWVAYISIXGGTTYYPDTVKGRFTISRDNAKNXLYLQMNSLXXEDTAVYYCARXGLPYYXDYWGQGTLVTVSS

SEQ ID NO: 60 - protein, 5 aa, synthetic construct
  REGION 1..5: note = Description of Artificial Sequence: Synthetic construct
SYTMS

SEQ ID NO: 61 - protein, 17 aa, synthetic construct
  REGION 1..17: note = Description of Artificial Sequence: Synthetic construct
  VARIANT 5: note = Glysine (G) or Lysine (K)
YISIXGGTTYYPDTVKG

SEQ ID NO: 62 - protein, 9 aa, synthetic construct
  REGION 1..9: note = Description of Artificial Sequence: Synthetic construct
  VARIANT 1: note = Alanine (A) or Glutamine (Q)
  VARIANT 7: note = Phenylalanine (F) or Glycine (G)
XGLPYYXDY

SEQ ID NO: 63 - protein, 107 aa, synthetic construct
  REGION 1..107: note = Description of Artificial Sequence: Synthetic construct
  VARIANT 31: note = Glutamate (E) or Threonine (T)
DIQMTQSPSSLSASVGDRVTITCKASQDVNXAVAWYQQKPGKAPKLLIYWASTRHTGVPSRFSGSGSGTDFTLTISSLQPEDFATYYCQQHYNTPLTFGQGTKVEIK

SEQ ID NO: 64 - protein, 11 aa, synthetic construct
  REGION 1..11: note = Description of Artificial Sequence: Synthetic construct
  VARIANT 8: note = Glutamate (E) or Threonine (T)
KASQDVNXAVA

SEQ ID NO: 65 - protein, 7 aa, synthetic construct
  REGION 1..7: note = Description of Artificial Sequence: Synthetic construct
WASTRHT

SEQ ID NO: 66 - protein, 9 aa, synthetic construct
  REGION 1..9: note = Description of Artificial Sequence: Synthetic construct
QQHYNTPLT

SEQ ID NO: 67 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARQGLPYYFDYWGQGTLVTVSS

SEQ ID NO: 68 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARQGLPYYGDYWGQGTLVTVSS

SEQ ID NO: 69 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARAGLPYYFDYWGQGTLVTVSS

SEQ ID NO: 70 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARAGLPYYFDYWGQGTLVTVSS

SEQ ID NO: 71 - protein, 118 aa, synthetic construct
  REGION 1..118: note = Description of Artificial Sequence: Synthetic construct
EVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARAGLPYYGDYWGQGTLVTVSS

SEQ ID NO: 72 - protein, 107 aa, synthetic construct
  REGION 1..107: note = Description of Artificial Sequence: Synthetic construct
DIQMTQSPSSLSASVGDRVTITCKASQDVNEAVAWYQQKPGKAPKLLIYWASTRHTGVPSRFSGSGSGTDFTLTISSLQPEDFATYYCQQHYNTPLTFGQGTKVEIK

SEQ ID NO: 73 - protein, 17 aa, synthetic construct
  REGION 1..17: note = Description of Artificial Sequence: Synthetic construct
YISIKGGTTYYPDTVKG

SEQ ID NO: 74 - protein, 9 aa, synthetic construct
  REGION 1..9: note = Description of Artificial Sequence: Synthetic construct
QGLPYYGDY

SEQ ID NO: 75 - protein, 9 aa, synthetic construct
  REGION 1..9: note = Description of Artificial Sequence: Synthetic construct
AGLPYYFDY

SEQ ID NO: 76 - protein, 9 aa, synthetic construct
  REGION 1..9: note = Description of Artificial Sequence: Synthetic construct
AGLPYYGDY

SEQ ID NO: 77 - protein, 11 aa, synthetic construct
  REGION 1..11: note = Description of Artificial Sequence: Synthetic construct
KASQDVNEAVA

SEQ ID NO: 78 - protein, 119 aa, synthetic construct
  REGION 1..119: note = Description of Artificial Sequence: Synthetic construct
  VARIANT 100: note = Apartate (D) or Glutamate (E)
  VARIANT 101: note = Gycine (G) or Isoleucine (I)
QVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDXXYGNRVSYWGQGTLVTVSS

SEQ ID NO: 79 - protein, 107 aa, synthetic construct
  REGION 1..107: note = Description of Artificial Sequence: Synthetic construct
  VARIANT 30: note = Asparagine (N) or Serine (S)
  VARIANT 31: note = Serine (S) or Threonine (T) or Aparagine (N)
  VARIANT 55: note = Valine (V) or Glutamine (Q)
  VARIANT 56: note = Aspartate (D) or Glutamate (E) or Serine (S)
DIQMTQSPSSLSASVGDRVTITCKASQDIXXYLSWFQQKPGKAPKTLIYRANRLXXGVPSRFSGSGSGTDFTLTISSLQPEDFATYYCLQHDEFPWTFGQGTKLEIK

SEQ ID NO: 80 - protein, 119 aa, synthetic construct
  REGION 1..119: note = Description of Artificial Sequence: Synthetic construct
QVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDDGYGNRVSYWGQGTLVTVSS

SEQ ID NO: 81 - protein, 119 aa, synthetic construct
  REGION 1..119: note = Description of Artificial Sequence: Synthetic construct
QVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDEGYGNRVSYWGQGTLVTVSS

SEQ ID NO: 82 - protein, 119 aa, synthetic construct
  REGION 1..119: note = Description of Artificial Sequence: Synthetic construct
QVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDDIYGNRVSYWGQGTLVTVSS

SEQ ID NO: 83 - protein, 107 aa, synthetic construct
  REGION 1..107: note = Description of Artificial Sequence: Synthetic construct
DIQMTQSPSSLSASVGDRVTITCKASQDINSYLSWFQQKPGKAPKTLIYRANRLVDGVPSRFSGSGSGTDFTLTISSLQPEDFATYYCLQHDEFPWTFGQGTKLEIK

SEQ ID NO: 84 - protein, 107 aa, synthetic construct
  REGION 1..107: note = Description of Artificial Sequence: Synthetic construct
DIQMTQSPSSLSASVGDRVTITCKASQDINTYLSWFQQKPGKAPKTLIYRANRLVEGVPSRFSGSGSGTDFTLTISSLQPEDFATYYCLQHDEFPWTFGQGTKLEIK

SEQ ID NO: 85 - protein, 107 aa, synthetic construct
  REGION 1..107: note = Description of Artificial Sequence: Synthetic construct
DIQMTQSPSSLSASVGDRVTITCKASQDISNYLSWFQQKPGKAPKTLIYRANRLQSGVPSRFSGSGSGTDFTLTISSLQPEDFATYYCLQHDEFPWTFGQGTKLEIK

SEQ ID NO: 86 - protein, 118 aa, Mus musculus
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARKHGNYYTMDYWGQGTSVTVSS

SEQ ID NO: 87 - protein, 106 aa, Mus musculus
DIQMTQTTSSLSASLGDRITISCRASQDISNYLNWYQQKPDGTVKLLIYYTSRLHSGVPSRFSGSGSGTDYSLTISNLEQEDIATYFCQQGYTLYTFGGGTKLEIK

SEQ ID NO: 88 - protein, 118 aa, Mus musculus
QIQLVQSGPELKKPGETVKISCKASGFTFTNYGMNWVKQAPGKGLKWMGWINTYIGEPTYADDFKGRFVFSLETSASTAYLQINNLKNEDMATYFCARELGPYYFDYWGQGTTLTVSS

SEQ ID NO: 89 - protein, 111 aa, Mus musculus
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPTFGSGTKLEIK

SEQ ID NO: 90 - protein, 118 aa, Mus musculus
EVQLVESGGGSVKPGGSLKLSCAASGFTFTDHYMYWVRQTPEKRLEWVATISDGGSFTSYPDSVKGRFTISRDIAKNNLYLQMSSLKSEDTAMYYCTRDESDRPFPYWGQGTLVTVSS

SEQ ID NO: 91 - protein, 107 aa, Mus musculus
DIVLTQSHRSMSTSVGDRVNITCKASQDVTTAVAWYQQKPGQSPKLLIFWASTRHAGVPDRFTGSGSGTDFTLTISSVQAGDLALYYCQQHYSTPYTFGGGTKLEIK

SEQ ID NO: 92 - protein, 118 aa, synthetic construct
  REGION 1..118: note = Description of Artificial Sequence: Synthetic construct
QVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSS

SEQ ID NO: 93 - protein, 107 aa, synthetic construct
  REGION 1..107: note = Description of Artificial Sequence: Synthetic construct
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIK

SEQ ID NO: 94 - protein, 127 aa, Mus musculus
QVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSS

SEQ ID NO: 95 - protein, 112 aa, Mus musculus
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 96 - protein, 120 aa, synthetic construct
  REGION 1..120: note = Description of Artificial Sequence: Synthetic construct
QVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSS

SEQ ID NO: 97 - protein, 120 aa, synthetic construct
  REGION 1..120: note = Description of Artificial Sequence: Synthetic construct
QVQLVQSGAEVKKPGASVKVSCKASGYTFTSYWMQWVRQAPGQGLEWMGTIYPGGGDTRYTQKFQGRVTITADKSTSTAYMELSSLRSEDTAVYYCARRGIPRLWYFDVWGQGTTVTVSS

SEQ ID NO: 98 - protein, 107 aa, synthetic construct
  REGION 1..107: note = Description of Artificial Sequence: Synthetic construct
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIK

SEQ ID NO: 99 - protein, 107 aa, synthetic construct
  REGION 1..107: note = Description of Artificial Sequence: Synthetic construct
DIQMTQSPSSLSASVGDRVTITCRASQSISSYLNWYQQKPGKAPKLLIYYTSRLQSGVPSRFSGSGSGTDFTLTISSLQPEDIATYYCQQGNTLPPTFGGGTKLEIK

SEQ ID NO: 100 - protein, 117 aa, synthetic construct
  REGION 1..117: note = Description of Artificial Sequence: Synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 101 - protein, 108 aa, synthetic construct
  REGION 1..108: note = Description of Artificial Sequence: Synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 102 - protein, 119 aa, synthetic construct
  REGION 1..119: note = Description of Artificial Sequence: Synthetic construct
QVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSS

SEQ ID NO: 103 - protein, 106 aa, synthetic construct
  REGION 1..106: note = Description of Artificial Sequence: Synthetic construct
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIK

SEQ ID NO: 104 - protein, 121 aa, synthetic construct
  REGION 1..121: note = Description of Artificial Sequence: Synthetic construct
QVQLQQSGPEVVRPGVSVKISCKGSGYTFTDYAMHWVKQSHAKSLEWIGLISTYSGDTKYNQNFKGKATMTVDKSASTAYMELSSLRSEDTAVYYCARGDYSGSRYWFAYWGQGTLVTVSS

SEQ ID NO: 105 - protein, 107 aa, synthetic construct
  REGION 1..107: note = Description of Artificial Sequence: Synthetic construct
DIQMTQSPSSLSASVGDRVTITCGASENIYGALNWYQRKPGKSPKLLIWGASNLADGMPSRFSGSGSGRQYTLTISSLQPEDVATYYCQNVLSSPYTFGGGTKLEIK

SEQ ID NO: 106 - protein, 121 aa, synthetic construct
  REGION 1..121: note = Description of Artificial Sequence: Synthetic construct
QVQLVESGGGVVQPGRSLRLSCSSSGFALTDYYMSWVRQAPGKGLEWLGFIANKANGHTTDYSPSVKGRFTISRDNSKNTLFLQMDSLRPEDTGVYFCARDMGIRWNFDVWGQGTPVTVSS

SEQ ID NO: 107 - protein, 107 aa, synthetic construct
  REGION 1..107: note = Description of Artificial Sequence: Synthetic construct
DIQLTQSPSSLSASVGDRVTMTCSASSRVSYIHWYQQKPGKAPKRWIYGTSTLASGVPARFSGSGSGTDFTFTISSLQPEDIATYYCQQWSYNPPTFGQGTKVEIKR

SEQ ID NO: 108 - protein, 120 aa, synthetic construct
  REGION 1..120: note = Description of Artificial Sequence: Synthetic construct
QVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSS

SEQ ID NO: 109 - protein, 106 aa, synthetic construct
  REGION 1..106: note = Description of Artificial Sequence: Synthetic construct
ENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIK

SEQ ID NO: 110 - protein, 120 aa, synthetic construct
  REGION 1..120: note = Description of Artificial Sequence: Synthetic construct
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 111 - protein, 113 aa, synthetic construct
  REGION 1..113: note = Description of Artificial Sequence: Synthetic construct
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 112 - protein, 122 aa, synthetic construct
  REGION 1..122: note = Description of Artificial Sequence: Synthetic construct
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSS

SEQ ID NO: 113 - protein, 107 aa, synthetic construct
  REGION 1..107: note = Description of Artificial Sequence: Synthetic construct
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIK

SEQ ID NO: 114 - protein, 121 aa, synthetic construct
  REGION 1..121: note = Description of Artificial Sequence: Synthetic construct
  VARIANT 37: note = Valine (V) or Isoleucine (I)
QEQLVESGGGLVQPGGSLRLSCAASGFTFSDYYMSWXRQAPGKGLEWVATIYPSSGKTYYADSAKGRLTISSDNAKDSLYLQMNSLRAEDTAVYYCTRDSYADDAALFDIWGQGTTVTVSS

SEQ ID NO: 115 - protein, 112 aa, synthetic construct
  REGION 1..112: note = Description of Artificial Sequence: Synthetic construct
QLVLTQSPSASASLGSSVKLTCTLSSGHKTDTIDWYQQQPGKAPRYLMKLEGSGSYNKGSGVPDRFSGSSSGADWYLTISSLQSEDEADYYCGTDYPGNYLFGGGTQLTVLG

SEQ ID NO: 116 - protein, 504 aa, synthetic construct
  REGION 1..504: note = Description of Artificial Sequence: Synthetic construct
DIQMTQSPSSLSASVGDRVTITCKASQDVNTAVAWYQQKPGKAPKLLIYWASTRHTGVPSRFSGSGSGTDFTLTISSLQPEDFATYYCQQHYNTPLTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIGGGTTYYPDTVKGRFTISRDNAKNTLYLQMNSLKTEDTAVYYCARQGLPYYFDYWGQGTLVTVSSGGCGGGKVAACKEKVAALKEKVAALKEKVAALKE

SEQ ID NO: 118 - protein, 505 aa, synthetic construct
  REGION 1..505: note = Description of Artificial Sequence: Synthetic construct
DIQMTQSPSSLSASVGDRVTITCKASQDVNTAVAWYQQKPGKAPKLLIYWASTRHTGVPSRFSGSGSGTDFTLTISSLQPEDFATYYCQQHYNTPLTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARQGLPYYFDYWGQGTLVTVSSGGCGGGKVAACKEKVAALKEKVAALKEKVAALKE

SEQ ID NO: 120 - protein, 505 aa, synthetic construct
  REGION 1..505: note = Description of Artificial Sequence: Synthetic construct
DIQMTQSPSSLSASVGDRVTITCKASQDVNEAVAWYQQKPGKAPKLLIYWASTRHTGVPSRFSGSGSGTDFTLTISSLQPEDFATYYCQQHYNTPLTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARQGLPYYGDYWGQGTLVTVSSGGCGGGKVAACKEKVAALKEKVAALKEKVAALKE

SEQ ID NO: 122 - protein, 268 aa, synthetic construct
  REGION 1..268: note = Description of Artificial Sequence: Synthetic construct
EIVMTQSPATLSLSPGERATLSCRASQSVSSNYLSWYQQKPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARQGLPYYGDYWGQGTLVTVSSGGCGGGKVAACKEKVAALKEKVAALKEKVAALKE

SEQ ID NO: 123 - protein, 268 aa, synthetic construct
  REGION 1..268: note = Description of Artificial Sequence: Synthetic construct
EIVMTQSPATLSLSPGERATLSCRASQSVSSNYLSWFQQKPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARQGLPYYGDYWGQGTLVTVSSGGCGGGKVAACKEKVAALKEKVAALKEKVAALKE

SEQ ID NO: 124 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARAGLPYYFDYWGQGTLVTVSSGGCGGGKVAACKEKVAALKEKVAALKEKVAALKE

SEQ ID NO: 125 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARAGLPYYFDYWGQGTLVTVSSGGCGGGKVAACKEKVAALKEKVAALKEKVAALKE

SEQ ID NO: 126 - protein, 268 aa, synthetic construct
  REGION 1..268: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARAGLPYYGDYWGQGTLVTVSSGGCGGGKVAACKEKVAALKEKVAALKEKVAALKE

SEQ ID NO: 127 - protein, 503 aa, synthetic construct
  REGION 1..503: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAALEKEVAALEKEVAALEKEVAALEKGGGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 273 aa, synthetic construct
  REGION 1..273: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGKVAALKEKVAALKEKVAALKEKVAALKE

SEQ ID NO: 129 - protein, 448 aa, synthetic construct
  REGION 1..448: note = Description of Artificial Sequence: Synthetic construct
EVQLVESGGGLVQPGGSLRLSCAASGFTFSSYTMSWVRQAPGKGLEWVAYISIGGGTTYYPDTVKGRFTISRDNAKNTLYLQMNSLKTEDTAVYYCARQGLPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 130 - protein, 214 aa, synthetic construct
  REGION 1..214: note = Description of Artificial Sequence: Synthetic construct
DIQMTQSPSSLSASVGDRVTITCKASQDVNTAVAWYQQKPGKAPKLLIYWASTRHTGVPSRFSGSGSGTDFTLTISSLQPEDFATYYCQQHYNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 448 aa, synthetic construct
  REGION 1..448: note = Description of Artificial Sequence: Synthetic construct
EVQLVESGGGLVQPGGSLRLSCAASGFTFSSYTMSWVRQAPGKGLEWVAYISIKGGTTYYPDTVKGRFTISRDNAKNSLYLQMNSLRAEDTAVYYCARQGLPYYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 132 - protein, 214 aa, synthetic construct
  REGION 1..214: note = Description of Artificial Sequence: Synthetic construct
DIQMTQSPSSLSASVGDRVTITCKASQDVNEAVAWYQQKPGKAPKLLIYWASTRHTGVPSRFSGSGSGTDFTLTISSLQPEDFATYYCQQHYNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 503 aa, synthetic construct
  REGION 1..503: note = Description of Artificial Sequence: Synthetic construct
EIVMTQSPATLSLSPGERATLSCRASQSVSSNYLSWYQQKPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAALEKEVAALEKEVAALEKEVAALEKGGGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 273 aa, synthetic construct
  REGION 1..273: note = Description of Artificial Sequence: Synthetic construct
EIVMTQSPATLSLSPGERATLSCRASQSVSSNYLSWYQQKPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGKVAALKEKVAALKEKVAALKEKVAALKE

SEQ ID NO: 135 - protein, 503 aa, synthetic construct
  REGION 1..503: note = Description of Artificial Sequence: Synthetic construct
EIVMTQSPATLSLSPGERATLSCRASQSVSSNYLSWFQQKPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAALEKEVAALEKEVAALEKEVAALEKGGGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 273 aa, synthetic construct
  REGION 1..273: note = Description of Artificial Sequence: Synthetic construct
EIVMTQSPATLSLSPGERATLSCRASQSVSSNYLSWFQQKPGQAPRLLIYGASTRATGIPARFSGSGSGTDFTLTISSLQPEDFAVYYCQQDYDLPWTFGQGTKVEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGKVAALKEKVAALKEKVAALKEKVAALKE

SEQ ID NO: 137 - protein, 120 aa, synthetic construct
  REGION 1..120: note = Description of Artificial Sequence: Synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 138 - protein, 106 aa, synthetic construct
  REGION 1..106: note = Description of Artificial Sequence: Synthetic construct
DIQMTQSPSTLSASVGDRVTITCRASQSVGYMHWYQQKPGKAPKLLIYDTSKLASGVPSRFSGSGSGTEFTLTISSLQPDDFATYYCFQGSGYPFTFGGGTKLEIK

SEQ ID NO: 139 - protein, 268 aa, synthetic construct
  REGION 1..268: note = Description of Artificial Sequence: Synthetic construct
EIVMTQSPATLSLSPGERATLSCRASQSVSSNYLSWFQQKPGQAPRLLIYGASTRATGIPARFSGSGSGTDFTLTISSLQPEDFAVYYCQQDYDLPWTFGQGTKVEIKGGGSGGGGEVQLVESGGGLVQPGGSLRLSCAASGFTFSSYTMSWVRQAPGKGLEWVAYISIGGGTTYYPDTVKGRFTISRDNAKNSLYLQMNSLRAEDTAVYYCARAGLPYYFDYWGQGTLVTVSSGGCGGGKVAACKEKVAALKEKVAALKEKVAALKE

SEQ ID NO: 140 - protein, 504 aa, synthetic construct
  REGION 1..504: note = Description of Artificial Sequence: Synthetic construct
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 270 aa, synthetic construct
  REGION 1..270: note = Description of Artificial Sequence: Synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSSGGCGGGKVAALKEKVAALKEKVAALKEKVAALKE

SEQ ID NO: 142 - protein, 227 aa, synthetic construct
  REGION 1..227: note = Description of Artificial Sequence: Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 143 - protein, 499 aa, synthetic construct
  REGION 1..499: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGQVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDDGYGNRVSYWGQGTLVTVSSGGCGGGEVAALEKEVAALEKEVAALEKEVAALEKGGGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 272 aa, synthetic construct
  REGION 1..272: note = Description of Artificial Sequence: Synthetic construct
DIQMTQSPSSLSASVGDRVTITCKASQDISNYLSWFQQKPGKAPKTLIYRANRLQSGVPSRFSGSGSGTDFTLTISSLQPEDFATYYCLQHDEFPWTFGQGTKLEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGKVAALKEKVAALKEKVAALKEKVAALKE

SEQ ID NO: 145 - protein, 227 aa, synthetic construct; identical to SEQ ID NO: 142
  REGION 1..227: note = Description of Artificial Sequence: Synthetic construct

SEQ ID NO: 146 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 147 - protein, 217 aa, synthetic construct
  REGION 1..217: note = Description of Artificial Sequence: Synthetic construct
  VARIANT 217: note = Lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 148 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 149 - protein, 217 aa, synthetic construct
  REGION 1..217: note = Description of Artificial Sequence: Synthetic construct
  VARIANT 217: note = Lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 150 - protein, 217 aa, synthetic construct
  REGION 1..217: note = Description of Artificial Sequence: Synthetic construct
  VARIANT 217: note = Lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGX

SEQ ID NO: 151 - protein, 505 aa, synthetic construct
  REGION 1..505: note = Description of Artificial Sequence: Synthetic construct
DIQMTQSPSSLSASVGDRVTITCKASQDISNYLSWFQQKPGKAPKTLIYRANRLQSGVPSRFSGSGSGTDFTLTISSLQPEDFATYYCLQHDEFPWTFGQGTKLEIKGGGSGGGGQVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTTVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 269 aa, synthetic construct
  REGION 1..269: note = Description of Artificial Sequence: Synthetic construct
EIVMTQSPATLSLTPGERATLSCRASQSVSSNYLSWFQQIPGQAPRLLIYGASTRATGIPARFSGSGSGTDFTLTISSLQPEDFAVYYCQQDYDLPWTFGQGTKVEIKGGGSGGGGQVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDDGYGNRVSYWGQGTLVTVSSGGCGGGKVAACKEKVAALKEKVAALKEKVAALKE

SEQ ID NO: 153 - protein, 449 aa, synthetic construct
  REGION 1..449: note = Description of Artificial Sequence: Synthetic construct
QVQLVQSGAEVKKPGASVKVSCKASGYTFTNYGMNWVRQAPGQGLEWMGWINTNIGEPTYTEEFKGRVTMTRDTSISTAYMELSRLRSDDTAVYYCARDDGYGNRVSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 154 - protein, 214 aa, synthetic construct
  REGION 1..214: note = Description of Artificial Sequence: Synthetic construct
DIQMTQSPSSLSASVGDRVTITCKASQDISNYLSWFQQKPGKAPKTLIYRANRLQSGVPSRFSGSGSGTDFTLTISSLQPEDFATYYCLQHDEFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 453 aa, synthetic construct
  REGION 1..453: note = Description of Artificial Sequence: Synthetic construct
QVQLQESGPGLVKPSETLSLTCTVSGGSISSYYWSWIRQPPGKGLEWIGRIYTSGSTNYNPSLKSRVTMSVDTSKNQFSLKLSSVTAADTAVYYCARDGWYDEDYNYYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 156 - protein, 134 aa, synthetic construct
  REGION 1..134: note = Description of Artificial Sequence: Synthetic construct
EDFAVYYCQQDYDLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 447 aa, synthetic construct
  REGION 1..447: note = Description of Artificial Sequence: Synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 158 - protein, 217 aa, synthetic construct
  REGION 1..217: note = Description of Artificial Sequence: Synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 451 aa, synthetic construct
  REGION 1..451: note = Description of Artificial Sequence: Synthetic construct
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 160 - protein, 214 aa, synthetic construct
  REGION 1..214: note = Description of Artificial Sequence: Synthetic construct
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS